SEQUENCE LISTING (1008 sequences)

SEQ ID NO: 1 - DNA, 22 bp, Homo sapiens
  source 1..22: mol_type = unassigned DNA
ttgaggtaaaggagatttcggt

SEQ ID NO: 2 - DNA, 23 bp, Homo sapiens
  source 1..23: mol_type = unassigned DNA
acatacgcctacgcaaattttta

SEQ ID NO: 3 - DNA, 21 bp, Homo sapiens
  source 1..21: mol_type = unassigned DNA
ttcggtgtttgcgaagggtta

SEQ ID NO: 4 - DNA, 23 bp, Homo sapiens
  source 1..23: mol_type = unassigned DNA
tcacaaccaacacaacgacactt

SEQ ID NO: 5 - DNA, 21 bp, Homo sapiens
  source 1..21: mol_type = unassigned DNA
acaaccaacacaacgacactt

SEQ ID NO: 6 - DNA, 20 bp, Homo sapiens
  source 1..20: mol_type = unassigned DNA
tcggtatttgttttcgcggt

SEQ ID NO: 7 - DNA, 22 bp, Homo sapiens
  source 1..22: mol_type = unassigned DNA
cgcctacgcaaatttttatcgc

SEQ ID NO: 8 - DNA, 22 bp, Homo sapiens
  source 1..22: mol_type = unassigned DNA
cgagagcgataaaaatttgcgt

SEQ ID NO: 9 - DNA, 20 bp, Homo sapiens
  source 1..20: mol_type = unassigned DNA
acccttcgcaaacaccgaaa

SEQ ID NO: 10 - DNA, 19 bp, Homo sapiens
  source 1..19: mol_type = unassigned DNA
ggtaatagcgtgtttttgc

SEQ ID NO: 11 - DNA, 22 bp, Homo sapiens
  source 1..22: mol_type = unassigned DNA
atattacatacgcctacgcaaa

SEQ ID NO: 12 - DNA, 20 bp, Homo sapiens
  source 1..20: mol_type = unassigned DNA
tttgtgtaaaatgcggcggt

SEQ ID NO: 13 - DNA, 22 bp, Homo sapiens
  source 1..22: mol_type = unassigned DNA
ctaccgcgaaaacaaataccga

SEQ ID NO: 14 - DNA, 20 bp, Homo sapiens
  source 1..20: mol_type = unassigned DNA
atttcggtgtttgcgaaggg

SEQ ID NO: 15 - DNA, 20 bp, Homo sapiens
  source 1..20: mol_type = unassigned DNA
acaaccaacacaacgacact

SEQ ID NO: 16 - DNA, 20 bp, Homo sapiens
  source 1..20: mol_type = unassigned DNA
tttcggttgtcgggtttgga

SEQ ID NO: 17 - DNA, 22 bp, Homo sapiens
  source 1..22: mol_type = unassigned DNA
tatttcggttgtcgggtttgga

SEQ ID NO: 18 - DNA, 20 bp, Homo sapiens
  source 1..20: mol_type = unassigned DNA
ccctcaatcgctcatcctcc

SEQ ID NO: 19 - DNA, 20 bp, Homo sapiens
  source 1..20: mol_type = unassigned DNA
tcgtcggtcggtttaggatg

SEQ ID NO: 20 - DNA, 22 bp, Homo sapiens
  source 1..22: mol_type = unassigned DNA
aaaaccgacgccaaacctacat

SEQ ID NO: 21 - DNA, 20 bp, Homo sapiens
  source 1..20: mol_type = unassigned DNA
aaccgacgccaaacctacat

SEQ ID NO: 22 - DNA, 20 bp, Homo sapiens
  source 1..20: mol_type = unassigned DNA
cggaggatgagcgattgagg

SEQ ID NO: 23 - DNA, 20 bp, Homo sapiens
  source 1..20: mol_type = unassigned DNA
taacgcgcacaccgaactaa

SEQ ID NO: 24 - DNA, 20 bp, Homo sapiens
  source 1..20: mol_type = unassigned DNA
cgagttggggtcgcgattat

SEQ ID NO: 25 - DNA, 20 bp, Homo sapiens
  source 1..20: mol_type = unassigned DNA
catcctaaaccgaccgacga

SEQ ID NO: 26 - DNA, 20 bp, Homo sapiens
  source 1..20: mol_type = unassigned DNA
cgacgcgttacggttgttta

SEQ ID NO: 27 - DNA, 20 bp, Homo sapiens
  source 1..20: mol_type = unassigned DNA
ccgcttctccgaaaccaaac

SEQ ID NO: 28 - DNA, 20 bp, Homo sapiens
  source 1..20: mol_type = unassigned DNA
taaggcggggtttttagagc

SEQ ID NO: 29 - DNA, 18 bp, Homo sapiens
  source 1..18: mol_type = unassigned DNA
taaaaactaacgcgcccg

SEQ ID NO: 30 - DNA, 18 bp, Homo sapiens
  source 1..18: mol_type = unassigned DNA
ggtttcggtgttattcgc

SEQ ID NO: 31 - DNA, 19 bp, Homo sapiens
  source 1..19: mol_type = unassigned DNA
ctcctctccgcgaaaaaat

SEQ ID NO: 32 - DNA, 20 bp, Homo sapiens
  source 1..20: mol_type = unassigned DNA
cggaggatgagcgattgagg

SEQ ID NO: 33 - DNA, 20 bp, Homo sapiens
  source 1..20: mol_type = unassigned DNA
taacgcgcacaccgaactaa

SEQ ID NO: 34 - DNA, 20 bp, Homo sapiens
  source 1..20: mol_type = unassigned DNA
tcgtcggtcggtttaggatg

SEQ ID NO: 35 - DNA, 20 bp, Homo sapiens
  source 1..20: mol_type = unassigned DNA
aaccgacgccaaacctacat

SEQ ID NO: 36 - DNA, 20 bp, Homo sapiens
  source 1..20: mol_type = unassigned DNA
gtcggacgcgttttagttgg

SEQ ID NO: 37 - DNA, 20 bp, Homo sapiens
  source 1..20: mol_type = unassigned DNA
tccctaccgacctcaacact

SEQ ID NO: 38 - DNA, 20 bp, Homo sapiens
  source 1..20: mol_type = unassigned DNA
tggttgggggattttgaggg

SEQ ID NO: 39 - DNA, 20 bp, Homo sapiens
  source 1..20: mol_type = unassigned DNA
aaacctccccgcctacctat

SEQ ID NO: 40 - DNA, 20 bp, Homo sapiens
  source 1..20: mol_type = unassigned DNA
gcggacggtttggagaaatg

SEQ ID NO: 41 - DNA, 20 bp, Homo sapiens
  source 1..20: mol_type = unassigned DNA
cgcgactcaatctcaccact

SEQ ID NO: 42 - DNA, 20 bp, Homo sapiens
  source 1..20: mol_type = unassigned DNA
ggaggttgggtttcgggatt

SEQ ID NO: 43 - DNA, 20 bp, Homo sapiens
  source 1..20: mol_type = unassigned DNA
gcgcccctaaactcgtatct

SEQ ID NO: 44 - DNA, 20 bp, Homo sapiens
  source 1..20: mol_type = unassigned DNA
gcggagtggtgagattgagt

SEQ ID NO: 45 - DNA, 20 bp, Homo sapiens
  source 1..20: mol_type = unassigned DNA
accgacttcttcgattcgcc

SEQ ID NO: 46 - DNA, 20 bp, Homo sapiens
  source 1..20: mol_type = unassigned DNA
ataggtaggcggggaggttt

SEQ ID NO: 47 - DNA, 19 bp, Homo sapiens
  source 1..19: mol_type = unassigned DNA
cgatcccccaactcaaccc

SEQ ID NO: 48 - DNA, 20 bp, Homo sapiens
  source 1..20: mol_type = unassigned DNA
tgagttggcggtttcgtttg

SEQ ID NO: 49 - DNA, 20 bp, Homo sapiens
  source 1..20: mol_type = unassigned DNA
cccgaatcccctcttatccc

SEQ ID NO: 50 - DNA, 20 bp, Homo sapiens
  source 1..20: mol_type = unassigned DNA
cgcgattttgtagtcggggt

SEQ ID NO: 51 - DNA, 20 bp, Homo sapiens
  source 1..20: mol_type = unassigned DNA
tttcctatcgccccaacacc

SEQ ID NO: 52 - DNA, 20 bp, Homo sapiens
  source 1..20: mol_type = unassigned DNA
ggaggttgggtttcgggatt

SEQ ID NO: 53 - DNA, 20 bp, Homo sapiens
  source 1..20: mol_type = unassigned DNA
gcgcccctaaactcgtatct

SEQ ID NO: 54 - DNA, 20 bp, Homo sapiens
  source 1..20: mol_type = unassigned DNA
gattgagtcgcgatggaacg

SEQ ID NO: 55 - DNA, 20 bp, Homo sapiens
  source 1..20: mol_type = unassigned DNA
gccgccttcaacccaaaata

SEQ ID NO: 56 - DNA, 20 bp, Homo sapiens
  source 1..20: mol_type = unassigned DNA
cgcgagcgtatagagtacga

SEQ ID NO: 57 - DNA, 20 bp, Homo sapiens
  source 1..20: mol_type = unassigned DNA
accctaaccaaccccgaaac

SEQ ID NO: 58 - DNA, 20 bp, Homo sapiens
  source 1..20: mol_type = unassigned DNA
tagcgtcgcgagcgtataga

SEQ ID NO: 59 - DNA, 20 bp, Homo sapiens
  source 1..20: mol_type = unassigned DNA
aaaaataacccgacgcccga

SEQ ID NO: 60 - DNA, 21 bp, Homo sapiens
  source 1..21: mol_type = unassigned DNA
ggtttcgtagaagaggttttc

SEQ ID NO: 61 - DNA, 20 bp, Homo sapiens
  source 1..20: mol_type = unassigned DNA
cgcgaaataataacgacttt

SEQ ID NO: 62 - DNA, 21 bp, Homo sapiens
  source 1..21: mol_type = unassigned DNA
agaagaggttttcgttggggg

SEQ ID NO: 63 - DNA, 20 bp, Homo sapiens
  source 1..20: mol_type = unassigned DNA
accaaccccgaaactcgaaa

SEQ ID NO: 64 - DNA, 20 bp, Homo sapiens
  source 1..20: mol_type = unassigned DNA
taggatttggggttggtgcg

SEQ ID NO: 65 - DNA, 20 bp, Homo sapiens
  source 1..20: mol_type = unassigned DNA
aacgcaacgacgaacgtaac

SEQ ID NO: 66 - DNA, 20 bp, Homo sapiens
  source 1..20: mol_type = unassigned DNA
tggtagtggggagatcgagg

SEQ ID NO: 67 - DNA, 20 bp, Homo sapiens
  source 1..20: mol_type = unassigned DNA
aaacgcccccaactctaacc

SEQ ID NO: 68 - DNA, 18 bp, Homo sapiens
  source 1..18: mol_type = unassigned DNA
gttgcggacggcgtagat

SEQ ID NO: 69 - DNA, 21 bp, Homo sapiens
  source 1..21: mol_type = unassigned DNA
acgctccccgaaacaataact

SEQ ID NO: 70 - DNA, 21 bp, Homo sapiens
  source 1..21: mol_type = unassigned DNA
ttgttagttttgttagcgcgg

SEQ ID NO: 71 - DNA, 21 bp, Homo sapiens
  source 1..21: mol_type = unassigned DNA
cgtccgcaacgattcatcatc

SEQ ID NO: 72 - DNA, 22 bp, Homo sapiens
  source 1..22: mol_type = unassigned DNA
tgtttaggagatggttcgtggt

SEQ ID NO: 73 - DNA, 20 bp, Homo sapiens
  source 1..20: mol_type = unassigned DNA
gcatctacgccgtccgcaac

SEQ ID NO: 74 - DNA, 18 bp, Homo sapiens
  source 1..18: mol_type = unassigned DNA
atctacgccgtccgcaac

SEQ ID NO: 75 - DNA, 20 bp, Homo sapiens
  source 1..20: mol_type = unassigned DNA
tgtttagacgtgggttgggg

SEQ ID NO: 76 - DNA, 20 bp, Homo sapiens
  source 1..20: mol_type = unassigned DNA
tcaactccactcaccccgta

SEQ ID NO: 77 - DNA, 20 bp, Homo sapiens
  source 1..20: mol_type = unassigned DNA
gaggagggtggagagggtag

SEQ ID NO: 78 - DNA, 20 bp, Homo sapiens
  source 1..20: mol_type = unassigned DNA
ataccgcacgtactcccaac

SEQ ID NO: 79 - DNA, 20 bp, Homo sapiens
  source 1..20: mol_type = unassigned DNA
ggagtggagtaggtagcggt

SEQ ID NO: 80 - DNA, 20 bp, Homo sapiens
  source 1..20: mol_type = unassigned DNA
ttcctaaccctctccgacca

SEQ ID NO: 81 - DNA, 20 bp, Homo sapiens
  source 1..20: mol_type = unassigned DNA
tttttgagcggtgaagggga

SEQ ID NO: 82 - DNA, 20 bp, Homo sapiens
  source 1..20: mol_type = unassigned DNA
aattattaacgcgaccgccg

SEQ ID NO: 83 - DNA, 24 bp, Homo sapiens
  source 1..24: mol_type = unassigned DNA
gtaataatttggtggtatcggggg

SEQ ID NO: 84 - DNA, 23 bp, Homo sapiens
  source 1..23: mol_type = unassigned DNA
tctactaaacgaacacgtaacgc

SEQ ID NO: 85 - DNA, 21 bp, Homo sapiens
  source 1..21: mol_type = unassigned DNA
ataatttggtggtatcggggg

SEQ ID NO: 86 - DNA, 25 bp, Homo sapiens
  source 1..25: mol_type = unassigned DNA
acgcgttattattctactaaacgaa

SEQ ID NO: 87 - DNA, 23 bp, Homo sapiens
  source 1..23: mol_type = unassigned DNA
tggggtttgttttaattgtggtt

SEQ ID NO: 88 - DNA, 22 bp, Homo sapiens
  source 1..22: mol_type = unassigned DNA
gcgaaacccgcgccttcttaat

SEQ ID NO: 89 - DNA, 20 bp, Homo sapiens
  source 1..20: mol_type = unassigned DNA
gaaacccgcgccttcttaat

SEQ ID NO: 90 - DNA, 27 bp, Homo sapiens
  source 1..27: mol_type = unassigned DNA
ggggaagtatagttatttaataagttg

SEQ ID NO: 91 - DNA, 23 bp, Homo sapiens
  source 1..23: mol_type = unassigned DNA
acaaaacatcraaccattaataa

SEQ ID NO: 92 - DNA, 20 bp, Homo sapiens
  source 1..20: mol_type = unassigned DNA
ttcgcgaaggagagcgtatc

SEQ ID NO: 93 - DNA, 20 bp, Homo sapiens
  source 1..20: mol_type = unassigned DNA
ccctacgtacacccccaaac

SEQ ID NO: 94 - DNA, 20 bp, Homo sapiens
  source 1..20: mol_type = unassigned DNA
cgtttgggggtgtacgtagg

SEQ ID NO: 95 - DNA, 20 bp, Homo sapiens
  source 1..20: mol_type = unassigned DNA
aaacccaatacacgcgacga

SEQ ID NO: 96 - DNA, 20 bp, Homo sapiens
  source 1..20: mol_type = unassigned DNA
tttgtcggggaggttggttt

SEQ ID NO: 97 - DNA, 20 bp, Homo sapiens
  source 1..20: mol_type = unassigned DNA
ttcctactaaacgccgacgc

SEQ ID NO: 98 - DNA, 20 bp, Homo sapiens
  source 1..20: mol_type = unassigned DNA
tagcgtttggttcgttcggt

SEQ ID NO: 99 - DNA, 20 bp, Homo sapiens
  source 1..20: mol_type = unassigned DNA
ataaaaacgcgaacgccgac

SEQ ID NO: 100 - DNA, 20 bp, Homo sapiens
  source 1..20: mol_type = unassigned DNA
gcgggcgtttcgattgattt

SEQ ID NO: 101 - DNA, 22 bp, Homo sapiens
  source 1..22: mol_type = unassigned DNA
ttgcgggcgtttcgattgattt

SEQ ID NO: 102 - DNA, 20 bp, Homo sapiens
  source 1..20: mol_type = unassigned DNA
taaaaaccgcccccactacc

SEQ ID NO: 103 - DNA, 20 bp, Homo sapiens
  source 1..20: mol_type = unassigned DNA
tgttcggcggtttaggtgtt

SEQ ID NO: 104 - DNA, 20 bp, Homo sapiens
  source 1..20: mol_type = unassigned DNA
aaatcaatcgaaacgcccgc

SEQ ID NO: 105 - DNA, 20 bp, Homo sapiens
  source 1..20: mol_type = unassigned DNA
tagttcgggtttcgtcgtgc

SEQ ID NO: 106 - DNA, 22 bp, Homo sapiens
  source 1..22: mol_type = unassigned DNA
aaaactaaaaaccgcccccact

SEQ ID NO: 107 - DNA, 20 bp, Homo sapiens
  source 1..20: mol_type = unassigned DNA
aactaaaaaccgcccccact

SEQ ID NO: 108 - DNA, 20 bp, Homo sapiens
  source 1..20: mol_type = unassigned DNA
gtgggtggtagtttgcgttg

SEQ ID NO: 109 - DNA, 20 bp, Homo sapiens
  source 1..20: mol_type = unassigned DNA
cactacctccccgccttaaa

SEQ ID NO: 110 - DNA, 20 bp, Homo sapiens
  source 1..20: mol_type = unassigned DNA
gcgtgcgttttcggttttga

SEQ ID NO: 111 - DNA, 22 bp, Homo sapiens
  source 1..22: mol_type = unassigned DNA
cggcgtgcgttttcggttttga

SEQ ID NO: 112 - DNA, 20 bp, Homo sapiens
  source 1..20: mol_type = unassigned DNA
aacgcaaactaccacccacc

SEQ ID NO: 113 - DNA, 21 bp, Homo sapiens
  source 1..21: mol_type = unassigned DNA
ggacgttgggttgagttagga

SEQ ID NO: 114 - DNA, 20 bp, Homo sapiens
  source 1..20: mol_type = unassigned DNA
acgaccctacaactccccta

SEQ ID NO: 115 - DNA, 20 bp, Homo sapiens
  source 1..20: mol_type = unassigned DNA
ggtgttcgaattgtacggcg

SEQ ID NO: 116 - DNA, 20 bp, Homo sapiens
  source 1..20: mol_type = unassigned DNA
ctacgcgccgctcataaaaa

SEQ ID NO: 117 - DNA, 20 bp, Homo sapiens
  source 1..20: mol_type = unassigned DNA
gcgcgtacggtttcgtatag

SEQ ID NO: 118 - DNA, 20 bp, Homo sapiens
  source 1..20: mol_type = unassigned DNA
atactcgctctttacgcccg

SEQ ID NO: 119 - DNA, 20 bp, Homo sapiens
  source 1..20: mol_type = unassigned DNA
tagagcggtaggtcggtagg

SEQ ID NO: 120 - DNA, 20 bp, Homo sapiens
  source 1..20: mol_type = unassigned DNA
aacaaaccgaaccgctacac

SEQ ID NO: 121 - DNA, 20 bp, Homo sapiens
  source 1..20: mol_type = unassigned DNA
gcggcgtgggaatgaatttt

SEQ ID NO: 122 - DNA, 22 bp, Homo sapiens
  source 1..22: mol_type = unassigned DNA
gggcggcgtgggaatgaatttt

SEQ ID NO: 123 - DNA, 20 bp, Homo sapiens
  source 1..20: mol_type = unassigned DNA
ctttccctcgcacccctaaa

SEQ ID NO: 124 - DNA, 20 bp, Homo sapiens
  source 1..20: mol_type = unassigned DNA
tgcgagggaaagtttgggtt

SEQ ID NO: 125 - DNA, 20 bp, Homo sapiens
  source 1..20: mol_type = unassigned DNA
ccgcgttacccgaaaaactt

SEQ ID NO: 126 - DNA, 20 bp, Homo sapiens
  source 1..20: mol_type = unassigned DNA
ttttaggggtgcgagggaaa

SEQ ID NO: 127 - DNA, 20 bp, Homo sapiens
  source 1..20: mol_type = unassigned DNA
cgcaaccgaactactcaccc

SEQ ID NO: 128 - DNA, 20 bp, Homo sapiens
  source 1..20: mol_type = unassigned DNA
gtgcgagggaaagtttgggt

SEQ ID NO: 129 - DNA, 19 bp, Homo sapiens
  source 1..19: mol_type = unassigned DNA
acccgcgttacccgaaaaa

SEQ ID NO: 130 - DNA, 19 bp, Homo sapiens
  source 1..19: mol_type = unassigned DNA
tttttttggttgtcgggtc

SEQ ID NO: 131 - DNA, 18 bp, Homo sapiens
  source 1..18: mol_type = unassigned DNA
cgaaacccgaaacacgta

SEQ ID NO: 132 - DNA, 20 bp, Homo sapiens
  source 1..20: mol_type = unassigned DNA
agagtggtcgggtgtttagc

SEQ ID NO: 133 - DNA, 21 bp, Homo sapiens
  source 1..21: mol_type = unassigned DNA
acgtaacccaaaaactcgaaa

SEQ ID NO: 134 - DNA, 18 bp, Homo sapiens
  source 1..18: mol_type = unassigned DNA
gtcgttttttaggggtgc

SEQ ID NO: 135 - DNA, 20 bp, Homo sapiens
  source 1..20: mol_type = unassigned DNA
taaacttcgcaaccgaacta

SEQ ID NO: 136 - DNA, 21 bp, Homo sapiens
  source 1..21: mol_type = unassigned DNA
tattaagtttgcgtttgggtc

SEQ ID NO: 137 - DNA, 20 bp, Homo sapiens
  source 1..20: mol_type = unassigned DNA
aaaaccgtctatccctacgc

SEQ ID NO: 138 - DNA, 20 bp, Homo sapiens
  source 1..20: mol_type = unassigned DNA
cgaggtaggaagttttgcgg

SEQ ID NO: 139 - DNA, 20 bp, Homo sapiens
  source 1..20: mol_type = unassigned DNA
cgactcctcccgcgaaataa

SEQ ID NO: 140 - DNA, 20 bp, Homo sapiens
  source 1..20: mol_type = unassigned DNA
cggggtgttgttgtagggtt

SEQ ID NO: 141 - DNA, 19 bp, Homo sapiens
  source 1..19: mol_type = unassigned DNA
aatcacacctacccacgcc

SEQ ID NO: 142 - DNA, 20 bp, Homo sapiens
  source 1..20: mol_type = unassigned DNA
tagggcggttaggtttgggg

SEQ ID NO: 143 - DNA, 20 bp, Homo sapiens
  source 1..20: mol_type = unassigned DNA
gacgaataaccccaccctcc

SEQ ID NO: 144 - DNA, 20 bp, Homo sapiens
  source 1..20: mol_type = unassigned DNA
ttgtcgcgttggtttttcgt

SEQ ID NO: 145 - DNA, 20 bp, Homo sapiens
  source 1..20: mol_type = unassigned DNA
acctttctctcgaccccaat

SEQ ID NO: 146 - DNA, 21 bp, Homo sapiens
  source 1..21: mol_type = unassigned DNA
cgttttgtcggttgcgtgtta

SEQ ID NO: 147 - DNA, 21 bp, Homo sapiens
  source 1..21: mol_type = unassigned DNA
attccccgacctacccaaaac

SEQ ID NO: 148 - DNA, 24 bp, Homo sapiens
  source 1..24: mol_type = unassigned DNA
ggtaggtgataacgttagtgggtt

SEQ ID NO: 149 - DNA, 20 bp, Homo sapiens
  source 1..20: mol_type = unassigned DNA
acctccatcccctacccaac

SEQ ID NO: 150 - DNA, 20 bp, Homo sapiens
  source 1..20: mol_type = unassigned DNA
agtagggggaggtggttttg

SEQ ID NO: 151 - DNA, 20 bp, Homo sapiens
  source 1..20: mol_type = unassigned DNA
tcctcctccccaacttaacc

SEQ ID NO: 152 - DNA, 20 bp, Homo sapiens
  source 1..20: mol_type = unassigned DNA
agtttgggtgtggcggttta

SEQ ID NO: 153 - DNA, 22 bp, Homo sapiens
  source 1..22: mol_type = unassigned DNA
accaacttcgccatattaacca

SEQ ID NO: 154 - DNA, 20 bp, Homo sapiens
  source 1..20: mol_type = unassigned DNA
cgcggtgtattgtgggtagt

SEQ ID NO: 155 - DNA, 20 bp, Homo sapiens
  source 1..20: mol_type = unassigned DNA
ccttccgacccgaatcatcc

SEQ ID NO: 156 - DNA, 19 bp, Homo sapiens
  source 1..19: mol_type = unassigned DNA
ggtcgtcggaacgtgatgt

SEQ ID NO: 157 - DNA, 20 bp, Homo sapiens
  source 1..20: mol_type = unassigned DNA
gccaacatcaacaccaaccc

SEQ ID NO: 158 - DNA, 20 bp, Homo sapiens
  source 1..20: mol_type = unassigned DNA
tcgttttgtcgttgtcgtcg

SEQ ID NO: 159 - DNA, 21 bp, Homo sapiens
  source 1..21: mol_type = unassigned DNA
ttaaataacccgctccctccg

SEQ ID NO: 160 - DNA, 20 bp, Homo sapiens
  source 1..20: mol_type = unassigned DNA
gtcgtgatgttagagcgggc

SEQ ID NO: 161 - DNA, 20 bp, Homo sapiens
  source 1..20: mol_type = unassigned DNA
accccgatcctccttaaacg

SEQ ID NO: 162 - DNA, 18 bp, Homo sapiens
  source 1..18: mol_type = unassigned DNA
ttaaggaggatcggggtc

SEQ ID NO: 163 - DNA, 22 bp, Homo sapiens
  source 1..22: mol_type = unassigned DNA
tcaatacgacgttaaataaccc

SEQ ID NO: 164 - DNA, 20 bp, Homo sapiens
  source 1..20: mol_type = unassigned DNA
tggagttaagcgggtggtag

SEQ ID NO: 165 - DNA, 21 bp, Homo sapiens
  source 1..21: mol_type = unassigned DNA
cccgctctaacatcacgactc

SEQ ID NO: 166 - DNA, 20 bp, Homo sapiens
  source 1..20: mol_type = unassigned DNA
gtattgtcgcgggttcgttc

SEQ ID NO: 167 - DNA, 20 bp, Homo sapiens
  source 1..20: mol_type = unassigned DNA
ctcaaccaatccccactccc

SEQ ID NO: 168 - DNA, 23 bp, Homo sapiens
  source 1..23: mol_type = unassigned DNA
gttttaggtttcgttagtatggg

SEQ ID NO: 169 - DNA, 29 bp, Homo sapiens
  source 1..29: mol_type = unassigned DNA
caaattaaaataaatcatttaacccataa

SEQ ID NO: 170 - DNA, 25 bp, Homo sapiens
  source 1..25: mol_type = unassigned DNA
ttaaaataaatcatttaacccataa

SEQ ID NO: 171 - DNA, 20 bp, Homo sapiens
  source 1..20: mol_type = unassigned DNA
cgaagatttcgtaggcgggt

SEQ ID NO: 172 - DNA, 22 bp, Homo sapiens
  source 1..22: mol_type = unassigned DNA
acgacgcaaataacgctacgca

SEQ ID NO: 173 - DNA, 20 bp, Homo sapiens
  source 1..20: mol_type = unassigned DNA
gacgcaaataacgctacgca

SEQ ID NO: 174 - DNA, 21 bp, Homo sapiens
  source 1..21: mol_type = unassigned DNA
tgttttagaagcgggagaaag

SEQ ID NO: 175 - DNA, 22 bp, Homo sapiens
  source 1..22: mol_type = unassigned DNA
aaataaaacccccgtatccaat

SEQ ID NO: 176 - DNA, 23 bp, Homo sapiens
  source 1..23: mol_type = unassigned DNA
aatgttttagaagcgggagaaag

SEQ ID NO: 177 - DNA, 24 bp, Homo sapiens
  source 1..24: mol_type = unassigned DNA
aaaaataaaacccccgtatccaat

SEQ ID NO: 178 - DNA, 28 bp, Homo sapiens
  source 1..28: mol_type = unassigned DNA
gcggcggttagcgttagtttttcggtag

SEQ ID NO: 179 - DNA, 28 bp, Homo sapiens
  source 1..28: mol_type = unassigned DNA
cgaaacgccaacgtatcataacgacgca

SEQ ID NO: 180 - DNA, 20 bp, Homo sapiens
  source 1..20: mol_type = unassigned DNA
acgttttagggacggcgaat

SEQ ID NO: 181 - DNA, 20 bp, Homo sapiens
  source 1..20: mol_type = unassigned DNA
aatcccaacgaccgtctacc

SEQ ID NO: 182 - DNA, 26 bp, Homo sapiens
  source 1..26: mol_type = unassigned DNA
tttcgttttgtatttatggtagatgt

SEQ ID NO: 183 - DNA, 20 bp, Homo sapiens
  source 1..20: mol_type = unassigned DNA
ccaacgaccgtctaccacta

SEQ ID NO: 184 - DNA, 20 bp, Homo sapiens
  source 1..20: mol_type = unassigned DNA
cgtggtatggatttcggggt

SEQ ID NO: 185 - DNA, 20 bp, Homo sapiens
  source 1..20: mol_type = unassigned DNA
actcctaaccctaaacgcga

SEQ ID NO: 186 - DNA, 22 bp, Homo sapiens
  source 1..22: mol_type = unassigned DNA
gtttttttcggtttttgttcga

SEQ ID NO: 187 - DNA, 22 bp, Homo sapiens
  source 1..22: mol_type = unassigned DNA
tttctcccaattccaatatcca

SEQ ID NO: 188 - DNA, 24 bp, Homo sapiens
  source 1..24: mol_type = unassigned DNA
tggtttttttcggtttttgttcga

SEQ ID NO: 189 - DNA, 24 bp, Homo sapiens
  source 1..24: mol_type = unassigned DNA
actttctcccaattccaatatcca

SEQ ID NO: 190 - DNA, 20 bp, Homo sapiens
  source 1..20: mol_type = unassigned DNA
gcgatcggcgattggttttt

SEQ ID NO: 191 - DNA, 20 bp, Homo sapiens
  source 1..20: mol_type = unassigned DNA
gcgacgacacacgacctaaa

SEQ ID NO: 192 - DNA, 20 bp, Homo sapiens
  source 1..20: mol_type = unassigned DNA
tgaggttttaggtcgtgtgt

SEQ ID NO: 193 - DNA, 22 bp, Homo sapiens
  source 1..22: mol_type = unassigned DNA
ggtgaggttttaggtcgtgtgt

SEQ ID NO: 194 - DNA, 25 bp, Homo sapiens
  source 1..25: mol_type = unassigned DNA
aaaaccttaatcgactcaaataaaa

SEQ ID NO: 195 - DNA, 20 bp, Homo sapiens
  source 1..20: mol_type = unassigned DNA
gggtgtagttgcgtagcgta

SEQ ID NO: 196 - DNA, 20 bp, Homo sapiens
  source 1..20: mol_type = unassigned DNA
ccgaaccctcctcaccaaaa

SEQ ID NO: 197 - DNA, 20 bp, Homo sapiens
  source 1..20: mol_type = unassigned DNA
tagttgcgtagcgtagggta

SEQ ID NO: 198 - DNA, 21 bp, Homo sapiens
  source 1..21: mol_type = unassigned DNA
tcaccaaaatcctcctaaaac

SEQ ID NO: 199 - DNA, 25 bp, Homo sapiens
  source 1..25: mol_type = unassigned DNA
acgtagtgttggtaagatttgtaga

SEQ ID NO: 200 - DNA, 24 bp, Homo sapiens
  source 1..24: mol_type = unassigned DNA
acaaaaaccgcttataaacgacga

SEQ ID NO: 201 - DNA, 22 bp, Homo sapiens
  source 1..22: mol_type = unassigned DNA
gtaggtttttgcgttggagatt

SEQ ID NO: 202 - DNA, 26 bp, Homo sapiens
  source 1..26: mol_type = unassigned DNA
attttcgttacttctctattcccaaa

SEQ ID NO: 203 - DNA, 20 bp, Homo sapiens
  source 1..20: mol_type = unassigned DNA
ggggtttcgcgttttgagtt

SEQ ID NO: 204 - DNA, 20 bp, Homo sapiens
  source 1..20: mol_type = unassigned DNA
aacaccaaaacccccgctaa

SEQ ID NO: 205 - DNA, 22 bp, Homo sapiens
  source 1..22: mol_type = unassigned DNA
aaaagtaattaatcggaacggt

SEQ ID NO: 206 - DNA, 23 bp, Homo sapiens
  source 1..23: mol_type = unassigned DNA
acactttcccaaatacaaaaaaa

SEQ ID NO: 207 - DNA, 20 bp, Homo sapiens
  source 1..20: mol_type = unassigned DNA
tttcgagtcggggcgtttta

SEQ ID NO: 208 - DNA, 20 bp, Homo sapiens
  source 1..20: mol_type = unassigned DNA
tacctaaccgctcgctctct

SEQ ID NO: 209 - DNA, 19 bp, Homo sapiens
  source 1..19: mol_type = unassigned DNA
gttcggttttgggattttt

SEQ ID NO: 210 - DNA, 19 bp, Homo sapiens
  source 1..19: mol_type = unassigned DNA
aatcccaaaaaccgactct

SEQ ID NO: 211 - DNA, 23 bp, Homo sapiens
  source 1..23: mol_type = unassigned DNA
gagggttcggttttgggattttt

SEQ ID NO: 212 - DNA, 22 bp, Homo sapiens
  source 1..22: mol_type = unassigned DNA
accaatcccaaaaaccgactct

SEQ ID NO: 213 - DNA, 20 bp, Homo sapiens
  source 1..20: mol_type = unassigned DNA
cggaggaatttgtgtcgtcg

SEQ ID NO: 214 - DNA, 20 bp, Homo sapiens
  source 1..20: mol_type = unassigned DNA
caccaaaacaacgctacccg

SEQ ID NO: 215 - DNA, 21 bp, Homo sapiens
  source 1..21: mol_type = unassigned DNA
ttgggaatttttttcgtttat

SEQ ID NO: 216 - DNA, 22 bp, Homo sapiens
  source 1..22: mol_type = unassigned DNA
tcctccgaataacttaaaaacc

SEQ ID NO: 217 - DNA, 24 bp, Homo sapiens
  source 1..24: mol_type = unassigned DNA
tcgttggatagtggtatttaatgt

SEQ ID NO: 218 - DNA, 20 bp, Homo sapiens
  source 1..20: mol_type = unassigned DNA
aaaatcaccgactcactcaa

SEQ ID NO: 219 - DNA, 19 bp, Homo sapiens
  source 1..19: mol_type = unassigned DNA
cggagtacggcggtaggaa

SEQ ID NO: 220 - DNA, 23 bp, Homo sapiens
  source 1..23: mol_type = unassigned DNA
aataccccgaaaacccgctaata

SEQ ID NO: 221 - DNA, 20 bp, Homo sapiens
  source 1..20: mol_type = unassigned DNA
accccgaaaacccgctaata

SEQ ID NO: 222 - DNA, 22 bp, Homo sapiens
  source 1..22: mol_type = unassigned DNA
atgatattttgtaggaaagcgt

SEQ ID NO: 223 - DNA, 22 bp, Homo sapiens
  source 1..22: mol_type = unassigned DNA
caaattccgtttctaaaaaaac

SEQ ID NO: 224 - DNA, 20 bp, Homo sapiens
  source 1..20: mol_type = unassigned DNA
gggttcgtatgcgggagtag

SEQ ID NO: 225 - DNA, 20 bp, Homo sapiens
  source 1..20: mol_type = unassigned DNA
acgaaactacaccaacgcct

SEQ ID NO: 226 - DNA, 21 bp, Homo sapiens
  source 1..21: mol_type = unassigned DNA
gggggttttcgttaggagtag

SEQ ID NO: 227 - DNA, 18 bp, Homo sapiens
  source 1..18: mol_type = unassigned DNA
aaaccgccctaaaccacc

SEQ ID NO: 228 - DNA, 20 bp, Homo sapiens
  source 1..20: mol_type = unassigned DNA
gaagggcggtagcgatagtt

SEQ ID NO: 229 - DNA, 22 bp, Homo sapiens
  source 1..22: mol_type = unassigned DNA
ctacgaattccgcaaaccgaaa

SEQ ID NO: 230 - DNA, 20 bp, Homo sapiens
  source 1..20: mol_type = unassigned DNA
acgaattccgcaaaccgaaa

SEQ ID NO: 231 - DNA, 18 bp, Homo sapiens
  source 1..18: mol_type = unassigned DNA
attgtttttgtcggcgtt

SEQ ID NO: 232 - DNA, 19 bp, Homo sapiens
  source 1..19: mol_type = unassigned DNA
tacactacgaattccgcaa

SEQ ID NO: 233 - DNA, 20 bp, Homo sapiens
  source 1..20: mol_type = unassigned DNA
gcgtgcgtatattcgcgttt

SEQ ID NO: 234 - DNA, 22 bp, Homo sapiens
  source 1..22: mol_type = unassigned DNA
cggcgtgcgtatattcgcgttt

SEQ ID NO: 235 - DNA, 20 bp, Homo sapiens
  source 1..20: mol_type = unassigned DNA
aaattccgcctcccctaacc

SEQ ID NO: 236 - DNA, 24 bp, Homo sapiens
  source 1..24: mol_type = unassigned DNA
tttagcgtttaatgtgtatgtaga

SEQ ID NO: 237 - DNA, 26 bp, Homo sapiens
  source 1..26: mol_type = unassigned DNA
cgaaattacaatcgaaacaaacttac

SEQ ID NO: 238 - DNA, 24 bp, Homo sapiens
  source 1..24: mol_type = unassigned DNA
aaattacaatcgaaacaaacttac

SEQ ID NO: 239 - DNA, 19 bp, Homo sapiens
  source 1..19: mol_type = unassigned DNA
gttttgagtagggtgcgag

SEQ ID NO: 240 - DNA, 19 bp, Homo sapiens
  source 1..19: mol_type = unassigned DNA
aaaaaaacaaattccgcct

SEQ ID NO: 241 - DNA, 22 bp, Homo sapiens
  source 1..22: mol_type = unassigned DNA
gatgttttgagtagggtgcgag

SEQ ID NO: 242 - DNA, 24 bp, Homo sapiens
  source 1..24: mol_type = unassigned DNA
aaacgaaaaaaacaaattccgcct

SEQ ID NO: 243 - DNA, 20 bp, Homo sapiens
  source 1..20: mol_type = unassigned DNA
tggtagcgttgtaaggtggg

SEQ ID NO: 244 - DNA, 20 bp, Homo sapiens
  source 1..20: mol_type = unassigned DNA
aaaaacaaacgcgaccctcg

SEQ ID NO: 245 - DNA, 22 bp, Homo sapiens
  source 1..22: mol_type = unassigned DNA
tcgagttttggtagcgttgtaa

SEQ ID NO: 246 - DNA, 24 bp, Homo sapiens
  source 1..24: mol_type = unassigned DNA
aataccccgcaaaaaaaatctaca

SEQ ID NO: 247 - DNA, 20 bp, Homo sapiens
  source 1..20: mol_type = unassigned DNA
ccccgcaaaaaaaatctaca

SEQ ID NO: 248 - DNA, 20 bp, Homo sapiens
  source 1..20: mol_type = unassigned DNA
acgagaaattggcgcgttga

SEQ ID NO: 249 - DNA, 20 bp, Homo sapiens
  source 1..20: mol_type = unassigned DNA
aacaacaccctttacgacgc

SEQ ID NO: 250 - DNA, 23 bp, Homo sapiens
  source 1..23: mol_type = unassigned DNA
tgtttgttagggttttgttttaa

SEQ ID NO: 251 - DNA, 22 bp, Homo sapiens
  source 1..22: mol_type = unassigned DNA
cgccaaaacgaatatttattta

SEQ ID NO: 252 - DNA, 26 bp, Homo sapiens
  source 1..26: mol_type = unassigned DNA
aattgtttgttagggttttgttttaa

SEQ ID NO: 253 - DNA, 25 bp, Homo sapiens
  source 1..25: mol_type = unassigned DNA
cgacgccaaaacgaatatttattta

SEQ ID NO: 254 - DNA, 20 bp, Homo sapiens
  source 1..20: mol_type = unassigned DNA
ggtagcgtagtggattcggg

SEQ ID NO: 255 - DNA, 20 bp, Homo sapiens
  source 1..20: mol_type = unassigned DNA
ctcgtcctccctccgaaaac

SEQ ID NO: 256 - DNA, 22 bp, Homo sapiens
  source 1..22: mol_type = unassigned DNA
gtttgttttatttcgtggggag

SEQ ID NO: 257 - DNA, 21 bp, Homo sapiens
  source 1..21: mol_type = unassigned DNA
ccaactccttaactcgctcaa

SEQ ID NO: 258 - DNA, 20 bp, Homo sapiens
  source 1..20: mol_type = unassigned DNA
tcgcgggaaacggttttagt

SEQ ID NO: 259 - DNA, 18 bp, Homo sapiens
  source 1..18: mol_type = unassigned DNA
gcccttaacgaccctccg

SEQ ID NO: 260 - DNA, 23 bp, Homo sapiens
  source 1..23: mol_type = unassigned DNA
ttttcgcgggaaacggttttagt

SEQ ID NO: 261 - DNA, 20 bp, Homo sapiens
  source 1..20: mol_type = unassigned DNA
gcgcccttaacgaccctccg

SEQ ID NO: 262 - DNA, 20 bp, Homo sapiens
  source 1..20: mol_type = unassigned DNA
cgttttgtaaagcgaagttt

SEQ ID NO: 263 - DNA, 26 bp, Homo sapiens
  source 1..26: mol_type = unassigned DNA
gttatacgttttgtaaagcgaagttt

SEQ ID NO: 264 - DNA, 24 bp, Homo sapiens
  source 1..24: mol_type = unassigned DNA
aaaccaatcaatcactaaactaca

SEQ ID NO: 265 - DNA, 20 bp, Homo sapiens
  source 1..20: mol_type = unassigned DNA
ggtttttgggcgtcgtgtta

SEQ ID NO: 266 - DNA, 20 bp, Homo sapiens
  source 1..20: mol_type = unassigned DNA
aaattcactctccaccgccc

SEQ ID NO: 267 - DNA, 22 bp, Homo sapiens
  source 1..22: mol_type = unassigned DNA
aggcgaataatgaaacggagga

SEQ ID NO: 268 - DNA, 20 bp, Homo sapiens
  source 1..20: mol_type = unassigned DNA
taacacgacgcccaaaaacc

SEQ ID NO: 269 - DNA, 20 bp, Homo sapiens
  source 1..20: mol_type = unassigned DNA
attttacggatggagtgatg

SEQ ID NO: 270 - DNA, 23 bp, Homo sapiens
  source 1..23: mol_type = unassigned DNA
ggaattttacggatggagtgatg

SEQ ID NO: 271 - DNA, 26 bp, Homo sapiens
  source 1..26: mol_type = unassigned DNA
cttatcccgactaaactactaaaaaa

SEQ ID NO: 272 - DNA, 20 bp, Homo sapiens
  source 1..20: mol_type = unassigned DNA
aattcgtttcgcgacgtgag

SEQ ID NO: 273 - DNA, 22 bp, Homo sapiens
  source 1..22: mol_type = unassigned DNA
ttaattcgtttcgcgacgtgag

SEQ ID NO: 274 - DNA, 22 bp, Homo sapiens
  source 1..22: mol_type = unassigned DNA
acacgccttaaaacctactcat

SEQ ID NO: 275 - DNA, 20 bp, Homo sapiens
  source 1..20: mol_type = unassigned DNA
cgtgagggagaatttaggag

SEQ ID NO: 276 - DNA, 26 bp, Homo sapiens
  source 1..26: mol_type = unassigned DNA
taaaaaaacacacgccttaaaaccta

SEQ ID NO: 277 - DNA, 20 bp, Homo sapiens
  source 1..20: mol_type = unassigned DNA
tcgtttagcgagcgttgttt

SEQ ID NO: 278 - DNA, 20 bp, Homo sapiens
  source 1..20: mol_type = unassigned DNA
gatccgccgttacgctattc

SEQ ID NO: 279 - DNA, 22 bp, Homo sapiens
  source 1..22: mol_type = unassigned DNA
tgttagaaatcggtatcgttta

SEQ ID NO: 280 - DNA, 20 bp, Homo sapiens
  source 1..20: mol_type = unassigned DNA
tctacgaaacgtttacaacc

SEQ ID NO: 281 - DNA, 27 bp, Homo sapiens
  source 1..27: mol_type = unassigned DNA
ttttttgttagaaatcggtatcgttta

SEQ ID NO: 282 - DNA, 24 bp, Homo sapiens
  source 1..24: mol_type = unassigned DNA
aaaatctacgaaacgtttacaacc

SEQ ID NO: 283 - DNA, 20 bp, Homo sapiens
  source 1..20: mol_type = unassigned DNA
tttagggatcgcgttcggag

SEQ ID NO: 284 - DNA, 22 bp, Homo sapiens
  source 1..22: mol_type = unassigned DNA
aaactcctttcccctctcatac

SEQ ID NO: 285 - DNA, 20 bp, Homo sapiens
  source 1..20: mol_type = unassigned DNA
cggagtttttaatcggatat

SEQ ID NO: 286 - DNA, 21 bp, Homo sapiens
  source 1..21: mol_type = unassigned DNA
tcccttctctttaaaactcct

SEQ ID NO: 287 - DNA, 22 bp, Homo sapiens
  source 1..22: mol_type = unassigned DNA
gtcggattttattttaatcgtg

SEQ ID NO: 288 - DNA, 26 bp, Homo sapiens
  source 1..26: mol_type = unassigned DNA
aaacaaaaaaatctcaaaaactaaaa

SEQ ID NO: 289 - DNA, 25 bp, Homo sapiens
  source 1..25: mol_type = unassigned DNA
gttgtcggattttattttaatcgtg

SEQ ID NO: 290 - DNA, 29 bp, Homo sapiens
  source 1..29: mol_type = unassigned DNA
cttaaacaaaaaaatctcaaaaactaaaa

SEQ ID NO: 291 - DNA, 20 bp, Homo sapiens
  source 1..20: mol_type = unassigned DNA
gttaggggcgaggcgtttat

SEQ ID NO: 292 - DNA, 20 bp, Homo sapiens
  source 1..20: mol_type = unassigned DNA
cgaaacctaaacgcgcgaaa

SEQ ID NO: 293 - DNA, 20 bp, Homo sapiens
  source 1..20: mol_type = unassigned DNA
aggttaataggtggcgcgtt

SEQ ID NO: 294 - DNA, 20 bp, Homo sapiens
  source 1..20: mol_type = unassigned DNA
cccgcaactccgcgataata

SEQ ID NO: 295 - DNA, 20 bp, Homo sapiens
  source 1..20: mol_type = unassigned DNA
agtcgtttttcgcgcgttta

SEQ ID NO: 296 - DNA, 22 bp, Homo sapiens
  source 1..22: mol_type = unassigned DNA
cgagtcgtttttcgcgcgttta

SEQ ID NO: 297 - DNA, 21 bp, Homo sapiens
  source 1..21: mol_type = unassigned DNA
gacccgacaccctaaactcat

SEQ ID NO: 298 - DNA, 22 bp, Homo sapiens
  source 1..22: mol_type = unassigned DNA
aggcgtttattggttaataggg

SEQ ID NO: 299 - DNA, 22 bp, Homo sapiens
  source 1..22: mol_type = unassigned DNA
cgacccgacaccctaaactcat

SEQ ID NO: 300 - DNA, 20 bp, Homo sapiens
  source 1..20: mol_type = unassigned DNA
acccgacaccctaaactcat

SEQ ID NO: 301 - DNA, 25 bp, Homo sapiens
  source 1..25: mol_type = unassigned DNA
tttaatttgggttttaagtttgagg

SEQ ID NO: 302 - DNA, 21 bp, Homo sapiens
  source 1..21: mol_type = unassigned DNA
acgctactaaaccccgcttat

SEQ ID NO: 303 - DNA, 18 bp, Homo sapiens
  source 1..18: mol_type = unassigned DNA
gcgtttaggtagcgacgc

SEQ ID NO: 304 - DNA, 19 bp, Homo sapiens
  source 1..19: mol_type = unassigned DNA
ataccccgacgaaaacgac

SEQ ID NO: 305 - DNA, 18 bp, Homo sapiens
  source 1..18: mol_type = unassigned DNA
tttgggatttggtcgagc

SEQ ID NO: 306 - DNA, 19 bp, Homo sapiens
  source 1..19: mol_type = unassigned DNA
aaaattaaatcccgcgtcg

SEQ ID NO: 307 - DNA, 19 bp, Homo sapiens
  source 1..19: mol_type = unassigned DNA
cgtttaggtttgtggacgc

SEQ ID NO: 308 - DNA, 22 bp, Homo sapiens
  source 1..22: mol_type = unassigned DNA
aaaaacgaaatcgctaatacgc

SEQ ID NO: 309 - DNA, 18 bp, Homo sapiens
  source 1..18: mol_type = unassigned DNA
tttttcgaatttttgcgc

SEQ ID NO: 310 - DNA, 20 bp, Homo sapiens
  source 1..20: mol_type = unassigned DNA
aacacgctccgactaacttc

SEQ ID NO: 311 - DNA, 24 bp, Homo sapiens
  source 1..24: mol_type = unassigned DNA
ggttgttttttcgaatttttgcgc

SEQ ID NO: 312 - DNA, 22 bp, Homo sapiens
  source 1..22: mol_type = unassigned DNA
taaacacgctccgactaacttc

SEQ ID NO: 313 - DNA, 19 bp, Homo sapiens
  source 1..19: mol_type = unassigned DNA
cgttcgcgttattatttgc

SEQ ID NO: 314 - DNA, 18 bp, Homo sapiens
  source 1..18: mol_type = unassigned DNA
cgcccaataacaactcgt

SEQ ID NO: 315 - DNA, 24 bp, Homo sapiens
  source 1..24: mol_type = unassigned DNA
attatcgttcgcgttattatttgc

SEQ ID NO: 316 - DNA, 21 bp, Homo sapiens
  source 1..21: mol_type = unassigned DNA
cctcgcccaataacaactcgt

SEQ ID NO: 317 - DNA, 20 bp, Homo sapiens
  source 1..20: mol_type = unassigned DNA
gtcgagtcgtcgttacgatc

SEQ ID NO: 318 - DNA, 21 bp, Homo sapiens
  source 1..21: mol_type = unassigned DNA
ctaccctcctcgaactctacg

SEQ ID NO: 319 - DNA, 22 bp, Homo sapiens
  source 1..22: mol_type = unassigned DNA
gttttcggatgggaaattttag

SEQ ID NO: 320 - DNA, 22 bp, Homo sapiens
  source 1..22: mol_type = unassigned DNA
aaaccatctacatcgaaatcgc

SEQ ID NO: 321 - DNA, 25 bp, Homo sapiens
  source 1..25: mol_type = unassigned DNA
gtggttttcggatgggaaattttag

SEQ ID NO: 322 - DNA, 26 bp, Homo sapiens
  source 1..26: mol_type = unassigned DNA
aacaaaaccatctacatcgaaatcgc

SEQ ID NO: 323 - DNA, 25 bp, Homo sapiens
  source 1..25: mol_type = unassigned DNA
tttttaggagtaagtattttgtgtg

SEQ ID NO: 324 - DNA, 21 bp, Homo sapiens
  source 1..21: mol_type = unassigned DNA
ccctcttcctcccctactaat

SEQ ID NO: 325 - DNA, 19 bp, Homo sapiens
  source 1..19: mol_type = unassigned DNA
taggtatatttcggtcggc

SEQ ID NO: 326 - DNA, 18 bp, Homo sapiens
  source 1..18: mol_type = unassigned DNA
aactcgaaacctcatccg

SEQ ID NO: 327 - DNA, 19 bp, Homo sapiens
  source 1..19: mol_type = unassigned DNA
acgggagggtaaatttagc

SEQ ID NO: 328 - DNA, 20 bp, Homo sapiens
  source 1..20: mol_type = unassigned DNA
taccaaacaattcgacgtta

SEQ ID NO: 329 - DNA, 18 bp, Homo sapiens
  source 1..18: mol_type = unassigned DNA
gcgtacgtcggtttattc

SEQ ID NO: 330 - DNA, 18 bp, Homo sapiens
  source 1..18: mol_type = unassigned DNA
acgctctacgcgatcaaa

SEQ ID NO: 331 - DNA, 22 bp, Homo sapiens
  source 1..22: mol_type = unassigned DNA
aagtgcgtacgtcggtttattc

SEQ ID NO: 332 - DNA, 21 bp, Homo sapiens
  source 1..21: mol_type = unassigned DNA
gcgacgctctacgcgatcaaa

SEQ ID NO: 333 - DNA, 20 bp, Homo sapiens
  source 1..20: mol_type = unassigned DNA
cgagcgattgtggggttaga

SEQ ID NO: 334 - DNA, 19 bp, Homo sapiens
  source 1..19: mol_type = unassigned DNA
caactcgcgaccgcctaaa

SEQ ID NO: 335 - DNA, 20 bp, Homo sapiens
  source 1..20: mol_type = unassigned DNA
ttcgggcgtttatagagttc

SEQ ID NO: 336 - DNA, 18 bp, Homo sapiens
  source 1..18: mol_type = unassigned DNA
aaaatcaaaacgcgaacg

SEQ ID NO: 337 - DNA, 19 bp, Homo sapiens
  source 1..19: mol_type = unassigned DNA
tagcgtcgcgttagaaagc

SEQ ID NO: 338 - DNA, 20 bp, Homo sapiens
  source 1..20: mol_type = unassigned DNA
atcgctcaaaacctaacgaa

SEQ ID NO: 339 - DNA, 22 bp, Homo sapiens
  source 1..22: mol_type = unassigned DNA
ttttagcgtcgcgttagaaagc

SEQ ID NO: 340 - DNA, 24 bp, Homo sapiens
  source 1..24: mol_type = unassigned DNA
aaaaatcgctcaaaacctaacgaa

SEQ ID NO: 341 - DNA, 20 bp, Homo sapiens
  source 1..20: mol_type = unassigned DNA
aggtttagtttcgaaatcgc

SEQ ID NO: 342 - DNA, 18 bp, Homo sapiens
  source 1..18: mol_type = unassigned DNA
aaccgaacgattccctaa

SEQ ID NO: 343 - DNA, 24 bp, Homo sapiens
  source 1..24: mol_type = unassigned DNA
gttaaggtttagtttcgaaatcgc

SEQ ID NO: 344 - DNA, 24 bp, Homo sapiens
  source 1..24: mol_type = unassigned DNA
ctaaaaaaccgaacgattccctaa

SEQ ID NO: 345 - DNA, 21 bp, Homo sapiens
  source 1..21: mol_type = unassigned DNA
gaatttttggaaaagtcggga

SEQ ID NO: 346 - DNA, 20 bp, Homo sapiens
  source 1..20: mol_type = unassigned DNA
ctcctacaaaaaaaactccc

SEQ ID NO: 347 - DNA, 25 bp, Homo sapiens
  source 1..25: mol_type = unassigned DNA
ttcggaatttttggaaaagtcggga

SEQ ID NO: 348 - DNA, 23 bp, Homo sapiens
  source 1..23: mol_type = unassigned DNA
cgactcctacaaaaaaaactccc

SEQ ID NO: 349 - DNA, 18 bp, Homo sapiens
  source 1..18: mol_type = unassigned DNA
agaaggtggtcggtaagc

SEQ ID NO: 350 - DNA, 22 bp, Homo sapiens
  source 1..22: mol_type = unassigned DNA
acgtaattataaaaaacacgcc

SEQ ID NO: 351 - DNA, 20 bp, Homo sapiens
  source 1..20: mol_type = unassigned DNA
ggagaaggtggtcggtaagc

SEQ ID NO: 352 - DNA, 25 bp, Homo sapiens
  source 1..25: mol_type = unassigned DNA
aaaacgtaattataaaaaacacgcc

SEQ ID NO: 353 - DNA, 20 bp, Homo sapiens
  source 1..20: mol_type = unassigned DNA
tagttttttggagggagagg

SEQ ID NO: 354 - DNA, 21 bp, Homo sapiens
  source 1..21: mol_type = unassigned DNA
atcctcgtcctcttaaaaaac

SEQ ID NO: 355 - DNA, 20 bp, Homo sapiens
  source 1..20: mol_type = unassigned DNA
gtcgagtttgggattttggt

SEQ ID NO: 356 - DNA, 21 bp, Homo sapiens
  source 1..21: mol_type = unassigned DNA
aaactcctactcgccctaacc

SEQ ID NO: 357 - DNA, 23 bp, Homo sapiens
  source 1..23: mol_type = unassigned DNA
ggggtcgagtttgggattttggt

SEQ ID NO: 358 - DNA, 23 bp, Homo sapiens
  source 1..23: mol_type = unassigned DNA
aaaaactcctactcgccctaacc

SEQ ID NO: 359 - DNA, 20 bp, Homo sapiens
  source 1..20: mol_type = unassigned DNA
tcgagttggataaggcgtac

SEQ ID NO: 360 - DNA, 20 bp, Homo sapiens
  source 1..20: mol_type = unassigned DNA
ccgataacacgaccgacata

SEQ ID NO: 361 - DNA, 20 bp, Homo sapiens
  source 1..20: mol_type = unassigned DNA
tgtatgtcggtcgtgttatc

SEQ ID NO: 362 - DNA, 20 bp, Homo sapiens
  source 1..20: mol_type = unassigned DNA
taaacgtactacctccgacc

SEQ ID NO: 363 - DNA, 25 bp, Homo sapiens
  source 1..25: mol_type = unassigned DNA
ggatgtgggaggttcggttcgggtg

SEQ ID NO: 364 - DNA, 25 bp, Homo sapiens
  source 1..25: mol_type = unassigned DNA
ccgctcgcccctcgctaaaactaca

SEQ ID NO: 365 - DNA, 30 bp, Homo sapiens
  source 1..30: mol_type = unassigned DNA
ggcgcgaggtagttttagtacgtagttttt

SEQ ID NO: 366 - DNA, 28 bp, Homo sapiens
  source 1..28: mol_type = unassigned DNA
ataacaacgtcgtcctttccgcaaaacg

SEQ ID NO: 367 - DNA, 28 bp, Homo sapiens
  source 1..28: mol_type = unassigned DNA
ggggattttgtaagttcgcgcgtggttt

SEQ ID NO: 368 - DNA, 28 bp, Homo sapiens
  source 1..28: mol_type = unassigned DNA
acactctccgcgcgacctatattaacga

SEQ ID NO: 369 - DNA, 28 bp, Homo sapiens
  source 1..28: mol_type = unassigned DNA
ggagacggtttgttatggttgttgcgtt

SEQ ID NO: 370 - DNA, 28 bp, Homo sapiens
  source 1..28: mol_type = unassigned DNA
acgccctttctaccgacctcacgaacta

SEQ ID NO: 371 - DNA, 27 bp, Homo sapiens
  source 1..27: mol_type = unassigned DNA
tttttcgggggcgtggtttgtatgttt

SEQ ID NO: 372 - DNA, 28 bp, Homo sapiens
  source 1..28: mol_type = unassigned DNA
tacctaacgaaacgctcactccacctcg

SEQ ID NO: 373 - DNA, 28 bp, Homo sapiens
  source 1..28: mol_type = unassigned DNA
ttttgcggttaggtgaaggcgtagaggt

SEQ ID NO: 374 - DNA, 28 bp, Homo sapiens
  source 1..28: mol_type = unassigned DNA
gaccgaataccccgctttctctctcgac

SEQ ID NO: 375 - DNA, 28 bp, Homo sapiens
  source 1..28: mol_type = unassigned DNA
gaggtcgagagagaaagcggggtattcg

SEQ ID NO: 376 - DNA, 28 bp, Homo sapiens
  source 1..28: mol_type = unassigned DNA
aacgctctcaacccaacccctaaactca

SEQ ID NO: 377 - DNA, 28 bp, Homo sapiens
  source 1..28: mol_type = unassigned DNA
aggatagtagcggtgagtcgttagcgtt

SEQ ID NO: 378 - DNA, 28 bp, Homo sapiens
  source 1..28: mol_type = unassigned DNA
cgctcccacttttctcctttctccctcc

SEQ ID NO: 379 - DNA, 28 bp, Homo sapiens
  source 1..28: mol_type = unassigned DNA
ttttgataaagtggggagggcgtagggg

SEQ ID NO: 380 - DNA, 28 bp, Homo sapiens
  source 1..28: mol_type = unassigned DNA
acactctcaaatacccgtcgcgctctat

SEQ ID NO: 381 - DNA, 28 bp, Homo sapiens
  source 1..28: mol_type = unassigned DNA
ttttgataaagtggggagggcgtagggg

SEQ ID NO: 382 - DNA, 28 bp, Homo sapiens
  source 1..28: mol_type = unassigned DNA
acactctcaaatacccgtcgcgctctat

SEQ ID NO: 383 - DNA, 28 bp, Homo sapiens
  source 1..28: mol_type = unassigned DNA
agcgtagcgtagttggagtagttgcgaa

SEQ ID NO: 384 - DNA, 30 bp, Homo sapiens
  source 1..30: mol_type = unassigned DNA
cgacgactctcttcccaatctaaaacccca

SEQ ID NO: 385 - DNA, 28 bp, Homo sapiens
  source 1..28: mol_type = unassigned DNA
cggagtttagaagggcgttcggttacgg

SEQ ID NO: 386 - DNA, 28 bp, Homo sapiens
  source 1..28: mol_type = unassigned DNA
ctccacgaatcgcatctttcaataccca

SEQ ID NO: 387 - DNA, 28 bp, Homo sapiens
  source 1..28: mol_type = unassigned DNA
aaaggtggttcgagtgaggaaattgcgg

SEQ ID NO: 388 - DNA, 28 bp, Homo sapiens
  source 1..28: mol_type = unassigned DNA
gcgtccctaaacgacacacgacgaaatc

SEQ ID NO: 389 - DNA, 28 bp, Homo sapiens
  source 1..28: mol_type = unassigned DNA
gtcgacggcggttttatcgtattgtcgc

SEQ ID NO: 390 - DNA, 28 bp, Homo sapiens
  source 1..28: mol_type = unassigned DNA
ccttctcccgaaccttccttcgtatcct

SEQ ID NO: 391 - DNA, 28 bp, Homo sapiens
  source 1..28: mol_type = unassigned DNA
ttagaggtatggcggggtttttgtgacg

SEQ ID NO: 392 - DNA, 28 bp, Homo sapiens
  source 1..28: mol_type = unassigned DNA
aatactccctaaacctcctaaccgcgcc

SEQ ID NO: 393 - DNA, 25 bp, Homo sapiens
  source 1..25: mol_type = unassigned DNA
gaggtttaattgtttcgttggtcgc

SEQ ID NO: 394 - DNA, 23 bp, Homo sapiens
  source 1..23: mol_type = unassigned DNA
acgcaaaaccgcgtatatcacct

SEQ ID NO: 395 - DNA, 28 bp, Homo sapiens
  source 1..28: mol_type = unassigned DNA
ggttttagggacgcggttggaatttggg

SEQ ID NO: 396 - DNA, 29 bp, Homo sapiens
  source 1..29: mol_type = unassigned DNA
cccaacgcctcgaccatattaaataactt

SEQ ID NO: 397 - DNA, 28 bp, Homo sapiens
  source 1..28: mol_type = unassigned DNA
gcgattaaggcgtatcggtgggtattgc

SEQ ID NO: 398 - DNA, 28 bp, Homo sapiens
  source 1..28: mol_type = unassigned DNA
aacaccacccgacgaactctcgactaac

SEQ ID NO: 399 - DNA, 28 bp, Homo sapiens
  source 1..28: mol_type = unassigned DNA
taggacgttgtttggttcgaagttcggg

SEQ ID NO: 400 - DNA, 28 bp, Homo sapiens
  source 1..28: mol_type = unassigned DNA
ctccgaaccgaccgaaaaacgcaacttt

SEQ ID NO: 401 - DNA, 21 bp, Homo sapiens
  source 1..21: mol_type = unassigned DNA
ggcggggtattgttttgtttc

SEQ ID NO: 402 - DNA, 24 bp, Homo sapiens
  source 1..24: mol_type = unassigned DNA
tctaccgatatcataacaccgact

SEQ ID NO: 403 - DNA, 28 bp, Homo sapiens
  source 1..28: mol_type = unassigned DNA
aaaggtagagggaaggagagttgttttt

SEQ ID NO: 404 - DNA, 29 bp, Homo sapiens
  source 1..29: mol_type = unassigned DNA
actcctacctcctccgaatcctaaaacct

SEQ ID NO: 405 - DNA, 28 bp, Homo sapiens
  source 1..28: mol_type = unassigned DNA
cggaatgaaggtgtttcgtaggaaggcg

SEQ ID NO: 406 - DNA, 27 bp, Homo sapiens
  source 1..27: mol_type = unassigned DNA
gctacgacacccaacgacccatcgaaa

SEQ ID NO: 407 - DNA, 28 bp, Homo sapiens
  source 1..28: mol_type = unassigned DNA
aatgattttgttgggttcggtggagcgg

SEQ ID NO: 408 - DNA, 28 bp, Homo sapiens
  source 1..28: mol_type = unassigned DNA
ccgacaacttccgcgccatctcttaaac

SEQ ID NO: 409 - DNA, 28 bp, Homo sapiens
  source 1..28: mol_type = unassigned DNA
ttgtgttttgagcgtaggttgcgcgtag

SEQ ID NO: 410 - DNA, 28 bp, Homo sapiens
  source 1..28: mol_type = unassigned DNA
gccttcccgtcgtaaaacaactccgaca

SEQ ID NO: 411 - DNA, 29 bp, Homo sapiens
  source 1..29: mol_type = unassigned DNA
gttttagggggttgggggtttgttaggga

SEQ ID NO: 412 - DNA, 28 bp, Homo sapiens
  source 1..28: mol_type = unassigned DNA
aacccgaaacgaaactatacaccccgca

SEQ ID NO: 413 - DNA, 28 bp, Homo sapiens
  source 1..28: mol_type = unassigned DNA
tcgatgttcgtagtgttgttgtagcggt

SEQ ID NO: 414 - DNA, 28 bp, Homo sapiens
  source 1..28: mol_type = unassigned DNA
ccatccccgcctaacgaaaactaaccct

SEQ ID NO: 415 - DNA, 28 bp, Homo sapiens
  source 1..28: mol_type = unassigned DNA
cgtttcgagaagaagtttcgcggttggt

SEQ ID NO: 416 - DNA, 28 bp, Homo sapiens
  source 1..28: mol_type = unassigned DNA
atctaaacccaaatcgaaaaccgccgcc

SEQ ID NO: 417 - DNA, 28 bp, Homo sapiens
  source 1..28: mol_type = unassigned DNA
ttggagttgtcgtagatcgtcgtggtgg

SEQ ID NO: 418 - DNA, 28 bp, Homo sapiens
  source 1..28: mol_type = unassigned DNA
aaatcgccccactaccgcatccttactc

SEQ ID NO: 419 - DNA, 28 bp, Homo sapiens
  source 1..28: mol_type = unassigned DNA
gcggttaggtgtggtaaagtagttggcg

SEQ ID NO: 420 - DNA, 28 bp, Homo sapiens
  source 1..28: mol_type = unassigned DNA
gcgcacaaccaacctataaactccgacg

SEQ ID NO: 421 - DNA, 22 bp, Homo sapiens
  source 1..22: mol_type = unassigned DNA
gggatttgtgaaggcggatttg

SEQ ID NO: 422 - DNA, 27 bp, Homo sapiens
  source 1..27: mol_type = unassigned DNA
aatattccgtcgataccgaaaacccga

SEQ ID NO: 423 - DNA, 27 bp, Homo sapiens
  source 1..27: mol_type = unassigned DNA
cggttggagcgcgttttcgagaagaat

SEQ ID NO: 424 - DNA, 28 bp, Homo sapiens
  source 1..28: mol_type = unassigned DNA
aacgcaaaacaaaccgcgaccgaaaata

SEQ ID NO: 425 - DNA, 22 bp, Homo sapiens
  source 1..22: mol_type = unassigned DNA
aggagaagtcgtagcgggcgtc

SEQ ID NO: 426 - DNA, 24 bp, Homo sapiens
  source 1..24: mol_type = unassigned DNA
gactaaaccttctaccgcccaccg

SEQ ID NO: 427 - DNA, 28 bp, Homo sapiens
  source 1..28: mol_type = unassigned DNA
tagttgcgatggggtgggaagttacgtt

SEQ ID NO: 428 - DNA, 28 bp, Homo sapiens
  source 1..28: mol_type = unassigned DNA
aaaaccatcgccatccacgaaaacgaca

SEQ ID NO: 429 - DNA, 30 bp, Homo sapiens
  source 1..30: mol_type = unassigned DNA
aggaggatgatagtttagaaagaagagggt

SEQ ID NO: 430 - DNA, 28 bp, Homo sapiens
  source 1..28: mol_type = unassigned DNA
cgcgaccgcgacgataacgataaaaact

SEQ ID NO: 431 - DNA, 28 bp, Homo sapiens
  source 1..28: mol_type = unassigned DNA
gaaacgtgtaggcgtcggcgtttatgag

SEQ ID NO: 432 - DNA, 28 bp, Homo sapiens
  source 1..28: mol_type = unassigned DNA
cgacctctcgaacgcctcctacaaacaa

SEQ ID NO: 433 - DNA, 21 bp, Homo sapiens
  source 1..21: mol_type = unassigned DNA
gtggaggaggaagttcgtttc

SEQ ID NO: 434 - DNA, 24 bp, Homo sapiens
  source 1..24: mol_type = unassigned DNA
aactactaccaaacacgaaacgca

SEQ ID NO: 435 - DNA, 23 bp, Homo sapiens
  source 1..23: mol_type = unassigned DNA
ggttagagtcggttgcgtagttt

SEQ ID NO: 436 - DNA, 27 bp, Homo sapiens
  source 1..27: mol_type = unassigned DNA
tttttgttaggcgaagtatagagagcg

SEQ ID NO: 437 - DNA, 27 bp, Homo sapiens
  source 1..27: mol_type = unassigned DNA
tttttcgattggtcgacggcgagagag

SEQ ID NO: 438 - DNA, 28 bp, Homo sapiens
  source 1..28: mol_type = unassigned DNA
tttccgaactacaaacgcacactaaaac

SEQ ID NO: 439 - DNA, 26 bp, Homo sapiens
  source 1..26: mol_type = unassigned DNA
gattcgtatgggttttatcgagtttc

SEQ ID NO: 440 - DNA, 25 bp, Homo sapiens
  source 1..25: mol_type = unassigned DNA
acttaaaaataaactcgcccgtacg

SEQ ID NO: 441 - DNA, 28 bp, Homo sapiens
  source 1..28: mol_type = unassigned DNA
tcgttggtgaaggtgtagtgttcgttcg

SEQ ID NO: 442 - DNA, 28 bp, Homo sapiens
  source 1..28: mol_type = unassigned DNA
taacgcgcgcgctcacaaataaaacgac

SEQ ID NO: 443 - DNA, 28 bp, Homo sapiens
  source 1..28: mol_type = unassigned DNA
tttttagtggttcgagcgtttgcgttgc

SEQ ID NO: 444 - DNA, 29 bp, Homo sapiens
  source 1..29: mol_type = unassigned DNA
tccgtcctcgaaataattctaaccgacgc

SEQ ID NO: 445 - DNA, 25 bp, Homo sapiens
  source 1..25: mol_type = unassigned DNA
cgtggtatagttaatcgcgcggcgt

SEQ ID NO: 446 - DNA, 28 bp, Homo sapiens
  source 1..28: mol_type = unassigned DNA
tacaaccccaacgccataactcgccaat

SEQ ID NO: 447 - DNA, 28 bp, Homo sapiens
  source 1..28: mol_type = unassigned DNA
tgtcgtcgtcgtcggggttttgttattt

SEQ ID NO: 448 - DNA, 28 bp, Homo sapiens
  source 1..28: mol_type = unassigned DNA
acgacgataaactcccgctaaacccgaa

SEQ ID NO: 449 - DNA, 27 bp, Homo sapiens
  source 1..27: mol_type = unassigned DNA
gaacgaggatttgcgtttttggatcgc

SEQ ID NO: 450 - DNA, 29 bp, Homo sapiens
  source 1..29: mol_type = unassigned DNA
cctaaactcctctacatattcctctacct

SEQ ID NO: 451 - DNA, 27 bp, Homo sapiens
  source 1..27: mol_type = unassigned DNA
gaatggttgcgatatggggttcgacgg

SEQ ID NO: 452 - DNA, 27 bp, Homo sapiens
  source 1..27: mol_type = unassigned DNA
ccacgatatccgccgcgatccaaaaac

SEQ ID NO: 453 - DNA, 25 bp, Homo sapiens
  source 1..25: mol_type = unassigned DNA
taagggtcggttgttgttttttttc

SEQ ID NO: 454 - DNA, 25 bp, Homo sapiens
  source 1..25: mol_type = unassigned DNA
accgacgccttccttataaaatacg

SEQ ID NO: 455 - DNA, 28 bp, Homo sapiens
  source 1..28: mol_type = unassigned DNA
tgtgggataatttggcgaagggagtaga

SEQ ID NO: 456 - DNA, 28 bp, Homo sapiens
  source 1..28: mol_type = unassigned DNA
aactcgaaattaactacgaacgcccgcc

SEQ ID NO: 457 - DNA, 28 bp, Homo sapiens
  source 1..28: mol_type = unassigned DNA
ggcgggggtagtttttgtattaaggcga

SEQ ID NO: 458 - DNA, 28 bp, Homo sapiens
  source 1..28: mol_type = unassigned DNA
cctacgctactactcttctcgacccccg

SEQ ID NO: 459 - DNA, 27 bp, Homo sapiens
  source 1..27: mol_type = unassigned DNA
cgtttcgttcgtcggtcgtagcgattg

SEQ ID NO: 460 - DNA, 28 bp, Homo sapiens
  source 1..28: mol_type = unassigned DNA
ccgacgaaacattttcgcaccacaacac

SEQ ID NO: 461 - DNA, 28 bp, Homo sapiens
  source 1..28: mol_type = unassigned DNA
cgcggtttcggggtatacggagtttttg

SEQ ID NO: 462 - DNA, 28 bp, Homo sapiens
  source 1..28: mol_type = unassigned DNA
gcaattcaatcgctacgaccgacgaacg

SEQ ID NO: 463 - DNA, 28 bp, Homo sapiens
  source 1..28: mol_type = unassigned DNA
cgtcgtagcgggtacggttaacgagttg

SEQ ID NO: 464 - DNA, 27 bp, Homo sapiens
  source 1..27: mol_type = unassigned DNA
tttctccactcgaaacgcccgacaacc

SEQ ID NO: 465 - DNA, 28 bp, Homo sapiens
  source 1..28: mol_type = unassigned DNA
cgggggagaacggtttgagtttcgagta

SEQ ID NO: 466 - DNA, 28 bp, Homo sapiens
  source 1..28: mol_type = unassigned DNA
tcatatttcaacctcgccgccgctaaac

SEQ ID NO: 467 - DNA, 28 bp, Homo sapiens
  source 1..28: mol_type = unassigned DNA
agtagggatggtcgttcgttgttcggtg

SEQ ID NO: 468 - DNA, 28 bp, Homo sapiens
  source 1..28: mol_type = unassigned DNA
gacaaacgaccgaaaatactcgcgcaac

SEQ ID NO: 469 - DNA, 28 bp, Homo sapiens
  source 1..28: mol_type = unassigned DNA
ttttacggtcggggcgatagttgaaggt

SEQ ID NO: 470 - DNA, 28 bp, Homo sapiens
  source 1..28: mol_type = unassigned DNA
tcacgccaatacccgctaatccctccta

SEQ ID NO: 471 - DNA, 27 bp, Homo sapiens
  source 1..27: mol_type = unassigned DNA
ggggatggataatttttaggcgttaac

SEQ ID NO: 472 - DNA, 22 bp, Homo sapiens
  source 1..22: mol_type = unassigned DNA
taacctcgtctttatccccgcg

SEQ ID NO: 473 - DNA, 29 bp, Homo sapiens
  source 1..29: mol_type = unassigned DNA
agtgtgtagtcgtttgtgggtgaggagtt

SEQ ID NO: 474 - DNA, 28 bp, Homo sapiens
  source 1..28: mol_type = unassigned DNA
caccgcgaaaaacgcccacaatcttacc

SEQ ID NO: 475 - DNA, 29 bp, Homo sapiens
  source 1..29: mol_type = unassigned DNA
cgcgggggagtttatttttgaggattcgg

SEQ ID NO: 476 - DNA, 28 bp, Homo sapiens
  source 1..28: mol_type = unassigned DNA
actcctcacccacaaacgactacacact

SEQ ID NO: 477 - DNA, 29 bp, Homo sapiens
  source 1..29: mol_type = unassigned DNA
tagtatttgtacggagtttttcggcggtc

SEQ ID NO: 478 - DNA, 29 bp, Homo sapiens
  source 1..29: mol_type = unassigned DNA
tacgacgcaaccaacgatactatcaccaa

SEQ ID NO: 479 - DNA, 27 bp, Homo sapiens
  source 1..27: mol_type = unassigned DNA
tagtgattggttatttgggcgcggggc

SEQ ID NO: 480 - DNA, 28 bp, Homo sapiens
  source 1..28: mol_type = unassigned DNA
aaacgacatccatcatctccctcgaccc

SEQ ID NO: 481 - DNA, 20 bp, Homo sapiens
  source 1..20: mol_type = unassigned DNA
aggtcgcgttttggtcgtgc

SEQ ID NO: 482 - DNA, 25 bp, Homo sapiens
  source 1..25: mol_type = unassigned DNA
acttaaaaataaactcgcccgtacg

SEQ ID NO: 483 - DNA, 28 bp, Homo sapiens
  source 1..28: mol_type = unassigned DNA
attttacgtagggtggggttgagggcgt

SEQ ID NO: 484 - DNA, 28 bp, Homo sapiens
  source 1..28: mol_type = unassigned DNA
atcctaaccgtcccgcctcaaaaccgta

SEQ ID NO: 485 - DNA, 26 bp, Homo sapiens
  source 1..26: mol_type = unassigned DNA
cgtcgtagtatttggcggcgcgtttc

SEQ ID NO: 486 - DNA, 28 bp, Homo sapiens
  source 1..28: mol_type = unassigned DNA
aacgtacctaatccccaaacccactcct

SEQ ID NO: 487 - DNA, 29 bp, Homo sapiens
  source 1..29: mol_type = unassigned DNA
tcgttgtgcgcgtttcgtttgttggatta

SEQ ID NO: 488 - DNA, 28 bp, Homo sapiens
  source 1..28: mol_type = unassigned DNA
tcgataatatctccgtcgcctccgcaaa

SEQ ID NO: 489 - DNA, 28 bp, Homo sapiens
  source 1..28: mol_type = unassigned DNA
gcgcgtttaatcgtgggatttttgggag

SEQ ID NO: 490 - DNA, 27 bp, Homo sapiens
  source 1..27: mol_type = unassigned DNA
caaattcgcgacaccctaccccaacac

SEQ ID NO: 491 - DNA, 21 bp, Homo sapiens
  source 1..21: mol_type = unassigned DNA
gggtgtcgcgaatttggggta

SEQ ID NO: 492 - DNA, 28 bp, Homo sapiens
  source 1..28: mol_type = unassigned DNA
ctaaacctctcccctcccaaatttacct

SEQ ID NO: 493 - DNA, 28 bp, Homo sapiens
  source 1..28: mol_type = unassigned DNA
atcgagtttttagcggtttttggggcgg

SEQ ID NO: 494 - DNA, 28 bp, Homo sapiens
  source 1..28: mol_type = unassigned DNA
actaacatcgcgcacttaaatctttccg

SEQ ID NO: 495 - DNA, 21 bp, Homo sapiens
  source 1..21: mol_type = unassigned DNA
ggtagcggcgggtaaaaagtc

SEQ ID NO: 496 - DNA, 22 bp, Homo sapiens
  source 1..22: mol_type = unassigned DNA
tacaactttttacctccgccgc

SEQ ID NO: 497 - DNA, 28 bp, Homo sapiens
  source 1..28: mol_type = unassigned DNA
cgtcgatttgcggaatttcgtcgtcgtt

SEQ ID NO: 498 - DNA, 28 bp, Homo sapiens
  source 1..28: mol_type = unassigned DNA
acatccgcgtaaactcgccctttaacac

SEQ ID NO: 499 - DNA, 28 bp, Homo sapiens
  source 1..28: mol_type = unassigned DNA
tttcgggattagggtttcggagggtgtc

SEQ ID NO: 500 - DNA, 28 bp, Homo sapiens
  source 1..28: mol_type = unassigned DNA
cgtatcgatccgtccctcccgcttaaaa

SEQ ID NO: 501 - DNA, 26 bp, Homo sapiens
  source 1..26: mol_type = unassigned DNA
cggttttggtggtagttttggtaatc

SEQ ID NO: 502 - DNA, 28 bp, Homo sapiens
  source 1..28: mol_type = unassigned DNA
aaaacctcccgaacgacgaaataatcca

SEQ ID NO: 503 - DNA, 21 bp, Homo sapiens
  source 1..21: mol_type = unassigned DNA
gtaggcggtcggaacgtgaac

SEQ ID NO: 504 - DNA, 29 bp, Homo sapiens
  source 1..29: mol_type = unassigned DNA
cgataaaaactacaataactcgacaacca

SEQ ID NO: 505 - DNA, 24 bp, Homo sapiens
  source 1..24: mol_type = unassigned DNA
gttgtgagggttttcggcggtatc

SEQ ID NO: 506 - DNA, 21 bp, Homo sapiens
  source 1..21: mol_type = unassigned DNA
cataacaacgcgcgaccccta

SEQ ID NO: 507 - DNA, 29 bp, Homo sapiens
  source 1..29: mol_type = unassigned DNA
tgattataaattagggggtttggtcgtcg

SEQ ID NO: 508 - DNA, 28 bp, Homo sapiens
  source 1..28: mol_type = unassigned DNA
aaaccctccaccctcgcaatactactcc

SEQ ID NO: 509 - DNA, 28 bp, Homo sapiens
  source 1..28: mol_type = unassigned DNA
tgtaggagataatgggagtgaagaggga

SEQ ID NO: 510 - DNA, 28 bp, Homo sapiens
  source 1..28: mol_type = unassigned DNA
ttccacgaaacgcgcgacttcctaacta

SEQ ID NO: 511 - DNA, 24 bp, Homo sapiens
  source 1..24: mol_type = unassigned DNA
gttgagttaggagaggtcgatagc

SEQ ID NO: 512 - DNA, 28 bp, Homo sapiens
  source 1..28: mol_type = unassigned DNA
cccgaaaacaacgactatcgaaatccaa

SEQ ID NO: 513 - DNA, 28 bp, Homo sapiens
  source 1..28: mol_type = unassigned DNA
ataaggtttggtggaagcgtaggagcgt

SEQ ID NO: 514 - DNA, 28 bp, Homo sapiens
  source 1..28: mol_type = unassigned DNA
acgccgaataaaaatcccgcaaccacaa

SEQ ID NO: 515 - DNA, 28 bp, Homo sapiens
  source 1..28: mol_type = unassigned DNA
ggaggggaggagatagcgttatttaggg

SEQ ID NO: 516 - DNA, 28 bp, Homo sapiens
  source 1..28: mol_type = unassigned DNA
aaacaaaacccgaaaccccacctacacc

SEQ ID NO: 517 - DNA, 28 bp, Homo sapiens
  source 1..28: mol_type = unassigned DNA
gcgtggtagttgaggatgtagacgtggt

SEQ ID NO: 518 - DNA, 28 bp, Homo sapiens
  source 1..28: mol_type = unassigned DNA
tccgaactacttaaaaatccccgccgcc

SEQ ID NO: 519 - DNA, 28 bp, Homo sapiens
  source 1..28: mol_type = unassigned DNA
tcgttggttgtgatttttatgcgggcgt

SEQ ID NO: 520 - DNA, 28 bp, Homo sapiens
  source 1..28: mol_type = unassigned DNA
acctctccgataaaccaaatcctccgcc

SEQ ID NO: 521 - DNA, 28 bp, Homo sapiens
  source 1..28: mol_type = unassigned DNA
cgggtgaggtttgtggttaatttcgcgt

SEQ ID NO: 522 - DNA, 28 bp, Homo sapiens
  source 1..28: mol_type = unassigned DNA
ctcaaccaaactacaacgttcccgcctc

SEQ ID NO: 523 - DNA, 28 bp, Homo sapiens
  source 1..28: mol_type = unassigned DNA
aatggaggcgtagattaacgagcggtgt

SEQ ID NO: 524 - DNA, 28 bp, Homo sapiens
  source 1..28: mol_type = unassigned DNA
atccttaacaaccccgccgactaacgtc

SEQ ID NO: 525 - DNA, 28 bp, Homo sapiens
  source 1..28: mol_type = unassigned DNA
acgggtacggagaaacgtcggatttagt

SEQ ID NO: 526 - DNA, 28 bp, Homo sapiens
  source 1..28: mol_type = unassigned DNA
tccccgcgacactctacctataacgtcc

SEQ ID NO: 527 - DNA, 22 bp, Homo sapiens
  source 1..22: mol_type = unassigned DNA
cgtttggcgggtattgttgttc

SEQ ID NO: 528 - DNA, 20 bp, Homo sapiens
  source 1..20: mol_type = unassigned DNA
cccgacgcaaactccctctc

SEQ ID NO: 529 - DNA, 25 bp, Homo sapiens
  source 1..25: mol_type = unassigned DNA
gaagttgttttttaggggtttgcgc

SEQ ID NO: 530 - DNA, 25 bp, Homo sapiens
  source 1..25: mol_type = unassigned DNA
actcaaatctaccctcgcttcaacg

SEQ ID NO: 531 - DNA, 28 bp, Homo sapiens
  source 1..28: mol_type = unassigned DNA
gcgcgggggtattttggagggttagtta

SEQ ID NO: 532 - DNA, 28 bp, Homo sapiens
  source 1..28: mol_type = unassigned DNA
tccctactcgcccgctacgactataaca

SEQ ID NO: 533 - DNA, 28 bp, Homo sapiens
  source 1..28: mol_type = unassigned DNA
cggattttgttttcgggagtcgttcggg

SEQ ID NO: 534 - DNA, 28 bp, Homo sapiens
  source 1..28: mol_type = unassigned DNA
aactaaacgcctaacccttccctcccac

SEQ ID NO: 535 - DNA, 28 bp, Homo sapiens
  source 1..28: mol_type = unassigned DNA
ttcgttttgtttttcggttggagcgggt

SEQ ID NO: 536 - DNA, 28 bp, Homo sapiens
  source 1..28: mol_type = unassigned DNA
tataacctaacccttcaaccgcgcctcg

SEQ ID NO: 537 - DNA, 28 bp, Homo sapiens
  source 1..28: mol_type = unassigned DNA
aggcggcggtttttggcgattgtttttc

SEQ ID NO: 538 - DNA, 27 bp, Homo sapiens
  source 1..27: mol_type = unassigned DNA
ttccgttaccataaaactacccgcccc

SEQ ID NO: 539 - DNA, 21 bp, Homo sapiens
  source 1..21: mol_type = unassigned DNA
gatttcgcgtatcgtcgtgtc

SEQ ID NO: 540 - DNA, 21 bp, Homo sapiens
  source 1..21: mol_type = unassigned DNA
taatatcccccgtaccccccg

SEQ ID NO: 541 - DNA, 28 bp, Homo sapiens
  source 1..28: mol_type = unassigned DNA
tttcgataatagcgtttttgcggcgtgg

SEQ ID NO: 542 - DNA, 28 bp, Homo sapiens
  source 1..28: mol_type = unassigned DNA
caaaaacacgcgacctacgccctcctaa

SEQ ID NO: 543 - DNA, 28 bp, Homo sapiens
  source 1..28: mol_type = unassigned DNA
cgagtcggagtgagcgttaagtgagggg

SEQ ID NO: 544 - DNA, 28 bp, Homo sapiens
  source 1..28: mol_type = unassigned DNA
cctatcaacgaccacccaactactccct

SEQ ID NO: 545 - DNA, 28 bp, Homo sapiens
  source 1..28: mol_type = unassigned DNA
tcgggtttagcgtcgtttgtagtttcgg

SEQ ID NO: 546 - DNA, 28 bp, Homo sapiens
  source 1..28: mol_type = unassigned DNA
aaaaacgtctccttaattccccgcgctt

SEQ ID NO: 547 - DNA, 30 bp, Homo sapiens
  source 1..30: mol_type = unassigned DNA
gcggttggagtagaagtgttagcggttaga

SEQ ID NO: 548 - DNA, 28 bp, Homo sapiens
  source 1..28: mol_type = unassigned DNA
tcaccctacaaaaaccgataaccgacga

SEQ ID NO: 549 - DNA, 28 bp, Homo sapiens
  source 1..28: mol_type = unassigned DNA
agttggttataggcggcgaattgggttt

SEQ ID NO: 550 - DNA, 28 bp, Homo sapiens
  source 1..28: mol_type = unassigned DNA
tcaacaccccctctcctaacctctccaa

SEQ ID NO: 551 - DNA, 28 bp, Homo sapiens
  source 1..28: mol_type = unassigned DNA
cggggaagagtttcggttcgcgttttag

SEQ ID NO: 552 - DNA, 28 bp, Homo sapiens
  source 1..28: mol_type = unassigned DNA
ccctcctataaccccgacctacccgaaa

SEQ ID NO: 553 - DNA, 28 bp, Homo sapiens
  source 1..28: mol_type = unassigned DNA
gcgcggtaggtgtttttcgggttgtaaa

SEQ ID NO: 554 - DNA, 28 bp, Homo sapiens
  source 1..28: mol_type = unassigned DNA
acctttacctaactacactcccatccaa

SEQ ID NO: 555 - DNA, 21 bp, Homo sapiens
  source 1..21: mol_type = unassigned DNA
agagtaggtcgtgggggattc

SEQ ID NO: 556 - DNA, 21 bp, Homo sapiens
  source 1..21: mol_type = unassigned DNA
cgcgctaaccgcgataaaaac

SEQ ID NO: 557 - DNA, 28 bp, Homo sapiens
  source 1..28: mol_type = unassigned DNA
aggagcgggagagggaaaaatagttaag

SEQ ID NO: 558 - DNA, 27 bp, Homo sapiens
  source 1..27: mol_type = unassigned DNA
actacgcccaaaactcaactactaaat

SEQ ID NO: 559 - DNA, 28 bp, Homo sapiens
  source 1..28: mol_type = unassigned DNA
tgggaacgggatagggacgcgttttaat

SEQ ID NO: 560 - DNA, 28 bp, Homo sapiens
  source 1..28: mol_type = unassigned DNA
gaatcccctaaactacccgccatcccac

SEQ ID NO: 561 - DNA, 29 bp, Homo sapiens
  source 1..29: mol_type = unassigned DNA
tgtacgcgtatttttggagggtggtttgc

SEQ ID NO: 562 - DNA, 28 bp, Homo sapiens
  source 1..28: mol_type = unassigned DNA
cgatctaatcgaccacctcctctcctcc

SEQ ID NO: 563 - DNA, 28 bp, Homo sapiens
  source 1..28: mol_type = unassigned DNA
aagtttcgtcgagttggggtcgttggtt

SEQ ID NO: 564 - DNA, 27 bp, Homo sapiens
  source 1..27: mol_type = unassigned DNA
gacccttcccgacaaccatctcgaaca

SEQ ID NO: 565 - DNA, 28 bp, Homo sapiens
  source 1..28: mol_type = unassigned DNA
gaaaattgcgcggttgggttagtagggg

SEQ ID NO: 566 - DNA, 28 bp, Homo sapiens
  source 1..28: mol_type = unassigned DNA
acctacaaataccgtccccacccgaaac

SEQ ID NO: 567 - DNA, 28 bp, Homo sapiens
  source 1..28: mol_type = unassigned DNA
aagaggggtgtgattcgcgagtttagat

SEQ ID NO: 568 - DNA, 23 bp, Homo sapiens
  source 1..23: mol_type = unassigned DNA
ccgcgcgcgactcgaacgaaaaa

SEQ ID NO: 569 - DNA, 28 bp, Homo sapiens
  source 1..28: mol_type = unassigned DNA
aagggtgcgatgttttcgtttaggatcg

SEQ ID NO: 570 - DNA, 29 bp, Homo sapiens
  source 1..29: mol_type = unassigned DNA
taactaactaaaaccgcgataaaacgact

SEQ ID NO: 571 - DNA, 28 bp, Homo sapiens
  source 1..28: mol_type = unassigned DNA
tggtaatcgcgtaggtgtgtgatagggc

SEQ ID NO: 572 - DNA, 28 bp, Homo sapiens
  source 1..28: mol_type = unassigned DNA
aaaatacaaaatacgcccccgaccccga

SEQ ID NO: 573 - DNA, 28 bp, Homo sapiens
  source 1..28: mol_type = unassigned DNA
tgaggagagattcggagtagttagtaga

SEQ ID NO: 574 - DNA, 28 bp, Homo sapiens
  source 1..28: mol_type = unassigned DNA
ccctatcacacacctacgcgattaccaa

SEQ ID NO: 575 - DNA, 28 bp, Homo sapiens
  source 1..28: mol_type = unassigned DNA
tttcgaaaagttggtagtcggcggttgg

SEQ ID NO: 576 - DNA, 28 bp, Homo sapiens
  source 1..28: mol_type = unassigned DNA
cattctactcccccgaatcgaaaccccc

SEQ ID NO: 577 - DNA, 28 bp, Homo sapiens
  source 1..28: mol_type = unassigned DNA
aagaggaagagttcgcgcgtcgagttta

SEQ ID NO: 578 - DNA, 28 bp, Homo sapiens
  source 1..28: mol_type = unassigned DNA
gaaatcgcgcgcccacgatactacaaaa

SEQ ID NO: 579 - DNA, 25 bp, Homo sapiens
  source 1..25: mol_type = unassigned DNA
ttattagtaggcggcgtcgggggtt

SEQ ID NO: 580 - DNA, 29 bp, Homo sapiens
  source 1..29: mol_type = unassigned DNA
cgaaaacccctactccgaaaaatcgtccg

SEQ ID NO: 581 - DNA, 28 bp, Homo sapiens
  source 1..28: mol_type = unassigned DNA
gttgagatatcgaggggttcgggttagg

SEQ ID NO: 582 - DNA, 30 bp, Homo sapiens
  source 1..30: mol_type = unassigned DNA
cgccaacaacgataaaataaataccgcgcc

SEQ ID NO: 583 - DNA, 29 bp, Homo sapiens
  source 1..29: mol_type = unassigned DNA
cgtttgttcgatcggggtcgtacgagtat

SEQ ID NO: 584 - DNA, 28 bp, Homo sapiens
  source 1..28: mol_type = unassigned DNA
tttccgcctcctaccttctaacccgact

SEQ ID NO: 585 - DNA, 28 bp, Homo sapiens
  source 1..28: mol_type = unassigned DNA
ggttgggggagtgggaggtaaattcgtt

SEQ ID NO: 586 - DNA, 28 bp, Homo sapiens
  source 1..28: mol_type = unassigned DNA
ctaaacgctccctcacgccttaccttca

SEQ ID NO: 587 - DNA, 28 bp, Homo sapiens
  source 1..28: mol_type = unassigned DNA
ttgagggaaacgcggtgggaatcgtttt

SEQ ID NO: 588 - DNA, 28 bp, Homo sapiens
  source 1..28: mol_type = unassigned DNA
ccgtaactcgcccgaaaaactaaccgaa

SEQ ID NO: 589 - DNA, 22 bp, Homo sapiens
  source 1..22: mol_type = unassigned DNA
tgattggttgcggggtagtttc

SEQ ID NO: 590 - DNA, 24 bp, Homo sapiens
  source 1..24: mol_type = unassigned DNA
acacccgctttaaaataccgctaa

SEQ ID NO: 591 - DNA, 28 bp, Homo sapiens
  source 1..28: mol_type = unassigned DNA
gcgtttcgggtcgttcgttttatttcgc

SEQ ID NO: 592 - DNA, 28 bp, Homo sapiens
  source 1..28: mol_type = unassigned DNA
cgacaacctacgccgaatatacgcacct

SEQ ID NO: 593 - DNA, 28 bp, Homo sapiens
  source 1..28: mol_type = unassigned DNA
gaagggatgaggttgaggttggaggtcg

SEQ ID NO: 594 - DNA, 29 bp, Homo sapiens
  source 1..29: mol_type = unassigned DNA
acctcctacccaccaattccgaaaaacaa

SEQ ID NO: 595 - DNA, 24 bp, Homo sapiens
  source 1..24: mol_type = unassigned DNA
ttacggagttttaggcggcgttac

SEQ ID NO: 596 - DNA, 23 bp, Homo sapiens
  source 1..23: mol_type = unassigned DNA
catttccctctctacgcgcgaac

SEQ ID NO: 597 - DNA, 28 bp, Homo sapiens
  source 1..28: mol_type = unassigned DNA
cgtaggttttgttggagcgagagatcgg

SEQ ID NO: 598 - DNA, 29 bp, Homo sapiens
  source 1..29: mol_type = unassigned DNA
acatataaaaccgcgctacccgaaaaccg

SEQ ID NO: 599 - DNA, 28 bp, Homo sapiens
  source 1..28: mol_type = unassigned DNA
tgaaaggggagaggggaatgttattgtt

SEQ ID NO: 600 - DNA, 28 bp, Homo sapiens
  source 1..28: mol_type = unassigned DNA
aatattctcgcaaacccaccgccaaacc

SEQ ID NO: 601 - DNA, 28 bp, Homo sapiens
  source 1..28: mol_type = unassigned DNA
aaagagattcgtgttgcggcggatgaag

SEQ ID NO: 602 - DNA, 28 bp, Homo sapiens
  source 1..28: mol_type = unassigned DNA
gatcaacactcgaacccgaactttccgc

SEQ ID NO: 603 - DNA, 28 bp, Homo sapiens
  source 1..28: mol_type = unassigned DNA
aagggaggagggtggatcgaaagcgtta

SEQ ID NO: 604 - DNA, 29 bp, Homo sapiens
  source 1..29: mol_type = unassigned DNA
cgaaaacctttacacgcgcacaaactacg

SEQ ID NO: 605 - DNA, 28 bp, Homo sapiens
  source 1..28: mol_type = unassigned DNA
tatgggttgcgtcgagggtaaggtagtg

SEQ ID NO: 606 - DNA, 29 bp, Homo sapiens
  source 1..29: mol_type = unassigned DNA
accatcgccgttcttacctttcgtctaca

SEQ ID NO: 607 - DNA, 28 bp, Homo sapiens
  source 1..28: mol_type = unassigned DNA
tttttaattcggttcggcgttgatttgt

SEQ ID NO: 608 - DNA, 21 bp, Homo sapiens
  source 1..21: mol_type = unassigned DNA
acaaccgcgcgctcccgatac

SEQ ID NO: 609 - DNA, 27 bp, Homo sapiens
  source 1..27: mol_type = unassigned DNA
tagcgcggaagttgtgagtttaaggcg

SEQ ID NO: 610 - DNA, 29 bp, Homo sapiens
  source 1..29: mol_type = unassigned DNA
tcctctaaacaccatcgaaacccccgaac

SEQ ID NO: 611 - DNA, 28 bp, Homo sapiens
  source 1..28: mol_type = unassigned DNA
agtggcgttcgttgagattagggaaggg

SEQ ID NO: 612 - DNA, 28 bp, Homo sapiens
  source 1..28: mol_type = unassigned DNA
accgtacgctaccgaaacgacctttaca

SEQ ID NO: 613 - DNA, 22 bp, Homo sapiens
  source 1..22: mol_type = unassigned DNA
gtttgtaattggtatgagcggc

SEQ ID NO: 614 - DNA, 18 bp, Homo sapiens
  source 1..18: mol_type = unassigned DNA
ataacgaaacgacgcctc

SEQ ID NO: 615 - DNA, 20 bp, Homo sapiens
  source 1..20: mol_type = unassigned DNA
gtaattggtatgagcggcgt

SEQ ID NO: 616 - DNA, 21 bp, Homo sapiens
  source 1..21: mol_type = unassigned DNA
gcctccgcgaaataaaaccat

SEQ ID NO: 617 - DNA, 21 bp, Homo sapiens
  source 1..21: mol_type = unassigned DNA
agttagagtgggttaggggat

SEQ ID NO: 618 - DNA, 20 bp, Homo sapiens
  source 1..20: mol_type = unassigned DNA
acgcgtaacacaaacacgac

SEQ ID NO: 619 - DNA, 20 bp, Homo sapiens
  source 1..20: mol_type = unassigned DNA
gggggattttaaagatcgtc

SEQ ID NO: 620 - DNA, 18 bp, Homo sapiens
  source 1..18: mol_type = unassigned DNA
gacgaacgcaatccacaa

SEQ ID NO: 621 - DNA, 20 bp, Homo sapiens
  source 1..20: mol_type = unassigned DNA
tggtggagttgtggattgcg

SEQ ID NO: 622 - DNA, 20 bp, Homo sapiens
  source 1..20: mol_type = unassigned DNA
tcccacccaaacctctctct

SEQ ID NO: 623 - DNA, 18 bp, Homo sapiens
  source 1..18: mol_type = unassigned DNA
ggtgcgtttacgggtttc

SEQ ID NO: 624 - DNA, 21 bp, Homo sapiens
  source 1..21: mol_type = unassigned DNA
acctaatccgatatttcccga

SEQ ID NO: 625 - DNA, 20 bp, Homo sapiens
  source 1..20: mol_type = unassigned DNA
ggtagggtttcggttgcgta

SEQ ID NO: 626 - DNA, 24 bp, Homo sapiens
  source 1..24: mol_type = unassigned DNA
tatttcccgaaaactccacatcca

SEQ ID NO: 627 - DNA, 20 bp, Homo sapiens
  source 1..20: mol_type = unassigned DNA
tcccgaaaactccacatcca

SEQ ID NO: 628 - DNA, 20 bp, Homo sapiens
  source 1..20: mol_type = unassigned DNA
atttggatgtttcgcgtttc

SEQ ID NO: 629 - DNA, 20 bp, Homo sapiens
  source 1..20: mol_type = unassigned DNA
tatcgctacgacccgactaa

SEQ ID NO: 630 - DNA, 24 bp, Homo sapiens
  source 1..24: mol_type = unassigned DNA
gttaatttggatgtttcgcgtttc

SEQ ID NO: 631 - DNA, 23 bp, Homo sapiens
  source 1..23: mol_type = unassigned DNA
gtttatcgctacgacccgactaa

SEQ ID NO: 632 - DNA, 20 bp, Homo sapiens
  source 1..20: mol_type = unassigned DNA
aggggagtcgcgtttttagg

SEQ ID NO: 633 - DNA, 20 bp, Homo sapiens
  source 1..20: mol_type = unassigned DNA
tcccgaccgaaacccaaatc

SEQ ID NO: 634 - DNA, 22 bp, Homo sapiens
  source 1..22: mol_type = unassigned DNA
gaataacggcgtaagtttttac

SEQ ID NO: 635 - DNA, 18 bp, Homo sapiens
  source 1..18: mol_type = unassigned DNA
atcctcccgaacgcaata

SEQ ID NO: 636 - DNA, 21 bp, Homo sapiens
  source 1..21: mol_type = unassigned DNA
ttgtacgtttgtgggtgtgga

SEQ ID NO: 637 - DNA, 21 bp, Homo sapiens
  source 1..21: mol_type = unassigned DNA
tcctcccgaacgcaataatcg

SEQ ID NO: 638 - DNA, 21 bp, Homo sapiens
  source 1..21: mol_type = unassigned DNA
ttaacggttaggttagatcgc

SEQ ID NO: 639 - DNA, 20 bp, Homo sapiens
  source 1..20: mol_type = unassigned DNA
caatctctaaaacgcgacac

SEQ ID NO: 640 - DNA, 21 bp, Homo sapiens
  source 1..21: mol_type = unassigned DNA
cgggtgtcgcgttttagagat

SEQ ID NO: 641 - DNA, 21 bp, Homo sapiens
  source 1..21: mol_type = unassigned DNA
ttctccgatctcataccccct

SEQ ID NO: 642 - DNA, 19 bp, Homo sapiens
  source 1..19: mol_type = unassigned DNA
gagaaaagttgtttcggtc

SEQ ID NO: 643 - DNA, 20 bp, Homo sapiens
  source 1..20: mol_type = unassigned DNA
gctacgtctctactatccga

SEQ ID NO: 644 - DNA, 22 bp, Homo sapiens
  source 1..22: mol_type = unassigned DNA
cgggagaaaagttgtttcggtc

SEQ ID NO: 645 - DNA, 22 bp, Homo sapiens
  source 1..22: mol_type = unassigned DNA
ccgctacgtctctactatccga

SEQ ID NO: 646 - DNA, 18 bp, Homo sapiens
  source 1..18: mol_type = unassigned DNA
ttcggggtgagggtagtc

SEQ ID NO: 647 - DNA, 18 bp, Homo sapiens
  source 1..18: mol_type = unassigned DNA
ccgacgcccaactaaaaa

SEQ ID NO: 648 - DNA, 21 bp, Homo sapiens
  source 1..21: mol_type = unassigned DNA
gagttcggggtgagggtagtc

SEQ ID NO: 649 - DNA, 23 bp, Homo sapiens
  source 1..23: mol_type = unassigned DNA
gaatcccgacgcccaactaaaaa

SEQ ID NO: 650 - DNA, 20 bp, Homo sapiens
  source 1..20: mol_type = unassigned DNA
ttttcgggttacgggtcgtt

SEQ ID NO: 651 - DNA, 20 bp, Homo sapiens
  source 1..20: mol_type = unassigned DNA
acgactcctccgaaaatccg

SEQ ID NO: 652 - DNA, 19 bp, Homo sapiens
  source 1..19: mol_type = unassigned DNA
gtcgatttttgttttgagc

SEQ ID NO: 653 - DNA, 22 bp, Homo sapiens
  source 1..22: mol_type = unassigned DNA
taaaaataatctaccgaatcgc

SEQ ID NO: 654 - DNA, 20 bp, Homo sapiens
  source 1..20: mol_type = unassigned DNA
ggcgttagcggggatttaga

SEQ ID NO: 655 - DNA, 20 bp, Homo sapiens
  source 1..20: mol_type = unassigned DNA
cgcatcaaacgaaaccctcc

SEQ ID NO: 656 - DNA, 28 bp, Homo sapiens
  source 1..28: mol_type = unassigned DNA
cgggaaggtgttcgtttaatggttcggt

SEQ ID NO: 657 - DNA, 28 bp, Homo sapiens
  source 1..28: mol_type = unassigned DNA
gtttccgctctaaatccccgctaacgcc

SEQ ID NO: 658 - DNA, 28 bp, Homo sapiens
  source 1..28: mol_type = unassigned DNA
ggaaaaaggaggaggataagaagcgcgg

SEQ ID NO: 659 - DNA, 28 bp, Homo sapiens
  source 1..28: mol_type = unassigned DNA
ctcgccgaaaatcacgacgcaatcctac

SEQ ID NO: 660 - DNA, 19 bp, Homo sapiens
  source 1..19: mol_type = unassigned DNA
taggggaggcgtcgagttc

SEQ ID NO: 661 - DNA, 20 bp, Homo sapiens
  source 1..20: mol_type = unassigned DNA
actcgctaaacgtcccaacc

SEQ ID NO: 662 - DNA, 18 bp, Homo sapiens
  source 1..18: mol_type = unassigned DNA
gagtttaggggtcgcgtc

SEQ ID NO: 663 - DNA, 20 bp, Homo sapiens
  source 1..20: mol_type = unassigned DNA
caataccgccgcctctacta

SEQ ID NO: 664 - DNA, 20 bp, Homo sapiens
  source 1..20: mol_type = unassigned DNA
gagagagtaggagcggatcg

SEQ ID NO: 665 - DNA, 20 bp, Homo sapiens
  source 1..20: mol_type = unassigned DNA
acaaatcaaccccgccctaa

SEQ ID NO: 666 - DNA, 20 bp, Homo sapiens
  source 1..20: mol_type = unassigned DNA
agtgagtgttcgggagtttc

SEQ ID NO: 667 - DNA, 20 bp, Homo sapiens
  source 1..20: mol_type = unassigned DNA
tcatttattaaaaacgcgcg

SEQ ID NO: 668 - DNA, 22 bp, Homo sapiens
  source 1..22: mol_type = unassigned DNA
gcagtgagtgctcgggagcccc

SEQ ID NO: 669 - DNA, 25 bp, Homo sapiens
  source 1..25: mol_type = unassigned DNA
ggttttcatttgttagaggcgcgcg

SEQ ID NO: 670 - DNA, 20 bp, Homo sapiens
  source 1..20: mol_type = unassigned DNA
cgggttcgcgtaggattagg

SEQ ID NO: 671 - DNA, 20 bp, Homo sapiens
  source 1..20: mol_type = unassigned DNA
actcctcatcccaacaccct

SEQ ID NO: 672 - DNA, 19 bp, Homo sapiens
  source 1..19: mol_type = unassigned DNA
gttcgtagttcgcggtttc

SEQ ID NO: 673 - DNA, 18 bp, Homo sapiens
  source 1..18: mol_type = unassigned DNA
cgatactctcctcgccct

SEQ ID NO: 674 - DNA, 22 bp, Homo sapiens
  source 1..22: mol_type = unassigned DNA
tcggttcgtagttcgcggtttc

SEQ ID NO: 675 - DNA, 21 bp, Homo sapiens
  source 1..21: mol_type = unassigned DNA
gcacgatactctcctcgccct

SEQ ID NO: 676 - DNA, 19 bp, Homo sapiens
  source 1..19: mol_type = unassigned DNA
ttgggcgagtgatagtttc

SEQ ID NO: 677 - DNA, 18 bp, Homo sapiens
  source 1..18: mol_type = unassigned DNA
gaccgctactacacccga

SEQ ID NO: 678 - DNA, 23 bp, Homo sapiens
  source 1..23: mol_type = unassigned DNA
cggtagaagaacgtgtatgaggt

SEQ ID NO: 679 - DNA, 20 bp, Homo sapiens
  source 1..20: mol_type = unassigned DNA
gctaccctcgaaaacccgaa

SEQ ID NO: 680 - DNA, 20 bp, Homo sapiens
  source 1..20: mol_type = unassigned DNA
gattttttttaaggtcgcgc

SEQ ID NO: 681 - DNA, 24 bp, Homo sapiens
  source 1..24: mol_type = unassigned DNA
ttacgattttttttaaggtcgcgc

SEQ ID NO: 682 - DNA, 22 bp, Homo sapiens
  source 1..22: mol_type = unassigned DNA
actatacctacctaccgccgtc

SEQ ID NO: 683 - DNA, 20 bp, Homo sapiens
  source 1..20: mol_type = unassigned DNA
atttcgaagaaggcgggtcg

SEQ ID NO: 684 - DNA, 20 bp, Homo sapiens
  source 1..20: mol_type = unassigned DNA
ctccaaacgatacgccaacg

SEQ ID NO: 685 - DNA, 18 bp, Homo sapiens
  source 1..18: mol_type = unassigned DNA
ttttgcgggtaagcgttc

SEQ ID NO: 686 - DNA, 19 bp, Homo sapiens
  source 1..19: mol_type = unassigned DNA
ccacaactctctcgacgac

SEQ ID NO: 687 - DNA, 22 bp, Homo sapiens
  source 1..22: mol_type = unassigned DNA
tgttttttgcgggtaagcgttc

SEQ ID NO: 688 - DNA, 21 bp, Homo sapiens
  source 1..21: mol_type = unassigned DNA
gcccacaactctctcgacgac

SEQ ID NO: 689 - DNA, 20 bp, Homo sapiens
  source 1..20: mol_type = unassigned DNA
atttcgggaaagggtgggtc

SEQ ID NO: 690 - DNA, 20 bp, Homo sapiens
  source 1..20: mol_type = unassigned DNA
accctaatcccccttcacca

SEQ ID NO: 691 - DNA, 20 bp, Homo sapiens
  source 1..20: mol_type = unassigned DNA
tttcgtttcgtttcggtcgc

SEQ ID NO: 692 - DNA, 20 bp, Homo sapiens
  source 1..20: mol_type = unassigned DNA
aacccgcccgaactcaaata

SEQ ID NO: 693 - DNA, 21 bp, Homo sapiens
  source 1..21: mol_type = unassigned DNA
attaggagcgtacgtttattc

SEQ ID NO: 694 - DNA, 18 bp, Homo sapiens
  source 1..18: mol_type = unassigned DNA
tacgcactcgaaacacaa

SEQ ID NO: 695 - DNA, 20 bp, Homo sapiens
  source 1..20: mol_type = unassigned DNA
cggcgcgttttaagggtttt

SEQ ID NO: 696 - DNA, 20 bp, Homo sapiens
  source 1..20: mol_type = unassigned DNA
attactctcacctccgcacg

SEQ ID NO: 697 - DNA, 19 bp, Homo sapiens
  source 1..19: mol_type = unassigned DNA
gattgcgggaagaaggtac

SEQ ID NO: 698 - DNA, 19 bp, Homo sapiens
  source 1..19: mol_type = unassigned DNA
aaacgaaaccaaacgacaa

SEQ ID NO: 699 - DNA, 21 bp, Homo sapiens
  source 1..21: mol_type = unassigned DNA
cggattgcgggaagaaggtac

SEQ ID NO: 700 - DNA, 23 bp, Homo sapiens
  source 1..23: mol_type = unassigned DNA
gacgaaacgaaaccaaacgacaa

SEQ ID NO: 701 - DNA, 20 bp, Homo sapiens
  source 1..20: mol_type = unassigned DNA
ttttcgagtttgaagcgttc

SEQ ID NO: 702 - DNA, 19 bp, Homo sapiens
  source 1..19: mol_type = unassigned DNA
cgactctcacctaatccgc

SEQ ID NO: 703 - DNA, 23 bp, Homo sapiens
  source 1..23: mol_type = unassigned DNA
cggttttcgagtttgaagcgttc

SEQ ID NO: 704 - DNA, 22 bp, Homo sapiens
  source 1..22: mol_type = unassigned DNA
taccgactctcacctaatccgc

SEQ ID NO: 705 - DNA, 20 bp, Homo sapiens
  source 1..20: mol_type = unassigned DNA
ttacgacggggagttcgttc

SEQ ID NO: 706 - DNA, 24 bp, Homo sapiens
  source 1..24: mol_type = unassigned DNA
cttaacaacgttcgcaaatcacga

SEQ ID NO: 707 - DNA, 20 bp, Homo sapiens
  source 1..20: mol_type = unassigned DNA
acaacgttcgcaaatcacga

SEQ ID NO: 708 - DNA, 19 bp, Homo sapiens
  source 1..19: mol_type = unassigned DNA
gttcgttatttcggaattc

SEQ ID NO: 709 - DNA, 21 bp, Homo sapiens
  source 1..21: mol_type = unassigned DNA
ccgaccgataaaatataattc

SEQ ID NO: 710 - DNA, 20 bp, Homo sapiens
  source 1..20: mol_type = unassigned DNA
gggagggatttaagcgggag

SEQ ID NO: 711 - DNA, 20 bp, Homo sapiens
  source 1..20: mol_type = unassigned DNA
cccccttcactaatcccgac

SEQ ID NO: 712 - DNA, 18 bp, Homo sapiens
  source 1..18: mol_type = unassigned DNA
agtgttgagagtcgacgc

SEQ ID NO: 713 - DNA, 20 bp, Homo sapiens
  source 1..20: mol_type = unassigned DNA
aataaaataacccgaaccgc

SEQ ID NO: 714 - DNA, 20 bp, Homo sapiens
  source 1..20: mol_type = unassigned DNA
gggttacggtttcgggttgt

SEQ ID NO: 715 - DNA, 20 bp, Homo sapiens
  source 1..20: mol_type = unassigned DNA
cgcgtcgactctcaacacta

SEQ ID NO: 716 - DNA, 19 bp, Homo sapiens
  source 1..19: mol_type = unassigned DNA
ggtcgagtcgagtcgttac

SEQ ID NO: 717 - DNA, 18 bp, Homo sapiens
  source 1..18: mol_type = unassigned DNA
aaaacgcctcctaacgaa

SEQ ID NO: 718 - DNA, 22 bp, Homo sapiens
  source 1..22: mol_type = unassigned DNA
attggtcgagtcgagtcgttac

SEQ ID NO: 719 - DNA, 23 bp, Homo sapiens
  source 1..23: mol_type = unassigned DNA
acaaaaaaacgcctcctaacgaa

SEQ ID NO: 720 - DNA, 21 bp, Homo sapiens
  source 1..21: mol_type = unassigned DNA
tatttttgggcgaaggcgttg

SEQ ID NO: 721 - DNA, 20 bp, Homo sapiens
  source 1..20: mol_type = unassigned DNA
gtaacgactcgactcgacca

SEQ ID NO: 722 - DNA, 18 bp, Homo sapiens
  source 1..18: mol_type = unassigned DNA
tcggcgttttcgtttttc

SEQ ID NO: 723 - DNA, 22 bp, Homo sapiens
  source 1..22: mol_type = unassigned DNA
cgacgacacaaccataaacttt

SEQ ID NO: 724 - DNA, 20 bp, Homo sapiens
  source 1..20: mol_type = unassigned DNA
aaggttttgtagttgcggcg

SEQ ID NO: 725 - DNA, 18 bp, Homo sapiens
  source 1..18: mol_type = unassigned DNA
tctcacgcgcaaccgaat

SEQ ID NO: 726 - DNA, 19 bp, Homo sapiens
  source 1..19: mol_type = unassigned DNA
gtggagttttaggtagcgc

SEQ ID NO: 727 - DNA, 19 bp, Homo sapiens
  source 1..19: mol_type = unassigned DNA
acccgcgataaactaaacc

SEQ ID NO: 728 - DNA, 22 bp, Homo sapiens
  source 1..22: mol_type = unassigned DNA
agggtggagttttaggtagcgc

SEQ ID NO: 729 - DNA, 23 bp, Homo sapiens
  source 1..23: mol_type = unassigned DNA
aacaacccgcgataaactaaacc

SEQ ID NO: 730 - DNA, 20 bp, Homo sapiens
  source 1..20: mol_type = unassigned DNA
tgtagtcgtggttgtcgtgg

SEQ ID NO: 731 - DNA, 20 bp, Homo sapiens
  source 1..20: mol_type = unassigned DNA
ccaaataaacgacgtcccgc

SEQ ID NO: 732 - DNA, 22 bp, Homo sapiens
  source 1..22: mol_type = unassigned DNA
attttatagtcgcgttaaaagc

SEQ ID NO: 733 - DNA, 20 bp, Homo sapiens
  source 1..20: mol_type = unassigned DNA
acttttattactcgcgatcc

SEQ ID NO: 734 - DNA, 20 bp, Homo sapiens
  source 1..20: mol_type = unassigned DNA
ggtagggtgagtttggtcgg

SEQ ID NO: 735 - DNA, 20 bp, Homo sapiens
  source 1..20: mol_type = unassigned DNA
cgccgaaccacgtaaaaact

SEQ ID NO: 736 - DNA, 20 bp, Homo sapiens
  source 1..20: mol_type = unassigned DNA
atttggggcgtttatgtttc

SEQ ID NO: 737 - DNA, 18 bp, Homo sapiens
  source 1..18: mol_type = unassigned DNA
ccctcgaaaaacgactcc

SEQ ID NO: 738 - DNA, 18 bp, Homo sapiens
  source 1..18: mol_type = unassigned DNA
aggggttgtagggtcggg

SEQ ID NO: 739 - DNA, 21 bp, Homo sapiens
  source 1..21: mol_type = unassigned DNA
tttaggggttgtagggtcggg

SEQ ID NO: 740 - DNA, 22 bp, Homo sapiens
  source 1..22: mol_type = unassigned DNA
attttacatttccctcccccgc

SEQ ID NO: 741 - DNA, 18 bp, Homo sapiens
  source 1..18: mol_type = unassigned DNA
agaataaaagtaggcggc

SEQ ID NO: 742 - DNA, 20 bp, Homo sapiens
  source 1..20: mol_type = unassigned DNA
tctcgaaaccaaaataaacg

SEQ ID NO: 743 - DNA, 20 bp, Homo sapiens
  source 1..20: mol_type = unassigned DNA
agtaggcggcggatttgtag

SEQ ID NO: 744 - DNA, 22 bp, Homo sapiens
  source 1..22: mol_type = unassigned DNA
ccgaaaatacgcgaaatcaacc

SEQ ID NO: 745 - DNA, 19 bp, Homo sapiens
  source 1..19: mol_type = unassigned DNA
gaggagataaaggtgtcgc

SEQ ID NO: 746 - DNA, 20 bp, Homo sapiens
  source 1..20: mol_type = unassigned DNA
aacgtacctaacccgaaaac

SEQ ID NO: 747 - DNA, 22 bp, Homo sapiens
  source 1..22: mol_type = unassigned DNA
tcggaggagataaaggtgtcgc

SEQ ID NO: 748 - DNA, 22 bp, Homo sapiens
  source 1..22: mol_type = unassigned DNA
ccaacgtacctaacccgaaaac

SEQ ID NO: 749 - DNA, 18 bp, Homo sapiens
  source 1..18: mol_type = unassigned DNA
gacggtttcggtagggtc

SEQ ID NO: 750 - DNA, 20 bp, Homo sapiens
  source 1..20: mol_type = unassigned DNA
ccgaaccgaatataaaacga

SEQ ID NO: 751 - DNA, 20 bp, Homo sapiens
  source 1..20: mol_type = unassigned DNA
cggacggtttcggtagggtc

SEQ ID NO: 752 - DNA, 23 bp, Homo sapiens
  source 1..23: mol_type = unassigned DNA
gcgccgaaccgaatataaaacga

SEQ ID NO: 753 - DNA, 19 bp, Homo sapiens
  source 1..19: mol_type = unassigned DNA
ttaggttcgtaaagagggc

SEQ ID NO: 754 - DNA, 19 bp, Homo sapiens
  source 1..19: mol_type = unassigned DNA
ttaaaaccacgtccgaata

SEQ ID NO: 755 - DNA, 20 bp, Homo sapiens
  source 1..20: mol_type = unassigned DNA
tttcgggcgggagttatagg

SEQ ID NO: 756 - DNA, 20 bp, Homo sapiens
  source 1..20: mol_type = unassigned DNA
acgcgctctaaactcaaccg

SEQ ID NO: 757 - DNA, 26 bp, Homo sapiens
  source 1..26: mol_type = unassigned DNA
gcgcgtggggttcgtagcgttttaag

SEQ ID NO: 758 - DNA, 25 bp, Homo sapiens
  source 1..25: mol_type = unassigned DNA
ttacccgaaacaccccgcgcccttc

SEQ ID NO: 759 - DNA, 28 bp, Homo sapiens
  source 1..28: mol_type = unassigned DNA
agatacggagatttagcgcgagatcggt

SEQ ID NO: 760 - DNA, 28 bp, Homo sapiens
  source 1..28: mol_type = unassigned DNA
aaattaaccgccgaacactcacaatacg

SEQ ID NO: 761 - DNA, 28 bp, Homo sapiens
  source 1..28: mol_type = unassigned DNA
ttgtagtgtcgcgttgcgagtcgattgt

SEQ ID NO: 762 - DNA, 29 bp, Homo sapiens
  source 1..29: mol_type = unassigned DNA
acaataacgtaacgcccataaaccgaacg

SEQ ID NO: 763 - DNA, 28 bp, Homo sapiens
  source 1..28: mol_type = unassigned DNA
gaggacgggttgaatcgtggtttgttgg

SEQ ID NO: 764 - DNA, 28 bp, Homo sapiens
  source 1..28: mol_type = unassigned DNA
actacgataatcaaaacgctccacgcga

SEQ ID NO: 765 - DNA, 27 bp, Homo sapiens
  source 1..27: mol_type = unassigned DNA
gtgcgcgttttagtagggcgagaatgg

SEQ ID NO: 766 - DNA, 28 bp, Homo sapiens
  source 1..28: mol_type = unassigned DNA
cgaaaaccaaatccgaaccaccgtctcc

SEQ ID NO: 767 - DNA, 28 bp, Homo sapiens
  source 1..28: mol_type = unassigned DNA
tgatttgggtggatgtagaggttgtggt

SEQ ID NO: 768 - DNA, 28 bp, Homo sapiens
  source 1..28: mol_type = unassigned DNA
tttcgaataacgctactccgaaccgcga

SEQ ID NO: 769 - DNA, 28 bp, Homo sapiens
  source 1..28: mol_type = unassigned DNA
ttgagagtagggattgtggtgcgtcgtc

SEQ ID NO: 770 - DNA, 28 bp, Homo sapiens
  source 1..28: mol_type = unassigned DNA
ctaactcccgaacgctacattcgctcca

SEQ ID NO: 771 - DNA, 27 bp, Homo sapiens
  source 1..27: mol_type = unassigned DNA
ggttgtggtgagtttggtttacgggcg

SEQ ID NO: 772 - DNA, 26 bp, Homo sapiens
  source 1..26: mol_type = unassigned DNA
cgtaaaacgcgaccaccgccaacata

SEQ ID NO: 773 - DNA, 28 bp, Homo sapiens
  source 1..28: mol_type = unassigned DNA
gggaggttattcgtaggatttggcgcgg

SEQ ID NO: 774 - DNA, 28 bp, Homo sapiens
  source 1..28: mol_type = unassigned DNA
atcctaacgactacgcactacttccgca

SEQ ID NO: 775 - DNA, 24 bp, Homo sapiens
  source 1..24: mol_type = unassigned DNA
gagttcgtttagttcgtcggcgtc

SEQ ID NO: 776 - DNA, 27 bp, Homo sapiens
  source 1..27: mol_type = unassigned DNA
aaccccgataaactccgataacgacct

SEQ ID NO: 777 - DNA, 28 bp, Homo sapiens
  source 1..28: mol_type = unassigned DNA
agagttgggggcggtatagttagggtgt

SEQ ID NO: 778 - DNA, 28 bp, Homo sapiens
  source 1..28: mol_type = unassigned DNA
ttcaatccctacgaccccaacgcctaaa

SEQ ID NO: 779 - DNA, 28 bp, Homo sapiens
  source 1..28: mol_type = unassigned DNA
cgtggatacgagttttggcggcgattat

SEQ ID NO: 780 - DNA, 28 bp, Homo sapiens
  source 1..28: mol_type = unassigned DNA
gccaccaaccctacctccttccatatcc

SEQ ID NO: 781 - DNA, 28 bp, Homo sapiens
  source 1..28: mol_type = unassigned DNA
tttttcggtttgagttatcgtggcggga

SEQ ID NO: 782 - DNA, 28 bp, Homo sapiens
  source 1..28: mol_type = unassigned DNA
cgaaccgtacttccaaccaaacgcaact

SEQ ID NO: 783 - DNA, 28 bp, Homo sapiens
  source 1..28: mol_type = unassigned DNA
taggaaggggtcgatgttggtttgggtt

SEQ ID NO: 784 - DNA, 28 bp, Homo sapiens
  source 1..28: mol_type = unassigned DNA
tctcaccaactcccatcgaattcgcaca

SEQ ID NO: 785 - DNA, 28 bp, Homo sapiens
  source 1..28: mol_type = unassigned DNA
gttttggtttcgtttcggagcgcgtaga

SEQ ID NO: 786 - DNA, 28 bp, Homo sapiens
  source 1..28: mol_type = unassigned DNA
tttctctaccgactcaactccccctccc

SEQ ID NO: 787 - DNA, 28 bp, Homo sapiens
  source 1..28: mol_type = unassigned DNA
tcggttgcgtaaatcgcgtttttggttg

SEQ ID NO: 788 - DNA, 28 bp, Homo sapiens
  source 1..28: mol_type = unassigned DNA
ttctcgataatatctccgtcgcctccgc

SEQ ID NO: 789 - DNA, 27 bp, Homo sapiens
  source 1..27: mol_type = unassigned DNA
gtttaggggtggaggtcggggttttga

SEQ ID NO: 790 - DNA, 28 bp, Homo sapiens
  source 1..28: mol_type = unassigned DNA
ccgaaccccgcgcaaataaaaacaacct

SEQ ID NO: 791 - DNA, 28 bp, Homo sapiens
  source 1..28: mol_type = unassigned DNA
ggggcgcgtttttatggaaagttagggt

SEQ ID NO: 792 - DNA, 28 bp, Homo sapiens
  source 1..28: mol_type = unassigned DNA
ctacgccctaaaacacgcctcgacttct

SEQ ID NO: 793 - DNA, 28 bp, Homo sapiens
  source 1..28: mol_type = unassigned DNA
tgtgcgaaagagacgcggggtttagtta

SEQ ID NO: 794 - DNA, 28 bp, Homo sapiens
  source 1..28: mol_type = unassigned DNA
cccgtaatcgctaaaacatccgccctta

SEQ ID NO: 795 - DNA, 26 bp, Homo sapiens
  source 1..26: mol_type = unassigned DNA
cgtcgggcgatgttggtttgttcgtg

SEQ ID NO: 796 - DNA, 29 bp, Homo sapiens
  source 1..29: mol_type = unassigned DNA
gcgacgctccaccgtaaacccaatattta

SEQ ID NO: 797 - DNA, 27 bp, Homo sapiens
  source 1..27: mol_type = unassigned DNA
cggggagggtcgagggttttgtttgag

SEQ ID NO: 798 - DNA, 28 bp, Homo sapiens
  source 1..28: mol_type = unassigned DNA
gcgtcccaaacttcattcaaccgacgac

SEQ ID NO: 799 - DNA, 28 bp, Homo sapiens
  source 1..28: mol_type = unassigned DNA
gcggacgtagtaatggattaaacgggga

SEQ ID NO: 800 - DNA, 28 bp, Homo sapiens
  source 1..28: mol_type = unassigned DNA
aaatccgactccctacactcccgacttt

SEQ ID NO: 801 - DNA, 29 bp, Homo sapiens
  source 1..29: mol_type = unassigned DNA
gggggttgtgttagttgtttgtttagcga

SEQ ID NO: 802 - DNA, 28 bp, Homo sapiens
  source 1..28: mol_type = unassigned DNA
cgaaactatttcccgccaaaccgaaccc

SEQ ID NO: 803 - DNA, 28 bp, Homo sapiens
  source 1..28: mol_type = unassigned DNA
tttcgggcgggagtatcgggttttgtag

SEQ ID NO: 804 - DNA, 28 bp, Homo sapiens
  source 1..28: mol_type = unassigned DNA
gctcctttaccccttctcgaccaactcc

SEQ ID NO: 805 - DNA, 30 bp, Homo sapiens
  source 1..30: mol_type = unassigned DNA
ttacggattttatttgtattcggaatcgta

SEQ ID NO: 806 - DNA, 29 bp, Homo sapiens
  source 1..29: mol_type = unassigned DNA
acgcatcaaactcgacacaaaatttcatc

SEQ ID NO: 807 - DNA, 28 bp, Homo sapiens
  source 1..28: mol_type = unassigned DNA
ggtgttttcgtaagacggggtagtgggt

SEQ ID NO: 808 - DNA, 28 bp, Homo sapiens
  source 1..28: mol_type = unassigned DNA
ttctcctccgctaaaaatccgaatacga

SEQ ID NO: 809 - DNA, 28 bp, Homo sapiens
  source 1..28: mol_type = unassigned DNA
agggattgtatttcgaggtggtcgaggt

SEQ ID NO: 810 - DNA, 28 bp, Homo sapiens
  source 1..28: mol_type = unassigned DNA
ccgacaaatcgaaaccttcgcccgaaac

SEQ ID NO: 811 - DNA, 28 bp, Homo sapiens
  source 1..28: mol_type = unassigned DNA
tcgcgggttataaatatttggttgcggc

SEQ ID NO: 812 - DNA, 28 bp, Homo sapiens
  source 1..28: mol_type = unassigned DNA
gaccgccactacctcgaaaacatttccc

SEQ ID NO: 813 - DNA, 28 bp, Homo sapiens
  source 1..28: mol_type = unassigned DNA
ggtaacggaaaagcgcgggaattataga

SEQ ID NO: 814 - DNA, 26 bp, Homo sapiens
  source 1..26: mol_type = unassigned DNA
cccacaacctatcccccgtccaaaaa

SEQ ID NO: 815 - DNA, 28 bp, Homo sapiens
  source 1..28: mol_type = unassigned DNA
ttttgtacgttgggttacgggggtttgg

SEQ ID NO: 816 - DNA, 28 bp, Homo sapiens
  source 1..28: mol_type = unassigned DNA
taaacgcgataaacccctacgaccccca

SEQ ID NO: 817 - DNA, 28 bp, Homo sapiens
  source 1..28: mol_type = unassigned DNA
tatcggttttcgtagttgcgggaggagg

SEQ ID NO: 818 - DNA, 28 bp, Homo sapiens
  source 1..28: mol_type = unassigned DNA
ccgaataaataccaaactcgcccgacgc

SEQ ID NO: 819 - DNA, 28 bp, Homo sapiens
  source 1..28: mol_type = unassigned DNA
cgggtagaggggaggtaggaattggaga

SEQ ID NO: 820 - DNA, 28 bp, Homo sapiens
  source 1..28: mol_type = unassigned DNA
ccgaataaacgtcacccctacacaccgc

SEQ ID NO: 821 - DNA, 28 bp, Homo sapiens
  source 1..28: mol_type = unassigned DNA
ttttgcggttaggtgaaggcgtagaggt

SEQ ID NO: 822 - DNA, 28 bp, Homo sapiens
  source 1..28: mol_type = unassigned DNA
gaccgaataccccgctttctctctcgac

SEQ ID NO: 823 - DNA, 27 bp, Homo sapiens
  source 1..27: mol_type = unassigned DNA
aggagtagtattgcgagggtggagggt

SEQ ID NO: 824 - DNA, 28 bp, Homo sapiens
  source 1..28: mol_type = unassigned DNA
taaacccgaaaaacaacgccaatcccgc

SEQ ID NO: 825 - DNA, 28 bp, Homo sapiens
  source 1..28: mol_type = unassigned DNA
ggggatttgttgtagagtcgtaggagaa

SEQ ID NO: 826 - DNA, 25 bp, Homo sapiens
  source 1..25: mol_type = unassigned DNA
ccgcatcccaccctttaaaactcta

SEQ ID NO: 827 - DNA, 28 bp, Homo sapiens
  source 1..28: mol_type = unassigned DNA
tatgggttgcgtcgagggtaaggtagtg

SEQ ID NO: 828 - DNA, 28 bp, Homo sapiens
  source 1..28: mol_type = unassigned DNA
tacgacgaccatcgccgttcttaccttt

SEQ ID NO: 829 - DNA, 28 bp, Homo sapiens
  source 1..28: mol_type = unassigned DNA
aaatttggattgggggagggacgaggtt

SEQ ID NO: 830 - DNA, 28 bp, Homo sapiens
  source 1..28: mol_type = unassigned DNA
cttcgcaaccgaactactcacccccgac

SEQ ID NO: 831 - DNA, 28 bp, Homo sapiens
  source 1..28: mol_type = unassigned DNA
ggtcgttggagtggtcgtttcggtttag

SEQ ID NO: 832 - DNA, 28 bp, Homo sapiens
  source 1..28: mol_type = unassigned DNA
cctcaaacccccgaacgcgctaaataaa

SEQ ID NO: 833 - DNA, 28 bp, Homo sapiens
  source 1..28: mol_type = unassigned DNA
gttcggggagggagggagattcgttttg

SEQ ID NO: 834 - DNA, 28 bp, Homo sapiens
  source 1..28: mol_type = unassigned DNA
aactcccgactttaacctcccaacccaa

SEQ ID NO: 835 - DNA, 28 bp, Homo sapiens
  source 1..28: mol_type = unassigned DNA
gttgtaggggtgtttggtcgggttggta

SEQ ID NO: 836 - DNA, 28 bp, Homo sapiens
  source 1..28: mol_type = unassigned DNA
atcaactactccgtaccccacgtaaccg

SEQ ID NO: 837 - DNA, 25 bp, Homo sapiens
  source 1..25: mol_type = unassigned DNA
agtcggggttggttggtggaagagg

SEQ ID NO: 838 - DNA, 28 bp, Homo sapiens
  source 1..28: mol_type = unassigned DNA
ccctctcaactcgattcaaaattccccc

SEQ ID NO: 839 - DNA, 28 bp, Homo sapiens
  source 1..28: mol_type = unassigned DNA
aaagtaataagtggtttcgggcggagtc

SEQ ID NO: 840 - DNA, 28 bp, Homo sapiens
  source 1..28: mol_type = unassigned DNA
accccgcatacctacgaaaacgaaaacc

SEQ ID NO: 841 - DNA, 27 bp, Homo sapiens
  source 1..27: mol_type = unassigned DNA
cgggattatggaggggtagagcggtcg

SEQ ID NO: 842 - DNA, 28 bp, Homo sapiens
  source 1..28: mol_type = unassigned DNA
acgtccttaacgaacacctacaacaacg

SEQ ID NO: 843 - DNA, 28 bp, Homo sapiens
  source 1..28: mol_type = unassigned DNA
aggttttgtagtagtaggcggacgaggc

SEQ ID NO: 844 - DNA, 28 bp, Homo sapiens
  source 1..28: mol_type = unassigned DNA
acgaatacgaaacccgaaaccgaaacgc

SEQ ID NO: 845 - DNA, 25 bp, Homo sapiens
  source 1..25: mol_type = unassigned DNA
cgtggtatagttaatcgcgcggcgt

SEQ ID NO: 846 - DNA, 28 bp, Homo sapiens
  source 1..28: mol_type = unassigned DNA
tacaaccccaacgccataactcgccaat

SEQ ID NO: 847 - DNA, 28 bp, Homo sapiens
  source 1..28: mol_type = unassigned DNA
tagcggggatttattaggggagaggtgg

SEQ ID NO: 848 - DNA, 28 bp, Homo sapiens
  source 1..28: mol_type = unassigned DNA
atcacctacgaacactatccctcacccg

SEQ ID NO: 849 - DNA, 28 bp, Homo sapiens
  source 1..28: mol_type = unassigned DNA
gcggaatagttcgcggttttggaatgtt

SEQ ID NO: 850 - DNA, 28 bp, Homo sapiens
  source 1..28: mol_type = unassigned DNA
aaacgtttccgctccccgaaaaacgaat

SEQ ID NO: 851 - DNA, 28 bp, Homo sapiens
  source 1..28: mol_type = unassigned DNA
ggagatagttttgagagggggaggtcgc

SEQ ID NO: 852 - DNA, 28 bp, Homo sapiens
  source 1..28: mol_type = unassigned DNA
cgctacctacgccgatcgtaaatcccaa

SEQ ID NO: 853 - DNA, 28 bp, Homo sapiens
  source 1..28: mol_type = unassigned DNA
gaatggttgcgatatggggttcgacgga

SEQ ID NO: 854 - DNA, 28 bp, Homo sapiens
  source 1..28: mol_type = unassigned DNA
cgcgatccaaaaacgcaaatcctcgttc

SEQ ID NO: 855 - DNA, 28 bp, Homo sapiens
  source 1..28: mol_type = unassigned DNA
ggttttggtcgagatttgggcgggtgag

SEQ ID NO: 856 - DNA, 28 bp, Homo sapiens
  source 1..28: mol_type = unassigned DNA
cccgaatcctacgccccaaccaaataaa

SEQ ID NO: 857 - DNA, 28 bp, Homo sapiens
  source 1..28: mol_type = unassigned DNA
tgagtgatttcggttcggggcgtagatt

SEQ ID NO: 858 - DNA, 29 bp, Homo sapiens
  source 1..29: mol_type = unassigned DNA
cgaaaatctctacaaatcccgcaacctcg

SEQ ID NO: 859 - DNA, 28 bp, Homo sapiens
  source 1..28: mol_type = unassigned DNA
atggtttcggggtgtttagcggcgattg

SEQ ID NO: 860 - DNA, 28 bp, Homo sapiens
  source 1..28: mol_type = unassigned DNA
aacgaaaccgaacgaaccccaatccgta

SEQ ID NO: 861 - DNA, 27 bp, Homo sapiens
  source 1..27: mol_type = unassigned DNA
gagtcggagtgagcgttaagtgagggg

SEQ ID NO: 862 - DNA, 28 bp, Homo sapiens
  source 1..28: mol_type = unassigned DNA
tccctccgaccgacccaaaataactacg

SEQ ID NO: 863 - DNA, 28 bp, Homo sapiens
  source 1..28: mol_type = unassigned DNA
ttcgttgttcgttttgggtaaagggaag

SEQ ID NO: 864 - DNA, 28 bp, Homo sapiens
  source 1..28: mol_type = unassigned DNA
aaactcgcttcccaaacttctaaaaatc

SEQ ID NO: 865 - DNA, 28 bp, Homo sapiens
  source 1..28: mol_type = unassigned DNA
ggggaggcgtcgagttcggagtttatta

SEQ ID NO: 866 - DNA, 28 bp, Homo sapiens
  source 1..28: mol_type = unassigned DNA
aaaactcgctaaacgtcccaaccgcatc

SEQ ID NO: 867 - DNA, 30 bp, Homo sapiens
  source 1..30: mol_type = unassigned DNA
cgggtattgttggtttaggttgtagtaggt

SEQ ID NO: 868 - DNA, 28 bp, Homo sapiens
  source 1..28: mol_type = unassigned DNA
cgaccctaaccaaccccgaaactcgaaa

SEQ ID NO: 869 - DNA, 28 bp, Homo sapiens
  source 1..28: mol_type = unassigned DNA
agggtagaaaggaagcggtagtagaaaa

SEQ ID NO: 870 - DNA, 28 bp, Homo sapiens
  source 1..28: mol_type = unassigned DNA
acaacaactcctcccttcgaacccaacc

SEQ ID NO: 871 - DNA, 28 bp, Homo sapiens
  source 1..28: mol_type = unassigned DNA
tgtgggagggaagggaaatcgagattgg

SEQ ID NO: 872 - DNA, 29 bp, Homo sapiens
  source 1..29: mol_type = unassigned DNA
acgacaaaacgaaacccacaatcctaccc

SEQ ID NO: 873 - DNA, 28 bp, Homo sapiens
  source 1..28: mol_type = unassigned DNA
attcggattggttagtttttgcggaagt

SEQ ID NO: 874 - DNA, 28 bp, Homo sapiens
  source 1..28: mol_type = unassigned DNA
ttcgccacgcaacaacctaaaacgctac

SEQ ID NO: 875 - DNA, 28 bp, Homo sapiens
  source 1..28: mol_type = unassigned DNA
ggttgcggcgtttatttagcgggaagtc

SEQ ID NO: 876 - DNA, 26 bp, Homo sapiens
  source 1..26: mol_type = unassigned DNA
ctcgccgaacccgcgacgaaatctac

SEQ ID NO: 877 - DNA, 28 bp, Homo sapiens
  source 1..28: mol_type = unassigned DNA
tagaggaatttaaagtgtgggttggggg

SEQ ID NO: 878 - DNA, 28 bp, Homo sapiens
  source 1..28: mol_type = unassigned DNA
accaacttctctccctttacgccttttt

SEQ ID NO: 879 - DNA, 29 bp, Homo sapiens
  source 1..29: mol_type = unassigned DNA
tgggttaagtatttgttatgtgttacgga

SEQ ID NO: 880 - DNA, 24 bp, Homo sapiens
  source 1..24: mol_type = unassigned DNA
cgctatccacccgaatacgcaact

SEQ ID NO: 881 - DNA, 26 bp, Homo sapiens
  source 1..26: mol_type = unassigned DNA
gcgcggcgttttgttatcggtggatt

SEQ ID NO: 882 - DNA, 30 bp, Homo sapiens
  source 1..30: mol_type = unassigned DNA
tctaaaataacccgcaccaaacaaactaca

SEQ ID NO: 883 - DNA, 28 bp, Homo sapiens
  source 1..28: mol_type = unassigned DNA
ttataaaggtcggaagcggttacggggg

SEQ ID NO: 884 - DNA, 28 bp, Homo sapiens
  source 1..28: mol_type = unassigned DNA
aacccctttcgctcccttcctaaaacga

SEQ ID NO: 885 - DNA, 28 bp, Homo sapiens
  source 1..28: mol_type = unassigned DNA
gtatggttgcgatttggggttggaaggg

SEQ ID NO: 886 - DNA, 28 bp, Homo sapiens
  source 1..28: mol_type = unassigned DNA
gccgcgatccaaaaacgcaaatcctaat

SEQ ID NO: 887 - DNA, 27 bp, Homo sapiens
  source 1..27: mol_type = unassigned DNA
tagggaatgtttggttgcgatttgggg

SEQ ID NO: 888 - DNA, 28 bp, Homo sapiens
  source 1..28: mol_type = unassigned DNA
tccttaccgtcgtaaacatactactcat

SEQ ID NO: 889 - DNA, 25 bp, Homo sapiens
  source 1..25: mol_type = unassigned DNA
gcgtaagtgcgaggttgtcggtagc

SEQ ID NO: 890 - DNA, 26 bp, Homo sapiens
  source 1..26: mol_type = unassigned DNA
tttcccgcaactctttccccctctct

SEQ ID NO: 891 - DNA, 28 bp, Homo sapiens
  source 1..28: mol_type = unassigned DNA
tgcggttaaagaattcgttcgcgttcgg

SEQ ID NO: 892 - DNA, 28 bp, Homo sapiens
  source 1..28: mol_type = unassigned DNA
ctaaacgctcccgcgaaacctccaaatc

SEQ ID NO: 893 - DNA, 28 bp, Homo sapiens
  source 1..28: mol_type = unassigned DNA
ttcgggtattttgaggttgtcgtcggga

SEQ ID NO: 894 - DNA, 26 bp, Homo sapiens
  source 1..26: mol_type = unassigned DNA
gacgacgacgcgtccgacgaatttta

SEQ ID NO: 895 - DNA, 28 bp, Homo sapiens
  source 1..28: mol_type = unassigned DNA
gttttggtcggggcgtcgtggatatttt

SEQ ID NO: 896 - DNA, 28 bp, Homo sapiens
  source 1..28: mol_type = unassigned DNA
aaaaaccaactaaaccccttcccgctcg

SEQ ID NO: 897 - DNA, 28 bp, Homo sapiens
  source 1..28: mol_type = unassigned DNA
gtgtggaaagggtttggcggttgttagg

SEQ ID NO: 898 - DNA, 28 bp, Homo sapiens
  source 1..28: mol_type = unassigned DNA
ctcgccaaatacgtccacccaaaaacga

SEQ ID NO: 899 - DNA, 28 bp, Homo sapiens
  source 1..28: mol_type = unassigned DNA
taggaggggacgtagagtttacggcgaa

SEQ ID NO: 900 - DNA, 27 bp, Homo sapiens
  source 1..27: mol_type = unassigned DNA
gaatacccgacccgacccatccatcac

SEQ ID NO: 901 - DNA, 28 bp, Homo sapiens
  source 1..28: mol_type = unassigned DNA
tgcgtttgtaggagaagtcgggttggtt

SEQ ID NO: 902 - DNA, 28 bp, Homo sapiens
  source 1..28: mol_type = unassigned DNA
actcttcctcgcctcgcactactaccta

SEQ ID NO: 903 - DNA, 28 bp, Homo sapiens
  source 1..28: mol_type = unassigned DNA
gtgcgattcggggtttcgaaaagttggt

SEQ ID NO: 904 - DNA, 28 bp, Homo sapiens
  source 1..28: mol_type = unassigned DNA
gaaactacgcgcgaacttacaacgcctc

SEQ ID NO: 905 - DNA, 22 bp, Homo sapiens
  source 1..22: mol_type = unassigned DNA
gagcgtagagcgttgagcgggg

SEQ ID NO: 906 - DNA, 22 bp, Homo sapiens
  source 1..22: mol_type = unassigned DNA
cgccgccgaataacacgcccac

SEQ ID NO: 907 - DNA, 28 bp, Homo sapiens
  source 1..28: mol_type = unassigned DNA
agcggggattttcggagttggagagttt

SEQ ID NO: 908 - DNA, 28 bp, Homo sapiens
  source 1..28: mol_type = unassigned DNA
ctccatccgcccgacctaaccctaaaaa

SEQ ID NO: 909 - DNA, 28 bp, Homo sapiens
  source 1..28: mol_type = unassigned DNA
gggaagtggcgtagtgggcgtttgtatc

SEQ ID NO: 910 - DNA, 28 bp, Homo sapiens
  source 1..28: mol_type = unassigned DNA
tacctccaacgaccacgcccacaaaata

SEQ ID NO: 911 - DNA, 28 bp, Homo sapiens
  source 1..28: mol_type = unassigned DNA
tggagcgttgagtcgaagttttgatttt

SEQ ID NO: 912 - DNA, 28 bp, Homo sapiens
  source 1..28: mol_type = unassigned DNA
tcttacccgaactttaaccccaaccgct

SEQ ID NO: 913 - DNA, 28 bp, Homo sapiens
  source 1..28: mol_type = unassigned DNA
ggttgggagttcggagttgtagtagagg

SEQ ID NO: 914 - DNA, 28 bp, Homo sapiens
  source 1..28: mol_type = unassigned DNA
ctttaaccgattcaaacaacaaacgcct

SEQ ID NO: 915 - DNA, 26 bp, Homo sapiens
  source 1..26: mol_type = unassigned DNA
gtagggcgcggggtttcgttagtttc

SEQ ID NO: 916 - DNA, 28 bp, Homo sapiens
  source 1..28: mol_type = unassigned DNA
atctaccgttctatcctcgtaaccgccg

SEQ ID NO: 917 - DNA, 28 bp, Homo sapiens
  source 1..28: mol_type = unassigned DNA
tcgtttgggagggatcgtttttgggaga

SEQ ID NO: 918 - DNA, 28 bp, Homo sapiens
  source 1..28: mol_type = unassigned DNA
aacccgaatactatccaactaccgccgc

SEQ ID NO: 919 - DNA, 28 bp, Homo sapiens
  source 1..28: mol_type = unassigned DNA
cgagcgtgggtattaagtcggtagtgga

SEQ ID NO: 920 - DNA, 28 bp, Homo sapiens
  source 1..28: mol_type = unassigned DNA
gacctcaaccccctacgcctaacctact

SEQ ID NO: 921 - DNA, 28 bp, Homo sapiens
  source 1..28: mol_type = unassigned DNA
gtagatcgcggttttgtaggggcgtttg

SEQ ID NO: 922 - DNA, 28 bp, Homo sapiens
  source 1..28: mol_type = unassigned DNA
ctaatttcgatttttccacccccgccgc

SEQ ID NO: 923 - DNA, 28 bp, Homo sapiens
  source 1..28: mol_type = unassigned DNA
gagtgtttgtcgagaaggttgagtaaat

SEQ ID NO: 924 - DNA, 28 bp, Homo sapiens
  source 1..28: mol_type = unassigned DNA
caccgcccaacgcattcgttctaaaata

SEQ ID NO: 925 - DNA, 26 bp, Homo sapiens
  source 1..26: mol_type = unassigned DNA
ataaaagtggggtgggtggcggaggg

SEQ ID NO: 926 - DNA, 28 bp, Homo sapiens
  source 1..28: mol_type = unassigned DNA
gcgccgaaataacaacccaacctaccaa

SEQ ID NO: 927 - DNA, 28 bp, Homo sapiens
  source 1..28: mol_type = unassigned DNA
tttatcggggaagttttcgagggtgggc

SEQ ID NO: 928 - DNA, 28 bp, Homo sapiens
  source 1..28: mol_type = unassigned DNA
tcccaactacctcctacgcacgaacgat

SEQ ID NO: 929 - DNA, 29 bp, Homo sapiens
  source 1..29: mol_type = unassigned DNA
atgaaatgtggttcgtggaaggtgtttgt

SEQ ID NO: 930 - DNA, 28 bp, Homo sapiens
  source 1..28: mol_type = unassigned DNA
acgacccgaacgttaatcctcttactac

SEQ ID NO: 931 - DNA, 29 bp, Homo sapiens
  source 1..29: mol_type = unassigned DNA
acgtagttttcgagttagtgtcgttagaa

SEQ ID NO: 932 - DNA, 23 bp, Homo sapiens
  source 1..23: mol_type = unassigned DNA
gacaaacgcctcaaacccgaccg

SEQ ID NO: 933 - DNA, 28 bp, Homo sapiens
  source 1..28: mol_type = unassigned DNA
aggagcgggagagggaaaaatagttaag

SEQ ID NO: 934 - DNA, 26 bp, Homo sapiens
  source 1..26: mol_type = unassigned DNA
cgctccaaactacgcccaaaactcaa

SEQ ID NO: 935 - DNA, 28 bp, Homo sapiens
  source 1..28: mol_type = unassigned DNA
attagagttgttttgcgtattgcggcgg

SEQ ID NO: 936 - DNA, 28 bp, Homo sapiens
  source 1..28: mol_type = unassigned DNA
caaataccccgtacacccgctaccccaa

SEQ ID NO: 937 - DNA, 28 bp, Homo sapiens
  source 1..28: mol_type = unassigned DNA
gggagttgaggtttacgcggtttcgttg

SEQ ID NO: 938 - DNA, 27 bp, Homo sapiens
  source 1..27: mol_type = unassigned DNA
gaccgccaacgcgatccacccattaac

SEQ ID NO: 939 - DNA, 28 bp, Homo sapiens
  source 1..28: mol_type = unassigned DNA
agttttggaagtagattcggtgcgggtg

SEQ ID NO: 940 - DNA, 24 bp, Homo sapiens
  source 1..24: mol_type = unassigned DNA
gccgcgcaatcgcctctttttcac

SEQ ID NO: 941 - DNA, 28 bp, Homo sapiens
  source 1..28: mol_type = unassigned DNA
agacgatagatggcgggtaggaagggag

SEQ ID NO: 942 - DNA, 28 bp, Homo sapiens
  source 1..28: mol_type = unassigned DNA
gccgcctacaaccgacgaactacaaatc

SEQ ID NO: 943 - DNA, 28 bp, Homo sapiens
  source 1..28: mol_type = unassigned DNA
tcgcgggtgaggtttgtggttaatttcg

SEQ ID NO: 944 - DNA, 28 bp, Homo sapiens
  source 1..28: mol_type = unassigned DNA
gctcaaccaaactacaacgttcccgcct

SEQ ID NO: 945 - DNA, 28 bp, Homo sapiens
  source 1..28: mol_type = unassigned DNA
tgagaggcgtattttgttggttacggtt

SEQ ID NO: 946 - DNA, 28 bp, Homo sapiens
  source 1..28: mol_type = unassigned DNA
cgaaaaccattccgctacccttccaact

SEQ ID NO: 947 - DNA, 29 bp, Homo sapiens
  source 1..29: mol_type = unassigned DNA
ggggcgttgggttatggagattacgtttt

SEQ ID NO: 948 - DNA, 28 bp, Homo sapiens
  source 1..28: mol_type = unassigned DNA
gtcccgcgcttaacgaattctacgaacg

SEQ ID NO: 949 - DNA, 22 bp, Homo sapiens
  source 1..22: mol_type = unassigned DNA
gttcgggtaggggtcgggggtc

SEQ ID NO: 950 - DNA, 28 bp, Homo sapiens
  source 1..28: mol_type = unassigned DNA
cccgaaacgacgtacttaacgacccgaa

SEQ ID NO: 951 - DNA, 28 bp, Homo sapiens
  source 1..28: mol_type = unassigned DNA
gggaggtttgagcgtcgaagttttcgtt

SEQ ID NO: 952 - DNA, 28 bp, Homo sapiens
  source 1..28: mol_type = unassigned DNA
gcccactaccccgcgaaaccttatcaac

SEQ ID NO: 953 - DNA, 28 bp, Homo sapiens
  source 1..28: mol_type = unassigned DNA
agtcgttaggttgttaaggcgcgttgtg

SEQ ID NO: 954 - DNA, 29 bp, Homo sapiens
  source 1..29: mol_type = unassigned DNA
acaaaaataaaatcgaacctaaccccacg

SEQ ID NO: 955 - DNA, 25 bp, Homo sapiens
  source 1..25: mol_type = unassigned DNA
cgtattaagggttaagcggcgcggt

SEQ ID NO: 956 - DNA, 29 bp, Homo sapiens
  source 1..29: mol_type = unassigned DNA
aactttctcgaacgactcgataaacctaa

SEQ ID NO: 957 - DNA, 28 bp, Homo sapiens
  source 1..28: mol_type = unassigned DNA
aggttttgggaatttggaagttcgcggg

SEQ ID NO: 958 - DNA, 28 bp, Homo sapiens
  source 1..28: mol_type = unassigned DNA
aaaaacgctcgaacccaaccaatcgacg

SEQ ID NO: 959 - DNA, 27 bp, Homo sapiens
  source 1..27: mol_type = unassigned DNA
aaaggaagatcgtgggtagttcgtgcg

SEQ ID NO: 960 - DNA, 28 bp, Homo sapiens
  source 1..28: mol_type = unassigned DNA
actacaactcacgtttcccctccaacac

SEQ ID NO: 961 - DNA, 29 bp, Homo sapiens
  source 1..29: mol_type = unassigned DNA
aggtttatttgacgttttaggtcgatagt

SEQ ID NO: 962 - DNA, 28 bp, Homo sapiens
  source 1..28: mol_type = unassigned DNA
cgatctctccctttcttccgcttcctaa

SEQ ID NO: 963 - DNA, 22 bp, Homo sapiens
  source 1..22: mol_type = unassigned DNA
ggcgtcggttgcggttttagat

SEQ ID NO: 964 - DNA, 30 bp, Homo sapiens
  source 1..30: mol_type = unassigned DNA
acgcgaaaatctaccttttaattacgaacc

SEQ ID NO: 965 - DNA, 23 bp, Homo sapiens
  source 1..23: mol_type = unassigned DNA
tcgtcggtggtcggcgcgttttt

SEQ ID NO: 966 - DNA, 28 bp, Homo sapiens
  source 1..28: mol_type = unassigned DNA
aacccgcaccaaacaaactacacgcaaa

SEQ ID NO: 967 - DNA, 28 bp, Homo sapiens
  source 1..28: mol_type = unassigned DNA
gaatggtagcgagaggttgcgggttagg

SEQ ID NO: 968 - DNA, 28 bp, Homo sapiens
  source 1..28: mol_type = unassigned DNA
ctctaccctcaaaatcgcgacgcaaacg

SEQ ID NO: 969 - DNA, 28 bp, Homo sapiens
  source 1..28: mol_type = unassigned DNA
aggagtagtattgcgagggtggagggtt

SEQ ID NO: 970 - DNA, 28 bp, Homo sapiens
  source 1..28: mol_type = unassigned DNA
cgccaatcccgctccgacactataacaa

SEQ ID NO: 971 - DNA, 28 bp, Homo sapiens
  source 1..28: mol_type = unassigned DNA
ataaggtttggtggaagcgtaggagcgt

SEQ ID NO: 972 - DNA, 28 bp, Homo sapiens
  source 1..28: mol_type = unassigned DNA
acgatattctaacctccgccgcgaaact

SEQ ID NO: 973 - DNA, 28 bp, Homo sapiens
  source 1..28: mol_type = unassigned DNA
ggtagagggatagggaagagtttggcgt

SEQ ID NO: 974 - DNA, 28 bp, Homo sapiens
  source 1..28: mol_type = unassigned DNA
attcaaaacgcgcgcgacgaaattcaac

SEQ ID NO: 975 - DNA, 28 bp, Homo sapiens
  source 1..28: mol_type = unassigned DNA
gcggagtgggagggttatattgggagag

SEQ ID NO: 976 - DNA, 28 bp, Homo sapiens
  source 1..28: mol_type = unassigned DNA
ccgaacaaaactacgacaccgccgaaaa

SEQ ID NO: 977 - DNA, 28 bp, Homo sapiens
  source 1..28: mol_type = unassigned DNA
acgacgggttgagataggtggttggatt

SEQ ID NO: 978 - DNA, 28 bp, Homo sapiens
  source 1..28: mol_type = unassigned DNA
cccgacgcgaaacaacgaactaaaacga

SEQ ID NO: 979 - DNA, 28 bp, Homo sapiens
  source 1..28: mol_type = unassigned DNA
tttttgtacgttttcggggtcggaggag

SEQ ID NO: 980 - DNA, 28 bp, Homo sapiens
  source 1..28: mol_type = unassigned DNA
aatcgccgtctaaacaaatcgcgaacta

SEQ ID NO: 981 - DNA, 29 bp, Homo sapiens
  source 1..29: mol_type = unassigned DNA
cggcggtcgcggtttgtagtttagaattg

SEQ ID NO: 982 - DNA, 28 bp, Homo sapiens
  source 1..28: mol_type = unassigned DNA
acgcgcttccactccgactaacaaatta

SEQ ID NO: 983 - DNA, 27 bp, Homo sapiens
  source 1..27: mol_type = unassigned DNA
ggcgtcgttcgggttaagtttggttgt

SEQ ID NO: 984 - DNA, 28 bp, Homo sapiens
  source 1..28: mol_type = unassigned DNA
cacaacttacgcgaaacaacaacctcgc

SEQ ID NO: 985 - DNA, 28 bp, Homo sapiens
  source 1..28: mol_type = unassigned DNA
tgggttggtgtcgcgcgaatttttgttt

SEQ ID NO: 986 - DNA, 28 bp, Homo sapiens
  source 1..28: mol_type = unassigned DNA
cctcctcccgcaactacgaaaaccgata

SEQ ID NO: 987 - DNA, 28 bp, Homo sapiens
  source 1..28: mol_type = unassigned DNA
gttgggggttatgtttggcgcggaatag

SEQ ID NO: 988 - DNA, 28 bp, Homo sapiens
  source 1..28: mol_type = unassigned DNA
cgcctatataaaacgtcgacgcgcgaaa

SEQ ID NO: 989 - DNA, 28 bp, Homo sapiens
  source 1..28: mol_type = unassigned DNA
gttcgggcgtcgcggtcgtttttatatt

SEQ ID NO: 990 - DNA, 28 bp, Homo sapiens
  source 1..28: mol_type = unassigned DNA
aaaacgcccattatacccgcgccaattc

SEQ ID NO: 991 - DNA, 28 bp, Homo sapiens
  source 1..28: mol_type = unassigned DNA
taagaatcggcggtagttagtaggcggg

SEQ ID NO: 992 - DNA, 28 bp, Homo sapiens
  source 1..28: mol_type = unassigned DNA
tcctacgccgcgacgaaaacaaaaactc

SEQ ID NO: 993 - DNA, 28 bp, Homo sapiens
  source 1..28: mol_type = unassigned DNA
aggtggggcgcgtttattagtttagggg

SEQ ID NO: 994 - DNA, 28 bp, Homo sapiens
  source 1..28: mol_type = unassigned DNA
acctctccatcgctaataccctaccgct

SEQ ID NO: 995 - DNA, 28 bp, Homo sapiens
  source 1..28: mol_type = unassigned DNA
gagtgtttcgagggtaggaggttgtcgg

SEQ ID NO: 996 - DNA, 28 bp, Homo sapiens
  source 1..28: mol_type = unassigned DNA
caaaaaccgcccgcaaaacgaaacctaa

SEQ ID NO: 997 - DNA, 28 bp, Homo sapiens
  source 1..28: mol_type = unassigned DNA
acggatcgatcgcggttttggtaaggat

SEQ ID NO: 998 - DNA, 28 bp, Homo sapiens
  source 1..28: mol_type = unassigned DNA
cgcaaaaacgaaaaactacgtacgcgct

SEQ ID NO: 999 - DNA, 27 bp, Homo sapiens
  source 1..27: mol_type = unassigned DNA
gttgtttgaggacgggtcgtttagcgg

SEQ ID NO: 1000 - DNA, 28 bp, Homo sapiens
  source 1..28: mol_type = unassigned DNA
acccctatcctacaaccctacgaacgca

SEQ ID NO: 1001 - DNA, 28 bp, Homo sapiens
  source 1..28: mol_type = unassigned DNA
tttcgggaggtgtggttacgtttggaga

SEQ ID NO: 1002 - DNA, 28 bp, Homo sapiens
  source 1..28: mol_type = unassigned DNA
cccctcctcccaacacccaacactaaaa

SEQ ID NO: 1003 - DNA, 30 bp, Homo sapiens
  source 1..30: mol_type = unassigned DNA
ggttgtggtttttaaaagggaaaattcggg

SEQ ID NO: 1004 - DNA, 28 bp, Homo sapiens
  source 1..28: mol_type = unassigned DNA
taaacgccgaaacccgaacgtaacaacc

SEQ ID NO: 1005 - DNA, 28 bp, Homo sapiens
  source 1..28: mol_type = unassigned DNA
aggtgattagaagggagagggggaggtt

SEQ ID NO: 1006 - DNA, 28 bp, Homo sapiens
  source 1..28: mol_type = unassigned DNA
tcattatacacgacgcgcccctccaaat

SEQ ID NO: 1007 - DNA, 28 bp, Homo sapiens
  source 1..28: mol_type = unassigned DNA
tacgtgggtgtaggttaggtcgggttga

SEQ ID NO: 1008 - DNA, 29 bp, Homo sapiens
  source 1..29: mol_type = unassigned DNA
accacgcgactaccgtataaacaaccgaa

(19) United States
(12) Patent Application Publication
(10) Pub. No.: US 2024/0274232 A1
(43) Pub. Date: Aug. 15, 2024

(54) CELL-FREE DETECTION OF METHYLATED BREAST TUMOR

(71) Applicants: QUEEN'S UNIVERSITY AT KINGSTON, Kingston (CA); INSTITUT CURIE, Paris (FR); INSTITUT NATIONAL DE LA SANTE ET DE LA RECHERCHE MEDICALE (INSERM), Paris (FR)

(72) Inventor: Christopher R. MUELLER, Kingston (CA)

(73) Assignees: QUEEN'S UNIVERSITY AT KINGSTON, Kingston (CA); INSTITUT CURIE, Paris (FR); INSTITUT NATIONAL DE LA SANTE ET DE LA RECHERCHE MEDICALE (INSERM), Paris (FR)

(21) Appl. No.: 18/632,749

(22) Filed: Apr. 11, 2024

Related U.S. Application Data
(63) Continuation of application No. 16/098,455, filed on Nov. 2, 2018.
(60) Provisional application No. 62/331,585, filed on May 4, 2016.

(51) Int. Cl. G16B 30/00; C12Q 1/6827; C12Q 1/6886; G16B 25/00; G16B 25/10; G16B 35/00; G16B 99/00

(52) U.S. Cl. CPC G16B 30/00; C12Q 1/6827; C12Q 1/6886; G16B 25/00; G16B 25/10; G16B 35/00; G16B 99/00; C12Q 2600/106; C12Q 2600/118; C12Q 2600/154

(57) ABSTRACT

Provided herein is a method for detecting a tumour that can be applied to cell-free samples, to cell-free detect circulating tumour DNA. The method utilizes detection of adjacent methylation signals within a single sequencing read as the basic positive tumour signal, thereby decreasing false positives. The method comprises extracting DNA from a cell-free sample obtained from a subject, bisulphite converting the DNA, amplifying regions methylated in cancer (CpG islands, CpG shores, and/or CpG shelves), generating sequencing reads, and detecting tumour signals. To increase sensitivity, biased primers designed based on bisulphite converted methylated sequences can be used. Target methylated regions can be selected from a pre-validated set according to the specific aim of the test. Absolute number, proportion, and/or distribution of tumour signals may be used for tumour detection or classification. The method is also useful in, predicting, prognosticating, and/or monitoring response to treatment, tumour load, relapse, cancer development, or risk.

### CROSS-REFERENCE TO RELATED APPLICATIONS

This application is a continuation of U.S. application Ser. No. 16/098,455, which is a U.S. National Stage entry of PCT/CA2017/000111, filed May 4, 2017, which claims the benefit of U.S. Provisional App. No. 62/331,585, filed May 4, 2016. The disclosure of each of the above applications is herein incorporated by reference in its entirety.

The instant application contains a Sequence Listing which has been submitted electronically in XML file format and is hereby incorporated by reference in its entirety. Said XML copy, created on Apr. 1, 2024, is named “P70051_SL.xml” and is 891,946 bytes in size.

### FIELD

This disclosure relates generally to tumour detection. More particularly, this disclosure relates to tumour-specific DNA methylation detection.

### BACKGROUND

Cancer screening and monitoring has helped to improve outcomes over the past few decades simply because early detection leads to a better outcome as the cancer can be eliminated before it has spread. In the case of breast cancer, for instance, physical breast exams, mammography, ultrasound and MRI (in high risk patients) have all played a role in improving early diagnosis. The cost/benefit of these modalities for general screening, particularly in relatively younger women, has been controversial.

A primary issue for any screening tool is the compromise between false positive and false negative results (or specificity and sensitivity) which lead to unnecessary investigations in the former case, and ineffectiveness in the latter case. An ideal test is one that has a high Positive Predictive Value (PPV), minimizing unnecessary investigations but detecting the vast majority of cancers. Another key factor is what is called “detection sensitivity”, to distinguish it from test sensitivity, and that is the lower limits of detection in terms of the size of the tumour. Screening mammography in breast cancer, for instance, is considered to have a sensitivity from 80 to 90% with a specificity of 90%. However the mean size of tumours detected by mammography remains in the range of 15 to 19 mm. It has been suggested that only 3-13% of women derive an improved treatment outcome from this screening suggesting that the detection of smaller tumours would provide increased benefit. For women at high risk of developing breast cancer the use of MRI has offered some benefit with sensitivities in the range of 75 to 97% and specificities in the area of 90 to 96% and in combination with mammography offering 93-94% sensitivity and 77 to 96% specificities. However, MRI is acknowledged to have a poor PPV, in the area of 10-20%, leading to a large number of false positives and as a consequence unnecessary invasive investigations. All of these screens have likely reached their limit of detection sensitivity (or size of the tumour) and in the case of mammography still involve exposure to radiation, which may be of particular concern in women with familial mutations which render them more sensitive to radiation damage. There are no effective blood based screens for breast cancer based on circulating analytes.

While the above discussion focusses on breast cancer as an example, many of the same challenges exist for other types of cancers as well.

The detection of circulating tumour DNA is increasingly acknowledged as a viable “liquid biopsy” allowing for the detection and informative investigation of tumours in a non-invasive manner. Typically using the identification of tumour specific mutations these techniques have been applied to colon, breast and prostate cancers. Due to the high background of normal DNA present in the circulation these techniques can be limited in sensitivity. As well, the variable nature of tumour mutations in terms of occurrence and location (such as p53 and KRAS mutations) has generally limited these approaches to detecting tumour DNA at 1% of the total DNA in serum. Advanced techniques such as BEAMing have increased sensitivity, but are still limited overall. Even with these limitations the detection of circulating tumour DNA has recently been shown to be useful for detecting metastasis in breast cancer patients.

The detection of tumour specific methylation in the blood has been proposed to offer distinct advantages over the detection of mutations¹⁻⁵. A number of single or multiple methylation biomarkers have been assessed in cancers including lung⁶⁻¹⁰, colon^{11,12} and breast¹³⁻¹⁶. These have suffered from low sensitivities as they have tended to be insufficiently prevalent in the tumours. Several multi-gene assays have been developed with improved performance. A more advanced multi-gene system using a combination of 10 different genes has been reported and uses a multiplexed PCR based assay¹⁷. It offers combined sensitivity and specificity of 91% and 96% respectively, due to the better coverage offered and it has been validated in a small cohort of stage IV patients. However, it has a very high background in normal blood which will limit its detection sensitivity. Methylated markers have been used to monitor the response to neoadjuvant therapy^{18,19}, and recently a methylation gene signature associated with metastatic tumours has been identified²⁰.

There remains a need for more sensitive and specific screening tools, as well as for straightforward tests that allow for the assessment of tumour burden, chemotherapy response, detection of residual disease, relapse and primary screening in high risk populations.

### SUMMARY

It is an object of this disclosure to obviate or mitigate at least one disadvantage of previous approaches.

In a first aspect, this disclosure provides a method for detecting a tumour, comprising: extracting DNA from a cell-free sample obtained from a subject, bisulphite converting at least a portion of the DNA, amplifying regions methylated in cancer from the bisulphite converted DNA, generating sequencing reads from the amplified regions, and detecting tumour signals comprising at least two adjacent methylated sites within a single sequencing read, wherein the detection of at least one of the tumour signals is indicative of a tumour.

In another aspect, there is provided a use of the method for determining response to treatment.

In another aspect, there is provided a use of the method for monitoring tumour load.

In another aspect, there is provided a use of the method for detecting residual tumour post-surgery.

In another aspect, there is provided a use of the method for detecting relapse.

In another aspect, there is provided a use of the method as a secondary screen.

In another aspect, there is provided a use of the method as a primary screen.

In another aspect, there is provided a use of the method for monitoring cancer development.

In another aspect, there is provided a use of the method for monitoring cancer risk.

In another aspect, there is provided a kit for detecting a tumour comprising reagents for carrying out the method, and instructions for detecting the tumour signals.

Other aspects and features of this disclosure will become apparent to those ordinarily skilled in the art upon review of the following description of specific embodiments in conjunction with the accompanying figures.

### BRIEF DESCRIPTION OF THE DRAWINGS

Embodiments of this disclosure will now be described, by way of example only, with reference to the attached Figures.

FIG. 1 depicts a schematic of the method.

FIG. 2 depicts a schematic of the amplification of multiple target regions.

FIG. 3 lists 47 CpG targets selected to identify differentially methylated regions, and shows the results of Receiver Operator Curve (ROC) analysis.

FIG. 4 depicts histograms showing the frequency of patients binned according to positive (methylated) probe frequency. Panel A depicts results for luminal tumours. Panel B depicts results for basal tumours.

FIG. 5 depicts sequencing results to assess methylation status of a region near the CHST11 gene (CHST11 Probe C) in breast cancer cell lines.

FIG. 6 depicts sequencing results to assess methylation status of CHST11 Probe A in breast cancer tumors and normal breast tissue.

FIG. 7 depicts sequencing results to assess methylation status of FOXA Probe A in breast cancer cell lines.

FIG. 8 depicts sequencing results to assess methylation status of CHST Probe A and Probe B in prostate cancer cell lines.

FIG. 9 depicts sequencing results to assess methylation status of FOXA Probe A in prostate cancer cell lines.

FIG. 10 depicts sequencing results to assess methylation status of NT5 Probe E in breast cancer cell lines.

FIG. 11 depicts a summary of BioAnalyzer electrophoresis summary for amplification product generated from various cell lines.

FIGS. 12A and 12B depict a numerical summary of validation data generated for 98 different probes by bisulphite sequencing six different cell lines. #Reads is indicative of the number of reads exported, and Mean Me is indicative of the mean methylation.

FIGS. 13A and 13B depict a numerical summary of generated methylation data for tumour samples. #Reads is indicative of the number of reads exported, and Mean Me is indicative of the mean methylation.

FIG. 14 depicts a numerical summary generated methylation data for prostate cell lines. #Reads is indicative of the number of reads exported, and Mean Me is indicative of the mean methylation.

FIG. 15 is a diagram showing validation of various uveal melanoma (UM) probes in two cell lines MP38 (with loss of 3p) and MP41 (3p WT). Negative controls were cell free DNA (cfDNA) consisting of a pool of 18 individuals without cancer and peripheral mononuclear cells (PBMC). Probes for the indicated regions were PCR amplified individually and sequenced. Darker shading indicates higher level of methylation. OST3F was methylated in PBMCs while LDL3F was not methylated in tumours, with the majority showing strong methylation in the UM lines but not in the PBMCs or cfDNA.

FIG. 16 is a diagram showing methylation of cfDNA from patients with metastatic uveal melanoma. Methylated reads for each probe were extracted and all reads were normalized for the total number of reads in the sample. Stacked columns represent the total reads from all of the individual probes with different probes identified by shading. The patients are sorted by PAP measurements with high values on the left and lower values on the right. cfDNA is a pool of cell free DNA from 18 normal donors.

FIG. 17 is a diagram showing methylation of cfDNA from patients with metastatic uveal melanoma. Methylated reads for each probe were extracted and all reads were normalized for the total number of reads in the sample. Stacked columns represent the total reads from all of the individual probes with different probes identified by shading. The patients are sorted by tumour volume with larger volume on the left and lower volume on the right, and the volume indicated at the bottom. PAP values obtained from these patients is indicated. <5 refers to no detection of ctDNA in these samples. cfDNA is a pool of cell free DNA from 18 normal donors.

FIGS. 18A and 18B are diagrams showing methylation of cfDNA from sequential blood samples of two patients who were part of the patient groups shown in FIGS. 17 and 18. In FIG. 19A the patient was retested after seven months and the tumour at that time was assessed as being 0.5 cm³ in volume. In FIG. 19B the patient was retested after four months where the initial tumour volume was 483 cm³.

FIG. 19 is a log-log plot showing assay values (methylated reads) are correlated with tumour volume. The character of the metastatic tumour such as whether it is a solid mass or dispersed (miliary) was not taken into account.

FIG. 20 is a log-log plot showing relationship between test results and PAP signal, where PAP and methylation signals were correlated at higher PAP levels (trend line), although below the detection threshold of PAP at 5 copies/ml (vertical dashed line) the PAP signals were not correlated (ellipse).

FIG. 21 is a heat map of gene methylation in indicated prostate cancer cell lines.

FIG. 22 is a heat map of multiplexed probes for each prostate cancer patient sample. Patient samples were taken before the initiation of ADT (START) and 12 months after (M12). A black square indicates that methylated reads having greater than 80% methylation per read were detected for that probe but does not take into consideration the number of reads for each.

FIG. 23 is a diagram showing number of methylated reads per probe for each prostate cancer patient sample. Different probes are shown in different shading. The number of reads that were at least 80% methylated were determined for each sample and all probes are stacked per sample. Patient samples were taken before the initiation of ADT (START) and 12 months after (M12).

FIG. 24 is a plot showing normalized methylation reads per sample verses PSA levels for each patient. The totals of normalized methylated reads for all probes are plotted with solid lines. Patients initiated androgen deprivation therapy (START) and PSA levels measured at that time and after 12 months of treatment (M12) and are indicated with dashed lines. The methylation detection of circulating tumour DNA (mDETECT) test was performed on 0.5 ml of plasma from these same time points. The Gleason score for each patient at initial diagnosis is shown along with grading, as is the treatment applied as primary therapy (RRP, radical retropubic prostatectomy; BT, brachytherapy; EBR, external beam radiation; RT, radiotherapy).

FIG. 25 is a plot of TCGA prostate cancer tumour data, showing the average methylation for each of various Gleason groups, as well as for normal tissue from breast, prostate, lung, and colon, verses position on the genome (in this case on chromosome 8 for the region upstream of the TCF24gene, a transcription factor of unknown function and PRSS3, a serine protease gene on chromosome 9).

FIGS. 26A, 26B, and 26C are charts showing regions used to develop a breast cancer test according to one embodiment. The chromosomal location and nucleotide position of the first CpG residue in the region is indicated. The TCGA breast cancer cohort was divided into sub-groups based on PAM-50 criteria. The fraction of each group that is positive for that probe is indicated. “Tissue” indicates results from normal tissue samples.

FIG. 27 shows theoretical area under the curve analyses of blood tests using the top 20 probes for each breast cancer subtype (LumA, LumB, Basal, HER2). These values were compared against normal tissue samples for the same probes.

FIG. 28 is a heatmap of multiplexed probes for each TNBC tumour sample and selected normal samples. A black square indicates that methylated reads having greater than 80% methylation per read were detected for that probe but does not take into consideration the number of reads for each.

FIG. 29 is a diagram showing results of a sensitivity test for TNBC to detect low levels of tumour DNA, using HCC1937 DNA diluted into a fixed amount of PBMC DNA (10 ng). Shaded squares indicate a distinct methylation signature.

FIG. 30 is a flowchart illustrating a method for determining biological methylation signatures, and for developing probes for their detection.

### DETAILED DESCRIPTION

Generally, this disclosure provides a method for detecting a tumour that can be applied to cell-free samples, e.g., to detect cell-free circulating tumour DNA. The method utilizes detection of adjacent methylation signals within a single sequencing read as the basic “positive” tumour signal.

In one aspect, there is provided a method for detecting a tumour, comprising: extracting DNA from a cell-free sample obtained from a subject, bisulphite converting at least a portion of the DNA, amplifying regions methylated in cancer from the bisulphite converted DNA, generating sequencing reads from the amplified regions, and detecting tumour signals comprising at least two adjacent methylated sites within a single sequencing read, wherein the detection of at least one of the tumour signals is indicative of a tumour.

By “cell-free DNA (cfDNA)” is meant DNA in a biological sample that is not contained in a cell. cfDNA may circulate freely in in a bodily fluid, such as in the bloodstream.

“Cell-free sample”, as used herein, is meant a biological sample that is substantially devoid of intact cells. This may be a derived from a biological sample that is itself substantially devoid of cells, or may be derived from a sample from which cells have been removed. Example cell-free samples include those derived from blood, such as serum or plasma; urine; or samples derived from other sources, such as semen, sputum, feces, ductal exudate, lymph, or recovered lavage.

“Circulating tumour DNA”, as used herein, accordingly refers to cfDNA originating from a tumour.

By “region methylated in cancer” is meant a segment of the genome containing methylation sites (CpG dinucleotides), methylation of which is associated with a malignant cellular state. Methylation of a region may be associated with more than one different type of cancer, or with one type of cancer specifically. Within this, methylation of a region may be associated with more than one subtype, or with one subtype specifically.

The terms cancer “type” and “subtype” are used relatively herein, such that one “type” of cancer, such as breast cancer, may be “subtypes” based on e.g., stage, morphology, histology, gene expression, receptor profile, mutation profile, aggressiveness, prognosis, malignant characteristics, etc. Likewise, “type” and “subtype” may be applied at a finer level, e.g., to differentiate one histological “type” into “subtypes”, e.g., defined according to mutation profile or gene expression.

By “adjacent methylated sites” is meant two methylated sites that are, sequentially, next to each other. It will be understood that this term does not necessarily require the sites to actually be directly beside each other in the physical DNA structure. Rather, in a sequence of DNA including spaced apart methylation sites A, B, and C in the context A-(n)n-B-(n)n-C, wherein (n)n refers to the number of base pairs (bp) (e.g., up to 300 bp), sites A and B would be recognized as “adjacent” as would sites B and C. Sites A and C, however, would not be considered to be adjacent methylated sites.

In one embodiment, the regions methylated in cancer comprise CpG islands.

“CpG islands” are regions of the genome having a high frequency of CpG sites. CpG islands are usually 300-3000 bp in length and are found at or near promotors of approximately 40% of mammalian genes. They show a tendency to occur upstream of so-called “housekeeping genes”. A concrete definition is elusive, but CpG islands may be said to have an absolute GC content of at least 50%, and a CpG dinucleotide content of at least 60% of what would be statistically expected. Their occurrence at or upstream of the 5′ end of genes may reflect a role in the regulation of transcription, and methylation of CpG sites within the promoters of genes may lead to silencing. Silencing of tumour suppressors by methylation is, in turn, a hallmark of a number of human cancers.

In one embodiment, the regions methylated in cancer comprise CpG shores.

“CpG shores” are regions extending short distances from CpG islands in which methylation may also occur. CpG shores may be found in the region 0 to 2 kb upstream and downstream of a CpG island.

In one embodiment, the regions methylated in cancer comprise CpG shelves.

“CpG shelves” are regions extending short distances from CpG shores in which methylation may also occur. CpG shelves may generally be found in the region between 2 kb and 4 kb upstream and downstream of a CpG island (i.e., extending a further 2 kb out from a CpG shore).

In one embodiment, the regions methylated in cancer comprise CpG islands and CpG shores.

In one embodiment, the regions methylated in cancer comprise CpG islands, CpG shores, and CpG shelves.

In one embodiment, the regions methylated in cancer comprise CpG islands and sequences 0 to 4 kb upstream and downstream. The regions methylated in cancer may also comprise CpG islands and sequences 0 to 3 kb upstream and downstream, 0 to 2 kb upstream and downstream, 0 to 1 kb upstream and downstream, 0 to 500 bp upstream and downstream, 0 to 400 bp upstream and downstream, 0 to 300 bp upstream and downstream, 0 to 200 bp upstream and downstream, or 0 to 100 bp upstream and downstream.

In one embodiment, the step of amplifying is carried out with primers designed to anneal to bisulphite converted target sequences having at least one methylated site therein. Bisulphite conversion results in unmethylated cytosines being converted to uracil, while 5-methylcytosine is unaffected. “Bisulphite converted target sequences” are thus understood to be sequences in which cytosines known to be methylation sites are fixed as “C” (cytosine), while cytosines known to be unmethylated are fixed as “U” (uracil; which can be treated as “T” (thymine) for primer design purposes). Primers designed to target such sequences may exhibit a degree of bias towards converted methylated sequences. However, in one embodiment, the primers are designed without preference as to location of the at least one methylated site within target sequences. Often, to achieve optimal discrimination, it may be desirable to place a discriminatory base at the ultimate or penultimate 3′ position of an oligonucleotide PCR primer. In this embodiment, however, no preference is given to the location of the discriminatory sites of methylation, such that overall primer design is optimized based on sequence (not discrimination). This results in a degree of bias for some primer sets, but usually not complete specificity towards methylated sequences (some individual primer pairs, however, may be specific if a discriminatory site is fortuitously placed). As will be described herein, this permits some embodiments of the method to be quantitative or semi-quantitative.

In one embodiment, the PCR primers are designed to be methylation specific. This may allow for greater sensitivity in some applications. For instance, primers may be designed to include a discriminatory nucleotide (specific to a methylated sequence following bisulphite conversion) positioned to achieve optimal discrimination, e.g. in PCR applications. The discriminatory may be positioned at the 3′ ultimate or penultimate position.

In one embodiment, the primers are designed to amplify DNA fragments 75 to 150 bp in length. This is the general size range known for circulating DNA, and optimizing primer design to take into account target size may increase the sensitivity of the method according to this embodiment. The primers may be designed to amplify regions that are 50 to 200, 75 to 150, or 100 or 125 bp in length.

In some embodiments, concordant results provide additional confidence in a positive tumour signal. By “concordant” or “concordance”, as used herein, is meant methylation status that is consistent by location and/or by repeated observation. As has already been stated, the basic “tumour signal” defined herein comprises at least two adjacent methylated sites within a single sequencing read. However, additional layers of concordance can be used to increase confidence for tumour detection, in some embodiments, and not all of these need be derived from the same sequencing read. Layers of concordance that may provide confidence in tumor detection may include, for example:
(a) detection of methylation of at least two adjacent methylation sites;
(b) detection of methylation of more than two adjacent methylation sites;
(c) detection of methylation at adjacent sites within the same section of a target region amplified by one primer pair;
(d) detection of methylation at non-adjacent sites within the same section of a region amplified by one primer pair;
(e) detection of methylation at adjacent sites within the same target region;
(f) detection of methylation at non-adjacent sites within the same target region;
(g) any one of (a) to (f) in the same sequencing read;
(h) any one of (a) to (f) in at least two sequencing reads;
(i) any one of (a) to (f) in a plurality of sequencing reads;
(j) detection over methylation at sets of adjacent sites that overlap;
(k) repeated observation of any one of (a) to (j); or
(l) any combination or subset of the above.

In one embodiment, each of the regions is amplified in sections using multiple primer pairs. In one embodiment, these sections are non-overlapping. The sections may be immediately adjacent or spaced apart (e.g. spaced apart up to 10, 20, 30, 40, or 50 bp). Since target regions (including CpG islands, CpG shores, and/or CpG shelves) are usually longer than 75 to 150 bp, this embodiment permits the methylation status of sites across more (or all) of a given target region to be assessed.

A person of ordinary skill in the art would be well aware of how to design primers for target regions using available tools such as Primer3, Primer3Plus, Primer-BLAST, etc. As discussed, bisulphite conversion results in cytosine converting to uracil and 5′-methyl-cytosine converting to thymine. Thus, primer positioning or targeting may make use of bisulphite converted methylate sequences, depending on the degree of methylation specificity required.

Target regions for amplification are designed to have at least two CpG dinucleotide methylation sites. In some embodiments, however, it may be advantageous to amplify regions having more than one CpG methylation site. For instance, the amplified regions may have 3, 4, 5, 6, 7, 8, 9, 10, 11, 12, 13, 14, or 15 CpG methylation sites. In one embodiment, the primers are designed to amplify DNA fragments comprising 3 to 12 CpG methylation sites. Overall this permits a larger number of adjacent methylation sites to be queried within a single sequencing read, and provides additional certainty (exclusion of false positives) because multiple concordant methylations can be detected within a single sequencing read. In one embodiment, the tumour signals comprise more than two adjacent methylation sites within the single sequencing read. Detecting more than two adjacent methylation sites provides additional concordance, and additional confidence that the tumour signal is not a false positive in this embodiment. For example, a tumour signal may be designated as 3, 4, 5, 6, 7, 8, 9, 10 or more adjacent detected methylation sites within a single sequencing read. In one embodiment, the detection of more than one of the tumour signals is indicative of a tumour. Detection of multiple tumour signals, in this embodiment, can increase confidence in tumour detection. Such signals can be at the same or at different sites. In one embodiment, the detection of more than one of the tumour signals at the same region is indicative of a tumour. Detection of multiple tumour signals indicative of methylation at the same site in the genome, in this embodiment, can increase confidence in tumour detection. So too can detection of methylation at adjacent sites in the genome, even if the signals are derived from different sequencing reads. This reflects another type of concordance. In one embodiment, the detection of adjacent or overlapping tumour signals across at least two different sequencing reads is indicative of a tumour. In one embodiment, the adjacent or overlapping tumour signals are within the same CpG island. In one embodiment, the detection of 5 to 25 adjacent methylated sites is indicative of a tumour.

Methylated regions can be selected according to the purpose of the intended assay. In one embodiment, the regions comprise at least one region listed Table 1 and/or Table 2. In one embodiment, the regions comprise all regions listed in Table 1 and/or Table 2.

Likewise, primer pairs can be designed based on the intended target regions.

In one embodiment, the step of amplification is carried out with more than 100 primer pairs. The step of amplification may be carried out with 10, 20, 30, 40, 50, 60, 70, 80, 90, 100, 110, 120, 130, 140, 150, or more primer pairs. In one embodiment, the step of amplification is a multiplex amplification. Multiplex amplification permits large amount of methylation information to be gathered from many target regions in the genome in parallel, even from cfDNA samples in which DNA is generally not plentiful. The multiplexing may be scaled up to a platform such as ION AmpliSeq™, in which, e.g. up to 24,000 amplicons may be queried simultaneously. In one embodiment, the step of amplification is nested amplification. A nested amplification may improve sensitivity and specificity.

The nested reaction may be part of a next generation sequencing approach. Barcode and/or sequencing primers may be added in the second (nested) amplification. Alternatively, these may added in the first amplification.

In one embodiment, the method further comprises quantifying the tumour signals, wherein a number in excess of a threshold is indicative of a tumour. In one embodiment, the steps of quantifying and comparing are carried out independently for each of the sites methylated in cancer. Accordingly, a count of positive tumour signals may be established for each site. In one embodiment, the method further comprises determining a proportion of the sequencing reads containing tumour signals, wherein the proportion in excess of a threshold is indicative of a tumour. In one embodiment, the step of determining is carried out independently for each of the sites methylated in cancer.

By “threshold”, as used herein, is meant a value that is selected to discriminate between a disease (e.g., malignant) state, and a non-disease (e.g., healthy) state. Thresholds can be set according to the disease in question, and may be based on earlier analysis, e.g., of a training set. Thresholds may also be set for a site according to the predictive value of methylation at a particular site. Thresholds may be different for each methylation site, and data from multiple sites can be combined in the end analysis.

Various design parameters may be used to select the regions subject to amplification in some embodiments. In one embodiment, the regions are not methylated in healthy tissue. Healthy tissue would be understood to be non-malignant. Healthy tissue is often selected based on the origin of the corresponding tumour.

Regions may be selected based on desired aims or required specificity, in some embodiments. For instance, it may be desirable to screen for more than one cancer type. Thus, in one embodiment, the regions are collectively methylated in more than one tumour type. It may be desirable to include regions methylated generally in a group of cancers, and regions methylated in specific cancers in order to provide different tiers of information. Thus, in one embodiment, the regions comprise regions that are specifically methylated in specific tumours, and regions that are methylated in more than one tumour type. Likewise, it may be desirably to include a second tier of regions that can differentiate between tumour types. In one embodiment, the regions specifically methylated in specific tumours comprise a plurality of groups, each specific to one tumour type. However, it may be desirable in some contexts to have a test that is focused on one type of cancer. Thus, in one embodiment, the regions are methylated specifically in one tumour type. In one embodiment, the regions are selected from those listed in Table 3 and the tumour is one carrying a BRCA1 mutation.

More specifically, in some embodiments regions may be selected that are methylated in particular subtypes of a cancer exhibiting particular histology, karyotype, gene expression (or profile thereof), gene mutation (or profile thereof), staging, etc. Accordingly, the regions to be amplified may comprise one or more groups of regions, each being established to be methylated in one particular cancer subtype. In one embodiment the regions to be amplified may be methylated in a cancer subtype bearing particular mutations. With breast cancer in mind, one example subtype defined by mutation is cancer bearing BRCA1 mutations. Another subtype is cancer bearing BRCA2 mutations. Other breast cancer subtypes for which methylated regions may be determined include Basal, Luminal A, Luminal B, HER2 and Normal-like tumours. For uveal melanoma, for example, subtypes may include tumours that have retained or lost chromosome 3 (monosomy 3).

Within the context of such a test of some embodiments, information about not only the presence, but also the pattern and distribution of tumour signals both within specific regions and between different regions may help to detect or validate the presence of a form of cancer. In one embodiment, the method further comprises determining a distribution of tumour signals across the regions, and comparing the distribution to at least one pattern associated with a cancer, wherein similarity between the distribution and the pattern is indicative of the cancer.

“Distribution”, as used herein in this context, is meant to indicate the number and location of tumour signals across the regions. Statistical analysis may be used to compare the observed distribution with, e.g., pre-established patterns (data) associated with a form of cancer. In other embodiments, the distribution may be compared to multiple patterns. In one embodiment, the method further comprises determining a distribution of tumour signals across the regions, and comparing the distribution to a plurality of patterns, each one associated with a cancer type, wherein similarity between the distribution and one of the plurality of patterns is indicative of the associated cancer type.

In one embodiment, the step of generating sequencing reads is carried out by next generation sequencing. This permits a very high depth of reads to be achieved for a given region. These are high-throughput methods that include, for example, Ilumina (Solexa) sequencing, Roche 454 sequencing, Ion Torrent sequencing, and SOLID sequencing. The depth of sequencing reads may be adjusted depending on desired sensitivity.

In one embodiment, the step of generating sequencing reads is carried out simultaneously for samples obtained from multiple patients, wherein the amplified CpG islands from is barcoded for each patient. This permits parallel analysis of a plurality of patients in one sequencing run.

A number of design parameters may be considered in the selection of regions methylated in cancer, according to some embodiments. Data for this selection process may be from a variety of sources such as, e.g., The Cancer Genome Atlas (TCGA) (http://cancergenome.nih.gov/), derived by the use of, e.g., Illumina Infinium HumanMethylation450 BeadChip (http://www.illumina.com/products/methylation_450_beadchip kits.html) for a wide range of cancers, or from other sources based on, e.g., bisulphite whole genome sequencing, or other methodologies. For instance, “methylation value” (understood herein as derived from TCGA level 3 methylation data, which is in turn derived from the beta-value, which ranges from −0.5 to 0.5) may be used to select regions. In one embodiment, the step of amplification is carried out with primer sets designed to amplify at least one methylation site having a methylation value of below −0.3 in normal issue. This can be established in a plurality of normal tissue samples, for example 4. The methylation value may be at or below −0.1, −0.2, −0.3, −0.4, or −0.5. In one embodiment, the primer sets are designed to amplify at least one methylation site having a difference between the average methylation value in the cancer and the normal tissue of greater than 0.3. The difference may be greater than 0.1, 0.2, 0.3, 0.4, or 0.5. Proximity of other methylation sites that meet this requirement may also play a role in selecting regions, in some embodiments. In one embodiment, the primer sets include primer pairs amplifying at least one methylation site having at least one methylation site within 200 bp that also has a methylation value of below −0.3 in normal issue, and a difference between the average methylation value in the cancer and the normal tissue of greater than 0.3. In another embodiment the adjacent site having these features may be 300 bp. The adjacent site may be within 100, 200, 300, 400, or 500 bp.

In some embodiments, target regions may be selected for amplification based on the number of tumours in the validation set having methylation at that site. For example, a region may be selected if it is methylated in at least 50%, 55%, 60%, 65%, 70%, 75%, 80, 85%, 90, or 95% of tumours tested. For example, regions may be selected if they are methylated in at least 75% of tumours tested, including within specific subtypes. For some validations, it will be appreciated that tumour-derived cell lines may be used for the testing.

In another embodiment, the method further comprises oxidative bisulphite conversion. In addition to the analysis of methylation of CpG residues, additional information that may be of clinical significance may be derived from the analysis of hydroxymethylation. Bisulphite sequencing results in the conversion of unmethylated cytosine residues into uracil/thymidine residues, while both methylated and hydroxymethylated cytosines remain unconverted. However, oxidative bisulphite treatment allows for the conversion of hydroxymethylated cytosines to uracil/thymidine allowing for the differential analysis of both types of modifications. By comparison of bisulphite to oxidative bisulphite treatments the presence of hydroxymethylation can be deduced. This information may be of significance as its presence or absence may be correlated with clinical features of the tumor which may be clinically useful either as a predictive or prognostic factor. Accordingly, in some embodiments, information about hydroxymethylation could additionally be used in the above-described embodiments.

In one aspect, the presence of specific patterns of methylation is linked to underlying characteristics of particular tumours. In these cases, the methylation patterns detected by the method are indicative of clinically relevant aspects of the tumours such as aggressiveness, likelihood of recurrence, and response to various therapies. Detection of these patterns in the blood may thus provide both prognostic and predictive information related to a patient's tumor.

In another aspect, the forgoing method may be applied to clinical applications involving the detection or monitoring of cancer.

In one embodiment, the forgoing method may be applied to determine and/or predict response to treatment.

In one embodiment, the forgoing method may be applied to monitor and/or predict tumour load.

In one embodiment, the forgoing method may be applied to detect and/or predict residual tumour post-surgery.

In one embodiment, the forgoing method may be applied to detect and/or predict relapse.

In one aspect, the forgoing method may be applied as a secondary screen.

In one aspect, the forgoing method may be applied as a primary screen.

In one aspect, the forgoing method may be applied to monitor cancer development.

In one aspect, the forgoing method may be applied to monitor and/or predict cancer risk.

In another aspect, there is provided a kit for detecting a tumour comprising reagents for carrying out the aforementioned method, and instructions for detecting the tumour signals. Reagents may include, for example, primer sets, PCR reaction components, and/or sequencing reagents.

In one embodiment of the forgoing methods, the regions comprise C2CD4A, COL19A1, DCDC2, DHRS3, GALNT3, HES5, KILLIN, MUC21, NR2E1/OSTM1, PAMR1, SCRN1, and SEZ6, and the tumour is uveal melanoma. In one embodiment, the probes comprise C2C5F, COL2F, DCD5F, DGR2F, GAL1F, GAL3F, HES1F, HES3F, HES4F, KIL5F, KIL6F, MUC2F, OST3F, OST4F, PAM4F, SCR2F, SEZ3F, and SEZ5F.

In one embodiment, the regions comprise ADCY4, ALDH1L1, ALOX5, AMOTL2, ANXA2, CHST11, EFS, EPSTI1, EYA4, HAAO, HAPLN3, HCG4P6, HES5, HIF3A, HLA-F, HLA-J, HOXA7, HSF4, KLK4, LOC376693, LRRC4, NBR1, PAH, PON3, PPM1H, PTRF, RARA, RARB, RHCG, RND2, TMP4, TXNRD1, and ZSCAN12, and the tumour is prostate cancer. In one embodiment, the probes comprise ADCY4-F, ALDH1L1-F, ALOX5-F, AMOTL2-F, ANXA2-F, CHST11-F, EFS-F, EPSTI1-F, EYA4-F, HAAO-F, HAPLN3-F, HCG4P6-F, HES5-F, HIF3A-F, HLA-F-F, HLA-J-1-F, HLA-J-2-F, HOXA7-F, HSF4-F, KLK4-F, LOC376693-F, LRRC4-F, NBR1-F, PAH-F, PON3-F, PPM1H-F, PTRF-F, RARA-F, RARB-F, RHCG-F, RND2-F, TMP4-F, TXNRD1-F, and ZSCAN12-F. In one embodiment, the probes additionally include C1Dtrim, C1 Etrim, CHSAtrim, DMBCtrim, FOXAtrim, FOXEtrim, SFRAtrim, SFRCtrim, SFREtrim, TTBAtrim, VWCJtrim, and VWCKtrim.

In one embodiment, the regions comprise ASAP1, BC030768, C18orf62, C6orf141, CADPS2, CORO1C, CYP27A1, CYTH4, DMRTA2, EMX1, HFE, HIST1H3G/1H2BI, HMGCLL1, KCNK4, KJ904227, KRT78, LINC240, Me3, MIR1292, NBPF1, NHLH2, NRN1, PPM1H, PPP2R5C, PRSS3, SFRP2, SLCO4C1, SOX2OT, TUBB2B, USP44, Intergenic (Chr1), Intergenic (Chr2), Intergenic (Chr3), Intergenic (Chr4), Intergenic (Chr8), and Intergenic (Chr10), and the tumour is aggressive prostate cancer. In one embodiment, the aggressive prostate cancer has a Gleason Score greater than 6. In one embodiment, the aggressive prostate cancer has a Gleason Score of 9 or greater. In one embodiment, the probes comprise ASAP1/p, BC030768/p, C18orf62/p, C6orf141/p-1, C6orf141/p-2, CADPS2/p, CORO1C/p-1, CORO1C/p-2, CYP27A1/p, CYTH4/p, DMRTA2/p, EMX1/p, HFE/p-1, HFE/p-2, HIST1H3G/1H2BI/p, HMGCLL1/p, KCNK4/p, KJ904227/p, KRT78/p, LINC240/p-1, LINC240/p-2, Me3/p-1, Me3/p-2, MIR129, NBPF1/p, NHLH2/p, NRN1/p, PPM1H/p-1, PPM1H/p-2, PPP2R5C/p, PRSS3/p, SFRP2/p-1, SFRP2/p-2, SLCO4C1/p, SOX2OT/p, TUBB2B/p, USP44/p, Chr1/p-1, Chr2/p-1, Chr3/p-1, Chr4/p-1, Chr8/p-1, and Chr10/p-1.

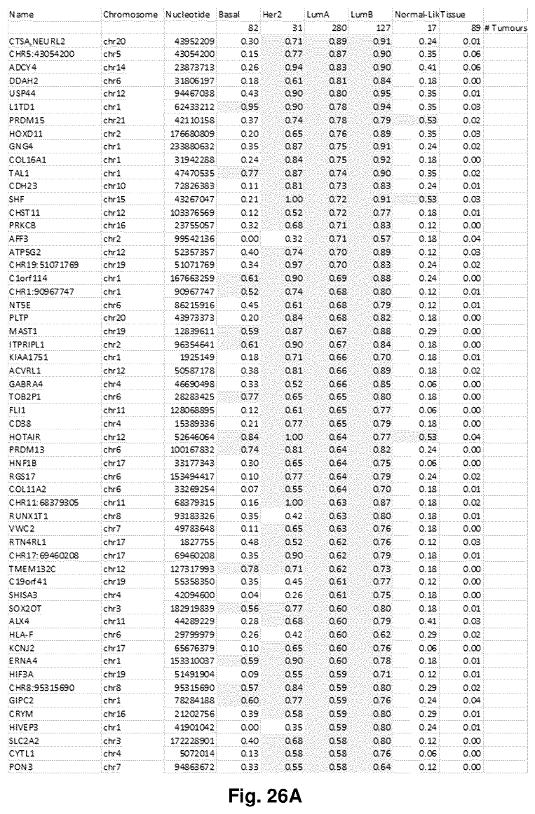

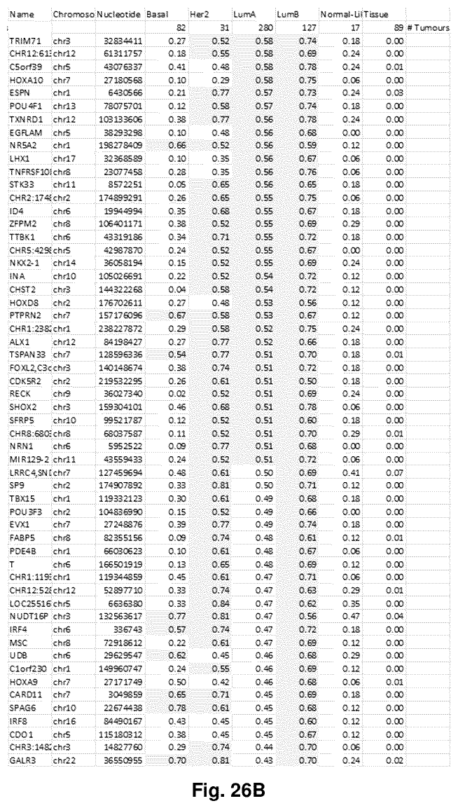

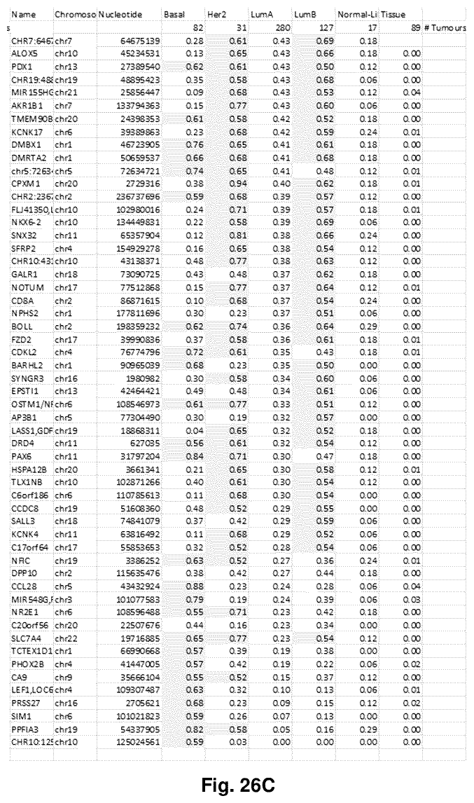

In one embodiment, the regions comprise the regions depicted in FIGS. 26A, 26B, and 26C, and the tumour is breast cancer.

In one embodiment, the regions comprise ALX1, ACVRL1, BRCA1,C1orf114, CA9, CARD11, CCL28, CD38, CDKL2, CHST11, CRYM, DMBX1, DPP10, DRD4, ERNA4, EPSTI1, EVX1, FABP5, FOXA3, GALR3, GIPC2, HINF1B, HOXA9, HOXB13, Intergenic5, Intergenic 8, IRF8, ITPRIPL1, LEF1, LOC641518, MAST1, BARHL2, BOLL, C5orf39, DDAH2, DMRTA2, GABRA4, ID4, IRF4, NT5E, SIM1, TBX15, NFIC, NPHS2, NR5A2, OTX2, PAX6, GNG4, SCAND3, TAL1, PDX1, PHOX2B, POU4F1, PFIA3, PRDM13, PRKCB, PRSS27, PTGDR, PTPRN2, SALL3, SLC7A4, SOX2OT, SPAG6, TCTEX1D1, TMEM132C, TMEM90B, TNFRSF10D, TOP2P1, TSPAN33, TTBK1, UDB, and VWC2, and the tumour is triple negative breast cancer (TNBC). In one embodiment, the probes comprise ALX1, AVCRL1, BRCA1-A, C1Dtrim, C1Etrim, CA9-A, CARD11-B, CCL28-A, CD38, CDKL2-A, CHSAtrim, CRYM-A, DMBCtrim, DMRTA2exp-A, DPP10-A, DPP10-B, DPP10-C,DRD4-A, EFNA4-B, EPSTI1, EVX1, FABP5, FOXAtrim, FOXEtrim, GALR3-A, GIPC2-A, HINF C trim, HOXAAtrim, HOXACtrim, HOXB13-A, Int5, Int8, IRF8-A, ITRIPL1, LEF1-A, MAST1 A trim, mbBARHL2 Trim, mbBOLL Trim, mbC5orf Trim, mbDDAH Trim, mbDMRTA Trim, mbGABRA A Trim, mbGABRA B Trim, mbGNG Trim, mbID4 Trim, mbIRF Trim, mbNT5E Trim, mbSIM A Trim, mbTBX15 Trim, NFIC-B, NFIC-A, NPSH2-B, NR5A2-B, OTX2-A, PAX6-A, pbDMRTA Trim, pbGNG Trim, pbSCAND Trim, pbTAL Trim, PDX1exp-B, PHOX2B-A, POU4F1 A trim, PPFIA3-A, PRDM13, PRKCB-A, PRKCB-C, PRSS27-A, PTGDR, PTPRN2-A, PTPRN2-B, SALL3-A, SALL3-B, SLC7A4-A, SOX2OT-B, SPAG6 A trim, TCTEX1D1-A, TMEM-A, TMEM-B, TMEM90B-A, TNFRSF10D, TOP2P1-B, TSPAN33-A, TTBAtrim, UBD-A, VWCJtrim, and VWCKtrim.

In one embodiment, each region is amplified with primer pairs listed for the respective region in Table 15.

In one embodiment, the method further comprises administering a treatment for the tumour detected.

In one aspect, there is provided a method for identifying a methylation signature indicative of a biological characteristic, the method comprising: obtaining data for a population comprising a plurality of genomic methylation data sets, each of said genomic methylation data sets associated with biological information for a corresponding sample, segregating the methylation data sets into a first group corresponding to one tissue or cell type possessing the biological characteristic and a second group corresponding to a plurality of tissue or cell types not possessing the biological characteristic, matching methylation data from the first group to methylation data from the second group on a site-by-site basis across the genome, identifying a set of CpG sites that meet a predetermined threshold for establishing differential methylation between the first and second groups, identifying, using the set of CpG sites, target genomic regions comprising at least two differentially methylated CpGs with 300 bp that meet said predetermined criteria, extending the target genomic regions to encompass at least one adjacent differentially methylated CpG site that does not meet the predetermined criteria, wherein the extended target genomic regions provide the methylation signature indicative of the biological trait.

In one embodiment, the method further comprises validating the extended target genomic regions by testing for differential methylation within the extended target genomic regions using DNA from at least one independent sample possessing the biological trait and DNA from at least one independent sample not possessing the biological sample.

In one embodiment, the step of identifying further comprises limiting the set of CpG sites to CpG sites that further exhibit differential methylation with peripheral blood mononuclear cells from a control sample.

In one embodiment, the plurality of tissue or cell types of the second group comprises at least some tissue or cells of the same type as the first group.

In one embodiment, the plurality of tissue or cell types of the second group comprises a plurality of non-diseased tissue or cell types.

In one embodiment, the predetermined threshold is indicative of methylation in the first group and non-methylation in the second group.

In one embodiment, the predetermined threshold is at least 50% methylation in the first group.

In one embodiment, the predetermined threshold is a difference in average methylation between the first and second groups of 0.3 or greater.

In one embodiment, the biological trait comprises malignancy.

In one embodiment, the biological trait comprises a cancer type.

In one embodiment, the biological trait comprises a cancer classification.

In one embodiment, the cancer classification comprises a cancer grade.

In one embodiment, the cancer classification comprises a histological classification.

In one embodiment, the biological trait comprises a metabolic profile.

In one embodiment, the biological trait comprises a mutation.

In one embodiment, the mutation is a disease-associated mutation.

In one embodiment, the biological trait comprises a clinical outcome.

In one embodiment, the biological trait comprises a drug response.

In one embodiment, the method further comprises designing a plurality of PCR primers pairs to amplify portions of the extended target genomic regions, each of the portions comprising at least one differentially methylated CpG site.

In one embodiment, the step of designing the plurality of primer pairs comprising converting non-methylated cytosines uracil, to simulate bisulphite conversion, and designing the primer pairs using the converted sequence.

In one embodiment, the primer pairs are designed to have a methylation bias.

In one embodiment, the primer pairs are methylation-specific.

In one embodiment, the primer pairs have no CpG residues within them having no preference for methylation status.

In one aspect, there is provided a method for synthesizing primer pairs specific to a methylation signature, the method comprising: carrying out the forgoing method, and synthesizing the designed primer pairs.

In one aspect, there is provided a non-transitory computer-readable medium comprising instructions that direct a processor to carry out the forgoing method.

In one aspect, there is provided a computing device comprising the computer-readable medium.

### Example 1

### Concept Summary

The embodiments detect circulating tumour DNA using a highly sensitive and specific methylation based assay with detection limits 100 times better than other techniques.

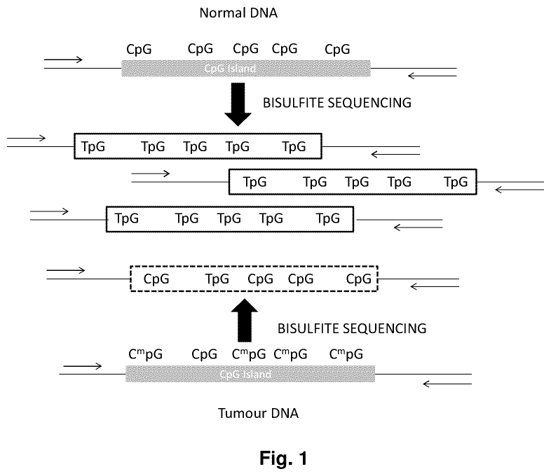

FIG. 1 depicts a schematic of the overall strategy. CpG dinucleotides are often clustered into concentrated regions in the genome referred to as CpG islands (grey box) and are often, but not always, associated with the promoter or enhancer regions of genes. These regions are known to become abnormally methylated in tumours (CmpG) as compared to normal tissue (CpG) which may be linked to the inactivation of tumour suppressor genes by this methylation event. Methylation of CpG islands and the boundary regions (CpG island shores) is extensive and co-ordinated such that most or all of the CpG residues in that region become methylated. The detection of this methylation typically involves bisulphite conversion, PCR amplification of the relevant region (arrows), and sequencing where un-methylated CpG residues are converted to TpG dinucleotides while methylated CpG residues are preserved as CpGs. Sequencing of these PCR-amplified “probes” (BISULFITE SEQUENCING) from tumour DNA (arrows) results in the detection of multiple CpG residues being methylated within the same DNA fragment (Dashed Box) which can easily be distinguished from DNA from normal tissue (Boxes). The co-ordinated/concordant nature of this methylation produces a strong signal which can be detected over random or background changes from DNA sequencing. This is accomplished by first identifying regions of tumour specific DNA methylation with multiple correlated CpG methylation sites within the same region.

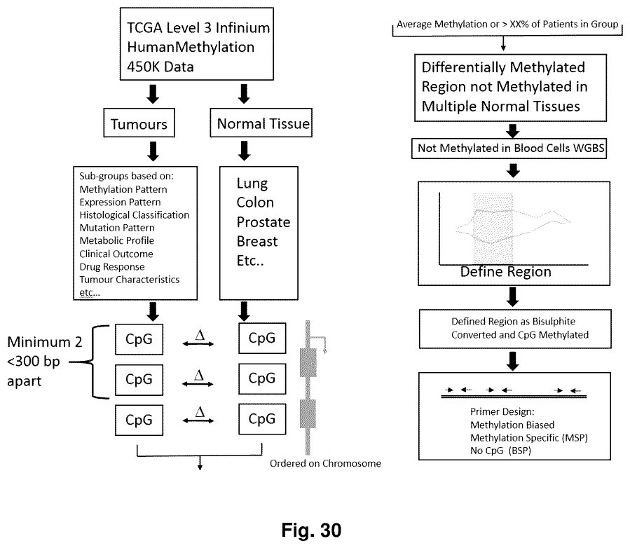

FIG. 30 depicts a flowchart showing how a methylation signature for a biological trait may be determined. One or more steps of this method may be implemented on a computer. Accordingly, another aspect of this disclosure relates to a non-transitory computer-readable medium comprising instructions that direct a processor to carry out steps of this method.

Generally “probe” is used herein to refer to a target region for amplification and/or the ensuing amplified PCR product. It will be understood that each probe is amplified by a “primer set” or “primer pair”.

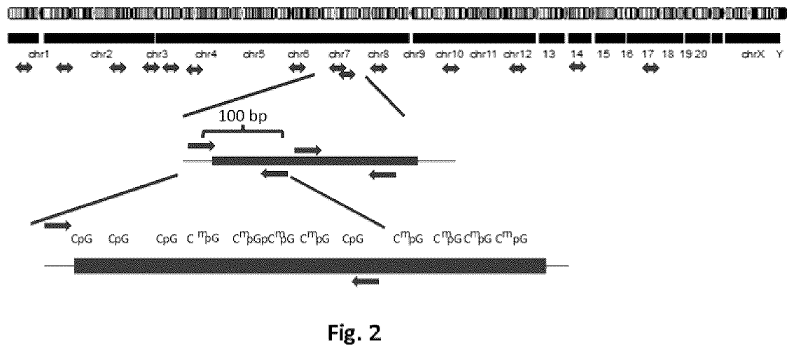

FIG. 2 depicts a schematic for amplification of target regions. Multiple regions from across the human genome have been identified as being differentially methylated in the DNA from various types of tumours compared to the normal DNA from a variety of different tissues. These regions can be fairly extensive spanning 100s to 1000s of base pairs of DNA. These target regions (black boxes, bottom) exhibit coordinated methylation where most or all of the CpG dinucleotides in these regions are methylated in tumour tissue with little or no methylation in normal tissues. As shown in FIG. 2, when sequencing across these regions (arrows) multiple CpG residues are seen to be methylated together in the tumour creating a concordant signal identifiable as being tumour specific. By targeting multiple PCR-amplified probes across individual regions (middle) and across the entire genome (top) large numbers of probes can be designed with the advantage that with more probes comes greater sensitivity due to the greater likelihood of detecting a tumour specific fragment in a given sample. Primers for these probes are designed to amplify regions from 75 to 150 bp in length, corresponding to the typical size of circulating tumour DNA. The primers may include CpG dinucleotides or not, which in the former case can make these primers biased towards the amplification of methylated DNA or exclusively amplify only methylated DNA.

Multiple methylation-biased PCR primer pairs can be created, which are able to preferentially amplify these regions. These multiple regions are sequenced using next generation sequencing (NGS) at a high read depth to detect multiple tumour specific methylation patterns in a single sample. As described herein, features have been incorporated into a blood based cancer detection system that provides advantages over other tests which have been developed, and provides an unprecedented level of sensitivity and specificity as well as enables the detection of minute quantities of DNA (detection sensitivity).

### Example 2

### Probe and Primer Set Development

The detection of circulating tumour DNA is hampered by both the presence of large amounts of normal DNA as well as by the very low concentrations of tumour DNA in the blood. Compounding this issue, both PCR and sequencing based approaches suffer from the introduction of single nucleotide changes due to the error prone nature of these processes. To deal with these issues, regions of the genome have been identified that exhibit concerted tumour specific methylation over a significant expanse of DNA so that each CpG residue is concordant²¹. Methylation-biased PCR primer pairs were designed for multiple segments of DNA across these regions each containing multiple CpG residues. Sample protocols for selection of differentially methylated regions and design of region specific PCR primers are provided.

### Protocol for the Selection of Differentially Methylated Regions

### Use of TCGA DATA for Identifying Breast Specific Probes

Level 3 (processed) Illumina Infinium HumanMethylation450 BeadChip array data (http://www.illumina.com/techniques/microarrays/methylation-arrays.html) was downloaded from The Tumour Genome Atlas (TCGA) site (https://tcga-data.nci.nih.gov/tcga/tcgaHome2.jsp) for the appropriate tumour types (e.g., breast, prostate, colon, lung, etc.). Tumour and normal samples were separated and the methylation values (from −0.5 to +0.5) for each group were averaged. The individual methylation probes were mapped to their respective genomic location. Probes that fulfilled the following example criteria were then identified:
1. The average methylation values for the normal breast, prostate, colon and lung tissues all below −0.3;
2. The difference between the average breast tumour and average breast normal values greater than 0.3, or at least 50% methylation in the tumour group; and
3. Two probes within 300 bp of each other fulfill criteria 1 and 2.

These criteria establish that the particular probe is not methylated in normal tissue, that the difference between the tumour and normal is significant, and that multiple probes in a relatively small area are co-ordinately methylated. Regions which had multiple positive consecutive probes (i.e., 3 or more) were prioritized for further analysis. Average values for approximately 10 other probes to either side of the positive region were plotted for all tumour and normal tissue samples to define the region exhibiting differential methylation. Regions exhibiting concerted differential methylation between tumour and normal for single or multiple tumour types were identified.

A secondary screen for a lack of methylation of these regions in blood was carried out by examining the methylation status of the defined regions in multiple tissues using nucleotide level genome wide bisulphite sequencing data. Specifically the UCSC Genome Browser (https://genome.ucsc.edu/) was used to examine methylation data from multiple sources.

Data was processed by the method described in Song Q, et al., A reference methylome database and analysis pipeline to facilitate integrative and comparative epigenomics. PLOS ONE 2013 8(12): e81148 (http://journals.plos.org/plosone/article?id=10.1371/journal.pone.0081148) for use in the UCSC Browser and to identify hypo-methylated regions (above blue lines).

The Following Data Sources were Used:
Gertz J, et al., Analysis of DNA methylation in a three-generation family reveals widespread genetic influence on epigenetic regulation. PLOS Genet. 2011 7(8):e1002228 (http://journals.plos.org/plosgenetics/article?id=10.1371/journal.pgen.1002228).
Heyn H, et al., Distinct DNA methylomes of newborns and centenarians. Proc. Natl. Acad. Sci. U.S.A. 2012 109(26):10522-7 (http://www.pnas.org/content/109/26/10522).
Hon G C, et al., Global DNA hypomethylation coupled to repressive chromatin domain formation and gene silencing in breast cancer. Genome Res. 2012 22(2):246-58 (http://genome.cshlp.org/content/22/2/246).
Heyn H, et al., Whole-genome bisulfite DNA sequencing of a DNMT3B mutant patient. Epigenetics. 2012 7(6):542-50 (http://www.tandfonline.com/doi/abs/10.4161/epi.20523 #.VsS_gdIUVIw).
Hon G C, et al., Global DNA hypomethylation coupled to repressive chromatin domain formation and gene silencing in breast cancer. Genome Res. 2012 22(2):246-58 (http://genome.cshlp.org/content/22/2/246).

All of the regions identified exhibited hypo-methylation in normal blood cells including Peripheral Blood Mononuclear Cells (PBMC), the prime source of non-tissue DNA in plasma.

### Protocol for the Design of Region Specific Primers for PCR Amplification and Next Generation Sequencing

For regions identified as being differentially methylated in tumours, PCR primers were designed that are able to recognize bisulphite converted DNA which is methylated. Using Methyprimer Express™ or PyroMark™, or other web based programs, the DNA sequence of the region was converted to the sequence obtained when fully methylated DNA is bisulphite converted (i.e., C residues in a CpG dinucleotide remain Cs, while all other C residues are converted to T residues). The converted DNA was then analysed using PrimerBlast™ (http://www.ncbi.nlm.nih.gov/tools/primer-blast/) to generate optimal primers. Primers were not expressly selected to contain CpG residues but due to the nature of the regions, generally CpG islands, most had 1 to 3 CpGs within them. This renders them biased towards the amplification of methylated DNA but in many cases they do recognize and amplify non-methylated DNA as well. The region between the primers includes 2 or more CpG residues. Primers were chosen to amplify regions from 75 to 150 base pairs in size with melting temperatures in the range of 52-68° C. Multiple primers were designed for each region to provide increased sensitivity by providing multiple opportunities to detect that region. Adapter sequences (CS1 and CS2) were included at the 5′ end of the primers to allow for barcoding and for sequencing on multiple sequencing platforms by the use of adaptor primers for secondary PCR.

Primers were characterized by PCR amplification of breast cancer cell line DNA and DNA from various primary tumours. PCR amplification was done with individual sets of primers and Next Generation Sequencing carried out to characterize the methylation status of specific regions. Primer sets exhibiting appropriate tumour specific methylation were then combined into a multiplex PCR reaction containing many primers.

### Results

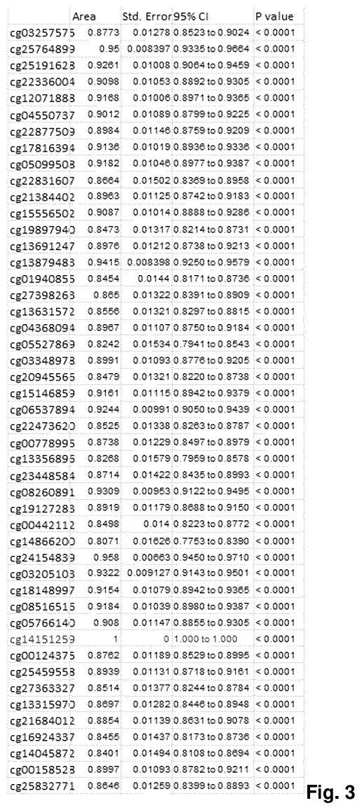

FIG. 3 lists the 47 CpG probes used to identify differentially methylated regions. These were analyzed by Receiver Operator Curve analysis (ROC). Normal and tumour samples from the entire TCGA breast cancer database were compared. The Area Under the Curve (AUC) analysis for each probe is shown with the standard error, 95% confidence interval and P-value. All of them where shown to have excellent discriminatory capabilities.

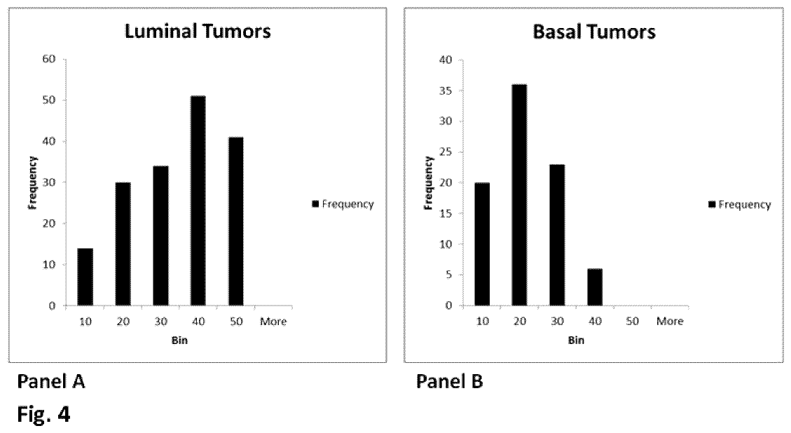

FIG. 4 depicts the results of analysis methylation level for each patient in the TCGA database for the 47 CpG. Those exceeding the threshold of −0.1 were considered to be positive for methylation in that patient. The number of probes exceeding this methylation threshold were calculated for each patient. Patients were divided into those with Luminal A and B subtypes (Luminal Tumours; FIG. 4, Panel A) and those with Basal cancers (Basal Tumours; FIG. 4, Panel B) or and the number of patients with a specific range of positive probes was calculated. The histogram shows the frequency of patents within each range of positive probes. While these probes give excellent coverage in both populations, there are more positive probes amongst the Luminal tumours than the Basal tumours. Additional probes specific to the different breast cancer subtypes have been identified and appropriate probe development and validation is underway.

### Example 3

### Selection of Regions for Cancer and Cancer Types

For breast cancer, 52 regions in the genome were identified that are highly methylated in tumours but where multiple normal tissues do not exhibit methylation of these regions. These serve as highly specific markers for the presence of a tumour with little or no background signal.

Table 1 depicts regions selected for breast cancer screening.

TABLE 1

| | | | | | |
|---|---|---|---|---|---|
| Chromo- | Start | End | General | | |
| some | (hg18) | (hg18) | Location | Tumour | Size |

2nd Generation

| | | | | | |
|---|---|---|---|---|---|
| chr1 | 167663259 | 167663533 | C1orf114 | P/B | 274 |
| chr7 | 49783577 | 49784309 | VWC2 | P/B/C | 732 |
| chr14 | 23873519 | 23873993 | ADCY4 | P/B/C | 474 |
| chr11 | 43559012 | 43559541 | MIR129-2 | B/C | 529 |

3rd Generation

| | | | | | |
|---|---|---|---|---|---|
| chr6 | 43319186 | 43319213 | TTBK1 | P/B | 27 |
| chr1 | 46723905 | 46724176 | DMBX1 | P/B/C | 271 |
| chr7 | 27171684 | 27172029 | HOXA9 | B | 345 |
| chr8 | 120720175 | 120720579 | ENPP2 | P/B | 404 |
| chr10 | 99521635 | 99521924 | SFRP5 | P/B | 289 |
| chr12 | 103376281 | 103376485 | CHST11 | P/B/C | 204 |
| chr19 | 51071603 | 51072234 | FOXA3 | P/B | 631 |

4th Generation

| | | | | | |
|---|---|---|---|---|---|
| chr1 | 47470535 | 47470713 | TAL1 | B | 178 |
| chr1 | 50658998 | 50659557 | DMRTA2 | B | 559 |
| chr1 | 66030610 | 66030634 | PDE4B | B | 24 |
| chr1 | 90967262 | 90967924 | BARHL2 | B | 662 |
| chr1 | 119331667 | 119332616 | TBX15 | B/C | 949 |
| chr1 | 153557070 | 153557585 | RUSC1, | B | 515 |
| | | | C1orf104 | | |
| chr1 | 233880632 | 233880962 | GNG4 | B | 330 |
| chr2 | 104836482 | 104837226 | POU3F3 | B | 744 |
| chr2 | 198359230 | 198359743 | BOLL | B/C | 513 |
| chr3 | 32834103 | 32834562 | TRIM71 | B/C | 459 |
| chr3 | 172228723 | 172228985 | SLC2A2 | B | 262 |
| chr4 | 5071985 | 5072137 | CYTL1 | B | 152 |
| chr4 | 42094549 | 42094615 | SHISA3 | B | 66 |
| chr4 | 46690266 | 46690578 | GABRA4 | B | 312 |
| chr5 | 38293273 | 38293312 | EGFLAM | B | 39 |
| chr5 | 43076195 | 43076642 | C5orf39 | B | 447 |
| chr5 | 115179918 | 115180393 | CDO1 | B | 475 |
| chr6 | 336189 | 337131 | IRF4 | B/C | 942 |
| chr6 | 19944994 | 19945298 | ID4 | B | 304 |
| chr6 | 28618285 | 28618318 | SCAND3 | B | 33 |
| chr6 | 31806197 | 31806205 | DDAH2 | B | 8 |
| chr6 | 33269254 | 33269355 | COL11A2 | B | 101 |
| chr6 | 86215822 | 86215929 | NT5E | B | 107 |
| chr6 | 101018889 | 101019751 | SIM1 | B | 862 |

5th Generation

| | | | | | |
|---|---|---|---|---|---|
| chr6 | 153493505 | 153494425 | RGS17 | B | 920 |
| chr7 | 121743738 | 121744126 | CAPDS2 | B | 388 |
| chr8 | 72918338 | 72918895 | MSC | B/C | 557 |
| chr10 | 22674438 | 22674584 | SPAG6 | B/C | 146 |
| chr10 | 105026601 | 105026737 | INA | B | 136 |
| chr11 | 128068895 | 128069316 | FLI1 | B/C | 421 |
| chr12 | 52357158 | 52357378 | ATP5G2 | B | 220 |
| chr12 | 94466892 | 94467095 | USP44 | B/C | 203 |
| chr13 | 78075521 | 78075764 | POU4F1 | B | 243 |
| chr14 | 55656275 | 55656325 | PELI2 | B | 50 |
| chr17 | 33176853 | 33178091 | HNF1B | B | 1238 |
| chr17 | 32368343 | 32368604 | LHX1 | B/C/L | 261 |
| chr17 | 44154844 | 44155027 | PRAC, | B/C | 183 |
| | | | C17orf93 | | |
| chr18 | 73090725 | 73091121 | GALR1 | B/C | 396 |
| chr19 | 12839383 | 12839805 | MAST1 | B | 422 |
| chr20 | 2729122 | 2729438 | CPXM1 | B/C | 316 |
| chr20 | 43952209 | 43952500 | CTSA, | B | 291 |
| | | | NEURL2 | | |

In Table 1, ‘Start’ and ‘End’ designate the coordinates of the target regions in the hg18 build of the human genome reference sequence. The ‘General Location’ field gives the name of one or more gene or ORF in the vicinity of the target region. Examination of these sequences relative to nearby genes indicates that they were found, e.g., in upstream, in 5′ promoters, in 5′ enhancers, in introns, in exons, in distal promoters, in coding regions, or in intergenic regions. The ‘Tumour’ field indicates whether a region is methylated in prostate (P), breast (B), colon (C), and/or lung (L) cancers. The ‘Size’ field indicates the size of the target region.

In the discussion here, it should be recognized that reference to genes such as CHST11, FOXA, and NT5 are not intended to be indicative of the genes in question per se, but rather to the associated methylated regions described in Table 1.

In total, 52 regions were found to be methylated in association with breast cancer, 17 were found to be methylated in association with prostate cancer, 9 were found to be methylated in association with prostate cancer, and 1 region was found to be methylated in association with lung cancer. Thus, some regions appear to be generally indicative of the various types of cancers assessed. Other regions methylated in subgroups of these, while others are specific for cancers. In the context of this assay and the types of cancers examined, regions may be described as being “specifically methylated in breast cancer”. However, it is noted that the same approach may be used to identify regions methylated specifically in other cancers.

Assays may be developed for cancer generally, or to detect groups of cancers or specific cancers. A multi-tiered assay may be developed using “general” regions (methylated in multiple cancers) and “specific” regions (methylated in only specific cancers). A multi-tiered test of this sort may be run together in one multiplex reaction, or may have its tiers executed separately.

### Probes for Breast Cancer

Over 150 different PCR primer pairs were developed to the 52 different regions in the genome shown to exhibit extensive methylation in multiple breast cancer samples from the TCGA database but with no or minimal methylation in multiple normal tissues and in blood cells (Peripheral Blood Mononuclear Cells and others).

As proof of concept, these were then used to amplify bisulphite converted DNA from breast cancer cell lines MCF-7 (ER+, PR+), T47-D (ER+, PR+), SK-BR-3 (HER2+), MDA-MD-231 (Triple Negative) and normal breast lines MCF-10A and 184-hTERT. Sequencing adapters were added and Next Generation Sequencing carried out on an Ion Torrent sequencer. The sequencing reads were then separated by region and the sequence reads were analyzed using the BiqAnalyzer HT program.

### Results

Example results of methylation analysis will be discussed herein. CHST11 is an example of a region methylated in prostate, breast, and colon cancer. FOXA is a region methylated in breast and prostate cancer. NT5 is a region methylated specifically in breast cancer.

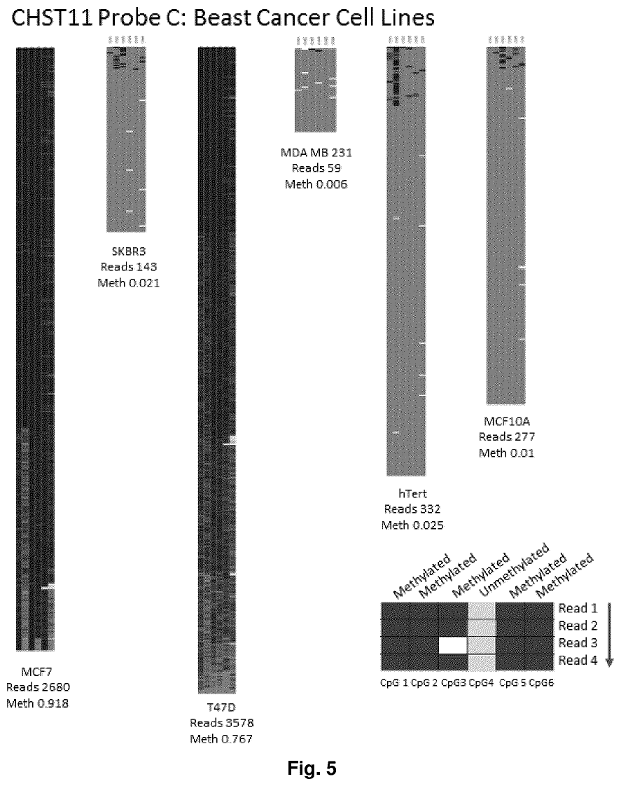

FIG. 5 depicts sequencing results from a region from near the CHST11 gene (Probe C) is shown. For each cell line the results of a single sequencing read is depicted as a horizontal bar with each box representing a single CpG residue from between the PCR primers (in this case there being 6 CpG residues, Illustration at bottom right). Methylated bases are shown in dark grey while un-methylated bases are shown in light grey. Where a CpG could not be identified by the alignment program it is shown as a white box. Multiple sequence reads are shown for each cell line, stacked on top of each other. The numbers at the bottom of each stack indicates the number of sequence reads (Reads) and the overall methylation level determined from these reads (Meth).

When sequenced, these probes produced strong concordant signals that consisted of multiple methylated CpGs (5 to 25) where there is a strong correlation between individual sites being methylated in tumours. This eliminates false positive results due to PCR and sequencing errors. These tumour specific multiple methylated sites can be detected against a high background of normal DNA, being limited only by the read depth of the sequencing. Based on bioinformatic analysis of TCGA tumours, this essentially eliminates false positive signals.

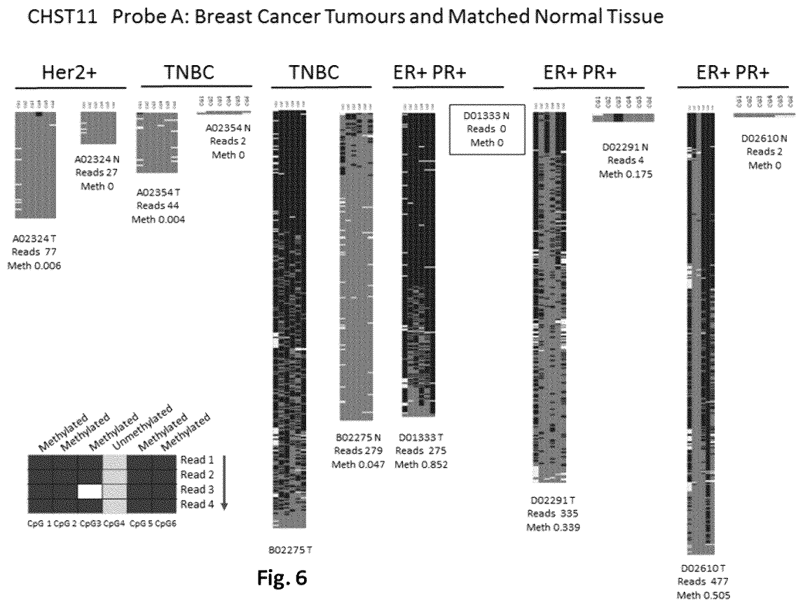

FIG. 6 depicts results for CHST11 Probe A. Methylation in the region was characterized for a variety of breast cancer tumour samples (T) and in normal breast tissue samples (N) from the same patient. As in FIG. 5 the methylated bases are shown in dark grey while un-methylated bases are shown in light grey (illustration bottom left). Tumours of various subtypes were analysed including A02324 which is positive for HER2 amplification (HER2+), A02354 and B02275 which are Triple Negative Breast Cancer (TNBC), and D01333, D02291, D02610 which are all Estrogen and Progesterone Receptor positive tumours (ER+PR+). The values below each column refer to the number of sequence reads obtained by Next Generation Sequencing (Reads) and the overall level of methylation of all of the CpG residues (Meth) based on these reads. Where no sequence reads were obtained for a given sample and box is shown as for sample D01333 N (Normal).

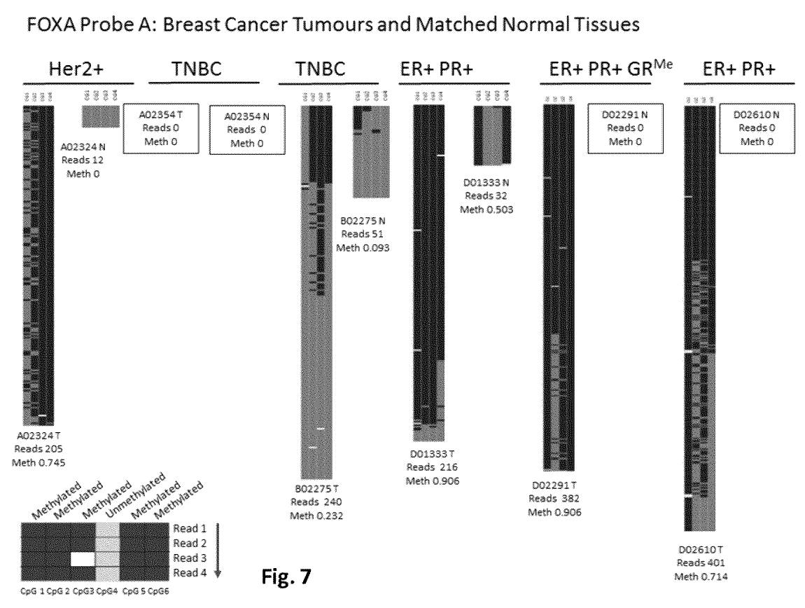

FIG. 7 depicts results of similar analysis of FOXA Probe A in breast cancer cell lines.

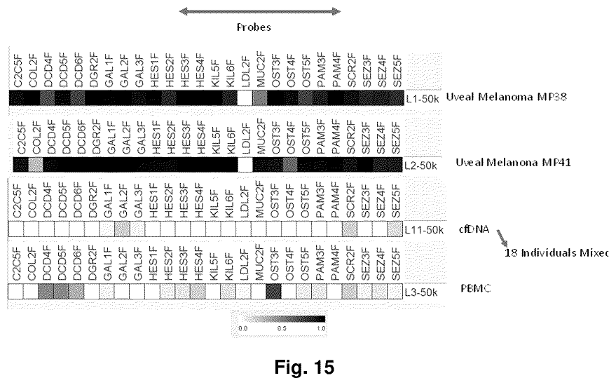

FIG. 15 depicts a numerical summary generated methylation data for prostate cell lines. #Reads is indicative of the number of reads exported, and Mean Me is indicative of the mean methylation.

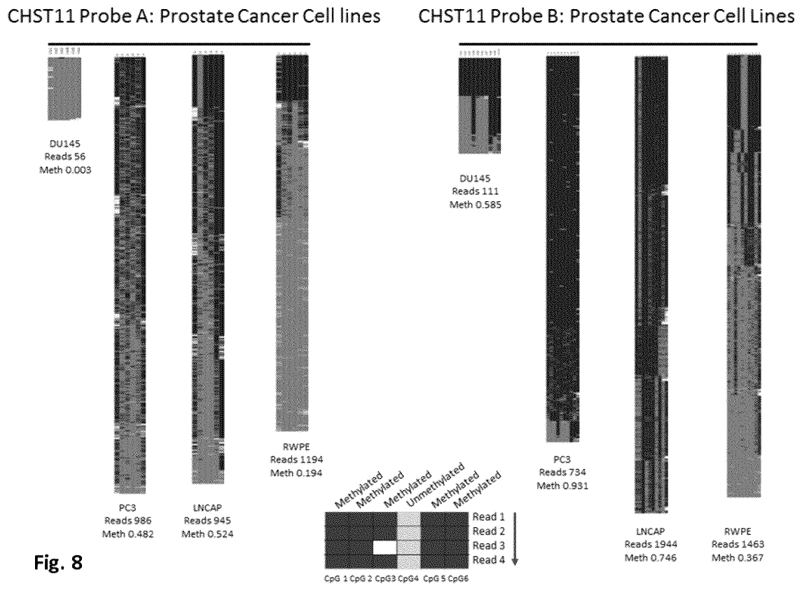

FIG. 8 depicts results of similar analysis of the CHST11 Probe A and CHST11 Probe B in prostate cancer cell lines. DU145 is an Androgen Receptor (AR−) negative cell line which is able to generate metastases in the mouse. PC3 is also AR- and also metastatic. LNCaP is an Androgen Receptor positive line (AR+) which does generate metastases in the mouse while RWPE cells are AR+ and non-metastatic.

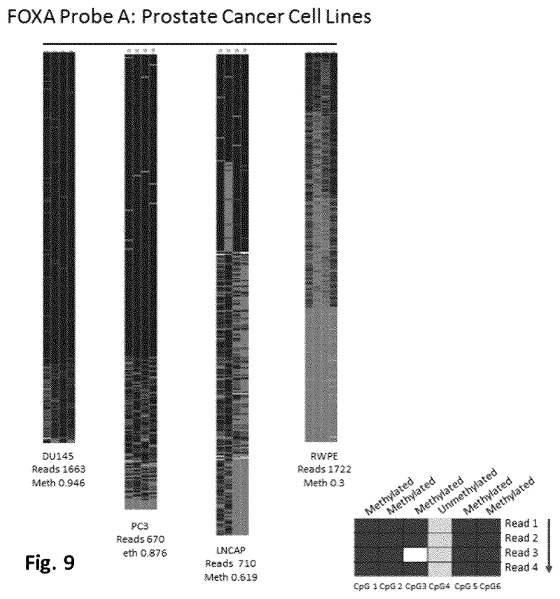

FIG. 9 depicts results of similar analysis of FOXA Probe A in prostate cell lines.

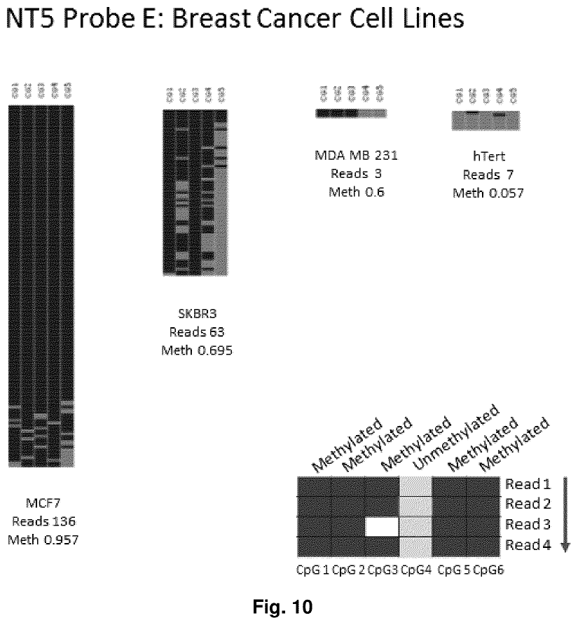

FIG. 10 depicts sequencing results to assess methylation status NET5 Probe E in breast cancer cell lines.

These results exemplify probes of differing specificities that can be selected using the approach outlined herein.

### Example 4

### Probes for Uveal Cancer

Using the above-described methodologies, regions were selected for uveal cancer screening. Table 2 depicts these regions.

TABLE 2

| Chromo- | | | General | | |
|---|---|---|---|---|---|
| some | Start | Stop | Location | Descriptor | Size |
| chr10 | 89611399 | 89611920 | PTEN, KILLIN | Shore CGI | 521 |
| chr11 | 35503400 | 35504124 | PAMR1 | small CGI | 724 |
| chr11 | 1.18E+08 | 1.18E+08 | MPZL2 | Prox Prom | 599 |
| chr15 | 60146043 | 60147120 | C2CD4A | Shore CGI | 1077 |
| chr17 | 24370858 | 24371386 | SEZ6 | small CGI | 528 |
| chr19 | 11060476 | 11060965 | LDLR | Prox Prom | 489 |
| chr2 | 1.66E+08 | 1.66E+08 | GALNT3 | CGI | 1465 |
| chr2 | 2.23E+08 | 2.23E+08 | ccdc140/pax3 | Shore CGI | 4724 |
| chr6 | 21774638 | 21775386 | FLI22536/casc15 | small CGI | 748 |
| chr6 | 24465699 | 24466545 | KAAG1, DCDC2 | CGI | 846 |
| chr6 | 31031220 | 31031651 | MUC21 | CGI | 431 |
| chr6 | 70632889 | 70633262 | COL19A1 | Proc Prom | 373 |
| chr6 | 1.09E+08 | 1.09E+08 | NR2E1/OSTM1 | small CGI | 1001 |
| chr7 | 29996242 | 29996333 | SCRN1 | Shore CGI | 91 |
| chr1 | 2450725 | 2452224 | HES5 | CGI | 1499 |
| chr1 | 12601228 | 12601893 | DHRS3 | Shore CGI | 665 |

### Example 5

### Tests for Breast Cancer Subtypes

The screen that has been described above, which originally incorporated all breast tumours in the TCGA database, can also be done on subsets of the tumour database.

BRCA1 carriers were taken out of the dataset and analyzed individually to identify target methylated regions specific to this subgroup. Breast cancer can also be divided in other ways: e.g., into five subtypes, Basal, Luminal A., Luminal B, HER2 and Normal-like. Patients in each of these groups were identified and analyzed to identify target methylated regions for each subset.

The screen can also be changed to look at individual patients using the previously described criteria to see who are positive or negative. Target methylated regions can then be ranked based on how many individuals are positive. This can help to remove biasing due to amalgamation (averaging). Targets can then be selected, e.g., if they are present in greater than 75% of patients for each subtype, and then rationalize amongst these.

### Test for BRCA Carriers

Current monitoring practices for women at high risk of developing breast cancer due to familial BRCA1 or 2 mutations involve yearly MRI, however the high false positive rates result in a large number of unnecessary biopsies. Using the methodology described herein, a test may be developed to serve as a secondary screen, e.g., to be employed after a positive MRI finding; or to be used for primary screening of high risk patients. The blood test is designed to detect all types of breast cancer but because ER+ breast cancer is the most frequent it is biased towards these cancers, though some of the constituent probes do recognize HER2+ and TNBC tumours. In order to provide optimal sensitivity for the monitoring of BRCA1 and 2 an assay optimized for these patients may be developed.

Both TNBC and BRCA1 and 2 patients were selected from the TOGA 450 k methylation database. Generally, most BRCA1 and 2 tumours will present as TNBC but many non-familial cancers are also TNBC. These patients were analyzed using the above-described tumour specific methylation region protocol on both the overall TNBC population and on the BRCA1 and 2 patients. 85 tumour specific regions were identified for TNBC, 67 for BRCA1 and 13 for BRCA2 populations. Of these 39 were present in any two populations and they constitute the starting point for the development of this assay. Appropriate regions for a BRCA1 specific test were identified and assessed in individual patients with known mutations. This population is surprisingly uniform and most patients are recognized by a large number of probes. AUCs for individual probes are for the most part very high. Based on these results, an assay can be developed to detect all three, i.e., TNBC, BRCA1 and 2. If additional detection sensitivity is required, then individual tests can be constructed. For high risk women who are BRCA1 or 2 mutation carriers, their mutation status should be known so that the appropriate test can be applied.

### Test for BRCA1 Carriers

Probes have been developed for the detection of cancer in carriers of the BRCA1 mutation. Methylation data from the TCGA Breast cancer cohort were selected from patients known to be carriers of pathogenic BRCA1 mutations. This data was then analyzed as described to identify regions of the genome specifically methylated in this sub-set of breast cancers. Table 3 lists appropriate regions identified and their genomic locations.

TABLE 3
Target Region (hg18 reference)

| chr | Nearest Gene | Start (nt) | End (nt) | Size |
|---|---|---|---|---|
| chr1 | LOC105378683 | 43,023,840 | 43,023,487 | 353 |
| chr1 | NPHS2 | 177,811,942 | 177,811,671 | 271 |
| chr1 | NR5A2 | 198,278,599 | 198,278,409 | 190 |
| chr11 | PAX6 | 31,783,955 | 31,782,545 | 1,410 |
| chr11 | KCNE3 | 73,856,332 | 73,855,762 | 570 |
| chr12 | KCNA6 | 4,789,491 | 4,789,342 | 149 |
| chr12 | TMEM132C | 127,318,539 | 127,317,001 | 1,538 |
| chr13 | PDX1 | 27,390,265 | 27,389,540 | 725 |
| chr13 | EPSTI1 | 42,464,618 | 42,463,901 | 717 |
| chr16 | A2BP1 | 6,009,930 | 6,009,020 | 910 |
| chr16 | CRYM | 21,202,914 | 21,202,448 | 466 |
| chr16 | PRKCB | 23,755,504 | 23,754,826 | 678 |
| chr16 | IRF8 | 84,490,354 | 84,490,167 | 187 |
| chr18 | SALL3 | 74,842,145 | 74,839,705 | 2,440 |
| chr19 | LYPD5 | 49,016,848 | 49,016,696 | 152 |
| chr2: | DPP10 | 115,636,420 | 115,635,215 | 1,205 |
| chr20 | C20orf56 | 22,507,867 | 22,507,676 | 191 |
| chr3 | SOX2OT | 182,919,993 | 182,919,839 | 154 |
| chr4 | CDKL2 | 76,774,880 | 76,774,658 | 222 |
| chr5 | March 11 | 16,233,072 | 16,232,633 | 439 |
| chr5 | CCL28 | 43,433,329 | 43,432,559 | 770 |
| chr5 | AP3B1 | 77,304,644 | 77,304,208 | 436 |
| chr7 | CARD11 | 3,050,299 | 3,049,859 | 440 |
| chr7 | BLACE | 154,859,799 | 154,859,051 | 748 |
| chr7 | PTPRN2 | 157,176,806 | 157,176,096 | 710 |
| chr8 | RUNX1T1 | 93,183,481 | 93,183,326 | 155 |

52 different probes were then developed to various parts of these regions and the methylation pattern in tumor cell lines was characterized, including MDA-MB-436 and HCC1937 which are known to carry BRCA1 mutations. These probes will be combined with previously characterized probes to other regions which are also methylated in tumours from BRCA1 patients. This would provide for a highly sensitive assay able to detect cancer in these high risk women at the earliest possible stage.

### Tests for Other Subtypes

A number of breast cell lines from women with known BRCA1 mutations have been isolated such as MDA-MB-436, HCC1937 and HCC1395 (all available from ATCC). These may be used to validate the assay as was done for the general blood test. For BRCA2 mutant lines there is only one ATCC cell line at present, HCC1937. There are several BRCA2 mutant ovarian cancer lines that have been identified and they may be used if the bioinformatic analysis confirms that these methylation markers are also found in ovarian cancer. The development of a single assay that detects both breast and ovarian cancer in BRCA2 carriers represents a distinct advantage as it would simultaneously monitor the two primary cancer risks in these patients.

The development of these assays follows the same course the above-described general assay proceeding from TCGA data to cells lines to patient samples. Tumour banks (some of which have mutation data) can be used for this, and analysis of these tumours provides an indication of their likely BRCA mutation. These samples can also be sequenced to confirm the prediction.

### Example 6

### Testing of Cell-Free Samples

Proof of concept testing was carried out using cell lines for ease of analysis. However, the assay can be applied to test for cell-free DNA, e.g., circulating cell-free tumour DNA in blood, and finds wide application in this context. A sample protocol for circulating tumour DNA is provided.

### Sample Protocol: Test for Circulating Tumour DNA

### DNA Preparation

The following example protocol may be used to detect circulating tumour DNA (tDNA).
Obtain DNA to be used for bisulfite conversion and downstream PCR amplification (i.e., cell line, tumour or normal DNA). Determine DNA purity on 0.8% agarose gel.
Determine genomic DNA (gDNA) for concentration in ug/uL by UV spectrophotometry.
Prepare a 1:100 dilution with TE buffer.
Remove RNA contaminates, if necessary, using the purification protocol for the GenElute Mammalian Genomic DNA Miniprep Kit, Sigma Aldrich, CAT #G1N350 (http://www.sigmaaldrich.com/technical-documents/protocols/biology/genelute-mammalian-genomic-dna-miniprep-kit.html). Follow purification protocol from steps A: 2a-3a, step 4-9.
OPTIONAL: For gDNA from a cell line, sonicate gDNA to approximately 90-120 bp (this represents general size of circulating tDNA). To do this, sonicate 5-10 ug of sample (50-100 ng/100 uL) using a sonicator. Use setting 4, and 15 pulses for 30 seconds with 30 seconds rest on ice in between. Determine sonicated DNA purity and bp size on 0.8% agarose gel.
Bisulfite convert DNA-EpiTect Fast Bisulfite Conversion Kit, QIAgen, CAT #59824 (https://www.qiagen.com/us/resources/resourcedetail?id=15863f2d-9d1c-4f12-b2e8-a0c6a82b2b1e&lang=en). Follow bisulfite conversion protocol on pages 1-18, 19-23. Refer to trouble shooting guide pages 30-32. Modifications to the protocol include: 1. Prepare reactions in 1.5 mL tubes, 2. High concentration samples at 2 ug, and low concentration samples at 500 ng-1 ug, 3. Perform the bisulfite conversion using 2 heat blocks set at 95° C. and 60ºC, 4. Incubation at 60° C. extended to 20 minutes, to achieve complete bisulfite conversion, 5a Elute DNA in 10-20 uL of elution buffer for ˜50-100 ng/uL final concentration, and 5b Dilute DNA to 10 ng/uL for use in PCR.
Perform nested PCR with Hot Star Taq Plus DNA Polymerase, Qiagen, CAT #203605 (https://www.qiagen.com/ca/resources/resourcedetail?id=c505b538-7399-43b7-ad10-d27643013d10&lang=en).

### Singleplex PCR Amplification

For singleplex PCR amplification of individual probes, carry out a primary PCR reaction with methylation-biased primers (MBP), (primer forward and reverse).

Table 4 recites reaction components.

TABLE 4

| Component | 1X (uL) |
|---|---|
| 10X PCR Buffer | 2.5 |
| 5 mM dNTP's | 1 |
| 5 U Hot Star Taq | 0.1 |
| 25 mM MgCl2 | 3 |
| PCR Grade H2O | 17 |
| [10 ng/uL] DNA | 1 |
| 10 pmol FWD Primer | 0.2 |
| 10 pmol REV Primer | 0.2 |
| Total | 25 |

Table 5 lists thermocycler conditions.

TABLE 5
Thermocycler Conditions

| | |
|---|---|
| Temp. | Time |

| | | | | |
|---|---|---|---|---|
| 95° C. | 15 | min | | |
| 95° C. | 30 | sec | | |
| 58° C. | 30 | sec | {close oversize bracket} | X 40 |
| 72° C. | 30 | sec | | |
| 72° C. | 7 | min | | |

| | |
|---|---|
| 4° C. | ∞ |

Carry out a secondary PCR reaction with universal primers CS1 (Barcode) and CS2 (P1 Adapter). To do this, remove an aliquot from the primary reaction, use as template DNA, this method serves as a two-step dilution PCR reaction

Table 6 recites reaction components.

TABLE 6

| Component | 1X (uL) |
|---|---|
| 10X PCR Buffer | 5 |
| 5 mM dNTP's | 2 |
| 5 U Hot Star Taq | 0.2 |
| 25 mM MgCl2 | 6 |
| PCR Grade H2O | 34.4 |
| MBP PCR Template | 2 |
| 10 pmol CS1 Primer | 0.2 |
| 10 pmol CS2 Primer | 0.2 |
| Total | 50 |

Table 7 recites thermocycler conditions.

TABLE 7
Thermocycler Conditions

| | |
|---|---|
| Temp. | Time |

| | | | | |
|---|---|---|---|---|
| 95° C. | 15 | min | | |
| 95° C. | 30 | sec | | |
| 58° C. | 30 | sec | {close oversize bracket} | X 3 |
| 72° C. | 30 | sec | | |
| 72° C. | 7 | min | | |

| | |
|---|---|
| 4° C. | ∞ |

Determine PCR specificity on 2% agarose gel. Run the methylation-biased PCR product and the CS1 CS2 sequencing PCR product beside one another on the agarose to visualize the banding pattern and increase in bp size. PCR product should be between 200-300 bp

For Singleplex PCR products, pool 5-10 uL of each PCR reaction (CS1 CS2 Secondary RXN) into a single tube for each sample type. Purify the pooled PCR with Agencourt AMPure XP beads at a 1.2:1 ratio (90 uL beads+75 uL sample), e.g., as below.

### Agencourt Ampure XP Bead Purification

Use freshly prepared 70% ethanol. Allow the beads and pooled DNA to equilibrate to room temperature.
1. Add indicated volume of Agencourt AMPure XP beads to each sample: 90 uL beads+75 uL Pool (1.2:1)
2. Pipet up and down 5 times to thoroughly mix the bead suspension with the DNA. Incubate the suspension at RT for 5 minutes.
3. Place the tube on a magnet for 5 minutes or until the solution clears. Carefully remove the supernatant and store until purified library has been confirmed.
4. Remove the tube from the magnet; add 200 uL of freshly prepared 70% EtOH. Place the tube back on the magnet and incubate for 30 seconds; turn the tube around twice in the magnet to move the beads through the EtOH solution. After the solution clears, remove and discard the supernatant without disturbing the pellet.
5. Repeat step #4 for a second EtOH wash.
6. To remove residual EtOH, pulse-spin the tube. Place the tube back on the magnet, and carefully remove any remaining EtOH with a 20 uL Pipette, without disturbing the pellet.
7. Keeping the tube on the magnet, air-dry the beads at RT for ˜5 minutes.
8. Remove the tube from the magnet; add 50 uL of TE directly to the pellet. Flick the tube to mix thoroughly. Incubate at RT for 5 minutes.
9. Pulse-spin and place the tube back on the magnet for ˜2 minutes or until the solution clears. Transfer the supernatant containing the eluted DNA to a new 1.5 mL Eppendorf LoBind tube.
10. Remove the tube from the magnet; add 50 uL of TE directly to the pellet. Flick the tube to mix thoroughly. Store the beads, along with the supernatant, at 4ºc until purified library has been confirmed.
11. Visualize the sample pre- and post-purification on an 8% acrylamide gel (higher resolution). Pooled PCR product should be visualized as multiple bands (as each PCR product is a slightly different bp size). Purified sample should eliminate product beneath 150 bp.

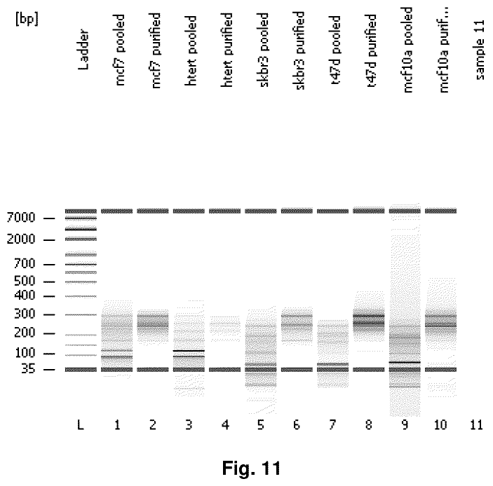

FIG. 11 depicts a summary of BioAnalyzer electrophoresis summary for amplification product generated from various cell lines.
12. Perform nested PCR with Multiplex PCR Plus Kit, Qiagen, CAT #206152 (https://www.qiagen.com/ca/resources/resourcedetail?id=beb1f99e-0580-42c5-85d4-ea5f37573c07&lang=en), e.g., as below.

### Multiplex PCR Amplification of Up to 50 Probes in a Single Reaction

Create multiplex primer mix by aliquot 1 uL of each forward and reverse primer at 10 pmol/uL into a single 1.5 mL tube. Calculate the final concentration of each primer by dividing the initial primer concentration by the final volume of primer mix in the tube, i.e., 15 probes to be multiplexed into a single reaction, would total 30 primers and at 1 uL each, 30 uL final volume. Thus ((10 pmol)(1 uL))/30 uL=0.333 pmol. Primer concentration requires optimization during PCR amplification, as the number of primers in a single reaction can influence the efficiency of the product, e.g.
15 primer sets ˜2 pmol final [ ] in PCR
50 primer sets ˜0.5 pmol final [ ] in PCR
Carry out primary PCR reaction with methylation-biased primers.

Table 8 lists reaction components for multiple amplifications of 15 probes, and Table 9 lists reaction components for multiple amplifications of 50 probes. Table 10 list reaction conditions.

TABLE 8
15 primer pairs at 2 pmol

| Component | 1X (uL) |
|---|---|
| 2X Multiplex MM | 25 |
| PCR H2O | 18 |
| Primer Mix | 6 |
| [10 ng/uL] DNA | 1 |
| Total | 50 |

TABLE 9
50 primer pairs at 0.5 pmol

| Component | 1X (uL) |
|---|---|
| 2X Multiplex MM | 25 |
| PCR H2O | 19 |
| Primer Mix | 5 |
| [10 ng/uL] DNA | 1 |
| Total | 50 |

TABLE 10
Thermocycling Conditions

| | |
|---|---|
| Temp. | Time |

| | | | | |
|---|---|---|---|---|
| 95° C. | 5 | min | | |
| 95° C. | 30 | sec | | |
| 58° C. | 90 | sec | {close oversize bracket} | X 35 |
| 72° C. | 90 | sec | | |
| 68° C. | 10 | in | | |

Determine PCR specificity on 2% agarose gel. Multiplex products should be visualized with multiple banding pattern between 100-300 bp.

Pooling is not required for multiplex products, as the probes have already been combined and amplified into a single tube/reaction.
Purify the pooled PCR with Agencourt AMPure XP beads at a 1.2:1 ratio (60 uL beads+50 uL sample) (refer within document for purification protocol).
After PCR amplification, along with pooling and purifying, the samples can be quantified by qPCR, e.g., Ion Library Quantification Kit, TaqMan assay quantification of lon Torrent libraries, Thermo Fisher Scientific, CAT #4468802 (https://tools.thermofisher.com/content/sfs/manuals/4468986_lonLibraryQuantitationKit_UG.pdf)
1. Create a standard curve of 6.8 pM, 0.68 pM, 0.068 pM, 0.0068 pM
2. Dilute samples 1:1000, and run in duplicate
3. Perform qPCR assay on the Step One Plus Real Time machine by Life Technologies
4. Sample libraries quantified ≥100 pM can proceed to be sequenced on the Life Technologies Ion Torrent Sequencing platform

### Life Technologies Ion Torrent PGM Sequencing

### Ion PGM Template OT2 200.

Perform template reaction with Ion PGM Template OT2 200 Kit, Thermo Fisher Scientific, CAT #4480974. Kit contents to be used on the One Touch 2 and Enrichment system (https://tools.thermofisher.com/content/sfs/manuals/MAN0007220_Ion_PGM_Template_OT2_2 00_Kit_UG.pdf
Utilizing library quant. obtained from qPCR, dilute libraries appropriately to 100 pM. Follow Life Technologies guide on how to further dilute libraries for input into final template reaction.
Follow reference guide to complete template reaction
   Run the Ion One Touch 2 instrument
   Recover the template positive ISPs
   Enrich the template positive ISPs with the Ion One Touch ES

### Ion PGM Sequencing 200

Perform sequencing reaction with Ion PGM Sequencing 200 kit, Thermo Fisher Scientific, CAT #4482006. Kit contents to be used on the Ion PGM system (https://tools.thermofisher.com/content/sfs/manuals/MAN0007273_lonPGMSequenc_200 Kit_v2 UG.pdf).
Plan sequencing run
   Select chip capacity (314, 316 or 318)
   Determine sequencing flows and bp read length (i.e., 500 flows and 200 bp read length)
Follow reference guide to complete PGM sequencing
   Prepare enriched template positive ISPs
   Anneal the sequencing primer
   Chip check
   Bind sequencing polymerase to the ISPs
   Load the chip
   Select the planned run and perform sequencing analysis

### Sequencing Data Analysis and Work Flow

Obtain run report generated by the PGM and Torrent Browser
Run report includes the following information
   ISP Density and loading quality
   Total reads generated and ISP summary
   Read length distribution graph
   Barcoded samples: reads generated per sample and mean read length
Obtain uBAM files generated by the PGM, available for download to an external hard drive
Bioinformatics data analysis
   Upload uBAM files to a web based bioinformatics platform, Galaxy GenAp
      Perform quality control analysis (i.e., basic statistics and sequence quality check)
      Convert data files: BAM SAM FastQ
      Filter FastQ file: select bp size to trim (i.e., trim sequence <100 bp)
      Convert data files: FastQ FastA
      Download FastA file
   Upload FastA files to BiqAnalyzer software platform
      Create project
      Add sample
      Load reference sequence
      Set gap extension penalty and minimal sequence identity
      Link in FastA files to samples and reference sequences
      Analyze and collect data files (pattern maps and pearl necklace diagrams)

### Example 7

### Uveal Melanoma Test

The molecular biology of uveal melanoma (UM) is simpler than that of breast cancer, with minimal mutations and rearrangements, and only two major sub-types which correspond to the retention or loss of chromosome 3p. A test was developed for UM which is superior to current state of the art blood assays.

Analysis of 450 k methylation TCGA data for 80 UMs allowed for the identification of regions of tumour specific methylation in both 3p- and 3 pWT tumours using our algorithm. Table 11 shows 16 hypermethylated regions in both 3p- and 3 pWT tumours used for probe development and testing, according to one embodiment.

TABLE 11

| Gene | Chr | start | stop | Size | CGI CpGs |
|---|---|---|---|---|---|
| PTEN, KILLIN | chr10 | 89611399 | 89611920 | 521 Shore CGI | 171 |
| PAMR1 | chr11 | 35503400 | 35504124 | 724 small CGI | 19 |
| MPZL2 | chr11 | 117640011 | 117640610 | 599 Prox Prom | |
| C2CD4A | chr15 | 60146043 | 60147120 | 1077 Shore CGI | 127 |
| SEZ6 | chr17 | 24370858 | 24371386 | 528 small CGI | 34 |
| LDLR | chr19 | 11060476 | 11060965 | 489 Prox Prom | |
| GALNT3 | chr2 | 166358156 | 166359621 | 1465 CGI | 98 |
| ccdc140/pax3 | chr2 | 222881305 | 222886029 | 4724 Shore CGI | 72 |
| FLI22536/casc15 | chr6 | 21774638 | 21775386 | 748 small CG | 18 |
| KAAG1, DCDC2 | chr6 | 24465699 | 24466545 | 846 CGI | 56 |
| MUC21 | chr6 | 31031220 | 31031651 | 431 CGI | 46 |
| COL19A1 | chr6 | 70632889 | 70633262 | 373 Proc Prom | |
| NR2E1/OSTM1 | chr6 | 108542808 | 108543809 | 1001 small CG | 34 |
| SCRN1 | chr7 | 29996242 | 29996333 | 91 Shore CGI | 133 |
| HES5 | chr1 | 2450725 | 2452224 | 1499 CGI | 111 |
| DHRS3 | chr1 | 12601228 | 12601893 | 665 Shore CGI | 133 |

The top 14 of these common regions were carried forward for probe development and a total of 26 different probes were characterized, with several regions having up to three probes targeting them. Each of these probes was then validated using six different UM cell lines to assess their methylation status. As negative controls, DNA from peripheral blood mononuclear cells (PBMCs), which are the main source of contaminating DNA in blood samples, as well as a pool of cell free DNA (cfDNA) from 16 individuals, were also tested (FIG. 15). These results indicated that the majority of the probes tested showed tumour specific methylation with little or no methylation in the negative controls. A total of 18 probes from 12 different regions were combined into a multiplex PCR reaction and used to analyze cell free DNA from plasma for a previously characterized cohort of metastatic UM patients.

The validated regions were C2CD4A, COL19A1, DCDC2, DHRS3, GALNT3, HES5, KILLIN, MUC21, NR2E1/OSTM1, PAMR1, SCRN1, and SEZ6. The validated probes were C2C5F, COL2F, DCD5F, DGR2F, GAL1F, GAL3F, HES1F, HES3F, HES4F, KIL5F, KIL6F, MUC2F, OST3F, OST4F, PAM4F, SCR2F, SEZ3F, and SEZ5F.

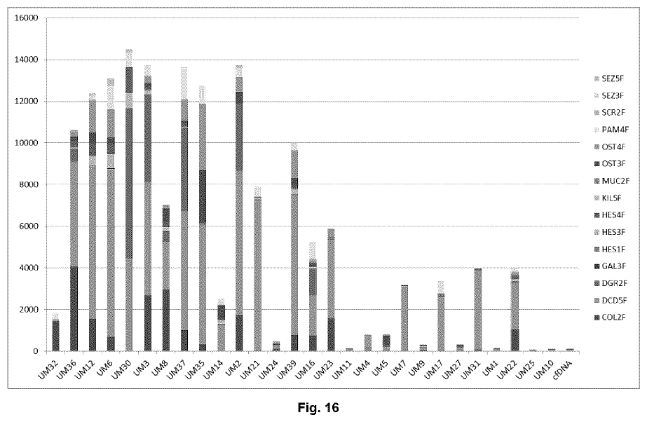

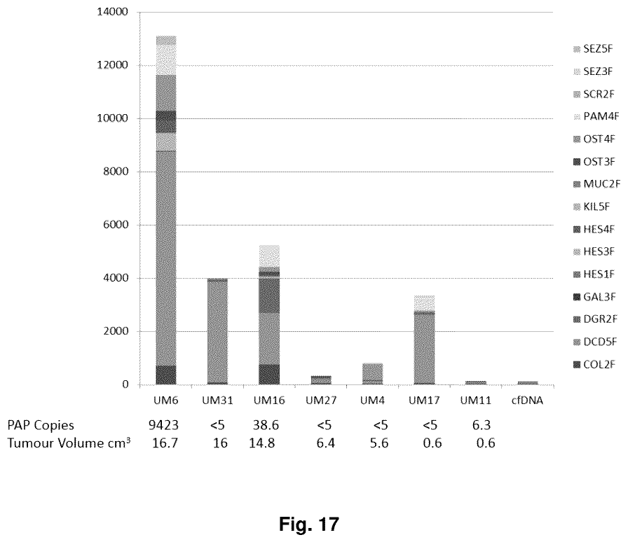

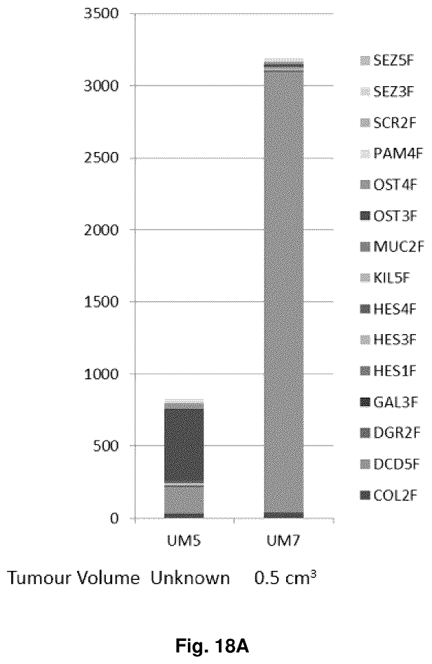

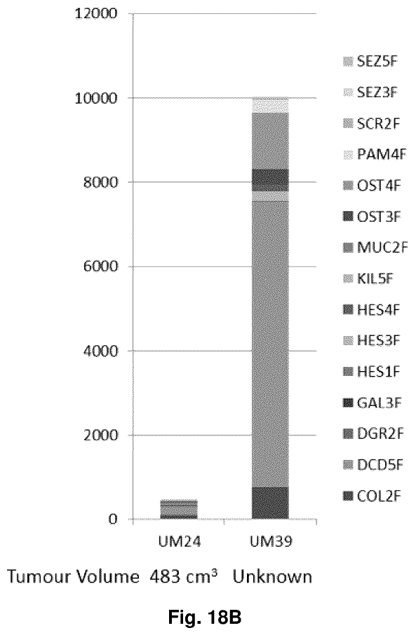

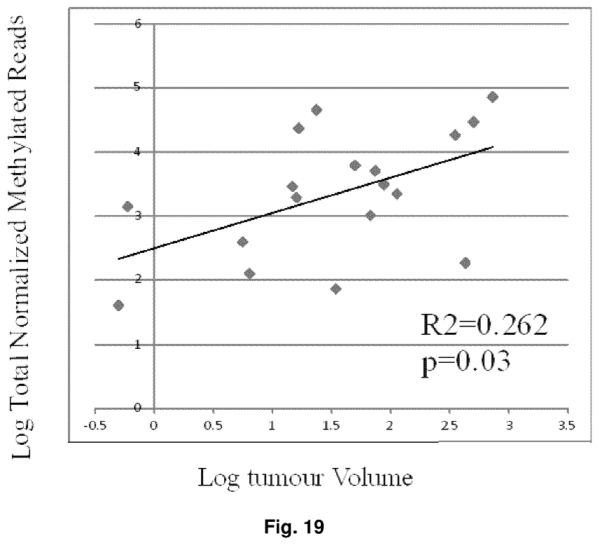

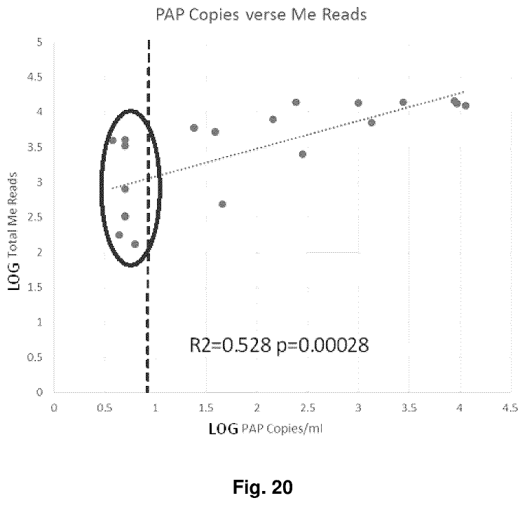

These patients were previously tested using the pyrophosphorolysis-activated polymerization (PAP) assay²⁶, which detects the frequent GNAQ or GNA11 mutations in UM27. In all cases the test detected cancer in these patients even when the PAP assay failed to register a signal (FIGS. 16 and 17). Most of the probes functioned like methylation specific PCR reactions, only giving product when there was tumour DNA present though with the additional validation that the specificity of each probe was guaranteed by the presence of multiple methylated CpG residues within each read. In two patients from which serial blood samples were obtained (FIGS. 18A and 18B) the test showed increased tumour levels over time even when the final tumour volume was 0.5 cm³ (FIG. 18A). The test was also generally correlated with the volume of tumour, though the nature of the metastatic tumour as either a solid mass or dispersed has not yet been accounted for (FIG. 19). The levels detected by the test were generally in line with those of the PAP assay and notably gave a signal where PAP failed due to the lack of a mutation (FIG. 16, UM32). Where no or limited amounts of tumour DNA were detected by PAP, the test still gave significant signals (FIG. 20). Even greater sensitivity is expected when the total number of reads analyzed per patient is increased, as this run had less than optimal overall reads due to the presence of large amounts of primer dimer, an issue that has now been resolved. The specificity of the test was demonstrated by the extremely low levels of methylation seen in the pool of 16 cfDNA controls. Overall, the test has been validated in a patient population, and it has been shown to be superior to a state of the art mutation based assay.

### Example 8

### Prostate Cancer Test

An important aspect of any test is that it should be applicable to all patients. Based on our experience it is essential to consider specific subtypes of a given cancer to ensure that all patients are detected by the assay. The TCGA analysis of a large prostate cohort revealed sub-groups based on specific mutations and transcriptional profiles²⁸. Four subtypes were identified based on the overall pattern of methylation found in these tumours. In this example the TCGA prostate cohort was divided into groups based on the methylation pattern and subjected to methylation analysis.

Table 12 lists 40 regions associated with all sub-types of prostate cancer.

TABLE 12

| | | | |
|---|---|---|---|
| HES5 | ANXA2 | HLA-F | HAAO |
| LOC376693 | RHCG | PON3 | RARB |
| CSRP1 | RARA | LRRC4 | ALDH1L1 |
| ALOX5 | PTRF | HLA-J | HIST1H3G |
| PPM1H | RND2 | PAH | ZSCAN12 |
| MON2 | TMP4 | EPSTI1 | HCG4P6 |
| KIAA0984 | HIF3A | ADCY4 | EYA4 |
| TXNRD1 | KLK5 | HAPLN3 | HOXA7 |
| CHST11 | AMOTL2 | AX747633 | HSF4 |
| EFS | SCGB3A1 | NBR1 | TMEM106A |

These regions common to all four methylation subtypes were identified and a total of 38 probes from 33 regions were selected and appropriate “biased” PCR probes were generated. These were characterized using four different prostate cancer lines. DU145 is an androgen receptor (AR−) negative cell line that is able to generate metastases in the mouse. PC3 is also AR- and also metastatic. LNCaP is an androgen receptor positive line (AR+) that is non-metastatic in the mouse while RWPE cells are AR+ and non-metastatic. DNA from PBMC was also tested as this represents the primary source of cell free DNA in the circulation.

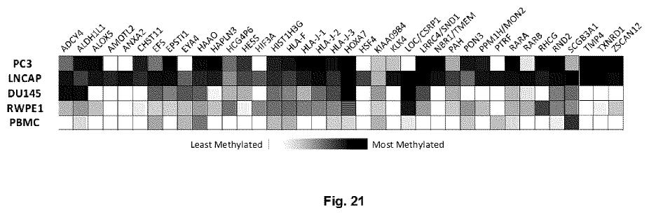

A total of 34 probes from 33 regions were validated in that they showed little or no methylation in PBMCs while showing large scale methylation in one or more of the tumour cell lines (FIG. 21).

The validated regions were ADCY4, ALDH1L1, ALOX5, AMOTL2, ANXA2, CHST11, EFS, EPSTI1, EYA4, HAAO, HAPLN3, HCG4P6, HES5, HIF3A, HLA-F, HLA-J, HOXA7, HSF4, KLK4, LOC376693, LRRC4, NBR1, PAH, PON3, PPM1H, PTRF, RARA, RARB, RHCG, RND2, TMP4, TXNRD1, and ZSCAN12.

The validated probes were ADCY4-F, ALDH1L1-F, ALOX5-F, AMOTL2-F, ANXA2-F, CHST11-F, EFS-F, EPSTI1-F, EYA4-F, HAAO-F, HAPLN3-F, HCG4P6-F, HES5-F, HIF3A-F, HLA-F-F, HLA-J-1-F, HLA-J-2-F, HOXA7-F, HSF4-F, KLK4-F, LOC376693-F, LRRC4-F, NBR1-F, PAH-F, PON3-F, PPM1H-F, PTRF-F, RARA-F, RARB-F, RHCG-F, RND2-F, TMP4-F, TXNRD1-F, and ZSCAN12-F.

To these 34 probes an additional 12 probes (from 7 regions) were added that had previously been characterized in breast cancer, which were also able to detect prostate cancer, for a total of 46 probes.

The added probes were C1Dtrim, C1Etrim, CHSAtrim, DMBCtrim, FOXAtrim, FOXEtrim, SFRAtrim, SFRCtrim, SFREtrim, TTBAtrim, VWCJtrim, and VWCKtrim.

These probes were multiplexed together and were then used to analyze plasma samples from five patients before they had initiated androgen deprivation therapy (ADT) and 12 months after starting treatment. These patients were part of a small cohort (˜40 patients) being followed for depression and the plasma samples at 0.5 ml were much smaller than normally used for the assay (2 mls). All of the patients were MO with no sign of metastatic disease when placed on ADT.

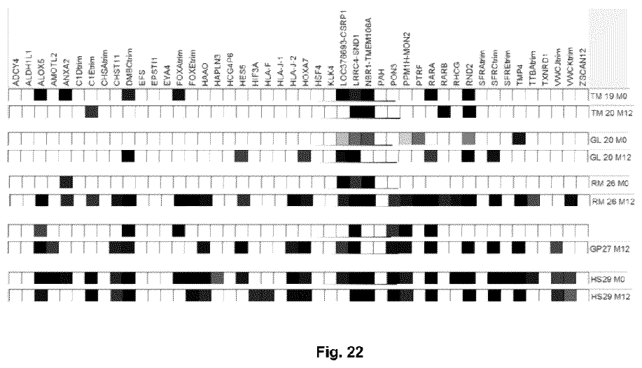

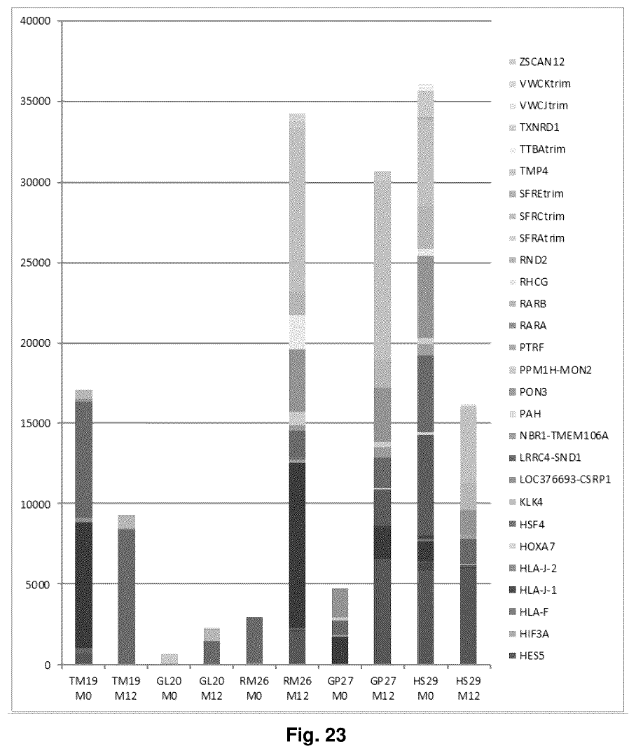

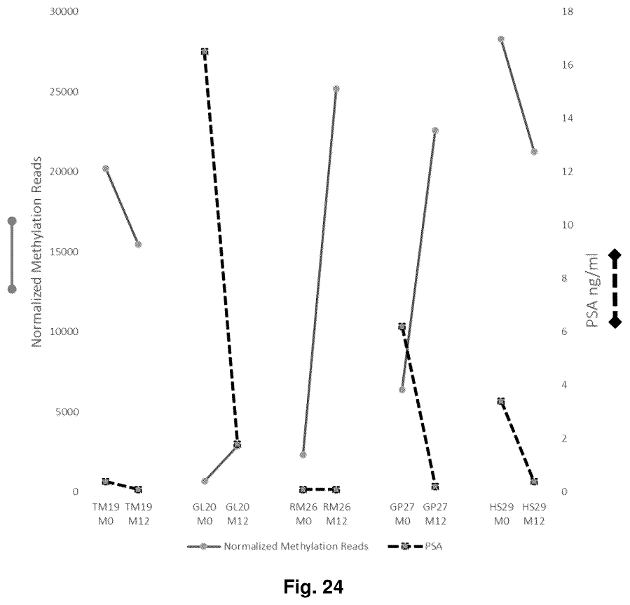

A variety of probes were positive depending on the particular patient (FIG. 22). The total number of positive probes was in keeping with the total number of methylated reads, which were normalized for total reads for each sample (FIG. 23). In all cases significant ctDNA signals were observed with results that were notably different than PSA results (FIG. 24). Two of the patients, TM19 and RM26 were started on ADT due to their aggressive diseases (T3A and T3B) despite having low PSA levels. PSA levels for both remained low but methylation detection of circulating tumour DNA (mDETECT) either decreased slightly (TM19) or rose dramatically (RM26) suggesting their diseases did not express PSA but had stable or increasing disease. HS29 showed decreased PSA levels which mDETECT paralleled. Both GL20 and GP27 trended in opposite directions to PSA levels with mDETECT increasing even with dramatic drops in PSA levels. GL20 did develop a radiation induced secondary cancer which may be what is detected. Ongoing analysis of additional clinical data is expected to help explain these results.

Based on the literature, three of these regions appear to have prognostic significance as well. C1orf114 or CCDC1 has been shown to be correlated with biochemical relapse. HES5 is a transcription factor that is regulated by the Notch pathway and methylation of its promoter occurs early in prostate cancer development. KLK5 is part of the Kallikrein gene complex that includes KLK3 (the PSA gene). We can demonstrate that KLK5 expression is correlated with methylation and KLK5 expression has previously been shown to be increased in higher grade tumours. These results strongly suggest that the examination of a large number of methylation markers may yield significant insight into the specific processes involved in prostate cancer development and produce diagnostic and prognostic information that would be vital for management of the disease.

### Example 9

### Predictive Prostate Cancer Methylation Biomarkers

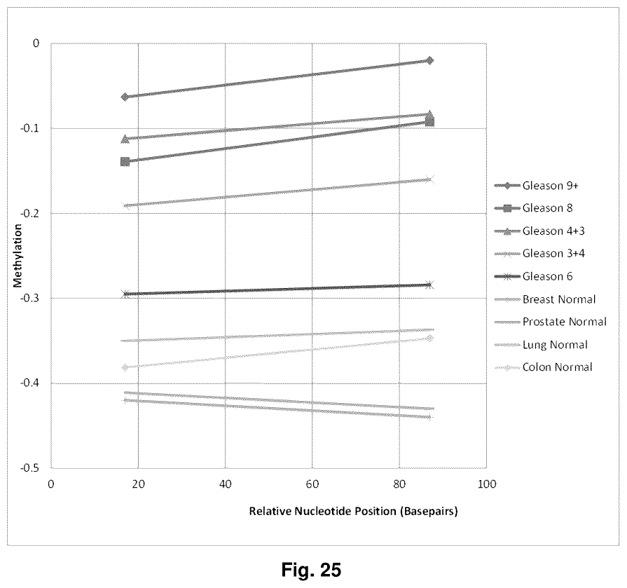

The 50 region assay according to embodiments described herein is sufficiently sensitive to easily detect metastatic disease and to follow changes in tumour size over time and, as indicated, has predictive value in itself. As described above, at least three regions, KLK5, HER5, and C1orf114 have potential to predict progression. In order to develop additional probes that are able to predict outcome in this patient population, the prostate cancer TCGA data was reanalysed to divide the patients by Gleason score. An inter-cohort comparison was conducted to identify regions frequently methylated in higher score cancers. Initially, Gleason grades 6 and 9 were compared as these typically represent less and more aggressive tumours and both groups had sufficient numbers of patients to ensure significance of the results. Probe development was carried out under the same criteria as with the original probe sets so that they could be used with ctDNA. No single probe will be absolutely specific for a given grade but a number of the probes showed excellent division between Gleason scores with the proportion of the cohort positive for a given grade increasing with increasing grade (FIG. 25). One of these, PSS3, is a gene whose expression has previously been associated with prostate cancer and particularly metastasis. It should be noted that not all methylation is associated with gene repression. Forty-three new probes were developed based on selection criteria to target the 36 regions shown in Table 13, which are associated with aggressive prostate cancer.

TABLE 13

| | | | |
|---|---|---|---|
| ASAP1 | EMX1 | MIR1292 | SOX2OT |
| BC030768 | HFE | NBPF1 | TUBB2B |
| C18orf62 | HIST1H3G/1H2BI | NHLH2 | USP44 |
| C6orf141 | HMGCLL1 | NRN1 | Intergenic (Chr1) |
| CADPS2 | KCNK4 | PPM1H | Intergenic (Chr8) |
| CORO1C | KJ904227 | PPP2R5C | Intergenic (Chr2) |
| CYP27A1 | KRT78 | PRSS3 | Intergenic (Chr3) |
| CYTH4 | LINC240 | SFRP2 | Intergenic (Chr4) |
| DMRTA2 | Me3 | SLCO4C1 | Intergenic (Chr10) |

The probes were ASAP1/p, BC030768/p, C18orf62/p, C6orf141/p-1, C6orf141/p-2, CADPS2/p, CORO1C/p-1, CORO1C/p-2, CYP27A1/p, CYTH4/p, DMRTA2/p, EMX1/p, HFE/p-1, HFE/p-2, HIST1H3G/1H2BI/p, HMGCLL1/p, KCNK4/p, KJ904227/p, KRT78/p, LINC240/p-1, LINC240/p-2, Me3/p-1, Me3/p-2, MIR129, NBPF1/p, NHLH2/p, NRN1/p, PPM1H/p-1, PPM1H/p-2, PPP2R5C/p, PRSS3/p, SFRP2/p-1, SFRP2/p-2, SLCO4C1/p, SOX2OT/p, TUBB2B/p, USP44/p, Chr1/p-1, Chr2/p-1, Chr3/p-1, Chr4/p-1, Chr8/p-1, and Chr10/p-1.

It is expected that it will be an overall pattern of hypermethylation, rather than a single probe, that will have the greatest predictive power.

### Example 10

### Breast Cancer Test

One approach described herein for identifying hypermethylated regions in breast cancer focused on the most frequently methylated regions within the TCGA database. Due to the large number of LumA and LumB patients in this dataset there was a significant under-detection particularly of the Basal class of tumours.

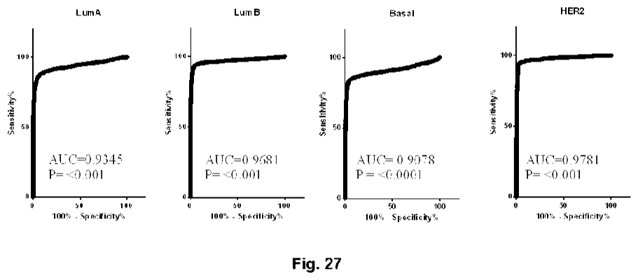

Accordingly, the data were reanalyzed based on the four molecular subtypes LumA, LumB, Her2 and Basal. The Normal-like subtype is not very frequent in the dataset and as expected is very close to normal tissue, however a small number of regions recognizing this subtype were also included. Overall, methods and probes were developed and tested for over 230 different regions (some with multiple probes), and these have been validated using a variety of breast cancer cell lines and tumour samples. Some regions are subtype-specific but most recognize multiple subtypes. These have been assembled into a single test incorporating 167 different probes which recognize all subtypes (FIGS. 26A, 26B, and 26C), with all patients being recognized by a significant number of probes. By looking at just the top 20 probes for each subtype this test has an area under the curve (AUC) per subgroup from 0.9078 to 0.9781, indicating that high detection rates have been achieved for all types of tumours (FIG. 27). This also means that the test is able to identify the subtype of tumour based on the distribution of probe methylation.

Another test specific for the triple negative breast cancer (TNBC) subtype was developed from the larger set of general regions identified as described above. This test incorporates 86 probes from 71 regions, listed in Table 14.

TABLE 14

| CCL28 | PTPRN2 | UDB | IRF4 | HOXA9 | HINF1B | POU4F1 |
|---|---|---|---|---|---|---|
| PAX6 | BARHL2 | TMEM90B | SOX2OT | NT5E | TNFRSF10D | VWC2 |
| PPFIA3 | PRSS27 | C1orf114 | TSPAN33 | DPP10 | CD38 | BRCA1 |
| SPAG6 | DMRTA2 | ITPRIPL1 | CA9 | FOXA3 | CHST11 | HOXB13 |
| TMEM132C | NR5A2 | GIPC2 | IRF8 | C5orf39 | FABP5 | OTX2 |
| DMBX1 | BOLL | ERNA4 | CRYM | PTGDR | Intergenic5 | |
| TAL1 | SLC7A4 | MAST1 | GNG4 | SALL3 | EVX1 | |
| TOP2P1 | LEF1 | DRD4 | DDAH2 | ID4 | ACVRL1 | |
| PRDM13 | CARD11 | Intergenic 8 | EPSTI1 | GABRA4 | TBX15 | |
| GALR3 | NFIC | TCTEX1D1 | TTBK1 | PRKCB | ALX1 | |
| CDKL2 | PDX1 | PHOX2B | SCAND3 | NPHS2 | SIM1 | |

The probes were ALX1, AVCRL1, BRCA1-A, C1Dtrim, C1 Etrim, CA9-A, CARD11-B, CCL28-A, CD38, CDKL2-A, CHSAtrim, CRYM-A, DMBCtrim, DMRTA2exp-A, DPP10-A, DPP10-B, DPP10-C,DRD4-A, EFNA4-B, EPSTI1, EVX1, FABP5, FOXAtrim, FOXEtrim, GALR3-A, GIPC2-A, HINF C trim, HOXAAtrim, HOXACtrim, HOXB13-A, Int5, Int8, IRF8-A, ITRIPL1, LEF1-A, MAST1 A trim, mbBARHL2 Trim, mbBOLL Trim, mbC5orf Trim, mbDDAH Trim, mbDMRTA Trim, mbGABRA A Trim, mbGABRA B Trim, mbGNG Trim, mbID4 Trim, mbIRF Trim, mbNT5E Trim, mbSIM A Trim, mbTBX15 Trim, NFIC-B, NFIC-A, NPSH2-B, NR5A2-B, OTX2-A, PAX6-A, pbDMRTA Trim, pbGNG Trim, pbSCAND Trim, pbTAL Trim, PDX1exp-B, PHOX2B-A, POU4F1 A trim, PPFIA3-A, PRDM13, PRKCB-A, PRKCB-C, PRSS27-A, PTGDR, PTPRN2-A, PTPRN2-B, SALL3-A, SALL3-B, SLC7A4-A, SOX2OT-B, SPAG6 A trim, TCTEX1D1-A, TMEM-A, TMEM-B, TMEM90B-A, TNFRSF10D, TOP2P1-B, TSPAN33-A, TTBAtrim, UBD-A, VWCJtrim, and VWCKtrim.

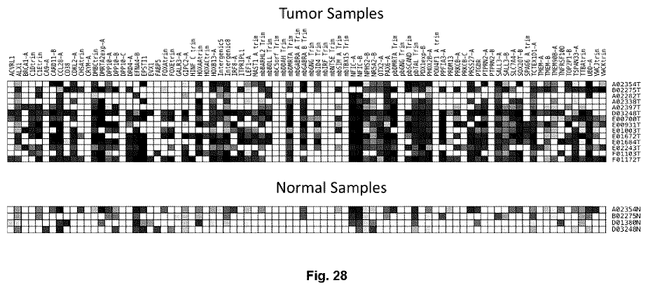

The ability of this test to detect TNBC was validated by the analysis of 14 TNBC primary tumours as well as matched normal tissue from four of these patients. Large scale methylation was observed for the majority of probes and was distinctly different from the normal samples (FIG. 28).

### Example 11

### Sensitivity of the Tests

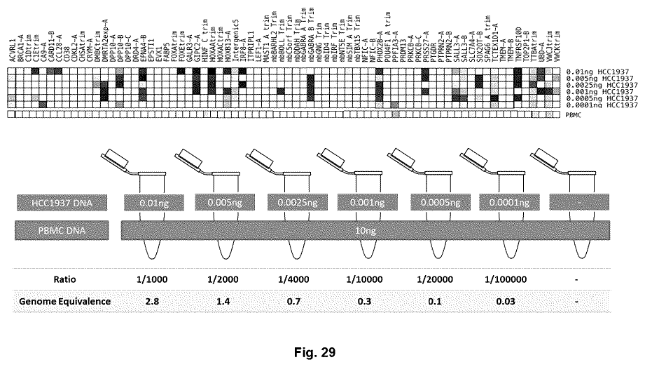

The tests described herein are designed to detect less than one genome's worth of DNA in a sample through the use of multiple regions where a single probe out of many can signal the presence of a tumour. The more regions and probes incorporated into a test the greater is the sensitivity. This is in contrast to mutation detection where the presence of a single mutation per genome equivalent means that random sampling effects rapidly limit sensitivity when the concentration of the tumour DNA falls below one genome equivalent per sample. The presence of large amounts of normal DNA in fluid samples also creates problems for the detection of mutations through the relatively high error rates for PCR and sequencing. To assess the limits of methods and tests described herein, a dilution experiment was performed wherein DNA from a TNBC cell line (HCC1937 DNA) was diluted into a constant amount of PBMC DNA (10 ng) from a normal patient (FIG. 29). These samples were then tested using the TNBC test. A conclusive signal was obtained from the test even when as little as 0.0001 ng of TNBC DNA was present in 10 ng of PBMC DNA. This represents a detection of 0.03 genome equivalents of tumour DNA against a background of 100,000 times more normal DNA.

### Example 12

### Discussion

The sensitivity of mutation based detection tests is limited by their detection of single unknown mutations in genes, such as p53 or ras. As only a single mutation is present per genome equivalent, this dramatically limits the sensitivity of these assays. Once the concentration of tumour DNA in the blood decreases to less than one genome equivalent per volume of blood analysed, the probability of detecting a mutation decreases dramatically as that particular segment of DNA may not be present in the blood sample. The assay described herein incorporates multiple probes for multiple regions from across the genome to dramatically increase sensitivity. For example, up to 100 or more probes may be incorporated into the assay, making it up to 100 or more times more sensitive than mutation based tests.

Circulating tumour DNA may be produced by the apoptotic or necrotic lysis of tumour cells. This produces very small DNA fragments in the blood. With this in mind, PCR primer pairs were designed to detect DNA in the range of 75 to 150 bp in length, which is optimal for the detection of circulating tumour DNA.

The use of DNA methylation offers one more advantage over mutation based approaches. Mutated genes are typically expressed in the cells (such as p53). They are thus in loosely compacted euchromatin, in comparison to methylated DNA which is in tightly compacted heterochromatin. This methylated and compacted DNA may be protected from apoptotic nucleases, increasing its concentration in the blood in comparison to these less compacted genes.

Extensive analysis of the genome wide methylation patterns in breast, colon, prostate and lung cancers and normal tissue in each of these organs based on TCGA data was carried out. 52 regions were identified for breast cancer which fulfill design criteria, which looks for an optimal difference in methylation between tumour and normal breast tissue, and where there is no methylation in any of the other normal tissues. As well, there should optimally be at least 2 CpG residues within 200 basepairs of each other. This ensured that regions of coordinated tumour specific methylation have been identified.

Within these 52 regions, 17 were found in common with colon cancer, and 9 in common with prostate cancer. Interestingly there were few appropriate regions identified in lung cancer, with only 1 overlapping with breast cancer. Most of these regions are associated with specific genes, though several are distantly intergenic, and almost all were found in CpG islands of various sizes. Probes were first developed for those regions with some commonality between cancers and designed PCR primers which recognize the methylated DNA sequence. This provides a bias in the amplification process for tumour DNA, enriching the tumour signal. These primer pairs amplify regions of 75 to 150 bp in accordance with our design criteria. Typically these regions contain from 3 to 12 CpG residues each, ensuring a robust positive signal when these regions are sequenced. Multiple non-overlapping probes were used as the CpG islands are generally larger than 150 bp, allowing for multiple probes for each appropriate region, providing more power to detect these regions and increasing the detection sensitivity of the assay.

Six different breast cancer lines were used in this validation analysis that have been shown to generally retain tumour specific methylation patterns²². MCF-7 and T47D lines are classic ER+ positive cell lines representing the most frequent class of breast cancer. SK-BR-3 cells are a HER2+ line and MDA-MB-231 cells represent a Triple Negative Breast cancer (TNBC), thus the 3 main categories of breast cancer are represented covering 95% of all tumours. Two “normal” lines were also used, the MCF10A line, though this line has been shown to contain some genomic anomalies, and the karyotypically normal 184-hTERT line. DNA was bisulphite converted, and the probes were amplified individually, barcoded then pooled according to cell line and subject to Next Generation Sequencing on an Ion Torrent sequencer. Not all PCR primer pairs produced a product due to the methylation-based nature of the primers, but in general, where a signal was detected, around 1000 reads were obtained per probe for each cell line. These reads were processed through our NGS pipeline using Galaxy and then loaded into the NGS methylation program BiqAnalyzer^{23,24}. This program extracts probe specific reads, aligns them against the probe reference sequence, and calls methylated and unmethylated CpGs. It also carries out quality control measures related to bisulphite conversion and alignment criteria. In all of these probes there are several CpG residues within the primer sequence producing a bias towards amplifying methylated DNA. The analysis shown only includes CpGs outside of the primers which are solely representative of the methylation status of the sample being analysed.

FIGS. 5 and 6 depict results for the CHST11 gene, which is a good example where robust PCR primers are able to recognize tumour specific methylation. Four different primer pairs were assessed, three of which amplify probes that partially overlap. In all four cases these regions are completely methylated at all CpGs (not including CpGs in the primers) and are essentially completely unmethylated in the normal lines. CHST11 primers do not recognize the Her2 or TNBC lines, but other primers such as ADCY and MIRD do. The corresponding probes cover a small region of the CpG island and information about the status of the rest of the CpG island is limited due to the relatively coarse resolution of the 450K methylation data. Clearly the remaining part of the CpG island can be developed for additional probes that would increase the sensitivity of detection.

FIG. 7 shows that FOXA probe A had similar characteristics and recognized all but one TNBC tumour. This proves that the target and probe development pipeline moving from TCGA data to cell lines and then to patient normal and tumour tissue successfully identified primer pairs that are able to specifically recognize tumour DNA based on their methylation patterns.

Validation work continues to validate potential probe regions. A further 24 regions were characterized using 52 different probes in the cell lines as an initial screen for their suitability.

FIG. 4 shows the results of analysis of all of the potential CpGs identified in the TCGA cohort for individual patients indicates most patients are recognized by a large proportion of these probes.

FIG. 3 shows the results of ROC analysis²⁵ and indicates each of these probes has a very high AUC, suggesting excellent performance individually and presumably even better when combined.

It has been noted that there does appear to be a population of patients with relatively few positive probes. This is not subtype specific and other probes specific for this population have been identified. As appropriate, additional probes will be developed for all suitable regions and expanded to include other parts of the associated CpG islands. Overall it is expected that 100-150 separate probes in the assay will provide optimal sensitivity.

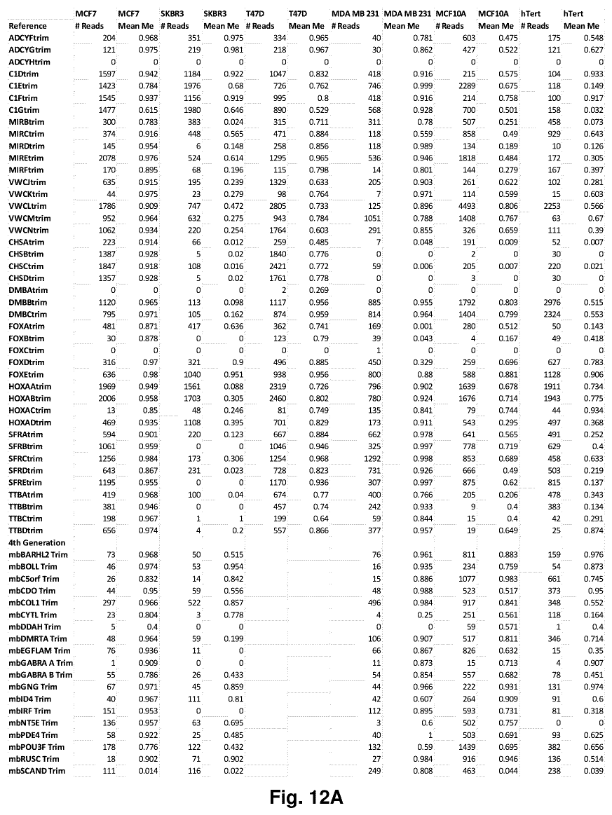

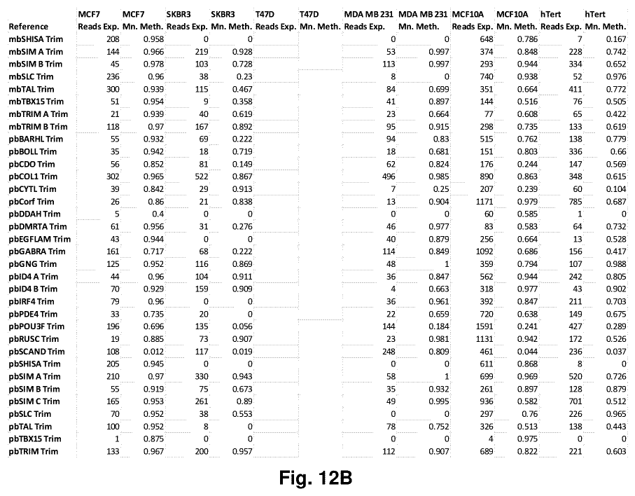

FIGS. 12A and 12B depict a numerical summary of validation data, wherein “#Reads” indicates the number of reads, and “Mean” Me indicates the mean methylation observed in results. Approximately half of the probes met the design criteria of having complete methylation of all CpG residues in the tumour samples and little or no methyation in the normal lines.

The next step in validating each of these probes was to examine their methylation patterns in actual patient tumour samples. A small cohort of patient samples was used to investigate GR methylation. From this group three ER+ tumours (one of which is positive for GR methylation), one HER2+ tumour and two TNBC tumours were chosen, as well as their corresponding normal controls. Taking the CHST11A probe as an example, FIG. 6 shows that all six of the normal breast tissue samples had either no reads due to the methylation biased amplification yielding no product or minimal methylation. In no case was there any concerted methylation signal where all CpGs were methylated. In contrast, in one TNBC and one ER+PR+ tumour a strong concordant methylation signal was seen at all six CpG sites. The other 2 ER+PR+ tumours also showed consistent methylation at four or five CpGs with their normal breast tissue controls having minimal reads with only one CpG showing any methylation.

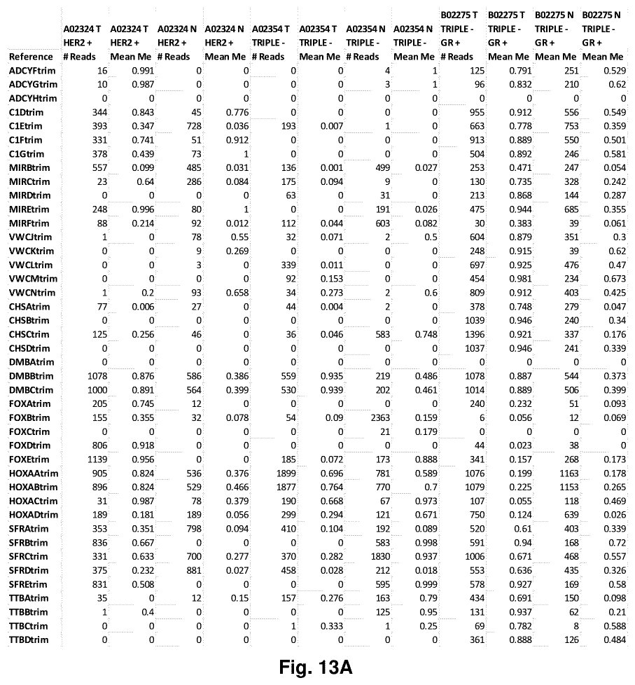

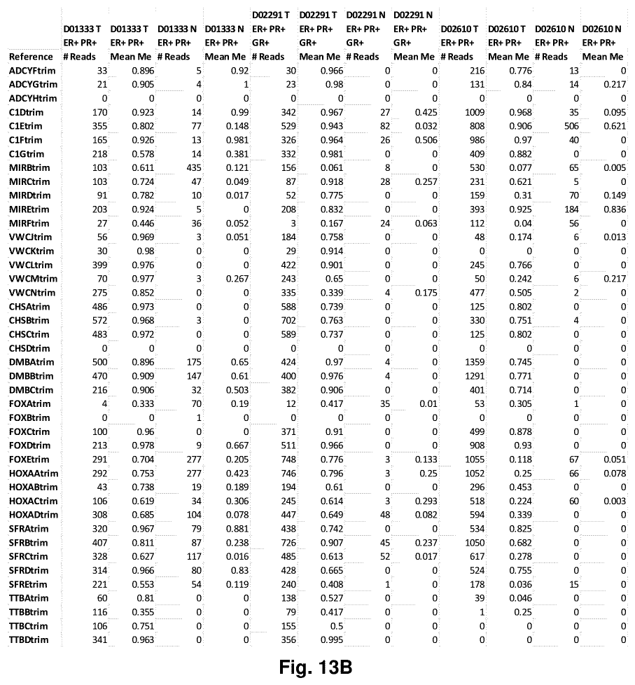

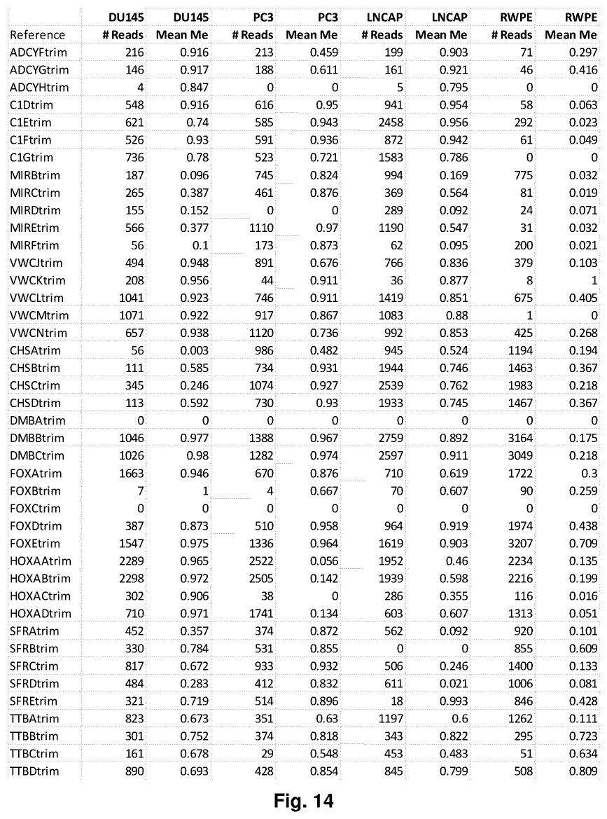

FIGS. 13A and 13B depict a numerical summary of generated methylation data for tumour samples for all probes tested to date. #Reads is indicative of the number of reads exported, and Mean Me is indicative of the mean methylation.

Initial proof of concept work involved mixing experiments where non-methylated and methylated DNA was mixed in increasing ratios. This demonstrated that based in the presence of multiple CpG signatures methylated DNA could easily be detected in the presence of at least a 500 fold excess of unmethylated DNA. These probes were amplified with PCR primers that were not methylation specific or biased, and the probes developed to date do incorporate a bias towards methylated DNA, which further increases the detection sensitivity. However, they do amplify non-methylated DNA (in part because primers were designed with no preference as to the location of methylation sites within the primers). This was done intentionally as it provides for a potential quantitative aspect to this assay. Some of the circulating normal DNA in blood samples is likely from the lysis of nucleated blood cells, which is why serum is preferred over plasma as a source of DNA. However the ratio of tumour to normal DNA in blood may provide some quantitation of the actual concentration of tumour DNA present in the blood, which is thought to be correlated with tumour load. Since tumour can be distinguished from normal DNA reads, the ratio between them can be used as a proxy for the tumour DNA concentration. The number of tumour specific reads per volume of blood, regardless of the number of normal reads, may also prove to be closely linked to circulating tumour DNA levels.

Optimizing this test may include multiplexing to allow all of the probes the opportunity to amplify their targets in a given sample of DNA. Through the use of limited concentrations of primers and cycles, excellent amplification of all probes was obtained within a set of 17 primer pairs. Expanding this to include all of the optimized primers is not expected to be an issue.

The test may be implemented as a blood based breast cancer detection system in patient blood samples.

Based on development and validation work to date, the assay offers significant advantages other current and developing tests based on sensitivity, specificity, and detection sensitivity.

Some potential applications of the embodiments described herein are listed below by level of detection sensitivity:
Determining response to neo-adjuvant chemotherapy;
Monitoring tumour load in diagnosed patients;
Detecting residual disease post-surgery;
Detecting relapse;
Secondary screen after positive MRI in high risk patients;
Direct monitoring of high risk patients; and
Primary population screening.

The analysis of patients with active breast cancer offers the ability to assess a number of different aspects of this blood based test. Patients with locally advanced disease can be recruited preferentially, as these patients generally have larger tumours, receive neo-adjuvant therapy, are more likely to have residual disease and are at higher risk of relapse. By analysing blood samples from these patients upon diagnosis, after any neo-adjuvant treatments, pre-surgery, and at followup visits post-surgery it is possible to follow the relative tumour burden in these patients over the course of treatment. This will allow the tumour size and type to be correlated with the results of the test described herein.

Patients can be recruited in the clinic after a biopsy confirmed positive diagnosis. Blood can be drawn in conjunction with other routine blood work at diagnosis, after neo-adjuvant treatment, before surgery, within a month after surgery and every 3-6 months following that. Blood from 50 aged matched women without disease can also be collected from the community to provide control samples for the patient cohort. Relevant clinical data can be collected including radiological assessments and/or pathology reports. In particular, the receptor status of the tumours, the size of the tumour based on both radiological assessment and examination of the excised tumour, as well as treatments and response to therapy can be correlated with the circulating DNA analysis.

The assay described herein is expected to be quantitative at different levels. At very low levels of tumour DNA, the random presence of the tumour DNA in a sample will result in a subset of individual probes being positive, with the number of positive probes increasing with greater tumour DNA levels. At higher levels of tumour DNA the number of tumour specific reads will increase, either as an absolute number or in relation to the number of normal DNA reads. As a result methylation data can be treated in three ways:
(1) As a binary outcome where each probe will be considered to be positive if it has any tumour specific methylation pattern present;
(2) An individual threshold of methylation will be established for each probe based on the minimum number of reads required to call a tumour; or
(3) Tumour specific reads per number of normal reads for each probe (or, e.g., per 100,000 total reads).

Each of these approaches may be used to carry out logistic regression on the patient and control sets. Receiver Operating Characteristic (ROC) analysis may be used to define thresholds for each probe that maximizes the sensitivity and sensitivity of the assay. The performance of the entire assay may be characterized using Area Under the Curve (AUC) analysis for overall sensitivity, specificity, classification accuracy and likelihood ratio. Pearson or Spearman correlations may be used to compare patient parameters with the test outcomes.

Changes in methylation may be important drivers of breast cancer development and that these occur very early during the process of transformation. This may explain why many of the observed methylations are common amongst different breast cancer sub-types, while some are even common to other cancers. This may mean that these changes predate the development of full malignancy and suggests that they could also have value in assessing the risk of a women developing breast cancer. It is envisaged that the assay described herein can be used to track the accumulation of risk in the form of increasing gene specific methylation levels and could be used to develop a risk assessment tool. This would be useful for the development and assessment of risk mitigation and prevention strategies.

Table 15 lists the primers used herein for each probe.

| | | SEQ | | | PCR |
|---|---|---|---|---|---|
| | | ID | 5′-3′ Primer Sequence | | Product |
| Gene | Probe | NO. | (Bisulfite) | Chr: Location | Length |
| C1orf114/ | C1Df | 1 | TTGAGGTAAAGGAGATTTCGGT | chr1: 167663228-167663361 | 134 |
| CCDC18 | C1Dr | 2 | ACATACGCCTACGCAAATTTTTA | | |
| | C1Ef | 3 | TTCGGTGTTTGCGAAGGGTTA | chr1: 167663398-167663508 | 111 |
| | +C1Er | 4 | TCACAACCAACACAACGACACTT | | |
| | C1Er | 5 | ACAACCAACACAACGACACTT | | |
| | C1Ff | 6 | TCGGTATTTGTTTTCGCGGT | chr1: 167663245-167663356 | 112 |
| | C1Fr | 7 | CGCCTACGCAAATTTTTATCGC | | |
| | C1Gf | 8 | CGAGAGCGATAAAAATTTGCGT | chr1: 167663330-167663417 | 88 |
| | C1Gr | 9 | ACCCTTCGCAAACACCGAAA | | |
| | C1 eAf | 10 | GGTAATAGCGTGTTTTTGC | chr1: 167663285-167663366 | 82 |
| | C1 eAr | 11 | ATATTACATACGCCTACGCAAA | | |
| | C1 eBf | 12 | TTTGTGTAAAATGCGGCGGT | chr1: 167663149-167663266 | 118 |
| | C1 eBr | 13 | CTACCGCGAAAACAAATACCGA | | |
| | C1 eCf | 14 | ATTTCGGTGTTTGCGAAGGG | chr1: 167663395-167663506 | 112 |
| | C1 eCr | 15 | ACAACCAACACAACGACACT | | |
| VWC2 | VWCJf | 16 | TTTCGGTTGTCGGGTTTGGA | | |
| | +VWCJf | 17 | TATTTCGGTTGTCGGGTTTGGA | chr7: 49783871-49784003 | 133 |
| | VWCJr | 18 | CCCTCAATCGCTCATCCTCC | | |
| | VWCKf | 19 | TCGTCGGTCGGTTTAGGATG | chr7: 49784151-49784279 | 129 |
| | +VWCKr | 20 | AAAACCGACGCCAAACCTACAT | | |
| | VWCKr | 21 | AACCGACGCCAAACCTACAT | | |
| | VWCLf | 22 | CGGAGGATGAGCGATTGAGG | chr7: 49783983-49784100 | 118 |
| | VWCLr | 23 | TAACGCGCACACCGAACTAA | | |
| | VWCMf | 24 | CGAGTTGGGGTCGCGATTAT | chr7: 49784021-49784170 | 150 |
| | VWCMr | 25 | CATCCTAAACCGACCGACGA | | |
| | VWCNf | 26 | CGACGCGTTACGGTTGTTTA | chr7: 49783849-49783973 | 125 |
| | VWCNr | 27 | CCGCTTCTCCGAAACCAAAC | | |
| | VWC2 eAf | 28 | TAAGGCGGGGTTTTTAGAGC | chr7: 49783687-49783792 | 106 |
| | VWC2 eAr | 29 | TAAAAACTAACGCGCCCG | | |
| | VWC2 eBf | 30 | GGTTTCGGTGTTATTCGC | chr7: 49783797-49783922 | 126 |
| | VWC2 eBr | 31 | CTCCTCTCCGCGAAAAAAT | | |
| | VWC2 eCf | 32 | CGGAGGATGAGCGATTGAGG | chr7: 49783983-49784100 | 118 |
| | VWC2 eCr | 33 | TAACGCGCACACCGAACTAA | | |
| | VWC2 eDf | 34 | TCGTCGGTCGGTTTAGGATG | chr7: 49784151-49784277 | 127 |
| | VWC2 eDr | 35 | AACCGACGCCAAACCTACAT | | |
| | VWC2 eEf | 36 | GTCGGACGCGTTTTAGTTGG | chr7: 49784315-49784424 | 110 |
| | VWC2 eEr | 37 | TCCCTACCGACCTCAACACT | | |
| MIR129-2 | MIRBf | 38 | TGGTTGGGGGATTTTGAGGG | chr11: 43559089-43559229 | 141 |
| | MIRBr | 39 | AAACCTCCCCGCCTACCTAT | | |
| | MIRCf | 40 | GCGGACGGTTTGGAGAAATG | chr11: 43559343-43559424 | 82 |
| | MIRCr | 41 | CGCGACTCAATCTCACCACT | | |
| | MIRDf | 42 | GGAGGTTGGGTTTCGGGATT | chr11: 43559257-43559383 | 127 |
| | MIRDr | 43 | GCGCCCCTAAACTCGTATCT | | |
| | MIREf | 44 | GCGGAGTGGTGAGATTGAGT | chr11: 43559401-43559513 | 113 |
| | MIREr | 45 | ACCGACTTCTTCGATTCGCC | | |
| | MIRFf | 46 | ATAGGTAGGCGGGGAGGTTT | chr11: 43559205-43559343 | 139 |
| | MIRFr | 47 | CGATCCCCCAACTCAACCC | | |
| | MIR eAf | 48 | TGAGTTGGCGGTTTCGTTTG | chr11: 43559004-43559125 | 122 |
| | MIR eAr | 49 | CCCGAATCCCCTCTTATCCC | | |
| | MIR eBf | 50 | CGCGATTTTGTAGTCGGGGT | chr11: 43559156-43559251 | 96 |
| | MIR eBr | 51 | TTTCCTATCGCCCCAACACC | | |
| | MIR eCf | 52 | GGAGGTTGGGTTTCGGGATT | chr11: 43559257-43559383 | 127 |
| | MIR eCr | 53 | GCGCCCCTAAACTCGTATCT | | |
| | MIR eDf | 54 | GATTGAGTCGCGATGGAACG | chr11: 43559413-43559494 | 81 |
| | MIR eDr | 55 | GCCGCCTTCAACCCAAAATA | | |
| ADCY4 | ADCYFf | 56 | CGCGAGCGTATAGAGTACGA | chr14: 23873573-23873735 | 163 |
| | ADCYFr | 57 | ACCCTAACCAACCCCGAAAC | | |
| | ADCYGf | 58 | TAGCGTCGCGAGCGTATAGA | chr14: 23873567-23873754 | 188 |
| | ADCYGr | 59 | AAAAATAACCCGACGCCCGA | | |
| | ADCYHf | 60 | GGTTTCGTAGAAGAGGTTTTC | chr14: 23873642-23873815 | 174 |
| | ADCYHr | 61 | CGCGAAATAATAACGACTTT | | |
| | ADCY4 eAf | 62 | AGAAGAGGTTTTCGTTGGGGG | chr14: 23873650-23873729 | 80 |
| | ADCY4 eAr | 63 | ACCAACCCCGAAACTCGAAA | | |
| | ADCY4 eBf | 64 | TAGGATTTGGGGTTGGTGCG | chr14: 23873975-23874115 | 141 |
| | ADCY4 eBr | 65 | AACGCAACGACGAACGTAAC | | |
| | ADCY4 eCf | 66 | TGGTAGTGGGGAGATCGAGG | chr14: 23874376-23874474 | 99 |
| | ADCY4 eCr | 67 | AAACGCCCCCAACTCTAACC | | |
| DMBX1 | DMBAf | 68 | GTTGCGGACGGCGTAGAT | chr1: 46723984-46724132 | 149 |
| | DMBAr | 69 | ACGCTCCCCGAAACAATAACT | | |
| | DMBBf | 70 | TTGTTAGTTTTGTTAGCGCGG | chr1: 46723919-46723993 | 75 |
| | DMBBr | 71 | CGTCCGCAACGATTCATCATC | | |
| | DMBCf | 72 | TGTTTAGGAGATGGTTCGTGGT | chr1: 46723889-46724003 | 115 |
| | +DMBCr | 73 | GCATCTACGCCGTCCGCAAC | | |
| | DMBCr | 74 | ATCTACGCCGTCCGCAAC | | |
| | DMBX1 eAf | 75 | TGTTTAGACGTGGGTTGGGG | chr1: 46723237-46723323 | 87 |
| | DMBX1 eAr | 76 | TCAACTCCACTCACCCCGTA | | |
| | DMBX1 eBf | 77 | GAGGAGGGTGGAGAGGGTAG | chr1: 46723478-46723610 | 133 |
| | DMBX1 eBr | 78 | ATACCGCACGTACTCCCAAC | | |
| | DMBX1 eCf | 79 | GGAGTGGAGTAGGTAGCGGT | chr1: 46723635-46723751 | 117 |
| | DMBX1 eCr | 80 | TTCCTAACCCTCTCCGACCA | | |
| | DMBX1 eDf | 81 | TTTTTGAGCGGTGAAGGGGA | chr1: 46723764-46723888 | 125 |
| | DMBX1 eDr | 82 | AATTATTAACGCGACCGCCG | | |
| HOXA9 | HOXAAf | 83 | GTAATAATTTGGTGGTATCGGGGG | chr7: 27171666-27171765 | 100 |
| | HOXAAr | 84 | TCTACTAAACGAACACGTAACGC | | |
| | HOXABf | 85 | ATAATTTGGTGGTATCGGGGG | chr7: 27171669-27171777 | 109 |
| | HOXABr | 86 | ACGCGTTATTATTCTACTAAACGAA | | |
| | HOXACf | 87 | TGGGGTTTGTTTTAATTGTGGTT | chr7: 27171878-27172029 | 152 |
| | +HOXACr | 88 | GCGAAACCCGCGCCTTCTTAAT | | |
| | HOXACr | 89 | GAAACCCGCGCCTTCTTAAT | | |
| | HOXADf | 90 | GGGGAAGTATAGTTATTTAATAAGTTG | chr7: 27171688-27171815 | 128 |
| | HOXADr | 91 | ACAAAACATCRAACCATTAATAA | | |
| | HOXA9 eAf | 92 | TTCGCGAAGGAGAGCGTATC | chr7: 27171234-27171334 | 101 |
| | HOXA9 eAr | 93 | CCCTACGTACACCCCCAAAC | | |
| | HOXA9 eBf | 94 | CGTTTGGGGGTGTACGTAGG | chr7: 27171314-27171401 | 88 |
| | HOXA9 eBr | 95 | AAACCCAATACACGCGACGA | | |
| | HOXA9 eCf | 96 | TTTGTCGGGGAGGTTGGTTT | chr7: 27171478-27171559 | 82 |
| | HOXA9 eCr | 97 | TTCCTACTAAACGCCGACGC | | |
| | HOXA9 eDf | 98 | TAGCGTTTGGTTCGTTCGGT | chr7: 27171611-27171733 | 123 |
| | HOXA9 eDr | 99 | ATAAAAACGCGAACGCCGAC | | |
| SFRP5 | SFRAf | 100 | GCGGGCGTTTCGATTGATTT | | |
| | +SFRAf | 101 | TTGCGGGCGTTTCGATTGATTT | chr10: 99521730-99521860 | 131 |
| | SFRAr | 102 | TAAAAACCGCCCCCACTACC | | |
| | SFRBf | 103 | TGTTCGGCGGTTTAGGTGTT | chr10: 99521628-99521751 | 124 |
| | SFRBr | 104 | AAATCAATCGAAACGCCCGC | | |
| | SFRCf | 105 | TAGTTCGGGTTTCGTCGTGC | chr10: 99521776-99521865 | 90 |
| | +SFRCr | 106 | AAAACTAAAAACCGCCCCCACT | | |
| | SFRCr | 107 | AACTAAAAACCGCCCCCACT | | |
| | SFRDf | 108 | GTGGGTGGTAGTTTGCGTTG | chr10: 99521713-99521847 | 135 |
| | SFRDr | 109 | CACTACCTCCCCGCCTTAAA | | |
| | SFREf | 110 | GCGTGCGTTTTCGGTTTTGA | | |
| | +SFREf | 111 | CGGCGTGCGTTTTCGGTTTTGA | chr10: 99521649-99521731 | 83 |
| | SFREr | 112 | AACGCAAACTACCACCCACC | | |
| | SFRP5 eAf | 113 | GGACGTTGGGTTGAGTTAGGA | chr10: 99520910-99521018 | 109 |
| | SFRP5 eAr | 114 | ACGACCCTACAACTCCCCTA | | |
| | SFRP5 eBf | 115 | GGTGTTCGAATTGTACGGCG | chr10: 99521073-99521179 | 107 |
| | SFRP5 eBr | 116 | CTACGCGCCGCTCATAAAAA | | |
| | SFRP5 eCf | 117 | GCGCGTACGGTTTCGTATAG | chr10: 99521183-99521257 | 75 |
| | SFRP5 eCr | 118 | ATACTCGCTCTTTACGCCCG | | |
| | SFRP5 eDf | 119 | TAGAGCGGTAGGTCGGTAGG | chr10: 99521393-99521471 | 79 |
| | SFRP5 eDr | 120 | AACAAACCGAACCGCTACAC | | |
| CHST11 | CHSAf | 121 | GCGGCGTGGGAATGAATTTT | | |
| | +CHSAf | 122 | GGGCGGCGTGGGAATGAATTTT | chr12: 103376278-103376397 | 120 |
| | CHSAr | 123 | CTTTCCCTCGCACCCCTAAA | | |
| | CHSBf | 124 | TGCGAGGGAAAGTTTGGGTT | chr12: 103376386-103376508 | 123 |
| | CHSBr | 125 | CCGCGTTACCCGAAAAACTT | | |
| | CHSCf | 126 | TTTTAGGGGTGCGAGGGAAA | chr12: 103376377-103376462 | 86 |
| | CHSCr | 127 | CGCAACCGAACTACTCACCC | | |
| | CHSDf | 128 | GTGCGAGGGAAAGTTTGGGT | chr12: 103376385-103376510 | 126 |
| | CHSDr | 129 | ACCCGCGTTACCCGAAAAA | | |
| | CHST11 eAf | 130 | TTTTTTTGGTTGTCGGGTC | chr12: 103375901-103376009 | 109 |
| | CHST11 eAr | 131 | CGAAACCCGAAACACGTA | | |
| | CHST11 eBf | 132 | AGAGTGGTCGGGTGTTTAGC | chr12: 103376031-103376179 | 149 |
| | CHST11 eBr | 133 | ACGTAACCCAAAAACTCGAAA | | |
| | CHST11 eCf | 134 | GTCGTTTTTTAGGGGTGC | chr12: 103376371-103376469 | 99 |
| | CHST11 eCr | 135 | TAAACTTCGCAACCGAACTA | | |
| | CHST11 eDf | 136 | TATTAAGTTTGCGTTTGGGTC | chr12: 103376781-103376889 | 109 |
| | CHST11 eDr | 137 | AAAACCGTCTATCCCTACGC | | |
| FOXA3 | FOXAf | 138 | CGAGGTAGGAAGTTTTGCGG | chr19: 51071936-51072038 | 103 |
| | FOXAr | 139 | CGACTCCTCCCGCGAAATAA | | |
| | FOXBf | 140 | CGGGGTGTTGTTGTAGGGTT | chr19: 51072158-51072250 | 93 |
| | FOXBr | 141 | AATCACACCTACCCACGCC | | |
| | FOXCf | 142 | TAGGGCGGTTAGGTTTGGGG | chr19: 51072076-51072203 | 128 |
| | FOXCr | 143 | GACGAATAACCCCACCCTCC | | |
| | FOXDf | 144 | TTGTCGCGTTGGTTTTTCGT | chr19: 51071765-51071867 | 103 |
| | FOXDr | 145 | ACCTTTCTCTCGACCCCAAT | | |
| | FOXEf | 146 | CGTTTTGTCGGTTGCGTGTTA | chr19: 51071734-51071824 | 91 |
| | FOXEr | 147 | ATTCCCCGACCTACCCAAAAC | | |
| | FOXA3 eAf | 148 | GGTAGGTGATAACGTTAGTGGGTT | chr19: 51068615-51068724 | 110 |
| | FOXA3 eAr | 149 | ACCTCCATCCCCTACCCAAC | | |
| | FOXA3 eBf | 150 | AGTAGGGGGAGGTGGTTTTG | chr19: 51069110-51069244 | 135 |
| | FOXA3 eBr | 151 | TCCTCCTCCCCAACTTAACC | | |
| | FOXA3 eCf | 152 | AGTTTGGGTGTGGCGGTTTA | chr19: 51070046-51070156 | 111 |
| | FOXA3 eCr | 153 | ACCAACTTCGCCATATTAACCA | | |
| TTBK1 | TTBAf | 154 | CGCGGTGTATTGTGGGTAGT | chr6: 43319189-43319287 | 99 |
| | TTBAr | 155 | CCTTCCGACCCGAATCATCC | | |
| | TTBBf | 156 | GGTCGTCGGAACGTGATGT | chr6: 43319101-43319186 | 86 |
| | TTBBr | 157 | GCCAACATCAACACCAACCC | | |
| | TTBCf | 158 | TCGTTTTGTCGTTGTCGTCG | chr6: 43319212-43319318 | 107 |
| | TTBCr | 159 | TTAAATAACCCGCTCCCTCCG | | |
| | TTBDf | 160 | GTCGTGATGTTAGAGCGGGC | chr6: 43319130-43319255 | 126 |
| | TTBDr | 161 | ACCCCGATCCTCCTTAAACG | | |
| | TTBK1 eAf | 162 | TTAAGGAGGATCGGGGTC | chr6: 43319239-43319329 | 91 |
| | TTBK1 eAr | 163 | TCAATACGACGTTAAATAACCC | | |
| | TTBK1 eBf | 164 | TGGAGTTAAGCGGGTGGTAG | chr6: 43319008-43319148 | 141 |
| | TTBK1 eBr | 165 | CCCGCTCTAACATCACGACTC | | |
| TAL1 | pbTAL f | 166 | GTATTGTCGCGGGTTCGTTC | chr1: 47470631-47470738 | 129 |
| | pbTAL r | 167 | CTCAACCAATCCCCACTCCC | | |
| | mbTAL f | 168 | GTTTTAGGTTTCGTTAGTATGGG | chr1: 47470570-47470698 | 129 |
| | +mbTAL r | 169 | CAAATTAAAATAAATCATTTAACCCATAA | | |
| | mbTAL r | 170 | TTAAAATAAATCATTTAACCCATAA | | |
| DMRTA2 | pbDMRTA f | 171 | CGAAGATTTCGTAGGCGGGT | chr1: 50659325-50659469 | 145 |
| | +pbDMRTA r | 172 | ACGACGCAAATAACGCTACGCA | | |
| | pbDMRTA r | 173 | GACGCAAATAACGCTACGCA | | |
| | mbDMRTA f | 174 | TGTTTTAGAAGCGGGAGAAAG | | |
| | mbDMRTA r | 175 | AAATAAAACCCCCGTATCCAAT | | |
| | +mbDMRTA f | 176 | AATGTTTTAGAAGCGGGAGAAAG | chr1: 50659041-50659153 | 113 |
| | +mbDMRTA r | 177 | AAAAATAAAACCCCCGTATCCAAT | | |
| | DMRTAexp Af | 178 | GCGGCGGTTAGCGTTAGTTTTTCGGTAG | chr1: 50659366-50659489 | 124 |
| | DMRTAexp Ar | 179 | CGAAACGCCAACGTATCATAACGACGCA | | |
| PDE4B | pbPDE f | 180 | ACGTTTTAGGGACGGCGAAT | chr1: 66030622-66030698 | 77 |
| | pbPDE r | 181 | AATCCCAACGACCGTCTACC | | |
| | mbPDE f | 182 | TTTCGTTTTGTATTTATGGTAGATGT | chr1: 66030580-66030694 | 115 |
| | mbPDE r | 183 | CCAACGACCGTCTACCACTA | | |
| BARHL2 | pbBARHL f | 184 | CGTGGTATGGATTTCGGGGT | chr1: 90967266-90967376 | 111 |
| | pbBARHL r | 185 | ACTCCTAACCCTAAACGCGA | | |
| | mbBARHL f | 186 | GTTTTTTTCGGTTTTTGTTCGA | | |
| | mbBARHL r | 187 | TTTCTCCCAATTCCAATATCCA | | |
| | +mbBARHL f | 188 | TGGTTTTTTTCGGTTTTTGTTCGA | chr1: 90967815-90967900 | 86 |
| | +mbBARHL r | 189 | ACTTTCTCCCAATTCCAATATCCA | | |
| TBX15 | pbTBX f | 190 | GCGATCGGCGATTGGTTTTT | chr1: 119331668-119331767 | 100 |
| | pbTBX r | 191 | GCGACGACACACGACCTAAA | | |
| | mbTBX f | 192 | TGAGGTTTTAGGTCGTGTGT | | |
| | +mbTBX f | 193 | GGTGAGGTTTTAGGTCGTGTGT | chr1: 119331740-119331881 | 142 |
| | mbTBX r | 194 | AAAACCTTAATCGACTCAAATAAAA | | |
| RUSC1, | pbRUSC f | 195 | GGGTGTAGTTGCGTAGCGTA | chr1: 153557280-153557421 | 142 |
| C1orf104 | pbRUSC r | 196 | CCGAACCCTCCTCACCAAAA | | |
| | mbRUSC f | 197 | TAGTTGCGTAGCGTAGGGTA | chr1: 153557285-153557410 | 126 |
| | mbRUSC r | 198 | TCACCAAAATCCTCCTAAAAC | | |
| GNG4 B | pbGNG f | 199 | ACGTAGTGTTGGTAAGATTTGTAGA | chr1: 233880823-233880971 | 149 |
| | pbGNG r | 200 | ACAAAAACCGCTTATAAACGACGA | | |
| | mbGNG f | 201 | GTAGGTTTTTGCGTTGGAGATT | chr1: 233880677-233880817 | 141 |
| | mbGNG r | 202 | ATTTTCGTTACTTCTCTATTCCCAAA | | |
| POU3F3 | pbPOU3F f | 203 | GGGGTTTCGCGTTTTGAGTT | chr2: 104836866-104836944 | 79 |
| | pbP0U3F r | 204 | AACACCAAAACCCCCGCTAA | | |
| | mbP0U3F f | 205 | AAAAGTAATTAATCGGAACGGT | chr2: 104836837-104836970 | 134 |
| | mbPOU3F r | 206 | ACACTTTCCCAAATACAAAAAAA | | |
| BOLL B/C | pbBOLL f | 207 | TTTCGAGTCGGGGCGTTTTA | chr2: 198359264-198359401 | 138 |
| | pbBOLL r | 208 | TACCTAACCGCTCGCTCTCT | | |
| | mbBOLL f | 209 | GTTCGGTTTTGGGATTTTT | | |
| | mbBOLL r | 210 | AATCCCAAAAACCGACTCT | | |
| | +mbBOLL f | 211 | GAGGGTTCGGTTTTGGGATTTTT | chr2: 198359331-198359461 | 131 |
| | +mbBOLL r | 212 | ACCAATCCCAAAAACCGACTCT | | |
| TRIM71 | pbTRIM f | 213 | CGGAGGAATTTGTGTCGTCG | chr3: 32834331-32834440 | 110 |
| | pbTRIM r | 214 | CACCAAAACAACGCTACCCG | | |
| | mbTRIM Af | 215 | TTGGGAATTTTTTTCGTTTAT | chr3: 32834188-32834337 | 150 |
| | mbTRIM Ar | 216 | TCCTCCGAATAACTTAAAAACC | | |
| | mbTRIM Bf | 217 | TCGTTGGATAGTGGTATTTAATGT | chr3: 32834348-32834497 | 150 |
| | mbTRIM Br | 218 | AAAATCACCGACTCACTCAA | | |
| SLC2A2 | pbSLC f | 219 | CGGAGTACGGCGGTAGGAA | chr3: 172228914-172228993 | 80 |
| | +pbSLC r | 220 | AATACCCCGAAAACCCGCTAATA | | |
| | pbSLC r | 221 | ACCCCGAAAACCCGCTAATA | | |
| | mbSLC f | 222 | ATGATATTTTGTAGGAAAGCGT | chr3: 172228748-172228850 | 103 |
| | mbSLC r | 223 | CAAATTCCGTTTCTAAAAAAAC | | |
| CYTL1 | pbCYTL f | 224 | GGGTTCGTATGCGGGAGTAG | chr4: 5071974-5072099 | 126 |
| | pbCYTL r | 225 | ACGAAACTACACCAACGCCT | | |
| | mbCYTL f | 226 | GGGGGTTTTCGTTAGGAGTAG | chr4: 5072020-5072142 | 123 |
| | mbCYTL r | 227 | AAACCGCCCTAAACCACC | | |
| SHISA3 | pbSHISA f | 228 | GAAGGGCGGTAGCGATAGTT | chr4: 42094543-42094650 | 108 |
| | +pbSHISA r | 229 | CTACGAATTCCGCAAACCGAAA | | |
| | pbSHISA r | 230 | ACGAATTCCGCAAACCGAAA | | |
| | mbSHISA f | 231 | ATTGTTTTTGTCGGCGTT | chr4: 42094569-42094654 | 86 |
| | mbSHISA r | 232 | TACACTACGAATTCCGCAA | | |
| GABRA4 | pbGAB f | 233 | GCGTGCGTATATTCGCGTTT | | |
| | +pbGAB f | 234 | CGGCGTGCGTATATTCGCGTTT | chr4: 46690291-46690385 | 95 |
| | pbGAB r | 235 | AAATTCCGCCTCCCCTAACC | | |
| | mbGAB Af | 236 | TTTAGCGTTTAATGTGTATGTAGA | chr4: 46690411-46690545 | 135 |
| | +mbGAB Ar | 237 | CGAAATTACAATCGAAACAAACTTAC | | |
| | mbGAB Ar | 238 | AAATTACAATCGAAACAAACTTAC | | |
| | mbGAB Bf | 239 | GTTTTGAGTAGGGTGCGAG | | |
| | mbGAB Br | 240 | AAAAAAACAAATTCCGCCT | | |
| | +mbGAB Bf | 241 | GATGTTTTGAGTAGGGTGCGAG | chr4: 46690248-46690398 | 151 |
| | +mbGAB Br | 242 | AAACGAAAAAAACAAATTCCGCCT | | |
| EGFLAM | pbEGF f | 243 | TGGTAGCGTTGTAAGGTGGG | chr5: 38293231-38293359 | 129 |
| | pbEGF r | 244 | AAAAACAAACGCGACCCTCG | | |
| | mbEGF f | 245 | TCGAGTTTTGGTAGCGTTGTAA | chr5: 38293223-38293306 | 84 |
| | +mbEGF r | 246 | AATACCCCGCAAAAAAAATCTACA | | |
| | mbEGF r | 247 | CCCCGCAAAAAAAATCTACA | | |
| C5orf39 | pbC5orf f | 248 | ACGAGAAATTGGCGCGTTGA | chr5: 43076304-43076404 | 101 |
| | pbC5orf r | 249 | AACAACACCCTTTACGACGC | | |
| | mbC5orf f | 250 | TGTTTGTTAGGGTTTTGTTTTAA | | |
| | mbC5orf r | 251 | CGCCAAAACGAATATTTATTTA | | |
| | +mbC5orf f | 252 | AATTGTTTGTTAGGGTTTTGTTTTAA | chr5: 43076267-43076390 | 124 |
| | +mbC5orf r | 253 | CGACGCCAAAACGAATATTTATTTA | | |
| CDO1 B | pbCDO f | 254 | GGTAGCGTAGTGGATTCGGG | chr5: 115180192-115180333 | 142 |
| | pbCDO r | 255 | CTCGTCCTCCCTCCGAAAAC | | |
| | mbCDO f | 256 | GTTTGTTTTATTTCGTGGGGAG | chr5: 115179983-115180067 | 85 |
| | mbCDO r | 257 | CCAACTCCTTAACTCGCTCAA | | |
| IRF4 B/C | pbIRF f | 258 | TCGCGGGAAACGGTTTTAGT | | |
| | pbIRF r | 259 | GCCCTTAACGACCCTCCG | | |
| | +pbIRF f | 260 | TTTTCGCGGGAAACGGTTTTAGT | chr6: 336451-336550 | 100 |
| | +pbIRF r | 261 | GCGCCCTTAACGACCCTCCG | | |
| | mbIRF f | 262 | CGTTTTGTAAAGCGAAGTTT | | |
| | +mbIRF f | 263 | GTTATACGTTTTGTAAAGCGAAGTTT | chr6: 336298-336405 | 108 |
| | mbIRF r | 264 | AAACCAATCAATCACTAAACTACA | | |
| ID4 B | pbID Af | 265 | GGTTTTTGGGCGTCGTGTTA | chr6: 19945064-19945170 | 107 |
| | pbID Ar | 266 | AAATTCACTCTCCACCGCCC | | |
| | pbID Bf | 267 | AGGCGAATAATGAAACGGAGGA | chr6: 19944950-19945083 | 134 |
| | pbID Br | 268 | TAACACGACGCCCAAAAACC | | |
| | mbID f | 269 | ATTTTACGGATGGAGTGATG | | |
| | +mbID f | 270 | GGAATTTTACGGATGGAGTGATG | chr6: 19945031-19945148 | 118 |
| | mbID r | 271 | CTTATCCCGACTAAACTACTAAAAAA | | |
| SCAND3, | pbSCAND f | 272 | AATTCGTTTCGCGACGTGAG | | |
| GPX5 | +pbSCAND f | 273 | TTAATTCGTTTCGCGACGTGAG | chr6: 28618249-28618359 | 111 |
| | pbSCAND r | 274 | ACACGCCTTAAAACCTACTCAT | | |
| | mbSCAND f | 275 | CGTGAGGGAGAATTTAGGAG | chr6: 28618265-28618368 | 104 |
| | mbSCAND r | 276 | TAAAAAAACACACGCCTTAAAACCTA | | |
| DDAH2 | pbDDAH f | 277 | TCGTTTAGCGAGCGTTGTTT | chr6: 31806112-31806210 | 99 |
| | pbDDAH r | 278 | GATCCGCCGTTACGCTATTC | | |
| | mbDDAH f | 279 | TGTTAGAAATCGGTATCGTTTA | | |
| | mbDDAH r | 280 | TCTACGAAACGTTTACAACC | | |
| | +mbDDAH f | 281 | TTTTTTGTTAGAAATCGGTATCGTTTA | chr6: 31806097-31806189 | 97 |
| | +mbDDAH r | 282 | AAAATCTACGAAACGTTTACAACC | | |
| COL11A2 | pbCOL f | 283 | TTTAGGGATCGCGTTCGGAG | chr6: 33269259-33269402 | 144 |
| | pbCOL r | 284 | AAACTCCTTTCCCCTCTCATAC | | |
| | mbCOL f | 285 | CGGAGTTTTTAATCGGATAT | chr6: 33269274-33269415 | 142 |
| | mbCOL r | 286 | TCCCTTCTCTTTAAAACTCCT | | |
| NT5E B | mbNT5E f | 287 | GTCGGATTTTATTTTAATCGTG | | |
| | mbNT5E r | 288 | AAACAAAAAAATCTCAAAAACTAAAA | | |
| | +mbNT5E f | 289 | GTTGTCGGATTTTATTTTAATCGTG | chr6: 86215769-86215912 | 144 |
| | +mbNT5E r | 290 | CTTAAACAAAAAAATCTCAAAAACTAAAA | | |
| SIM1 B | pbSIM Af | 291 | GTTAGGGGCGAGGCGTTTAT | chr6: 101019614-101019695 | 82 |
| | pbSIM Ar | 292 | CGAAACCTAAACGCGCGAAA | | |
| | pbSIM Bf | 293 | AGGTTAATAGGTGGCGCGTT | chr6: 101019077-101019171 | 95 |
| | pbSIM Br | 294 | CCCGCAACTCCGCGATAATA | | |
| | pbSIM Cf | 295 | AGTCGTTTTTCGCGCGTTTA | | |
| | +pbSIM Cf | 296 | CGAGTCGTTTTTCGCGCGTTTA | chr6: 101019667-101019756 | 90 |
| | pbSIM Cr | 297 | GACCCGACACCCTAAACTCAT | | |
| | mbSIM Af | 298 | AGGCGTTTATTGGTTAATAGGG | chr6: 101019624-101019757 | 134 |
| | +mbSIM Ar | 299 | CGACCCGACACCCTAAACTCAT | | |
| | mbSIM Ar | 300 | ACCCGACACCCTAAACTCAT | | |
| | mbSIM Bf | 301 | TTTAATTTGGGTTTTAAGTTTGAGG | chr6: 101018944-101019075 | 132 |
| | mbSIM Br | 302 | ACGCTACTAAACCCCGCTTAT | | |
| RGS17 | RGS17 Af | 303 | GCGTTTAGGTAGCGACGC | chr6: 153493700-153493820 | 121 |
| | RGS17 Ar | 304 | ATACCCCGACGAAAACGAC | | |
| | RGS17 Bf | 305 | TTTGGGATTTGGTCGAGC | chr6: 153493620-153493730 | 111 |
| | RGS17 Br | 306 | AAAATTAAATCCCGCGTCG | | |
| CAPDS2 | CAPDS Af | 307 | CGTTTAGGTTTGTGGACGC | chr7: 121743823-121743951 | 129 |
| | CAPDS Ar | 308 | AAAAACGAAATCGCTAATACGC | | |
| MSC | MSC Af | 309 | TTTTTCGAATTTTTGCGC | | |
| | MSC Ar | 310 | AACACGCTCCGACTAACTTC | | |
| | +MSC Af | 311 | GGTTGTTTTTTCGAATTTTTGCGC | chr8: 72918397-72918531 | 135 |
| | +MSC Ar | 312 | TAAACACGCTCCGACTAACTTC | | |
| | MSC Bf | 313 | CGTTCGCGTTATTATTTGC | | |
| | MSC Br | 314 | CGCCCAATAACAACTCGT | | |
| | +MSC Bf | 315 | ATTATCGTTCGCGTTATTATTTGC | chr8: 72918698-72918852 | 155 |
| | +MSC Br | 316 | CCTCGCCCAATAACAACTCGT | | |
| SPAG6 | SPAG6 Af | 317 | GTCGAGTCGTCGTTACGATC | chr10: 22674453-22674529 | 77 |
| | SPAG6 Ar | 318 | CTACCCTCCTCGAACTCTACG | | |
| INA | INA Af | 319 | GTTTTCGGATGGGAAATTTTAG | | |
| | INA Ar | 320 | AAACCATCTACATCGAAATCGC | | |
| | +INA Af | 321 | GTGGTTTTCGGATGGGAAATTTTAG | chr10: 105026593-105026715 | 123 |
| | +INA Ar | 322 | AACAAAACCATCTACATCGAAATCGC | | |
| FLI | FLI Af | 323 | TTTTTAGGAGTAAGTATTTTGTGTG | chr11: 128068870-128068981 | 112 |
| | FLI Ar | 324 | CCCTCTTCCTCCCCTACTAAT | | |
| ATP5G2 | ATP5G2 Af | 325 | TAGGTATATTTCGGTCGGC | chr12: 52357363-52357478 | 116 |
| | ATP5G2 Ar | 326 | AACTCGAAACCTCATCCG | | |
| USP44 | USP44 Af | 327 | ACGGGAGGGTAAATTTAGC | chr12: 94466977-94467090 | 114 |
| | USP44 Ar | 328 | TACCAAACAATTCGACGTTA | | |
| POU4F1 | POU4F1 Af | 329 | GCGTACGTCGGTTTATTC | | |
| | POU4F1 Ar | 330 | ACGCTCTACGCGATCAAA | | |
| | +POU4F1 Af | 331 | AAGTGCGTACGTCGGTTTATTC | chr13: 78075512-78075652 | 141 |
| | +POU4F1 Ar | 332 | GCGACGCTCTACGCGATCAAA | | |
| LHX1 | LHX Af | 333 | CGAGCGATTGTGGGGTTAGA | chr17: 32368543-32368624 | 82 |
| | LHX Ar | 334 | CAACTCGCGACCGCCTAAA | | |
| HINF1B | HINF Af | 335 | TTCGGGCGTTTATAGAGTTC | chr17: 33176898-33177017 | 120 |
| | HINF Ar | 336 | AAAATCAAAACGCGAACG | | |
| | HINF Bf | 337 | TAGCGTCGCGTTAGAAAGC | | |
| | HINF Br | 338 | ATCGCTCAAAACCTAACGAA | | |
| | +HINF Bf | 339 | TTTTAGCGTCGCGTTAGAAAGC | chr17: 33177225-33177341 | 117 |
| | +HINF Br | 340 | AAAAATCGCTCAAAACCTAACGAA | | |
| | HINF Cf | 341 | AGGTTTAGTTTCGAAATCGC | | |
| | HINF Cr | 342 | AACCGAACGATTCCCTAA | | |
| | +HINF Cf | 343 | GTTAAGGTTTAGTTTCGAAATCGC | chr17: 33177654-33177773 | 120 |
| | +HINF Cr | 344 | CTAAAAAACCGAACGATTCCCTAA | | |
| GALR1 | GALR1 Af | 345 | GAATTTTTGGAAAAGTCGGGA | | |
| | GALR1 Ar | 346 | CTCCTACAAAAAAAACTCCC | | |
| | +GALR1 Af | 347 | TTCGGAATTTTTGGAAAAGTCGGGA | chr18: 73090886-73090989 | 104 |
| | +GALR1 Ar | 348 | CGACTCCTACAAAAAAAACTCCC | | |
| MAST1 | MAST1 Af | 349 | AGAAGGTGGTCGGTAAGC | | |
| | MAST1 Ar | 350 | ACGTAATTATAAAAAACACGCC | | |
| | +MAST1 Af | 351 | GGAGAAGGTGGTCGGTAAGC | chr19: 12839386-12839533 | 148 |
| | +MAST1 Ar | 352 | AAAACGTAATTATAAAAAACACGCC | | |
| | MAST1 Bf | 353 | TAGTTTTTTGGAGGGAGAGG | chr19: 12839568-12839670 | 103 |
| | MAST1 Br | 354 | ATCCTCGTCCTCTTAAAAAAC | | |
| CPXM1 | CPXM1 Af | 355 | GTCGAGTTTGGGATTTTGGT | | |
| | CPXM1 Ar | 356 | AAACTCCTACTCGCCCTAACC | | |
| | +CPXM1 Af | 357 | GGGGTCGAGTTTGGGATTTTGGT | chr20: 2729097-2729214 | 118 |
| | +CPXM1 Ar | 358 | AAAAACTCCTACTCGCCCTAACC | | |
| NEURL2 | NEURL2 Af | 359 | TCGAGTTGGATAAGGCGTAC | chr20: 43952304-43952445 | 142 |
| | NEURL2 Ar | 360 | CCGATAACACGACCGACATA | | |
| | NEURL2 Bf | 361 | TGTATGTCGGTCGTGTTATC | chr20: 43952424-43952505 | 82 |
| | NEURL2 Br | 362 | TAAACGTACTACCTCCGACC | | |
| ACVRL1 | ACVRL1f | 363 | GGATGTGGGAGGTTCGGTTCGGGTG | chr12:50587308-50587443 | 136 |
| | ACVRL1r | 364 | CCGCTCGCCCCTCGCTAAAACTACA | | |
| AFF3 | AFF3f | 365 | GGCGCGAGGTAGTTTTAGTACGTAGTTTTT | chr2: 99542180-99542257 | 78 |
| | AFF3r | 366 | ATAACAACGTCGTCCTTTCCGCAAAACG | | |
| AKR1B1 | AKR1B1f | 367 | GGGGATTTTGTAAGTTCGCGCGTGGTTT | chr7: 133794143-133794250 | 108 |
| | AKR1B1r | 368 | ACACTCTCCGCGCGACCTATATTAACGA | | |
| | AKR1B1R_f | 369 | GGAGACGGTTTGTTATGGTTGTTGCGTT | chr15: 43266838-43266959 | 122 |
| | AKR1B1R_r | 370 | ACGCCCTTTCTACCGACCTCACGAACTA | | |
| ALDOC | ALDOCf | 371 | TTTTTCGGGGGCGTGGTTTGTATGTTT | chr17: 23928071-23928193 | 123 |
| | ALDOCr | 372 | TACCTAACGAAACGCTCACTCCACCTCG | | |
| ALOX5 | ALOX5f | 373 | TTTTGCGGTTAGGTGAAGGCGTAGAGGT | chr10: 45234654-45234759 | 106 |
| | ALOX5r | 374 | GACCGAATACCCCGCTTTCTCTCTCGAC | | |
| | ALOX5R_f | 375 | GAGGTCGAGAGAGAAAGCGGGGTATTCG | chr10: 45234729-45234838 | 110 |
| | ALOX5R_r | 376 | AACGCTCTCAACCCAACCCCTAAACTCA | | |
| ALX1 | ALX1f | 377 | AGGATAGTAGCGGTGAGTCGTTAGCGTT | chr12: 84198385-84198501 | 117 |
| | ALX1r | 378 | CGCTCCCACTTTTCTCCTTTCTCCCTCC | | |
| ALX4 | ALX4f | 379 | TTTTGATAAAGTGGGGAGGGCGTAGGGG | chr11: 44289270-44289375 | 106 |
| | ALX4r | 380 | ACACTCTCAAATACCCGTCGCGCTCTAT | | |
| C1orf230 | C1orf230f | 381 | TTTTGATAAAGTGGGGAGGGCGTAGGGG | chr1: 149960830-149960921 | 92 |
| | C1orf230r | 382 | ACACTCTCAAATACCCGTCGCGCTCTAT | | |
| | C1orf230R_f | 383 | AGCGTAGCGTAGTTGGAGTAGTTGCGAA | chr1: 149960685-149960805 | 121 |
| | C1orf230R_r | 384 | CGACGACTCTCTTCCCAATCTAAAACCCCA | | |
| C6orf186 | C6orf186f | 385 | CGGAGTTTAGAAGGGCGTTCGGTTACGG | chr6: 110785585-110785700 | 116 |
| | C6orf186r | 386 | CTCCACGAATCGCATCTTTCAATACCCA | | |
| C17orf64 | C17orf64f | 387 | AAAGGTGGTTCGAGTGAGGAAATTGCGG | chr17: 55853711-55853789 | 79 |
| | C17orf64r | 388 | GCGTCCCTAAACGACACACGACGAAATC | | |
| | C17orf64R_f | 389 | GTCGACGGCGGTTTTATCGTATTGTCGC | chr17: 55853578-55853689 | 112 |
| | C17orf64R_r | 390 | CCTTCTCCCGAACCTTCCTTCGTATCCT | | |
| C19orf41 | C19orf41f | 391 | TTAGAGGTATGGCGGGGTTTTTGTGACG | chr19: 55358254-55358348 | 95 |
| | C19orf41r | 392 | AATACTCCCTAAACCTCCTAACCGCGCC | | |
| CCDC67 | CCDC67f | 393 | GAGGTTTAATTGTTTCGTTGGTCGC | chr11: 92703424-92703546 | 123 |
| | CCDC67r | 394 | ACGCAAAACCGCGTATATCACCT | | |
| CCDC8 | CCDC8f | 395 | GGTTTTAGGGACGCGGTTGGAATTTGGG | chr19: 51608460-51608548 | 89 |
| | CCDC8r | 396 | CCCAACGCCTCGACCATATTAAATAACTT | | |
| CD38 | CD38f | 397 | GCGATTAAGGCGTATCGGTGGGTATTGC | chr4: 15389377-15389501 | 125 |
| | CD38r | 398 | AACACCACCCGACGAACTCTCGACTAAC | | |
| CD8A | CD8Af | 399 | TAGGACGTTGTTTGGTTCGAAGTTCGGG | chr2: 86871471-86871569 | 99 |
| | CD8Ar | 400 | CTCCGAACCGACCGAAAAACGCAACTTT | | |
| CDH23 | CDH23f | 401 | GGCGGGGTATTGTTTTGTTTC | chr10: 72826313-72826423 | 111 |
| | CDH23r | 402 | TCTACCGATATCATAACACCGACT | | |
| CDK5R2 | CDK5R2f | 403 | AAAGGTAGAGGGAAGGAGAGTTGTTTTT | chr2: 219532251-219532354 | 104 |
| | CDK5R2r | 404 | ACTCCTACCTCCTCCGAATCCTAAAACCT | | |
| CHST2 | CHST2f | 405 | CGGAATGAAGGTGTTTCGTAGGAAGGCG | chr3: 144322486-144322636 | 151 |
| | CHST2r | 406 | GCTACGACACCCAACGACCCATCGAAA | | |
| CLCN1 | CLCN1f | 407 | AATGATTTTGTTGGGTTCGGTGGAGCGG | chr7: 142752740-142752852 | 113 |
| | CLCN1r | 408 | CCGACAACTTCCGCGCCATCTCTTAAAC | | |
| | CLCN1R_f | 409 | TTGTGTTTTGAGCGTAGGTTGCGCGTAG | chr7: 142752798-142752874 | 77 |
| | CLCN1R_r | 410 | GCCTTCCCGTCGTAAAACAACTCCGACA | | |
| COL16Af | COL16A1f | 411 | GTTTTAGGGGGTTGGGGGTTTGTTAGGGA | chr1: 31942237-31942382 | 146 |
| | COL16A1r | 412 | AACCCGAAACGAAACTATACACCCCGCA | | |
| CPNE8 | CPNE8f | 413 | TCGATGTTCGTAGTGTTGTTGTAGCGGT | chr12: 37585569-37585689 | 121 |
| | CPNE8r | 414 | CCATCCCCGCCTAACGAAAACTAACCCT | | |
| DIO3 | DIO3f | 415 | CGTTTCGAGAAGAAGTTTCGCGGTTGGT | chr14: 101095917-101096005 | 89 |
| | DIO3r | 416 | ATCTAAACCCAAATCGAAAACCGCCGCC | | |
| DNM3 | DNM3f | 417 | TTGGAGTTGTCGTAGATCGTCGTGGTGG | chr1: 170077504-170077626 | 123 |
| | DNM3r | 418 | AAATCGCCCCACTACCGCATCCTTACTC | | |
| | DNM3R_f | 419 | GCGGTTAGGTGTGGTAAAGTAGTTGGCG | chr1: 170077283-170077405 | 123 |
| | DNM3R_r | 420 | GCGCACAACCAACCTATAAACTCCGACG | | |
| DUOX1 | DUOX1f | 421 | GGGATTTGTGAAGGCGGATTTG | chr15: 43209229-43209307 | 79 |
| | DUOX1r | 422 | AATATTCCGTCGATACCGAAAACCCGA | | |
| EMX1 | EMX1f | 423 | CGGTTGGAGCGCGTTTTCGAGAAGAAT | chr2: 73005041-73005163 | 123 |
| | EMX1r | 424 | AACGCAAAACAAACCGCGACCGAAAATA | | |
| EMX2OS | EMX2OSf | 425 | AGGAGAAGTCGTAGCGGGCGTC | chr10: 119291932-119292032 | 101 |
| | EMX2OSr | 426 | GACTAAACCTTCTACCGCCCACCG | | |
| ESPN | ESPNf | 427 | TAGTTGCGATGGGGTGGGAAGTTACGTT | chr1: 6430246-6430357 | 112 |
| | ESPNr | 428 | AAAACCATCGCCATCCACGAAAACGACA | | |
| EVX1 | EVX1f | 429 | AGGAGGATGATAGTTTAGAAAGAAGAGGGT | chr7: 27248900-27249019 | 120 |
| | EVX1r | 430 | CGCGACCGCGACGATAACGATAAAAACT | | |
| FABP5 | FABP5f | 431 | GAAACGTGTAGGCGTCGGCGTTTATGAG | chr8: 82355078-82355157 | 80 |
| | FABP5r | 432 | CGACCTCTCGAACGCCTCCTACAAACAA | | |
| FBRSL1 | FBRSL1f | 433 | GTGGAGGAGGAAGTTCGTTTC | chr12: 131575948-131576052 | 105 |
| | FBRSL1r | 434 | AACTACTACCAAACACGAAACGCA | | |
| FLI41350 | FLIf | 435 | GGTTAGAGTCGGTTGCGTAGTTT | chr10: 102979731-102979855 | 125 |
| | FLIr | 436 | TTTTTGTTAGGCGAAGTATAGAGAGCG | | |
| FOXG1 | FOXG1f | 437 | TTTTTCGATTGGTCGACGGCGAGAGAG | chr14: 28305617-28305740 | 124 |
| | FOXG1r | 438 | TTTCCGAACTACAAACGCACACTAAAAC | | |
| FOXL2 | FOXL2f | 439 | GATTCGTATGGGTTTTATCGAGTTTC | chr3: 140148670-140148764 | 95 |
| | FOXL2r | 440 | ACTTAAAAATAAACTCGCCCGTACG | | |
| FZD2 | FZD2f | 441 | TCGTTGGTGAAGGTGTAGTGTTCGTTCG | chr17: 39990814-39990938 | 125 |
| | FZD2r | 442 | TAACGCGCGCGCTCACAAATAAAACGAC | | |
| | FZD2R_f | 443 | TTTTTAGTGGTTCGAGCGTTTGCGTTGC | chr17: 39990969-39991059 | 91 |
| | FZD2R_r | 444 | TCCGTCCTCGAAATAATTCTAACCGACGC | | |
| HIF3A | HIF3Af | 445 | CGTGGTATAGTTAATCGCGCGGCGT | chr19: 51492066-51492190 | 125 |
| | HIF3Ar | 446 | TACAACCCCAACGCCATAACTCGCCAAT | | |
| HIVEP3 | HIVEP3f | 447 | TGTCGTCGTCGTCGGGGTTTTGTTATTT | chr1: 41901039-41901114 | 76 |
| | HIVEP3r | 448 | ACGACGATAAACTCCCGCTAAACCCGAA | | |
| | HIVEP3R_f | 449 | GAACGAGGATTTGCGTTTTTGGATCGC | chr1: 41901096-41901175 | 80 |
| | HIVEP3R_r | 450 | CCTAAACTCCTCTACATATTCCTCTACCT | | |
| HLA-F | HLA-Ff | 451 | GAATGGTTGCGATATGGGGTTCGACGG | chr6: 946778-946902 | 125 |
| | HLA-Fr | 452 | CCACGATATCCGCCGCGATCCAAAAAC | | |
| HOTAIR | HOTAIRf | 453 | TAAGGGTCGGTTGTTGTTTTTTTTC | chr12: 52645919-52646034 | 116 |
| | HOTAIRr | 454 | ACCGACGCCTTCCTTATAAAATACG | | |
| HOXA10 | HOXA10f | 455 | TGTGGGATAATTTGGCGAAGGGAGTAGA | chr7: 27180403-27180526 | 124 |
| | HOXA10r | 456 | AACTCGAAATTAACTACGAACGCCCGCC | | |
| HOXD11 | HOXD11f | 457 | GGCGGGGGTAGTTTTTGTATTAAGGCGA | chr2: 176680987-176681111 | 125 |
| | HOXD11r | 458 | CCTACGCTACTACTCTTCTCGACCCCCG | | |
| HOXD8 | HOXD8f | 459 | CGTTTCGTTCGTCGGTCGTAGCGATTG | chr2: 176702636-176702749 | 114 |
| | HOXD8r | 460 | CCGACGAAACATTTTCGCACCACAACAC | | |
| | HOXD8R_f | 461 | CGCGGTTTCGGGGTATACGGAGTTTTTG | chr2: 176702549-176702668 | 120 |
| | HOXD8R_r | 462 | GCAATTCAATCGCTACGACCGACGAACG | | |
| HSPA12B | HSPA12Bf | 463 | CGTCGTAGCGGGTACGGTTAACGAGTTG | chr20: 3661361-3661485 | 125 |
| | HSPA12Br | 464 | TTTCTCCACTCGAAACGCCCGACAACC | | |
| ISL1 | ISL1f | 465 | CGGGGGAGAACGGTTTGAGTTTCGAGTA | chr5: 50714776-50714885 | 110 |
| | ISL1r | 466 | TCATATTTCAACCTCGCCGCCGCTAAAC | | |
| Intergenic1 | Int1f | 467 | AGTAGGGATGGTCGTTCGTTGTTCGGTG | chr11: 68379573-68379679 | 107 |
| | Int1r | 468 | GACAAACGACCGAAAATACTCGCGCAAC | | |
| | Int1R_f | 469 | TTTTACGGTCGGGGCGATAGTTGAAGGT | chr11: 68379395-68379493 | 99 |
| | Int1R_r | 470 | TCACGCCAATACCCGCTAATCCCTCCTA | | |
| Intergenic2 | Int2f | 471 | GGGGATGGATAATTTTTAGGCGTTAAC | chr17: 69460223-69460339 | 117 |
| | Int2r | 472 | TAACCTCGTCTTTATCCCCGCG | | |
| Intergenic3 | Int3f | 473 | AGTGTGTAGTCGTTTGTGGGTGAGGAGTT | chr8: 95315865-95315994 | 130 |
| | Int3r | 474 | CACCGCGAAAAACGCCCACAATCTTACC | | |
| | Int3R_f | 475 | CGCGGGGGAGTTTATTTTTGAGGATTCGG | chr8: 95315775-95315892 | 118 |
| | Int3R_r | 476 | ACTCCTCACCCACAAACGACTACACACT | | |
| Intergenic4 | Int4f | 477 | TAGTATTTGTACGGAGTTTTTCGGCGGTC | chr5: 43054172-43054263 | 92 |
| | Int4r | 478 | TACGACGCAACCAACGATACTATCACCAA | | |
| Intergenic5 | Int5f | 479 | TAGTGATTGGTTATTTGGGCGCGGGGC | chr10: 43138416-43138530 | 115 |
| | Int5r | 480 | AAACGACATCCATCATCTCCCTCGACCC | | |
| Intergenic6 | Int6f | 481 | AGGTCGCGTTTTGGTCGTGC | chr3: 14827613-14827688 | 76 |
| | Int6r | 482 | ACTTAAAAATAAACTCGCCCGTACG | | |
| Intergenic7 | Int7f | 483 | ATTTTACGTAGGGTGGGGTTGAGGGCGT | chr12: 52897799-52897910 | 112 |
| | Int7r | 484 | ATCCTAACCGTCCCGCCTCAAAACCGTA | | |
| Intergenic8 | Int8f | 485 | CGTCGTAGTATTTGGCGGCGCGTTTC | chr2: 236737778-236737883 | 106 |
| | Int8r | 486 | AACGTACCTAATCCCCAAACCCACTCCT | | |
| Intergenic9 | Int9f | 487 | TCGTTGTGCGCGTTTCGTTTGTTGGATTA | chr6: 778755-778846 | 92 |
| | Int9r | 488 | TCGATAATATCTCCGTCGCCTCCGCAAA | | |
| Intergenic10 | Int10f | 489 | GCGCGTTTAATCGTGGGATTTTTGGGAG | chr2: 174899379-174899494 | 116 |
| | Int10r | 490 | CAAATTCGCGACACCCTACCCCAACAC | | |
| | Int10R_f | 491 | GGGTGTCGCGAATTTGGGGTA | chr2: 174899479-174899602 | 124 |
| | Int10R_r | 492 | CTAAACCTCTCCCCTCCCAAATTTACCT | | |
| Intergenic12 | Int12f | 493 | ATCGAGTTTTTAGCGGTTTTTGGGGCGG | chr1: 119344866-119344974 | 109 |
| | Int12r | 494 | ACTAACATCGCGCACTTAAATCTTTCCG | | |
| Intergenic13 | Int13f | 495 | GGTAGCGGCGGGTAAAAAGTC | chr7: 64675119-64675225 | 107 |
| | Int13r | 496 | TACAACTTTTTACCTCCGCCGC | | |
| Intergenic14 | Int14f | 497 | CGTCGATTTGCGGAATTTCGTCGTCGTT | chr1: 238227938-238228045 | 108 |
| | Int14r | 498 | ACATCCGCGTAAACTCGCCCTTTAACAC | | |
| | Int14R_f | 499 | TTTCGGGATTAGGGTTTCGGAGGGTGTC | chr1: 238227822-238227913 | 92 |
| | Int14R_r | 500 | CGTATCGATCCGTCCCTCCCGCTTAAAA | | |
| Intergenic15 | Int15f | 501 | CGGTTTTGGTGGTAGTTTTGGTAATC | chr19: 48895723-48895802 | 80 |
| | Int15r | 502 | AAAACCTCCCGAACGACGAAATAATCCA | | |
| | Int15R_f | 503 | GTAGGCGGTCGGAACGTGAAC | chr19: 48895536-48895660 | 125 |
| | Int15R_r | 504 | CGATAAAAACTACAATAACTCGACAACCA | | |
| Intergenic16 | Int16f | 505 | GTTGTGAGGGTTTTCGGCGGTATC | chr1: 54713046-54713165 | 120 |
| | Int16r | 506 | CATAACAACGCGCGACCCCTA | | |
| Intergenic17 | Int17f | 507 | TGATTATAAATTAGGGGGTTTGGTCGTCG | chr12: 61311832-61311945 | 114 |
| | Int17r | 508 | AAACCCTCCACCCTCGCAATACTACTCC | | |
| Intergenic18 | Int18f | 509 | TGTAGGAGATAATGGGAGTGAAGAGGGA | chr6: 4971256-4971338 | 83 |
| | Int18r | 510 | TTCCACGAAACGCGCGACTTCCTAACTA | | |
| | Int18R_f | 511 | GTTGAGTTAGGAGAGGTCGATAGC | chr6: 4971467-4971570 | 104 |
| | Int18R_r | 512 | CCCGAAAACAACGACTATCGAAATCCAA | | |
| Intergenic19 | Int19f | 513 | ATAAGGTTTGGTGGAAGCGTAGGAGCGT | chr6: 3177175-3177289 | 115 |
| | Int19r | 514 | ACGCCGAATAAAAATCCCGCAACCACAA | | |
| Intergenic20 | Int20f | 515 | GGAGGGGAGGAGATAGCGTTATTTAGGG | chr10: 118912740-118912842 | 103 |
| | Int20r | 516 | AAACAAAACCCGAAACCCCACCTACACC | | |
| Intergenic21 | Int21f | 517 | GCGTGGTAGTTGAGGATGTAGACGTGGT | chr16: 45381613-45381736 | 124 |
| | Int21r | 518 | TCCGAACTACTTAAAAATCCCCGCCGCC | | |
| Intergenic22 | Int22f | 519 | TCGTTGGTTGTGATTTTTATGCGGGCGT | chr8: 68037259-68037357 | 99 |
| | Int22r | 520 | ACCTCTCCGATAAACCAAATCCTCCGCC | | |
| | Int22R_f | 521 | CGGGTGAGGTTTGTGGTTAATTTCGCGT | chr8: 68037556-68037675 | 120 |
| | Int22R_r | 522 | CTCAACCAAACTACAACGTTCCCGCCTC | | |
| Intergenic23 | Int23f | 523 | AATGGAGGCGTAGATTAACGAGCGGTGT | chr5: 42987147-42987254 | 108 |
| | Int23r | 524 | ATCCTTAACAACCCCGCCGACTAACGTC | | |
| | Int23R_f | 525 | ACGGGTACGGAGAAACGTCGGATTTAGT | chr5: 42987852-42987946 | 95 |
| | Int23R_r | 526 | TCCCCGCGACACTCTACCTATAACGTCC | | |
| KCNH8 | KCNH8f | 527 | CGTTTGGCGGGTATTGTTGTTC | chr3: 19164879-19164971 | 93 |
| | KCNH8r | 528 | CCCGACGCAAACTCCCTCTC | | |
| KCNJ2 | KCNJ2f | 529 | GAAGTTGTTTTTTAGGGGTTTGCGC | chr17: 65676355-65676440 | 86 |
| | KCNJ2r | 530 | ACTCAAATCTACCCTCGCTTCAACG | | |
| KCKN4 | KCNK4f | 531 | GCGCGGGGGTATTTTGGAGGGTTAGTTA | chr11: 63816449-63816549 | 101 |
| | KCNK4r | 532 | TCCCTACTCGCCCGCTACGACTATAACA | | |
| KCNK17 | KCNK17f | 533 | CGGATTTTGTTTTCGGGAGTCGTTCGGG | chr6: 39390031-39390150 | 120 |
| | KCNK17r | 534 | AACTAAACGCCTAACCCTTCCCTCCCAC | | |
| KIAA1751 | KIAAf | 535 | TTCGTTTTGTTTTTCGGTTGGAGCGGGT | chr1: 1925171-1925288 | 118 |
| | KIAAr | 536 | TATAACCTAACCCTTCAACCGCGCCTCG | | |
| | KIAA1751R_f | 537 | AGGCGGCGGTTTTTGGCGATTGTTTTTC | chr1: 1925065-1925140 | 76 |
| | KIAA1751R_r | 538 | TTCCGTTACCATAAAACTACCCGCCCC | | |
| LASS1 | LASS1f | 539 | GATTTCGCGTATCGTCGTGTC | chr19: 18868171-18868273 | 103 |
| | LASS1r | 540 | TAATATCCCCCGTACCCCCCG | | |
| LOC255167 | LOCf | 541 | TTTCGATAATAGCGTTTTTGCGGCGTGG | chr5: 6636474-6636619 | 146 |
| | LOCr | 542 | CAAAAACACGCGACCTACGCCCTCCTAA | | |
| LRRC4 | LRRC4f | 543 | CGAGTCGGAGTGAGCGTTAAGTGAGGGG | chr7: 127459680-127459780 | 101 |
| | LRRC4r | 544 | CCTATCAACGACCACCCAACTACTCCCT | | |
| MIR155HG | MIR155HGf | 545 | TCGGGTTTAGCGTCGTTTGTAGTTTCGG | chr21: 25856335-25856430 | 96 |
| | MIR155HGr | 546 | AAAAACGTCTCCTTAATTCCCCGCGCTT | | |
| NEXN | NEXNf | 547 | GCGGTTGGAGTAGAAGTGTTAGCGGTTAGA | chr1: 78126913-78127036 | 124 |
| | NEXNr | 548 | TCACCCTACAAAAACCGATAACCGACGA | | |
| NKX2-1 | NKX2-1f | 549 | AGTTGGTTATAGGCGGCGAATTGGGTTT | chr14: 36057307-36057397 | 91 |
| | NKX2-1r | 550 | TCAACACCCCCTCTCCTAACCTCTCCAA | | |
| NKX6-2 | NXX6-2f | 551 | CGGGGAAGAGTTTCGGTTCGCGTTTTAG | chr10: 134449988-134450110 | 123 |
| | NXX6-2r | 552 | CCCTCCTATAACCCCGACCTACCCGAAA | | |
| | NKX6-2R_f | 553 | GCGCGGTAGGTGTTTTTCGGGTTGTAAA | chr10: 1344419796-134449892 | 97 |
| | NKX6-2R_r | 554 | ACCTTTACCTAACTACACTCCCATCCAA | | |
| NOTUM | NOTUMf | 555 | AGAGTAGGTCGTGGGGGATTC | chr17: 77512836-77512922 | 87 |
| | NOTUMr | 556 | CGCGCTAACCGCGATAAAAAC | | |
| NRN1 | NRN1f | 557 | AGGAGCGGGAGAGGGAAAAATAGTTAAG | chr6: 5952635-5952759 | 125 |
| | NRN1r | 558 | ACTACGCCCAAAACTCAACTACTAAAT | | |
| PLTP | PLTPf | 559 | TGGGAACGGGATAGGGACGCGTTTTAAT | chr20: 43974093-43974184 | 92 |
| | PLTPr | 560 | GAATCCCCTAAACTACCCGCCATCCCAC | | |
| | PLTPR_f | 561 | TGTACGCGTATTTTTGGAGGGTGGTTTGC | chr20: 43973871-43973950 | 80 |
| | PLTPR_r | 562 | CGATCTAATCGACCACCTCCTCTCCTCC | | |
| PRDM13 | PRDM13f | 563 | AAGTTTCGTCGAGTTGGGGTCGTTGGTT | chr6: 100168753-100168844 | 92 |
| | PRDM13r | 564 | GACCCTTCCCGACAACCATCTCGAACA | | |
| PRDM15 | PRDM15f | 565 | GAAAATTGCGCGGTTGGGTTAGTAGGGG | chr21: 42110148-42110259 | 112 |
| | PRDM15r | 566 | ACCTACAAATACCGTCCCCACCCGAAAC | | |
| PTGDR | TGDRf | 567 | AAGAGGGGTGTGATTCGCGAGTTTAGAT | chr14: 51804089-51804198 | 110 |
| | TGDRr | 568 | CCGCGCGCGACTCGAACGAAAAA | | |
| RECK | RECKf | 569 | AAGGGTGCGATGTTTTCGTTTAGGATCG | chr9: 36027398-36027485 | 88 |
| | RECKr | 570 | TAACTAACTAAAACCGCGATAAAACGACT | | |
| RTN4RL1 | RTN4f | 571 | TGGTAATCGCGTAGGTGTGTGATAGGGC | chr17: 1827825-1827931 | 107 |
| | RTN4r | 572 | AAAATACAAAATACGCCCCCGACCCCGA | | |
| | RTN4RL1R_f | 573 | TGAGGAGAGATTCGGAGTAGTTAGTAGA | chr17: 1827743-1827851 | 109 |
| | RTN4RL1R_r | 574 | CCCTATCACACACCTACGCGATTACCAA | | |
| SFRP5 | SFRP5f | 575 | TTTCGAAAAGTTGGTAGTCGGCGGTTGG | chr4: 154929548-154929670 | 123 |
| | SFRP5r | 576 | CATTCTACTCCCCCGAATCGAAACCCCC | | |
| | SFRP5R_f | 577 | AAGAGGAAGAGTTCGCGCGTCGAGTTTA | chr4: 154929355-154929454 | 100 |
| | SFRP5R_r | 578 | GAAATCGCGCGCCCACGATACTACAAAA | | |
| SHF | SHFf | 579 | TTATTAGTAGGCGGCGTCGGGGGTT | chr15: 43266978-43267127 | 150 |
| | SHFr | 580 | CGAAAACCCCTACTCCGAAAAATCGTCCG | | |
| | SHFR_f | 581 | GTTGAGATATCGAGGGGTTCGGGTTAGG | chr15: 43266838-43266959 | 122 |
| | SHFR_r | 582 | CGCCAACAACGATAAAATAAATACCGCGCC | | |
| SHOX2 | SHOX2f | 583 | CGTTTGTTCGATCGGGGTCGTACGAGTAT | chr3: 159304063-159304162 | 100 |
| | SHOX2r | 584 | TTTCCGCCTCCTACCTTCTAACCCGACT | | |
| SNCA | SNCAf | 585 | GGTTGGGGGAGTGGGAGGTAAATTCGTT | chr4: 90977105-90977221 | 117 |
| | SNCAr | 586 | CTAAACGCTCCCTCACGCCTTACCTTCA | | |
| SNX32 | SNX32f | 587 | TTGAGGGAAACGCGGTGGGAATCGTTTT | chr11: 65357939-65358057 | 119 |
| | SNX32r | 588 | CCGTAACTCGCCCGAAAAACTAACCGAA | | |
| SP9 | SP9f | 589 | TGATTGGTTGCGGGGTAGTTTC | chr2: 174907826-174907911 | 86 |
| | SP9r | 590 | ACACCCGCTTTAAAATACCGCTAA | | |
| STK33 | STK33f | 591 | GCGTTTCGGGTCGTTCGTTTTATTTCGC | chr11: 8572140-8572262 | 123 |
| | STK33r | 592 | CGACAACCTACGCCGAATATACGCACCT | | |
| SYNGR3 | SYNGR3f | 593 | GAAGGGATGAGGTTGAGGTTGGAGGTCG | chr16: 1981075-1981195 | 121 |
| | SYNGR3r | 594 | ACCTCCTACCCACCAATTCCGAAAAACAA | | |
| T | Tf | 595 | TTACGGAGTTTTAGGCGGCGTTAC | chr6: 166501979-166502099 | 121 |
| | Tr | 596 | CATTTCCCTCTCTACGCGCGAAC | | |
| THBS2 | THBS2f | 597 | CGTAGGTTTTGTTGGAGCGAGAGATCGG | chr6: 169395805-169395898 | 94 |
| | THBS2r | 598 | ACATATAAAACCGCGCTACCCGAAAACCG | | |
| TLX1NB | TLX1NBf | 599 | TGAAAGGGGAGAGGGGAATGTTATTGTT | chr10: 102871413-102871518 | 106 |
| | TLX1NBr | 600 | AATATTCTCGCAAACCCACCGCCAAACC | | |
| TMEM22 | TMEM22f | 601 | AAAGAGATTCGTGTTGCGGCGGATGAAG | chr3: 138021575-138021691 | 117 |
| | TMEM22r | 602 | GATCAACACTCGAACCCGAACTTTCCGC | | |
| TNFRSF10D | TNFRSf | 603 | AAGGGAGGAGGGTGGATCGAAAGCGTTA | chr8: 23077397-23077475 | 79 |
| | TNIFRSr | 604 | CGAAAACCTTTACACGCGCACAAACTACG | | |
| TXNRD1 | TXNDR1f | 605 | TATGGGTTGCGTCGAGGGTAAGGTAGTG | chr12: 103133710-103133788 | 79 |
| | TXNDR1r | 606 | ACCATCGCCGTTCTTACCTTTCGTCTACA | | |
| VSTM2B | VSTM2Bf | 607 | TTTTTAATTCGGTTCGGCGTTGATTTGT | chr19: 34711435-34711559 | 125 |
| | VSTM2Br | 608 | ACAACCGCGCGCTCCCGATAC | | |
| ZFPM2 | ZFPM2f | 609 | TAGCGCGGAAGTTGTGAGTTTAAGGCG | chr8: 106401146-106401241 | 96 |
| | ZFPM2r | 610 | TCCTCTAAACACCATCGAAACCCCCGAAC | | |
| ZNF280B | ZNF280Bf | 611 | AGTGGCGTTCGTTGAGATTAGGGAAGGG | chr22: 21192757-21192877 | 121 |
| | ZNF280Br | 612 | ACCGTACGCTACCGAAACGACCTTTACA | | |
| LOC105378683 | LOC105 Af | 613 | GTTTGTAATTGGTATGAGCGGC | chr1: 43023566-43023673 | 108 |
| | LOC105 Ar | 614 | ATAACGAAACGACGCCTC | | |
| | LOC105 Bf | 615 | GTAATTGGTATGAGCGGCGT | chr1: 43023570-43023660 | 91 |
| | LOC105 Br | 616 | GCCTCCGCGAAATAAAACCAT | | |
| | LOC105 Cf | 617 | AGTTAGAGTGGGTTAGGGGAT | chr1: 43023464-43023613 | 150 |
| | LOC105 Cr | 618 | ACGCGTAACACAAACACGAC | | |
| NPHS2 | NPHS2 Af | 619 | GGGGGATTTTAAAGATCGTC | chr1: 177811721-177811842 | 122 |
| | NPHS2 Ar | 620 | GACGAACGCAATCCACAA | | |
| | NPHS2 Bf | 621 | TGGTGGAGTTGTGGATTGCG | chr1: 177811817-177811891 | 75 |
| | NPHS2 Br | 622 | TCCCACCCAAACCTCTCTCT | | |
| NR5A2 | NR5A2 Af | 623 | GGTGCGTTTACGGGTTTC | chr1: 198278389-198278538 | 150 |
| | NR5A2 Ar | 624 | ACCTAATCCGATATTTCCCGA | | |
| | NR5A2 Bf | 625 | GGTAGGGTTTCGGTTGCGTA | chr1: 198278432-198278527 | 139 |
| | +NR5A2 Br | 626 | TATTTCCCGAAAACTCCACATCCA | | |
| | NR5A2 Br | 627 | TCCCGAAAACTCCACATCCA | | |
| PAX6 | PAX6 Af | 628 | ATTTGGATGTTTCGCGTTTC | | |
| | PAX6 Ar | 629 | TATCGCTACGACCCGACTAA | | |
| | +PAX6 Af | 630 | GTTAATTTGGATGTTTCGCGTTTC | chr11: 31783206-31783322 | 117 |
| | +PAX6 Ar | 631 | GTTTATCGCTACGACCCGACTAA | | |
| | PAX6 Bf | 632 | AGGGGAGTCGCGTTTTTAGG | chr11: 31782520-31782652 | 133 |
| | PAX6 Br | 633 | TCCCGACCGAAACCCAAATC | | |
| KCNE3 | KCNE3 Af | 634 | GAATAACGGCGTAAGTTTTTAC | chr11: 73855818-73855915 | 98 |
| | KCNE3 Ar | 635 | ATCCTCCCGAACGCAATA | | |
| | KCNE3 Bf | 636 | TTGTACGTTTGTGGGTGTGGA | chr11: 73855765-73855914 | 150 |
| | KCNE3 Br | 637 | TCCTCCCGAACGCAATAATCG | | |
| KCNA6 | KCNA6 Af | 638 | TTAACGGTTAGGTTAGATCGC | chr12: 4789322-4789421 | 100 |
| | KCNA6 Ar | 639 | CAATCTCTAAAACGCGACAC | | |
| | KCNA6 Bf | 640 | CGGGTGTCGCGTTTTAGAGAT | chr12: 4789399-4789482 | 84 |
| | KCNA6 Br | 641 | TTCTCCGATCTCATACCCCCT | | |
| TMEM132C | TMEM Af | 642 | GAGAAAAGTTGTTTCGGTC | | |
| | TMEM Ar | 643 | GCTACGTCTCTACTATCCGA | | |
| | +TMEM Af | 644 | CGGGAGAAAAGTTGTTTCGGTC | chr12: 127317663-127317786 | 124 |
| | +TMEM Ar | 645 | CCGCTACGTCTCTACTATCCGA | | |
| | TMEM Bf | 646 | TTCGGGGTGAGGGTAGTC | | |
| | TMEM Br | 647 | CCGACGCCCAACTAAAAA | | |
| | +TMEM Bf | 648 | GAGTTCGGGGTGAGGGTAGTC | chr12: 127318043-127318179 | 137 |
| | +TMEM Br | 649 | GAATCCCGACGCCCAACTAAAAA | | |
| | TMEM Cf | 650 | TTTTCGGGTTACGGGTCGTT | chr12: 127317330-127317424 | 95 |
| | TMEM Cr | 651 | ACGACTCCTCCGAAAATCCG | | |
| PDX1 | PDX1 Af | 652 | GTCGATTTTTGTTTTGAGC | chr13: 27390195-27390280 | 86 |
| | PDX1 Ar | 653 | TAAAAATAATCTACCGAATCGC | | |
| | PDX1 Bf | 654 | GGCGTTAGCGGGGATTTAGA | chr13: 27389563-27389694 | 132 |
| | PDX1 Br | 655 | CGCATCAAACGAAACCCTCC | | |
| | PDX1exp Af | 656 | CGGGAAGGTGTTCGTTTAATGGTTCGGT | chr13: 27389489-27389590 | 102 |
| | PDX1exp Ar | 657 | GTTTCCGCTCTAAATCCCCGCTAACGCC | | |
| | PDX1exp Bf | 658 | GGAAAAAGGAGGAGGATAAGAAGCGCGG | chr13: 27396588-27396685 | 98 |
| | PDX1exp Br | 659 | CTCGCCGAAAATCACGACGCAATCCTAC | | |
| EPSTI1 | EPSTI1 Af | 660 | TAGGGGAGGCGTCGAGTTC | chr13: 42464253-42464369 | 117 |
| | EPSTI1 Ar | 661 | ACTCGCTAAACGTCCCAACC | | |
| A2BP1 | A2BP1 Af | 662 | GAGTTTAGGGGTCGCGTC | chr16: 6009425-6009564 | 140 |
| | A2BP1 Ar | 663 | CAATACCGCCGCCTCTACTA | | |
| | A2BP1 Bf | 664 | GAGAGAGTAGGAGCGGATCG | chr16: 6009706-6009842 | 137 |
| | A2BP1 Br | 665 | ACAAATCAACCCCGCCCTAA | | |
| CRYM | CRYM Af | 666 | AGTGAGTGTTCGGGAGTTTC | | |
| | CRYM Ar | 667 | TCATTTATTAAAAACGCGCG | | |
| | +CRYM Af | 668 | GCAGTGAGTGCTCGGGAGCCCC | chr16: 21202786-21202934 | 149 |
| | +CRYM Ar | 669 | GGTTTTCATTTGTTAGAGGCGCGCG | | |
| | CRYM Bf | 670 | CGGGTTCGCGTAGGATTAGG | chr16: 21202650-21202732 | 83 |
| | CRYM Br | 671 | ACTCCTCATCCCAACACCCT | | |
| PRKCB | PRKCB Af | 672 | GTTCGTAGTTCGCGGTTTC | | |
| | PRKCB Ar | 673 | CGATACTCTCCTCGCCCT | | |
| | +PRKCB Af | 674 | TCGGTTCGTAGTTCGCGGTTTC | chr16: 23754928-23755052 | 125 |
| | +PRKCB Ar | 675 | GCACGATACTCTCCTCGCCCT | | |
| | PRKCB Bf | 676 | TTGGGCGAGTGATAGTTTC | chr16: 23754821-23754909 | 89 |
| | PRKCB Br | 677 | GACCGCTACTACACCCGA | | |
| | PRKCB Cf | 678 | CGGTAGAAGAACGTGTATGAGGT | chr16: 23755076-23755216 | 141 |
| | PRKCB Cr | 679 | GCTACCCTCGAAAACCCGAA | | |
| IRF8 | IRF8 Af | 680 | GATTTTTTTTAAGGTCGCGC | chr16: 84490230-84490341 | 112 |
| | +IRF8 Af | 681 | TTACGATTTTTTTTAAGGTCGCGC | | |
| | IRF8 Ar | 682 | ACTATACCTACCTACCGCCGTC | | |
| | IRF8 Bf | 683 | ATTTCGAAGAAGGCGGGTCG | chr16: 84490149-84490276 | 128 |
| | IRF8 Br | 684 | CTCCAAACGATACGCCAACG | | |
| SALL3 | SALL3 Af | 685 | TTTTGCGGGTAAGCGTTC | | |
| | SALL3 Ar | 686 | CCACAACTCTCTCGACGAC | | |
| | +SALL3 Af | 687 | TGTTTTTTGCGGGTAAGCGTTC | chr18: 74841456-74841551 | 96 |
| | +SALL3 Ar | 688 | GCCCACAACTCTCTCGACGAC | | |
| | SALL3 Bf | 689 | ATTTCGGGAAAGGGTGGGTC | chr18: 74840051-74840163 | 113 |
| | SALL3 Br | 690 | ACCCTAATCCCCCTTCACCA | | |
| | SALL3 Cf | 691 | TTTCGTTTCGTTTCGGTCGC | chr18: 74840452-74840573 | 122 |
| | SALL3 Cr | 692 | AACCCGCCCGAACTCAAATA | | |
| LYPD5 | LYPD5 Af | 693 | ATTAGGAGCGTACGTTTATTC | chr19: 49016646-49016788 | 143 |
| | LYPD5 Ar | 694 | TACGCACTCGAAACACAA | | |
| | LYPD5 Bf | 695 | CGGCGCGTTTTAAGGGTTTT | chr19: 49016738-49016863 | 126 |
| | LYPD5 Br | 696 | ATTACTCTCACCTCCGCACG | | |
| DPP10 | DPP10 Af | 697 | GATTGCGGGAAGAAGGTAC | | |
| | DPP10 Ar | 698 | AAACGAAACCAAACGACAA | | |
| | +DPP10 Af | 699 | CGGATTGCGGGAAGAAGGTAC | chr2: 115635638-115635739 | 102 |
| | +DPP10 Ar | 700 | GACGAAACGAAACCAAACGACAA | | |
| | DPP10 Bf | 701 | TTTTCGAGTTTGAAGCGTTC | | |
| | DPP10 Br | 702 | CGACTCTCACCTAATCCGC | | |
| | +DPP10 Bf | 703 | CGGTTTTCGAGTTTGAAGCGTTC | chr2: 115635947-115636088 | 142 |
| | +DPP10 Br | 704 | TACCGACTCTCACCTAATCCGC | | |
| | DPP10 Cf | 705 | TTACGACGGGGAGTTCGTTC | chr2: 115635821-115635943 | 123 |
| | +DPP10 Cr | 706 | CTTAACAACGTTCGCAAATCACGA | | |
| | DPP10 Cr | 707 | ACAACGTTCGCAAATCACGA | | |
| C20orf56 | C20orf Af | 708 | GTTCGTTATTTCGGAATTC | chr20: 22507658-22507804 | 147 |
| | C20orf Ar | 709 | CCGACCGATAAAATATAATTC | | |
| | C20orf Bf | 710 | GGGAGGGATTTAAGCGGGAG | chr20: 22507684-22507819 | 136 |
| | C20orf Br | 711 | CCCCCTTCACTAATCCCGAC | | |
| SOX2OT | SOX2OT Af | 712 | AGTGTTGAGAGTCGACGC | chr3: 182919951-182920042 | 92 |
| | SOX2OT Ar | 713 | AATAAAATAACCCGAACCGC | | |
| | SOX2OT Bf | 714 | GGGTTACGGTTTCGGGTTGT | chr3: 182919884-182919969 | 86 |
| | SOX2OT Br | 715 | CGCGTCGACTCTCAACACTA | | |
| CDKL2 | CDKL2 Af | 716 | GGTCGAGTCGAGTCGTTAC | | |
| | CDKL2 Ar | 717 | AAAACGCCTCCTAACGAA | | |
| | +CDKL2 Af | 718 | ATTGGTCGAGTCGAGTCGTTAC | chr4: 76774785-76774935 | 151 |
| | +CDKL2 Ar | 719 | ACAAAAAAACGCCTCCTAACGAA | | |
| | CDKL2 Bf | 720 | TATTTTTGGGCGAAGGCGTTG | chr4: 76774698-76774806 | 109 |
| | CDKL2 Br | 721 | GTAACGACTCGACTCGACCA | | |
| MARCH11 | MARCH11 Af | 722 | TCGGCGTTTTCGTTTTTC | chr5: 16232623-16232697 | 75 |
| | MARCH11 Ar | 723 | CGACGACACAACCATAAACTTT | | |
| | MARCH11 Bf | 724 | AAGGTTTTGTAGTTGCGGCG | chr5: 16232839-16232935 | 97 |
| | MARCH11 Br | 725 | TCTCACGCGCAACCGAAT | | |
| CCL28 | CCL28 Af | 726 | GTGGAGTTTTAGGTAGCGC | | |
| | CCL28 Ar | 727 | ACCCGCGATAAACTAAACC | | |
| | +CCL28 Af | 728 | AGGGTGGAGTTTTAGGTAGCGC | chr5: 43433001-43433128 | 128 |
| | +CCL28 Ar | 729 | AACAACCCGCGATAAACTAAACC | | |
| | CCL28 Bf | 730 | TGTAGTCGTGGTTGTCGTGG | chr5: 43432695-43432834 | 140 |
| | CCL28 Br | 731 | CCAAATAAACGACGTCCCGC | | |
| AP3B1 | AP3B1 Af | 732 | ATTTTATAGTCGCGTTAAAAGC | chr5: 77304383-77304519 | 137 |
| | AP3B1 Ar | 733 | ACTTTTATTACTCGCGATCC | | |
| | AP3B1 Bf | 734 | GGTAGGGTGAGTTTGGTCGG | chr5: 77304339-77304484 | 146 |
| | AP3B1 Br | 735 | CGCCGAACCACGTAAAAACT | | |
| CARD11 | CARD11 Af | 736 | ATTTGGGGCGTTTATGTTTC | chr7: 3049825-3049944 | 120 |
| | CARD11 Ar | 737 | CCCTCGAAAAACGACTCC | | |
| | CARD11 Bf | 738 | AGGGGTTGTAGGGTCGGG | | |
| | +CARD11 Bf | 739 | TTTAGGGGTTGTAGGGTCGGG | chr7: 3049955-3050087 | 133 |
| | CARD11 Br | 740 | ATTTTACATTTCCCTCCCCCGC | | |
| BLACE | BLACE Af | 741 | AGAATAAAAGTAGGCGGC | chr7: 154859246-154859384 | 139 |
| | BLACE Ar | 742 | TCTCGAAACCAAAATAAACG | | |
| | BLACE Bf | 743 | AGTAGGCGGCGGATTTGTAG | chr7: 154859254-154859357 | 104 |
| | BLACE Br | 744 | CCGAAAATACGCGAAATCAACC | | |
| PTPRN2 | PTPRN2 Af | 745 | GAGGAGATAAAGGTGTCGC | | |
| | PTPRN2 Ar | 746 | AACGTACCTAACCCGAAAAC | | |
| | +PTPRN2 Af | 747 | TCGGAGGAGATAAAGGTGTCGC | chr7: 157176188-157176342 | 155 |
| | +PTPRN2 Ar | 748 | CCAACGTACCTAACCCGAAAAC | | |
| | PTPRN2 Bf | 749 | GACGGTTTCGGTAGGGTC | | |
| | PTPRN2 Br | 750 | CCGAACCGAATATAAAACGA | | |
| | +PTPRN2 Bf | 751 | CGGACGGTTTCGGTAGGGTC | chr7: 157176379-157176463 | 85 |
| | +PTPRN2 Br | 752 | GCGCCGAACCGAATATAAAACGA | | |
| RUNX1T1 | RUNX1T1 Af | 753 | TTAGGTTCGTAAAGAGGGC | chr8: 93183286-93183401 | 116 |
| | RUNX1T1 Ar | 754 | TTAAAACCACGTCCGAATA | | |
| | RUNX1T1 Bf | 755 | TTTCGGGCGGGAGTTATAGG | chr8: 93183412-93183529 | 118 |
| | RUNX1T1 Br | 756 | ACGCGCTCTAAACTCAACCG | | |
| L1TD1 | L1TD1 Af | 757 | GCGCGTGGGGYFCGTAGCGTTTTAAG | chr1: 62433357-62433465 | 109 |
| | L1TD1 Ar | 758 | TTACCCGAAACACCCCGCGCCCTTC | | |
| PPFIA3 | PPFIA3 Af | 759 | AGATACGGAGATTTAGCGCGAGATCGGT | chr19: 54337953-54338094 | 143 |
| | PPFIA3 Ar | 760 | AAATTAACCGCCGAACACTCACAATACG | | |
| FILIP1L | FILIP1L Af | 761 | TTGTAGTGTCGCGTTGCGAGTCGATTGT | chr3: 101077651-101077753 | 103 |
| | FILIP1L Ar | 762 | ACAATAACGTAACGCCCATAAACCGAACG | | |
| NUDT16P | NUDT Af | 763 | GAGGACGGGTTGAATCGTGGTTTGTTGG | chr3: 132563775-132563858 | 84 |
| | NUDT Ar | 764 | ACTACGATAATCAAAACGCTCCACGCGA | | |
| TOP2P1 | TOP Af | 765 | GTGCGCGTTTTAGTAGGGCGAGAATGG | chr6: 28283268-28283417 | 150 |
| | TOP Ar | 766 | CGAAAACCAAATCCGAACCACCGTCTCC | | |
| | TOP Bf | 767 | TGATTTGGGTGGATGTAGAGGTTGTGGT | chr6: 28283447-28283568 | 122 |
| | TOP Br | 768 | TTTCGAATAACGCTACTCCGAACCGCGA | | |
| UNKWN1 | UNKWN1 Af | 769 | TTGAGAGTAGGGATTGTGGTGCGTCGTC | chr5: 72634694-72634838 | 145 |
| | UNKWN1 Ar | 770 | CTAACTCCCGAACGCTACATTCGCTCCA | | |
| GALR3 | GALR3 Af | 771 | GGTTGTGGTGAGTTTGGTTTACGGGCG | chr22: 36550907-36551049 | 143 |
| | GALR3 Ar | 772 | CGTAAAACGCGACCACCGCCAACATA | | |
| PRSS27 | PRSS Af | 773 | GGGAGGTTATTCGTAGGATTTGGCGCGG | chr16: 2705610-2705748 | 139 |
| | PRSS Ar | 774 | ATCCTAACGACTACGCACTACTTCCGCA | | |
| SLC7A4 | SLC Af | 775 | GAGTTCGTTTAGTTCGTCGGCGTC | chr22: 19716858-19717005 | 148 |
| | SLC Ar | 776 | AACCCCGATAAACTCCGATAACGACCT | | |
| LEF1 | LEF1 Af | 777 | AGAGTTGGGGGCGGTATAGTTAGGGTGT | chr4: 109307444-109307547 | 104 |
| | LEF1 Ar | 778 | TTCAATCCCTACGACCCCAACGCCTAAA | | |
| NFIC | NFIC Af | 779 | CGTGGATACGAGTTTTGGCGGCGATTAT | chr19: 3386117-3386219 | 103 |
| | NFIC Ar | 780 | GCCACCAACCCTACCTCCTTCCATATCC | | |
| | NFIC Bf | 781 | TTTTTCGGTTTGAGTTATCGTGGCGGGA | chr19: 3386234-3386379 | 146 |
| | NFIC Br | 782 | CGAACCGTACTTCCAACCAAACGCAACT | | |
| TMEM90B | TMEM90 Af | 783 | TAGGAAGGGGTCGATGTTGGTTTGGGTT | chr20: 24398648-24398747 | 100 |
| | TMEM90 Ar | 784 | TCTCACCAACTCCCATCGAATTCGCACA | | |
| | TMEM90 Bf | 785 | GTTTTGGTTTCGTTTCGGAGCGCGTAGA | chr20: 24398510-24398642 | 133 |
| | TMEM90 Br | 786 | TTTCTCTACCGACTCAACTCCCCCTCCC | | |
| UBD | UBD Af | 787 | TCGGTTGCGTAAATCGCGTTTTTGGTTG | chr6: 29629437-29629564 | 128 |
| | UBD Ar | 788 | TTCTCGATAATATCTCCGTCGCCTCCGC | | |
| GIPC2 | GIPC Af | 789 | GTTTAGGGGTGGAGGTCGGGGTTTTGA | chr1: 78284199-78284289 | 91 |
| | GIPC Ar | 790 | CCGAACCCCGCGCAAATAAAAACAACCT | | |
| EFNA4 | ERNA Af | 791 | GGGGCGCGTTTTTATGGAAAGTTAGGGT | chr1: 153310423-153310549 | 127 |
| ERNA4 | ERNA Ar | 792 | CTACGCCCTAAAACACGCCTCGACTTCT | | |
| | ERNA Bf | 793 | TGTGCGAAAGAGACGCGGGGTTTAGTTA | chr1: 153310139-153310288 | 150 |
| | ERNA Br | 794 | CCCGTAATCGCTAAAACATCCGCCCTTA | | |
| DRD4 | DRD4 Af | 795 | CGTCGGGCGATGTTGGTTTGTTCGTG | chr11: 627035-627175 | 141 |
| | DRD4 Ar | 796 | GCGACGCTCCACCGTAAACCCAATATTTA | | |
| TCTEX1D1 | TCTEX Af | 797 | CGGGGAGGGTCGAGGGTTTTGTTTGAG | chr1: 66990668-66990782 | 101 |
| | TCTEX Ar | 798 | GCGTCCCAAACTTCATTCAACCGACGAC | | |
| PHOX2B | PHOX Af | 799 | GCGGACGTAGTAATGGATTAAACGGGGA | chr4: 41447111-41447255 | 145 |
| | PHOX Ar | 800 | AAATCCGACTCCCTACACTCCCGACTTT | | |
| TSPAN33 | TSPAN Af | 801 | GGGGGTTGTGTTAGTTGTTTGTTTAGCGA | chr7: 128596487-128596593 | 107 |
| | TSPAN Ar | 802 | CGAAACTATTTCCCGCCAAACCGAACCC | | |
| CA9 | CA9 Af | 803 | TTTCGGGCGGGAGTATCGGGTTTTGTAG | chr9: 35666101-35666239 | 139 |
| | CA9 Ar | 804 | GCTCCTTTACCCCTTCTCGACCAACTCC | | |
| UNKWN2 | UNKWN2 Af | 805 | TTACGGATTTTATTTGTATTCGGAATCGTA | chr10: 102409232-102409335 | 104 |
| | UNKWN2 Ar | 806 | ACGCATCAAACTCGACACAAAATTTCATC | | |
| WT1 | WT1 Af | 807 | GGTGTTTTCGTAAGACGGGGTAGTGGGT | chr11: 32406776-32406869 | 94 |
| | WT1 Ar | 808 | TTCTCCTCCGCTAAAAATCCGAATACGA | | |
| OTX2 | OTX2 Af | 809 | AGGGATTGTATTTCGAGGTGGTCGAGGT | chr14: 56331673-56331781 | 109 |
| | OTX2 Ar | 810 | CCGACAAATCGAAACCTTCGCCCGAAAC | | |
| HOXB13 | HOXB13 Af | 811 | TCGCGGGTTATAAATATTTGGTTGCGGC | chr17: 44157793-44157885 | 93 |
| | HOXB13 Ar | 812 | GACCGCCACTACCTCGAAAACATTTCCC | | |
| BRCA1 | BRCA1 Af | 813 | GGTAACGGAAAAGCGCGGGAATTATAGA | chr17: 38530874-38530968 | 95 |
| | BRCA1 Ar | 814 | CCCACAACCTATCCCCCGTCCAAAAA | | |
| ITPRIPL1 | ITPRIPL1f | 815 | TTTTGTACGTTGGGTTACGGGGGTTTGG | chr2: 96354715-96354857 | 143 |
| | ITPRIPL1r | 816 | TAAACGCGATAAACCCCTACGACCCCCA | | |
| HES5 | HES5-F | 817 | TATCGGTTTTCGTAGTTGCGGGAGGAGG | Chr1: 2451323-2451386 | 118 |
| | HES5-R | 818 | CCGAATAAATACCAAACTCGCCCGACGC | | |
| CSRP1/ | CSRP1/ | 819 | CGGGTAGAGGGGAGGTAGGAATTGGAGA | Chr1: 199775889-199775914 | 80 |
| LOC376693 | LOC376693-F | | | | |
| | CSRP1/ | 820 | CCGAATAAACGTCACCCCTACACACCGC | | |
| | LOC376693-R | | | | |
| ALOX5 | ALOX5-F | 821 | TTTTGCGGTTAGGTGAAGGCGTAGAGGT | Chr10: 45234681-45234732 | 106 |
| | ALOX5-R | 822 | GACCGAATACCCCGCTTTCTCTCTCGAC | | |
| PPM1H/ | PPM1H/MON2-F | 823 | AGGAGTAGTATTGCGAGGGTGGAGGGT | Chr12: 61311943-61312001 | 112 |
| MON2 | PPM1H/MON2-R | 824 | TAAACCCGAAAAACAACGCCAATCCCGC | | |
| KIAA0984 | KIAA0984-F | 825 | GGGGATTTGTTGTAGAGTCGTAGGAGAA | Chr12: 63515983-63516043 | 62 |
| | KIAA0984-R | 826 | CCGCATCCCACCCTTTAAAACTCTA | | |
| TXNRD1 | TXNRD1-F | 827 | TATGGGTTGCGTCGAGGGTAAGGTAGTG | Chr12: 103133737-103133768 | 86 |
| | TXNRD1-R | 828 | TACGACGACCATCGCCGTTCTTACCTTT | | |
| CHST11 | CHST11-F | 829 | AAATTTGGATTGGGGGAGGGACGAGGTT | Chr12: 103376469-103376538 | 124 |
| | CHST11-R | 830 | CTTCGCAACCGAACTACTCACCCCCGAC | | |
| EFS | EFS-F | 831 | GGTCGTTGGAGTGGTCGTTTCGGTTTAG | Chr14: 22904743-22904785 | 98 |
| | EFS-R | 832 | CCTCAAACCCCCGAACGCGCTAAATAAA | | |
| ANXA2 | ANXA2-F | 833 | GTTCGGGGAGGGAGGGAGATTCGTTTTG | Chr15: 58478046-58478098 | 107 |
| | ANXA2-R | 834 | AACTCCCGACTTTAACCTCCCAACCCAA | | |
| RHCG | RHCG-F | 835 | GTTGTAGGGGTGTTTGGTCGGGTTGGTA | Chr15: 87840807-87840869 | 118 |
| | RHCG-R | 836 | ATCAACTACTCCGTACCCCACGTAACCG | | |
| RARA | RARA-F | 837 | AGTCGGGGTTGGTTGGTGGAAGAGG | Chr17: 35718896-35718981 | 137 |
| | RARA-R | 838 | CCCTCTCAACTCGATTCAAAATTCCCCC | | |
| PTRF | PTRF-F | 839 | AAAGTAATAAGTGGTTTCGGGCGGAGTC | Chr17: 37827277-37827326 | 104 |
| | PTRF-R | 840 | ACCCCGCATACCTACGAAAACGAAAACC | | |
| RND2 | RND2-F | 841 | CGGGATTATGGAGGGGTAGAGCGGTCG | Chr17: 38430910-38430955 | 99 |
| | RND2-R | 842 | ACGTCCTTAACGAACACCTACAACAACG | | |
| TMP4 | TMP4-F | 843 | AGGTTTTGTAGTAGTAGGCGGACGAGGC | Chr19: 16048446-16048512 | 121 |
| | TMP4-R | 844 | ACGAATACGAAACCCGAAACCGAAACGC | | |
| HIF3A | HIF3A-F | 845 | CGTGGTATAGTTAATCGCGCGGCGT | Chr19: 51492259-51492376 | 118 |
| | HIF3A-R | 846 | TACAACCCCAACGCCATAACTCGCCAAT | | |
| KLK5 | KLK4-F | 847 | TAGCGGGGATTTATTAGGGGAGAGGTGG | Chr19: 56107959-56108027 | 123 |
| | KLK4-R | 848 | ATCACCTACGAACACTATCCCTCACCCG | | |
| AMOTL2 | AMOTL2-F | 849 | GCGGAATAGTTCGCGGTTTTGGAATGTT | Chr3: 135565786-135565856 | 125 |
| | AMOTL2-R | 850 | AAACGTTTCCGCTCCCCGAAAAACGAAT | | |
| SCGB3A1 | SCGB3A1-F | 851 | GGAGATAGTTTTGAGAGGGGGAGGTCGC | Chr5: 179950858-179950923 | 120 |
| | SCGB3A1-R | 852 | CGCTACCTACGCCGATCGTAAATCCCAA | | |
| HLA-F | HLA-F-F | 853 | GAATGGTTGCGATATGGGGTTCGACGGA | Chr6: 29799978-29800035 | 112 |
| | HLA-F-R | 854 | CGCGATCCAAAAACGCAAATCCTCGTTC | | |
| HLA-J-1 | HLA-J, | 855 | GGTTTTGGTCGAGATTTGGGCGGGTGAG | Chr6: 30082430-30082476 | 101 |
| | NCRNA00171-1-F | | | | |
| | HLA-J, | 856 | CCCGAATCCTACGCCCCAACCAAATAAA | | |
| | NCRNA00171-1-R | | | | |
| HLA-J-3 | HLA-J, | 857 | TGAGTGATTTCGGTTCGGGGCGTAGATT | Chr6: 30083115-30083168 | 125 |
| | NCRNA00171-2-F | | | | |
| | HLA-J, | 858 | CGAAAATCTCTACAAATCCCGCAACCTCG | | |
| | NCRNA00171-2-E | | | | |
| PON3 | PON3-F | 859 | ATGGTTTCGGGGTGTTTAGCGGCGATTG | Chr7: 94863624-94863674 | 105 |
| | PON3-R | 860 | AACGAAACCGAACGAACCCCAATCCGTA | | |
| LRRC4/SND1 | LRRC4-F | 861 | GAGTCGGAGTGAGCGTTAAGTGAGGGG | Chr7: 127459707-127459730 | 77 |
| | LRRC4-R | 862 | TCCCTCCGACCGACCCAAAATAACTACG | | |
| PAH | PAH-F | 863 | TTCGTTGTTCGTTTTGGGTAAAGGGAAG | Chr12: 101835348-101835409 | 116 |
| | PAH-R | 864 | AAACTCGCTTCCCAAACTTCTAAAAATC | | |
| EPSTI1 | EPSTI1-F | 865 | GGGGAGGCGTCGAGTTCGGAGTTTATTA | Chr13: 42464282-42464345 | 117 |
| | EPSTI1-R | 866 | AAAACTCGCTAAACGTCCCAACCGCATC | | |
| ADCY4 | ADCY4-F | 867 | CGGGTATTGTTGGTTTAGGTTGTAGTAGGT | Chr14: 23873644-23873710 | 123 |
| | ADCY4-R | 868 | CGACCCTAACCAACCCCGAAACTCGAAA | | |
| HAPLN3 | HAPLN3-F | 869 | AGGGTAGAAAGGAAGCGGTAGTAGAAAA | Chr15: 87239811-87239872 | 116 |
| | HAPLN3-R | 870 | ACAACAACTCCTCCCTTCGAACCCAACC | | |
| HSF4 | HSF4-F | 871 | TGTGGGAGGGAAGGGAAATCGAGATTGG | Chr16: 65762053-65762164 | 113 |
| | HSF4-R | 872 | ACGACAAAACGAAACCCACAATCCTACCC | | |
| NBR1/ | NBR1/TMEM106A-F | 873 | ATTCGGATTGGTTAGTTTTTGCGGAAGT | Chr17: 38719260-38719296 | 91 |
| TMEM106A | NBR1/TMEM1061-R | 874 | TTCGCCACGCAACAACCTAAAACGCTAC | | |
| HAAO | HAAO-F | 875 | GGTTGCGGCGTTTATTTAGCGGGAAGTC | Chr2: 42873761-42873822 | 114 |
| | HAAO-R | 876 | CTCGCCGAACCCGCGACGAAATCTAC | | |
| RARB | RARB-F | 877 | TAGAGGAATTTAAAGTGTGGGTTGGGGG | Chr3: 25444371-25444441 | 125 |
| | RARB-R | 878 | ACCAACTTCTCTCCCTTTACGCCTTTTT | | |
| ALDH1L1 | ALDH1L1-F | 879 | TGGGTTAAGTATTTGTTATGTGTTACGGA | Chr3: 127382511-127382580 | 121 |
| | ALDH1L1-R | 880 | CGCTATCCACCCGAATACGCAACT | | |
| HIST1H3G | HIST1H3G-F | 881 | GCGCGGCGTTTTGTTATCGGTGGATT | Chr6: 26379588-26379647 | 60 |
| | HIST1H3G-R | 882 | TCTAAAATAACCCGCACCAAACAAACTACA | | |
| ZSCAN12 | ZSCAN12-F | 883 | TTATAAAGGTCGGAAGCGGTTACGGGGG | Chr6: 28475534-28475572 | 93 |
| | ZSCAN12-R | 884 | AACCCCTTTCGCTCCCTTCCTAAAACGA | | |
| HCG4P6 | HCG4P6-F | 885 | GTATGGTTGCGATTTGGGGTTGGAAGGG | Chr6: 30002983-30003042 | 114 |
| | HCG4P6-R | 886 | GCCGCGATCCAAAAACGCAAATCCTAAT | | |
| HLA-J-3 | HLA-J, | 887 | TAGGGAATGTTTGGTTGCGATTTGGGG | Chr6: 30083115-30083168 | 80 |
| | NCRNA00171-3-F | | | | |
| | HLA-J, | 888 | TCCTTACCGTCGTAAACATACTACTCAT | | |
| | NCRNA00171-3-R | | | | |
| EYA4 | EYA4-F | 889 | GCGTAAGTGCGAGGTTGTCGGTAGC | Chr6: 133604154-133604229 | 125 |
| | EYA4-R | 890 | TTTCCCGCAACTCTTTCCCCCTCTCT | | |
| HOXA7 | HOXA7-F | 891 | TGCGGTTAAAGAATTCGTTCGCGTTCGG | Chr7: 27162955-27162982 | 82 |
| | HOXA7-R | 892 | CTAAACGCTCCCGCGAAACCTCCAAATC | | |
| USP44 | USP44/p-F | 893 | TTCGGGTATTTTGAGGTTGTCGTCGGGA | Chr12: 94466379-94466481 | 103 |
| | USP44/p-R | 894 | GACGACGACGCGTCCGACGAATTTTA | | |
| CYP27A1 | CYP27A1/p-F | 895 | GTTTTGGTCGGGGCGTCGTGGATATTTT | Chr2: 219354932-219355042 | 111 |
| | CYP27A1/p-R | 896 | AAAAACCAACTAAACCCCTTCCCGCTCG | | |
| PRSS3 | PRSS3/p-F | 897 | GTGTGGAAAGGGTTTGGCGGTTGTTAGG | Chr9: 33740574-33740686 | 113 |
| | PRSS3/p-R | 898 | CTCGCCAAATACGTCCACCCAAAAACGA | | |
| C18orf62 | C18orf62/p-F | 899 | TAGGAGGGGACGTAGAGTTTACGGCGAA | Chr18: 71296729-71296833 | 105 |
| | C18orf62/p-R | 900 | GAATACCCGACCCGACCCATCCATCAC | | |
| SFRP2 | SFRP2/p-1-F | 901 | TGCGTTTGTAGGAGAAGTCGGGTTGGTT | Chr4: 154929326-154929408 | 83 |
| | SFRP2/p-1-R | 902 | ACTCTTCCTCGCCTCGCACTACTACCTA | | |
| | SFRP2/p-2-F | 903 | GTGCGATTCGGGGTTTCGAAAAGTTGGT | Chr4: 154929535-154929641 | 107 |
| | SFRP2/p-2-R | 904 | GAAACTACGCGCGAACTTACAACGCCTC | | |
| SLCO4C1 | SLCO4C1/p-F | 905 | GAGCGTAGAGCGTTGAGCGGGG | Chr5: 101660047-101660169 | 123 |
| | SLCO4C1/p-R | 906 | CGCCGCCGAATAACACGCCCAC | | |
| CORO1C | CORO1C/p-1-F | 907 | AGCGGGGATTTTCGGAGTTGGAGAGTTT | Chr12: 107686622-107686733 | 112 |
| | CORO1C/p-1-R | 908 | CTCCATCCGCCCGACCTAACCCTAAAAA | | |
| | CORO1C/p-2-F | 909 | GGGAAGTGGCGTAGTGGGCGTTTGTATC | Chr12: 107686752-107686848 | 97 |
| | CORO1C/p-2-R | 910 | TACCTCCAACGACCACGCCCACAAAATA | | |
| KJ904227 | KJ904227/p-F | 911 | TGGAGCGTTGAGTCGAAGTTTTGATTTT | Chr3: 127489474-127489582 | 109 |
| | KJ904227/p-R | 912 | TCTTACCCGAACTTTAACCCCAACCGCT | | |
| C6orf141 | C6orf141/ | 913 | GGTTGGGAGTTCGGAGTTGTAGTAGAGG | Chr6: 49626357-49626455 | 99 |
| | p-1-F | | | | |
| | C6orf141/ | 914 | CTTTAACCGATTCAAACAACAAACGCCT | | |
| | p-1-R | | | | |
| | C6orf141/ | 915 | GTAGGGCGCGGGGTTTCGTTAGTTTC | Chr6: 49626570-49626668 | 99 |
| | p-2-F | | | | |
| | C6orf141/ | 916 | ATCTACCGTTCTATCCTCGTAACCGCCG | | |
| | p-2-R | | | | |
| BC030768 | BC030768/p-F | 917 | TCGTTTGGGAGGGATCGTTTTTGGGAGA | Chr1: 26424688-26424767 | 80 |
| | BC030768/p-R | 918 | AACCCGAATACTATCCAACTACCGCCGC | | |
| DMRTA2 | DMRTA2/p-F | 919 | CGAGCGTGGGTATTAAGTCGGTAGTGGA | Chr1: 50657067-50657169 | 103 |
| | DMRTA2/p-R | 920 | GACCTCAACCCCCTACGCCTAACCTACT | | |
| HFE | HFE/p-1-F | 921 | GTAGATCGCGGTTTTGTAGGGGCGTTTG | Chr6: 26195692-26195783 | 92 |
| | HFE/p-1-R | 922 | CTAATTTCGATTTTTCCACCCCCGCCGC | | |
| | HFE/p-2-F | 923 | GAGTGTTTGTCGAGAAGGTTGAGTAAAT | Chr6: 26196140-26196221 | 82 |
| | HFE/p-2-R | 924 | CACCGCCCAACGCATTCGTTCTAAAATA | | |
| CADPS2 | CADPS2/p-F | 925 | ATAAAAGTGGGGTGGGTGGCGGAGGG | Chr7: 121744063-121744166 | 104 |
| | CADPS2/p-R | 926 | GCGCCGAAATAACAACCCAACCTACCAA | | |
| CYTH4 | CYTH4/p-F | 927 | TTTATCGGGGAAGTTTTCGAGGGTGGGC | Chr22: 36050993-36051112 | 120 |
| | CYTH4/p-R | 928 | TCCCAACTACCTCCTACGCACGAACGAT | | |
| Intergenic | Chr4/p-1-F | 929 | ATGAAATGTGGTTCGTGGAAGGTGTTTGT | Chr4: 186174475-186174549 | 75 |
| (Chr4) | Chr4/p-1-R | 930 | ACGACCCGAACGTTAATCCTCTTACTAC | | |
| NHLH2 | NHLH2/p-F | 931 | ACGTAGTTTTCGAGTTAGTGTCGTTAGAA | Chr1: 116172677-116172793 | 117 |
| | NHLH2/p-R | 932 | GACAAACGCCTCAAACCCGACCG | | |
| NRN1 | NRN1/p-F | 933 | AGGAGCGGGAGAGGGAAAAATAGTTAAG | Chr6: 5952635-5952767 | 133 |
| | NRN1/p-R | 934 | CGCTCCAAACTACGCCCAAAACTCAA | | |
| HMGCLL1 | HMGCLL1/p-F | 935 | ATTAGAGTTGTTTTGCGTATTGCGGCGG | Chr6: 55551934-55552030 | 97 |
| | HMGCLL1/p-R | 936 | CAAATACCCCGTACACCCGCTACCCCAA | | |
| Me3 | Me3/p-1-F | 937 | GGGAGTTGAGGTTTACGCGGTTTCGTTG | Chr11: 86061026-86061124 | 99 |
| | Me3/p-1-R | 938 | GACCGCCAACGCGATCCACCCATTAAC | | |
| | Me3/p-2-F | 939 | AGTTTTGGAAGTAGATTCGGTGCGGGTG | Chr11: 86060867-86060948 | 82 |
| | Me3/p-2-R | 940 | GCCGCGCAATCGCCTCTTTTTCAC | | |
| Intergenic | Chr3/p-1-F | 941 | AGACGATAGATGGCGGGTAGGAAGGGAG | Chr3: 135608250-135608374 | 125 |
| (Chr3) | Chr3/p-1-R | 942 | GCCGCCTACAACCGACGAACTACAAATC | | |
| Intergenic | Chr8/p-1-F | 943 | TCGCGGGTGAGGTTTGTGGTTAATTTCG | Chr8: 68037553-68037676 | 124 |
| (Chr8) | Chr8/p-1-R | 944 | GCTCAACCAAACTACAACGTTCCCGCCT | | |
| NBPF1 | NBPF1/p-F | 945 | TGAGAGGCGTATTTTGTTGGTTACGGTT | Chr1: 146219493-146219574 | 82 |
| | NBPF1/p-R | 946 | CGAAAACCATTCCGCTACCCTTCCAACT | | |
| Intergenic | Chr10/p-1-F | 947 | GGGGCGTTGGGTTATGGAGATTACGTTTT | Chr10: 42748953-42749053 | 101 |
| (Chr10) | Chr10/p-1-R | 948 | GTCCCGCGCTTAACGAATTCTACGAACG | | |
| ASAP1 | ASAP1/p-F | 949 | GTTCGGGTAGGGGTCGGGGGTC | Chr8: 131524437-131524546 | 110 |
| | ASAP1/p-R | 950 | CCCGAAACGACGTACTTAACGACCCGAA | | |
| Intergenic | Chr1/p-1-F | 951 | GGGAGGTTTGAGCGTCGAAGTTTTCGTT | Chr1: 119352428-119352549 | 122 |
| (Chr1) | Chr1/p-1-R | 952 | GCCCACTACCCCGCGAAACCTTATCAAC | | |
| PPP2R5C | PPP2R5C/p-F | 953 | AGTCGTTAGGTTGTTAAGGCGCGTTGTG | Chr14: 101317476-101317534 | 59 |
| | PPP2R5C/p-R | 954 | ACAAAAATAAAATCGAACCTAACCCCACG | | |
| Intergenic | Chr2/p-1-F | 955 | CGTATTAAGGGTTAAGCGGCGCGGT | Chr22: 44883312-44883404 | 93 |
| (Chr2) | Chr2/p-1-R | 956 | AACTTTCTCGAACGACTCGATAAACCTAA | | |
| KRT78 | KRT78/p-F | 957 | AGGTTTTGGGAATTTGGAAGTTCGCGGG | Chr12: 51554274-51554370 | 97 |
| | KRT78/p-R | 958 | AAAAACGCTCGAACCCAACCAATCGACG | | |
| LINC240 | LINC240/ | 959 | AAAGGAAGATCGTGGGTAGTTCGTGCG | Chr6: 27167780-27167859 | 80 |
| | p-1-F | | | | |
| | LINC240/ | 960 | ACTACAACTCACGTTTCCCCTCCAACAC | | |
| | p-1-R | | | | |
| | LINC240/ | 961 | AGGTTTATTTGACGTTTTAGGTCGATAGT | Chr6: 27172709-27172830 | 122 |
| | p-2-F | | | | |
| | LINC240/ | 962 | CGATCTCTCCCTTTCTTCCGCTTCCTAA | | |
| | p-2-R | | | | |
| Intergenic | Chr16/p-1-F | 963 | GGCGTCGGTTGCGGTTTTAGAT | Chr16: 53648145-53648269 | 125 |
| (Chr16) | Chr16/p-1-R | 964 | ACGCGAAAATCTACCTTTTAATTACGAACC | | |
| HIST1H3G/ | HIST1H3G/ | 965 | TCGTCGGTGGTCGGCGCGTTTTT | Chr6: 26379488-26379589 | 102 |
| 1H2BI | 1H2BI/p-F | | | | |
| | HIST1H3G/ | 966 | AACCCGCACCAAACAAACTACACGCAAA | | |
| | 1H2BI/p-R | | | | |
| PPM1H | PPM1H/p-1-F | 967 | GAATGGTAGCGAGAGGTTGCGGGTTAGG | Chr12: 61312222-61312310 | 89 |
| | PPM1H/p-1-R | 968 | CTCTACCCTCAAAATCGCGACGCAAACG | | |
| | PPM1H/p-2-F | 969 | AGGAGTAGTATTGCGAGGGTGGAGGGTT | Chr12: 61311917-61312012 | 96 |
| | PPM1H/p-2-R | 970 | CGCCAATCCCGCTCCGACACTATAACAA | | |
| TUBB2B | TUBB2B/p-F | 971 | ATAAGGTTTGGTGGAAGCGTAGGAGCGT | Chr12: 3177175-3177262 | 88 |
| | TUBB2B/p-R | 972 | ACGATATTCTAACCTCCGCCGCGAAACT | | |
| C2CD4A | C2C5F | 973 | GGTAGAGGGATAGGGAAGAGTTTGGCGT | Chr15: 60146378-60146528 | 150 |
| | C2C5R | 974 | ATTCAAAACGCGCGCGACGAAATTCAAC | | |
| COL19A1 | COL2F | 975 | GCGGAGTGGGAGGGTTATATTGGGAGAG | Chr6: 70633134-70633240 | 106 |
| | COL2R | 976 | CCGAACAAAACTACGACACCGCCGAAAA | | |
| DCDC2 | DCD5F | 977 | ACGACGGGTTGAGATAGGTGGTTGGATT | Chr6: 24465938-24466027 | 90 |
| | DCD5R | 978 | CCCGACGCGAAACAACGAACTAAAACGA | | |
| DHRS3 | DGR2F | 979 | TTTTTGTACGTTTTCGGGGTCGGAGGAG | Chr1: 12601840-12601942 | 102 |
| | DHR2R | 980 | AATCGCCGTCTAAACAAATCGCGAACTA | | |
| GALNT3 | GAL1F | 981 | CGGCGGTCGCGGTTTGTAGTTTAGAATTG | Chr2: 166358281-166358431 | 150 |
| | GAL1R | 982 | ACGCGCTTCCACTCCGACTAACAAATTA | | |
| | GAL3F | 983 | GGCGTCGTTCGGGTTAAGTTTGGTTGT | Chr2: 166359152-166359230 | 78 |
| | GAL3R | 984 | CACAACTTACGCGAAACAACAACCTCGC | | |
| HES5 | HES1F | 985 | TGGGTTGGTGTCGCGCGAATTTTTGTTT | Chr1: 2451234-2451350 | 116 |
| | HES1R | 986 | CCTCCTCCCGCAACTACGAAAACCGATA | | |
| | HES3F | 987 | GTTGGGGGTTATGTTTGGCGCGGAATAG | Chr1: 2451478-2451622 | 144 |
| | HES3R | 988 | CGCCTATATAAAACGTCGACGCGCGAAA | | |
| | HES4F | 989 | GTTCGGGCGTCGCGGTCGTTTTTATATT | Chr1: 2453144-2453266 | 122 |
| | HES4R | 990 | AAAACGCCCATTATACCCGCGCCAATTC | | |
| KILLIN | KIL5F | 991 | TAAGAATCGGCGGTAGTTAGTAGGCGGG | Chr10: 89611638-89611783 | 145 |
| | KIL5R | 992 | TCCTACGCCGCGACGAAAACAAAAACTC | | |
| | KIL6F | 993 | AGGTGGGGCGCGTTTATTAGTTTAGGGG | Chr10: 89611428-89611578 | 150 |
| | KIL6R | 994 | ACCTCTCCATCGCTAATACCCTACCGCT | | |
| MUC21 | MUC2F | 995 | GAGTGTTTCGAGGGTAGGAGGTTGTCGG | Chr6: 31031426-31031559 | 133 |
| | MUC2R | 996 | CAAAAACCGCCCGCAAAACGAAACCTAA | | |
| NR2E1/ | OST3F | 997 | ACGGATCGATCGCGGTTTTGGTAAGGAT | Chr6: 108542828-108542915 | 87 |
| OSTM1 | OST3R | 998 | CGCAAAAACGAAAAACTACGTACGCGCT | | |
| | OST4F | 999 | GTTGTTTGAGGACGGGTCGTTTAGCGG | Chr6: 108543090-108543189 | 99 |
| | OST4R | 1000 | ACCCCTATCCTACAACCCTACGAACGCA | | |
| PAMR1 | PAM4F | 1001 | TTTCGGGAGGTGTGGTTACGTTTGGAGA | Chr11: 35503958-35504077 | 119 |
| | PAM4R | 1002 | CCCCTCCTCCCAACACCCAACACTAAAA | | |
| SCRN1 | SCR2F | 1003 | GGTTGTGGTTTTTAAAAGGGAAAATTCGGG | Chr7: 29996282-29996388 | 106 |
| | SCR2R | 1004 | TAAACGCCGAAACCCGAACGTAACAACC | | |
| SEZ6 | SEZ3F | 1005 | AGGTGATTAGAAGGGAGAGGGGGAGGTT | Chr17: 24371083-24371180 | 97 |
| | SEZ3R | 1006 | TCATTATACACGACGCGCCCCTCCAAAT | | |
| | SEZ5F | 1007 | TACGTGGGTGTAGGTTAGGTCGGGTTGA | Chr17: 24371224-24371345 | 121 |
| | SEZ5R | 1008 | ACCACGCGACTACCGTATAAACAACCGAA | | |

### EQUIVALENTS

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

### REFERENCES

1. How K A, Nielsen H M, Tost J. DNA methylation based biomarkers: practical considerations and applications. Biochimie 2012; 94: 2314-37.
2. Mikeska T, Craig J M. DNA methylation biomarkers: cancer and beyond. Genes (Basel) 2014; 5: 821-64.
3. Noehammer C, Pulverer W, Hassler M R, Hofner M, Wielscher M, Vierlinger K, et al. Strategies for validation and testing of DNA methylation biomarkers. Epigenomics. 2014; 6: 603-22.
4. Warton K, Samimi G. Methylation of cell-free circulating DNA in the diagnosis of cancer. Front Mol. Biosci. 2015; 2: 13.
5. Wittenberger T, Sleigh S, Reisel D, Zikan M, Wahl B, Alunni-Fabbroni M, et al. DNA methylation markers for early detection of women's cancer: promise and challenges. Epigenomics. 2014; 6: 311-27.
6. Usadel H, Brabender J, Danenberg K D, Jeronimo C, Harden S, Engles J, et al. Quantitative adenomatous polyposis coli promoter methylation analysis in tumour tissue, serum, and plasma DNA of patients with lung cancer. Cancer Res. 2002; 62: 371-5.
7. Esteller M, Sanchez-Cespedes M, Rosell R, Sidransky D, Baylin S B, Herman J G. Detection of aberrant promoter hypermethylation of tumour suppressor genes in serum DNA from non-small cell lung cancer patients. Cancer Res. 1999; 59: 67-70.
8. Mazurek A, Pierzyna M, Giglok M, Dworzecka U, Suwinski R, Ma U E. Quantification of concentration and assessment of EGFR mutation in circulating DNA. Cancer Biomark. 2015; 15: 515-24.
9. Ostrow K L, Hoque M O, Loyo M, Brait M, Greenberg A, Siegfried J M, et al. Molecular analysis of plasma DNA for the early detection of lung cancer by quantitative methylation-specific PCR. Clin. Cancer Res. 2010; 16: 3463-72.
10. Powrozek T, Krawczyk P, Kucharczyk T, Milanowski J. Septin 9 promoter region methylation in free circulating DNA-potential role in noninvasive diagnosis of lung cancer: preliminary report. Med. Oncol. 2014; 31: 917.
11. Lin P C, Lin J K, Lin C H, Lin H H, Yang S H, Jiang J K, et al. Clinical Relevance of Plasma DNA Methylation in Colorectal Cancer Patients Identified by Using a Genome-Wide High-Resolution Array. Ann. Surg. Oncol. 2014.
12. Philipp A B, Nagel D, Stieber P, Lamerz R, Thalhammer I, Herbst A, et al. Circulating cell-free methylated DNA and lactate dehydrogenase release in colorectal cancer. BMC. Cancer 2014; 14: 245.
13. Chimonidou M, Strati A, Malamos N, Georgoulias V, Lianidou E S. SOX17 promoter methylation in circulating tumour cells and matched cell-free DNA isolated from plasma of patients with breast cancer. Clin. Chem. 2013; 59: 270-9.
14. Chimonidou M, Tzitzira A, Strati A, Sotiropoulou G, Sfikas C, Malamos N, et al. CST6 promoter methylation in circulating cell-free DNA of breast cancer patients. Clin. Biochem. 2013; 46: 235-40.
15. Martinez-Galan J, Torres-Torres B, Nunez M I, Lopez-Penalver J, Del M R, Ruiz De Almodovar J M, et al. ESR1 gene promoter region methylation in free circulating DNA and its correlation with estrogen receptor protein expression in tumour tissue in breast cancer patients. BMC. Cancer 2014; 14: 59.
16. Matuschek C, Bolke E, Lammering G, Gerber P A, Peiper M, Budach W, et al. Methylated APC and GSTP1 genes in serum DNA correlate with the presence of circulating blood tumour cells and are associated with a more aggressive and advanced breast cancer disease. Eur. J. Med. Res. 2010; 15: 277-86.
17. Fackler M J, Lopez B Z, Umbricht C, Teo W W, Cho S, Zhang Z, et al. Novel methylated biomarkers and a robust assay to detect circulating tumour DNA in metastatic breast cancer. Cancer Res. 2014; 74: 2160-70.
18. Avraham A, Uhlmann R, Shperber A, Birnbaum M, Sandbank J, Sella A, et al. Serum DNA methylation for monitoring response to neoadjuvant chemotherapy in breast cancer patients. Int. J. Cancer 2012; 131: E1166-E1172.
19. Sharma G, Mirza S, Parshad R, Gupta S D, Ralhan R. DNA methylation of circulating DNA: a marker for monitoring efficacy of neoadjuvant chemotherapy in breast cancer patients. Tumour. Biol. 2012; 33: 1837-43.
20. Legendre C, Gooden G C, Johnson K, Martinez R A, Liang W S, Salhia B. Whole-genome bisulfite sequencing of cell-free DNA identifies signature associated with metastatic breast cancer. Clin. Epigenetics. 2015; 7: 100.
21. Jones P A. Functions of DNA methylation: islands, start sites, gene bodies and beyond. Nat. Rev. Genet. 2012; 13: 484-92.
22. Cope L M, Fackler M J, Lopez-Bujanda Z, Wolff A C, Visvanathan K, Gray J W, et al. Do breast cancer cell lines provide a relevant model of the patient tumour methylome? PLOS. One. 2014; 9: e105545.
23. Becker D, Lutsik P, Ebert P, Bock C, Lengauer T, Walter J. BiQ Analyzer HiMod: an interactive software tool for high-throughput locus-specific analysis of 5-methylcytosine and its oxidized derivatives. Nucleic Acids Res. 2014; 42: W501-W507
24. Lutsik P, Feuerbach L, Arand J, Lengauer T, Walter J, Bock C. BiQ Analyzer HT: locus-specific analysis of DNA methylation by high-throughput bisulfite sequencing. Nucleic Acids Res. 2011; 39: W551-W556.
25. Soreide K. Receiver-operating characteristic curve analysis in diagnostic, prognostic and predictive biomarker research. J. Clin. Pathol. 2009; 62: 1-5.
26. Madic, J. et al. Pyrophosphorolysis-activated polymerization detects circulating tumor DNA in metastatic uveal melanoma. Clinical Cancer Research: an official journal of the American Association for Cancer Research. 2012; 18: 3934-3941.
27. Bidard, F. C. et al. Detection rate and prognostic value of circulating tumor cells and circulating tumor DNA in metastatic uveal melanoma. International Journal of Cancer 2014; 134: 1207-1213.
28 The Molecular Taxonomy of Primary Prostate Cancer. Cell. 2015; 163(4): 1011-25.

All references referred to herein are expressly incorporated by reference in their entireties.

1. A method of detecting methylated DNA in a tumour of a human subject having breast cancer, comprising:
extracting DNA from a cell-free sample;
amplifying multiple regions of the DNA with a plurality of PCR primer pairs;
wherein the multiple regions are selected from two or more regions of Clorf114, VWC2, ADCY4, MIR129-2, TTBK1, DMBX1, HOXA9, SFRP5, CHST11, FOXA3, TAL1, DMRTA2, PDE4B, BARHL2, TBX15, RUSCI, Clorf104, GNG4, POU3F3, BOLL, TRIM71, SLC2A2, CYTL1, SHISA3, GABRA4, EGFLAM, C5orf39, CDO1, IRF4, ID4, SCAND3, DDAH2, COL11A2, NT5E, SIM1, RGS17, CAPDS2, MSC, SPAG6, INA, FLI1, ATP5G2, USP44, POU4F1, HNF1B, LHX1, GALR1, MAST1, CPXMI, NEURL2, LOC105378683, NPHS2, NR5A2, PAX6, KCNE3, KCNA6, TMEM132C, PDX1, EPSTI1, A2BP1, CRYM, PRKCB, IRF8, SALL3, LYPD5, DPP10, C20orf56, SOX2OT, CDKL2, March 11, CCL28, AP3B1, CARD11, BLACE, PTPRN2, RUNX1T1, UBD, HINFIB, TMEM90B, TNFRSF10D, PPFIA3, PRSS27, TSPAN33, CD38, BRCA1, ITPRIPL1, CA9, HOXB13, GIPC2, FABP5, OTX2, ERNA4, PTGDR, Intergenic5, SLC7A4, EVX1, TOP2P1, LEF1, DRD4, ACVRL1, PRDM13, Intergenic 8, NFIC, TCTEX1D1, ALX1, and PHOX2B;
wherein individual primer pairs of the plurality of PCR primer pairs are modified to include non-native DNA sequences corresponding to methylation specific versions of the multiple regions by selecting C residues to be replaced with T residues according to their methylation status within individual regions of the multiple selected regions;
generating sequencing reads from each of the amplified regions;
aligning the sequencing reads with a reference sequence for each region, wherein the reference sequence is obtained from a cell-free sample obtained from a healthy human subject and the aligning is performed by a computer; and
detecting with a computer a level of methylation of CpG residues within the two or more selected regions in the DNA extracted from the cell-free sample.

2. The method according to claim 1, wherein the cell-free sample is a blood sample.

3. The method according to claim 2, wherein the breast cancer is triple negative breast cancer and the multiple regions are two or more regions of ALX1, ACVRL1, BRCA1, Clorf114, CA9, CARD11, CCL28, CD38, CDKL2, CHST11, CRYM, DMBX1, DPP10, DRD4, ERNA4, EPSTI1, EVX1, FABP5, FOXA3, GALR3, GIPC2, HINFIB, HOXA9, HOXB13, Intergenic5, Intergenic 8, IRF8, ITPRIPL1, LEF1, MAST1, BARHL2, BOLL, C5orf39, DDAH2, DMRTA2, GABRA4, ID4, IRF4, NT5E, SIM1, TBX15, NFIC, NPHS2, NR5A2, OTX2, PAX6, GNG4, SCAND3, TAL1, PDX1, PHOX2B, POU4F1, PFIA3, PRDM13, PRKCB, PRSS27, PTGDR, PTPRN2, SALL3, SLC7A4, SOX2OT, SPAG6, TCTEX1D1, TMEM132C, TMEM90B, TNFRSF10D, TOP2P1, TSPAN33, TTBK1, UDB, VWC2, and PPFIA3.

4. The method according to claim 2, wherein the breast cancer is BRCA1 breast cancer and the multiple regions are two or more regions of LOC105378683, NPHS2, NR5A2, PAX6, KCNE3, KCNA6, TMEM132C, PDX1, EPSTI1, A2BP1, CRYM, PRKCB, IRF8, SALL3, LYPD5, DPP10, C20orf56, SOX2OT, CDKL2, March 11, CCL28, AP3B1, CARD11, BLACE, PTPRN2, and RUNX1T1.

5. The method according to claim 2, wherein the breast cancer is triple negative breast cancer and the multiple regions are ALX1, ACVRL1, BRCA1, Clorf114, CA9, CARD11, CCL28, CD38, CDKL2, CHST11, CRYM, DMBX1, DPP10, DRD4, ERNA4, EPST11, EVX1, FABP5, FOXA3, GALR3, GIPC2, HINFIB, HOXA9, HOXB13, Intergenic5, Intergenic 8, IRF8, ITPRIPL1, LEF1, MAST1, BARHL2, BOLL, C5orf39, DDAH2, DMRTA2, GABRA4, ID4, IRF4, NT5E, SIM1, TBX15, NFIC, NPHS2, NR5A2, OTX2, PAX6, GNG4, SCAND3, TAL1, PDX1, PHOX2B, POU4F1, PFIA3, PRDM13, PRKCB, PRSS27, PTGDR, PTPRN2, SALL3, SLC7A4, SOX2OT, SPAG6, TCTEX1D1, TMEM132C, TMEM90B, TNFRSF10D, TOP2P1, TSPAN33, TTBK1, UDB, VWC2, and PPFIA3.

6. The method according to claim 2, wherein the breast cancer is BRCA1 breast cancer and the multiple regions are LOC105378683, NPHS2, NR5A2, PAX6, KCNE3, KCNA6, TMEM132C, PDX1, EPSTI1, A2BP1, CRYM, PRKCB, IRF8, SALL3, LYPD5, DPP10, C20orf56, SOX2OT, CDKL2, March 11, CCL28, AP3B1, CARD11, BLACE, PTPRN2, and RUNXIT1.

7. The method according to claim 1, wherein the plurality of PCR primer pairs are selected from the group consisting of SEQ ID NOs: 1-364, 377-378, 397-398, 429-432, 479-480, 485-486, 563-564, 567-568, 603-604, 613-758, 765-768, 773-804, and 809-816.

8. The method according to claim 3, wherein the plurality of PCR primer pairs are selected from the group consisting of SEQ ID NOs: 1-37, 68-99, 121-179, 184-194, 199-202, 207-212, 233-242, 248-253, 258-282, 287-302, 317-318, 329-332, 335-344, 349-354, 363-364, 397-398, 429-432, 479-480, 485-486, 563-564, 567-568, 603-604, 619-633, 642-661, 666-692, 697-707, 712-721, 726-731, 736-740, 745-752, 757-758, 765-768, 771-804, and 809-816.

9. The method according to claim 4, wherein the plurality of PCR primer pairs are selected from the group consisting of SEQ ID NOs: 613-756.

10. The method according to claim 5, wherein the plurality of PCR primer pairs are SEQ ID NOs: 1-37, 68-99, 121-179, 184-194, 199-202, 207-212, 233-242, 248-253, 258-282, 287-302, 317-318, 329-332, 335-344, 349-354, 363-364, 377-378, 397-398, 429-432, 479-480, 485-486, 563-564, 567-568, 603-604, 619-633, 642-661, 666-692, 697-707, 712-721, 726-731, 736-740, 745-752, 757-758, 765-768, 771-804, and 809-816.

11. The method according to claim 6, wherein the plurality of PCR primer pairs are SEQ ID NOs: 613-756.

12. A method of detecting breast cancer, comprising:
extracting DNA from a cell-free sample obtained from a human subject;
amplifying multiple regions of the DNA with a plurality of PCR primer pairs;
wherein the multiple regions are selected from two or more regions of Clorf114, VWC2, ADCY4, MIR129-2, TTBK1, DMBX1, HOXA9, SFRP5, CHST11, FOXA3, TAL1, DMRTA2, PDE4B, BARHL2, TBX15, RUSCI, Clorf104, GNG4, POU3F3, BOLL, TRIM71, SLC2A2, CYTL1, SHISA3, GABRA4, EGFLAM, C5orf39, CDO1, IRF4, ID4, SCAND3, DDAH2, COL11A2, NT5E, SIM1, RGS17, CAPDS2, MSC, SPAG6, INA, FLI1, ATP5G2, USP44, POU4F1, HNFIB, LHX1, GALR1, MAST1, CPXMI, NEURL2, LOC105378683, NPHS2, NR5A2, PAX6, KCNE3, KCNA6, TMEM132C, PDX1, EPSTI1, A2BP1, CRYM, PRKCB, IRF8, SALL3, LYPD5, DPP10, C20orf56, SOX2OT, CDKL2, March 11, CCL28, AP3B1, CARD11, BLACE, PTPRN2, RUNXIT1, UBD, HINFIB, TMEM90B, TNFRSF10D, PPFIA3, PRSS27, TSPAN33, CD38, BRCA1, ITPRIPL1, CA9, HOXB13, GIPC2, FABP5, OTX2, ERNA4, PTGDR, Intergenic5, SLC7A4, EVX1, TOP2P1, LEF1, DRD4, ACVRL1, PRDM13, Intergenic 8, NFIC, TCTEX1D1, ALX1, and PHOX2B;
wherein individual primer pairs of the plurality of PCR primer pairs are modified to include non-native DNA sequences corresponding to methylation specific versions of the multiple regions by selecting C residues to be replaced with T residues according to their methylation status within individual regions of the multiple selected regions;
generating sequencing reads from each of the amplified regions;
aligning the sequencing reads with a reference sequence for each region, wherein the reference sequence is obtained from a cell-free sample obtained from a healthy human subject and the aligning is performed by a computer; and
detecting at least two adjacent CpG residues that are methylated in each of the two or more selected regions in the DNA extracted from the cell-free sample;
determining that a level of methylation within the two or more selected regions is greater than a level of methylation within the two or more selected regions in subjects without breast cancer, which is indicative of the human subject having breast cancer.

13. The method according to claim 12, wherein the cell-free sample is a blood sample.

14. The method according to claim 13, wherein the breast cancer is triple negative breast cancer and the multiple regions are two or more regions of ALX1, ACVRL1, BRCA1, Clorf114, CA9, CARD11, CCL28, CD38, CDKL2, CHST11, CRYM, DMBX1, DPP10, DRD4, ERNA4, EPSTI1, EVX1, FABP5, FOXA3, GALR3, GIPC2, HINFIB, HOXA9, HOXB13, Intergenic5, Intergenic 8, IRF8, ITPRIPL1, LEF1, MAST1, BARHL2, BOLL, C5orf39, DDAH2, DMRTA2, GABRA4, ID4, IRF4, NT5E, SIM1, TBX15, NFIC, NPHS2, NR5A2, OTX2, PAX6, GNG4, SCAND3, TAL1, PDX1, PHOX2B, POU4F1, PFIA3, PRDM13, PRKCB, PRSS27, PTGDR, PTPRN2, SALL3, SLC7A4, SOX2OT, SPAG6, TCTEX1D1, TMEM132C, TMEM90B, TNFRSF10D, TOP2P1, TSPAN33, TTBK1, UDB, VWC2, and PPFIA3.

15. The method according to claim 13, wherein the breast cancer is BRCA1 breast cancer and the multiple regions are two or more regions of LOC105378683, NPHS2, NR5A2, PAX6, KCNE3, KCNA6, TMEM132C, PDX1, EPSTI1, A2BP1, CRYM, PRKCB, IRF8, SALL3, LYPD5, DPP10, C20orf56, SOX2OT, CDKL2, March 11, CCL28, AP3B1, CARD11, BLACE, PTPRN2, and RUNX1T1.

16. The method according to claim 13, wherein the breast cancer is triple negative breast cancer and the multiple regions are ALX1, ACVRL1, BRCA1, Clorf114, CA9, CARD11, CCL28, CD38, CDKL2, CHST11, CRYM, DMBX1, DPP10, DRD4, ERNA4, EPSTI1, EVX1, FABP5, FOXA3, GALR3, GIPC2, HINFIB, HOXA9, HOXB13, Intergenic5, Intergenic 8, IRF8, ITPRIPL1, LEF1, MAST1, BARHL2, BOLL, C5orf39, DDAH2, DMRTA2, GABRA4, ID4, IRF4, NT5E, SIM1, TBX15, NFIC, NPHS2, NR5A2, OTX2, PAX6, GNG4, SCAND3, TAL1, PDX1, PHOX2B, POU4F1, PFIA3, PRDM13, PRKCB, PRSS27, PTGDR, PTPRN2, SALL3, SLC7A4, SOX2OT, SPAG6, TCTEX1D1, TMEM132C, TMEM90B, TNFRSF10D, TOP2P1, TSPAN33, TTBK1, UDB, VWC2, and PPFIA3.

17. The method according to claim 13, wherein the breast cancer is BRCA1 breast cancer and the multiple regions are LOC105378683, NPHS2, NR5A2, PAX6, KCNE3, KCNA6, TMEM132C, PDX1, EPSTI1, A2BP1, CRYM, PRKCB, IRF8, SALL3, LYPD5, DPP10, C20orf56, SOX2OT, CDKL2, March 11, CCL28, AP3B1, CARD11, BLACE, PTPRN2, and RUNXIT1.

18. The method according to claim 12, wherein the plurality of PCR primer pairs are selected from the group consisting of SEQ ID NOs: 1-364, 377-378, 397-398, 429-432, 479-480, 485-486, 563-564, 567-568, 603-604, 613-758, 765-768, 773-804, and 809-816.

19. The method according to claim 14, wherein the plurality of PCR primer pairs are selected from the group consisting of SEQ ID NOs: 1-37, 68-99, 121-179, 184-194, 199-202, 207-212, 233-242, 248-253, 258-282, 287-302, 317-318, 329-332, 335-344, 349-354, 363-364, 397-398, 429-432, 479-480, 485-486, 563-564, 567-568, 603-604, 619-633, 642-661, 666-692, 697-707, 712-721, 726-731, 736-740, 745-752, 757-758, 765-768, 771-804, and 809-816.

20. A kit for detecting breast cancer in tumour DNA extracted from a cell-free sample obtained from a human subject, comprising:
a plurality of PCR primers, wherein the PCR primers are primer pairs for amplifying two or more regions selected from Clorf114, VWC2, ADCY4, MIR129-2, TTBK1, DMBX1, HOXA9, SFRP5, CHST11, FOXA3, TAL1, DMRTA2, PDE4B, BARHL2, TBX15, RUSCI, Clorf104, GNG4, POU3F3, BOLL, TRIM71, SLC2A2, CYTL1, SHISA3, GABRA4, EGFLAM, C5orf39, CDO1, IRF4, ID4, SCAND3, DDAH2, COL11A2, NT5E, SIM1, RGS17, CAPDS2, MSC, SPAG6, INA, FLI1, ATP5G2, USP44, POU4F1, HNFIB, LHX1, GALR1, MAST1, CPXMI, NEURL2, LOC105378683, NPHS2, NR5A2, PAX6, KCNE3, KCNA6, TMEM132C, PDX1, EPSTI1, A2BP1, CRYM, PRKCB, IRF8, SALL3, LYPD5, DPP10, C20orf56, SOX2OT, CDKL2, March 11, CCL28, AP3B1, CARD11, BLACE, PTPRN2, RUNXIT1, UBD, HINF1B, TMEM90B, TNFRSF10D, PPFIA3, PRSS27, TSPAN33, CD38, BRCA1, ITPRIPL1, CA9, HOXB13, GIPC2, FABP5, OTX2, ERNA4, PTGDR, Intergenic5, SLC7A4, EVX1, TOP2P1, LEF1, DRD4, ACVRL1, PRDM13, Intergenic 8, NFIC, TCTEX1D1, ALX1, and PHOX2B;
wherein the primers are modified to include non-native DNA sequences corresponding to methylation specific versions of the multiple regions by selecting C residues to be replaced with T residues according to their methylation status such that the primers anneal to a methylated sequence.
